(19) United States
(12) Patent Application Publication
(10) Pub. No.: US 2014/0072613 A1
(43) Pub. Date: Mar. 13, 2014

(54) Compositions and Methods for Treating Cutaneous Scarring

(71) Applicants: Cynthia Lander, Mendham, NJ (US); Caryn Peterson, Encinitas, CA (US); Colleen Brophy, Nashville, TN (US)

(72) Inventors: Cynthia Lander, Mendham, NJ (US); Caryn Peterson, Encinitas, CA (US); Colleen Brophy, Nashville, TN (US)

(21) Appl. No.: 13/829,876

(22) Filed: Mar. 14, 2013

Related U.S. Application Data
(60) Provisional application No. 61/699,160, filed on Sep. 10, 2012.

(51) Int. Cl. A61K 38/17

(52) U.S. Cl. CPC A61K 38/1709
USPC 424/446; 514/18.6; 514/8.9; 514/17.2

(57) ABSTRACT

The described invention provides compositions, dressings and methods for treating a cutaneous scar in a subject. The compositions of the derived invention contains a pharmaceutical composition comprising a therapeutic amount of a Mitogen-Activated Protein Kinase-Activated Protein Kinase 2 (MK2) inhibitor comprising an MK2 polypeptide inhibitor or a functional equivalent thereof, and a pharmaceutically acceptable carrier.

### CROSS-REFERENCE TO RELATED APPLICATIONS

This application claims priority from U.S. provisional patent application Ser. No. 61/699,160, filed Sep. 10, 2012, the entire disclosure of which is incorporated herein by reference.

### FIELD OF INVENTION

The described invention relates to the fields of cell and molecular biology, polypeptides, and therapeutic methods of use.

### BACKGROUND

### 1. Kinases

Kinases are a ubiquitous group of enzymes that catalyze the phosphoryl transfer reaction from a phosphate donor (usually adenosine-5′-triphosphate (ATP)) to a receptor substrate. Although all kinases catalyze essentially the same phosphoryl transfer reaction, they display remarkable diversity in their substrate specificity, structure, and the pathways in which they participate. A recent classification of all available kinase sequences (approximately 60,000 sequences) indicates kinases can be grouped into 25 families of homologous (meaning derived from a common ancestor) proteins. These kinase families are assembled into 12 fold groups based on similarity of structural fold. Further, 22 of the 25 families (approximately 98.8% of all sequences) belong to 10 fold groups for which the structural fold is known. Of the other 3 families, polyphosphate kinase forms a distinct fold group, and the 2 remaining families are both integral membrane kinases and comprise the final fold group. These fold groups not only include some of the most widely spread protein folds, such as Rossmann-like fold (three or more parallel β strands linked by two α helices in the topological order β-α-β-α-β), ferredoxin-like fold (a common α+β protein fold with a signature βαββαβ secondary structure along its backbone), TIM-barrel fold (meaning a conserved protein fold consisting of eight α-helices and eight parallel β-strands that alternate along the peptide backbone), and antiparallel β-barrel fold (a beta barrel is a large beta-sheet that twists and coils to form a closed structure in which the first strand is hydrogen bonded to the last), but also all major classes (all α, all β, α+β, α/β) of protein structures. Within a fold group, the core of the nucleotide-binding domain of each family has the same architecture, and the topology of the protein core is either identical or related by circular permutation. Homology between the families within a fold group is not implied.

Group I (23,124 sequences) kinases incorporate protein S/T-Y kinase, atypical protein kinase, lipid kinase, and ATP grasp enzymes and further comprise the protein S/T-Y kinase, and atypical protein kinase family (22,074 sequences). These kinases include: choline kinase (EC 2.7.1.32); protein kinase (EC 2.7.137); phosphorylase kinase (EC 2.7.1.38); homoserine kinase (EC 2.7.1.39); I-phosphatidylinositol 4-kinase (EC 2.7.1.67); streptomycin 6-kinase (EC 2.7.1.72); ethanolamine kinase (EC 2.7.1.82); streptomycin 3′-kinase (EC 2.7.1.87); kanamycin kinase (EC 2.7.1.95); 5-methylthioribose kinase (EC 2.7.1.100); viomycin kinase (EC 2.7.1.103); [hydroxymethylglutaryl-CoA reductase (NADPH2)] kinase (EC 2.7.1.109); protein-tyrosine kinase (EC 2.7.1.112); [isocitrate dehydrogenase (NADP+)] kinase (EC 2.7.1.116); [myosin light-chain] kinase (EC 2.7.1.117); hygromycin-B kinase (EC 2.7.1.119); calcium/calmodulin-dependent protein kinase (EC 2.7.1.123); rhodopsin kinase (EC 2.7.1.125); [beta-adrenergic-receptor] kinase (EC 2.7.1.126); [myosin heavy-chain] kinase (EC 2.7.1.129); [Tau protein] kinase (EC 2.7.1.135); macrolide 2′-kinase (EC 2.7.1.136); 1-phosphatidylinositol 3-kinase (EC 2.7.1.137); [RNA-polymerase]-subunit kinase (EC 2.7.1.141); phosphatidylinositol-4,5-bisphosphate 3-kinase (EC 2.7.1.153); and phosphatidylinositol-4-phosphate 3-kinase (EC 2.7.1.154). Group I further comprises the lipid kinase family (321 sequences). These kinases include: I-phosphatidylinositol-4-phosphate 5-kinase (EC 2.7.1.68); I D-myo-inositol-triphosphate 3-kinase (EC 2.7.1.127); inositol-tetrakisphosphate 5-kinase (EC 2.7.1.140); I-phosphatidylinositol-5-phosphate 4-kinase (EC 2.7.1.149); 1-phosphatidylinositol-3-phosphate 5-kinase (EC 2.7.1.150); inositol-polyphosphate multikinase (EC 2.7.1.151); and inositol-hexakiphosphate kinase (EC 2.7.4.21). Group I further comprises the ATP-grasp kinases (729 sequences) which include inositol-tetrakisphosphate I-kinase (EC 2.7.1.134); pyruvate, phosphate dikinase (EC 2.7.9.1); and pyruvate, water dikinase (EC 2.7.9.2).

Group II (17,071 sequences) kinases incorporate the Rossman-like kinases. Group II comprises the P-loop kinase family (7,732 sequences). These include gluconokinase (EC 2.7.1.12); phosphoribulokinase (EC 2.7.1.19); thymidine kinase (EC 2.7.1.21); ribosylnicotinamide kinase (EC 2.7.1.22); dephospho-CoA kinase (EC 2.7.1.24); adenylylsulfate kinase (EC 2.7.1.25); pantothenate kinase (EC 2.7.1.33); protein kinase (bacterial) (EC 2.7.1.37); uridine kinase (EC 2.7.1.48); shikimate kinase (EC 2.7.1.71); deoxycytidine kinase (EC 2.7.1.74); deoxyadenosine kinase (EC 2.7.1.76); polynucleotide 5′-hydroxyl-kinase (EC 2.7.1.78); 6-phosphofructo-2-kinase (EC 2.7.1.105); deoxyguanosine kinase (EC 2.7.1.113); tetraacyldisaccharide 4′-kinase (EC 2.7.1.130); deoxynucleoside kinase (EC 2.7.1.145); adenosylcobinamide kinase (EC 2.7.1.156); polyphosphate kinase (EC 2.7.4.1); phosphomevalonate kinase (EC 2.7.4.2); adenylate kinase (EC 2.7.4.3); nucleoside-phosphate kinase (EC 2.7.4.4); guanylate kinase (EC 2.7.4.8); thymidylate kinase (EC 2.7.4.9); nucleoside-triphosphate-adenylate kinase (EC 2.7.4.10); (deoxy)nucleoside-phosphate kinase (EC 2.7.4.13); cytidylate kinase (EC 2.7.4.14); and uridylate kinase (EC 2.7.4.22). Group II further comprises the phosphoenolpyruvate carboxykinase family (815 sequences). These enzymes include protein kinase (HPr kinase/phosphatase) (EC 2.7.1.37); phosphoenolpyruvate carboxykinase (GTP) (EC 4.1.1.32); and phosphoenolpyruvate carboxykinase (ATP) (EC 4.1.1.49). Group II further comprises the phosphoglycerate kinase (1,351 sequences) family. These enzymes include phosphoglycerate kinase (EC 2.7.2.3) and phosphoglycerate kinase (GTP) (EC 2.7.2.10). Group II further comprises the aspartokinase family (2,171 sequences). These enzymes include carbamate kinase (EC 2.7.2.2); aspartate kinase (EC 2.7.2.4); acetylglutamate kinase (EC 2.7.2.8 1); glutamate 5-kinase (EC 2.7.2.1) and uridylate kinase (EC 2.7.4.). Group II further comprises the phosphofructokinase-like kinase family (1,998 sequences). These enzymes include 6-phosphofrutokinase (EC 2.7.1.11); NAD(+) kinase (EC 2.7.1.23); 1-phosphofructokinase (EC 2.7.1.56); diphosphate-fructose-6-phosphate I-phosphotransferase (EC 2.7.1.90); sphinganine kinase (EC 2.7.1.91); diacylglycerol kinase (EC 2.7.1.107); and ceramide kinase (EC 2.7.1.138). Group II further comprises the ribokinase-like family (2,722 sequences). These enzymes include: glucokinase (EC 2.7.1.2); ketohexokinase (EC 2.7.1.3); fructokinase (EC 2.7.1.4); 6-phosphofructokinase (EC 2.7.1.11); ribokinase (EC 2.7.1.15); adenosine kinase (EC 2.7.1.20); pyridoxal kinase (EC 2.7.1.35); 2-dehydro-3-deoxygluconokinase (EC 2.7.1.45); hydroxymethylpyrimidine kinase (EC 2.7.1.49); hydroxyethylthiazole kinase (EC 2.7.1.50); I-phosphofructokinase (EC 2.7.1.56); inosine kinase (EC 2.7.1.73); 5-dehydro-2-deoxygluconokinase (EC 2.7.1.92); tagatose-6-phosphate kinase (EC 2.7.1.144); ADP-dependent phosphofructokinase (EC 2.7.1.146); ADP-dependent glucokinase (EC 2.7.1.147); and phosphomethylpyrimidine kinase (EC 2.7.4.7). Group II further comprises the thiamin pyrophosphokinase family (175 sequences) which includes thiamin pyrophosphokinase (EC 2.7.6.2). Group II further comprises the glycerate kinase family (107 sequences) which includes glycerate kinase (EC 2.7.1.31).

Group III kinases (10,973 sequences) comprise the ferredoxin-like fold kinases. Group III further comprises the nucleoside-diphosphate kinase family (923 sequences). These enzymes include nucleoside-diphosphate kinase (EC 2.7.4.6). Group III further comprises the HPPK kinase family (609 sequences). These enzymes include 2-amino-4-hydroxy-6-hydroxymethyldihydropteridine pyrophosphokinase (EC 2.7.6.3). Group III further comprises the guanido kinase family (324 sequences). These enzymes include guanidoacetate kinase (EC 2.7.3.1); creatine kinase (EC 2.7.3.2); arginine kinase (EC 2.7.3.3); and lombricine kinase (EC 2.7.3.5). Group III further comprises the histidine kinase family (9,117 sequences). These enzymes include protein kinase (histidine kinase) (EC 2.7.1.37); [pyruvate dehydrogenase (lipoamide)] kinase (EC 2.7.1.99); and [3-methyl-2-oxybutanoate dehydrogenase(lipoamide)] kinase (EC 2.7.1.115).

Group IV kinases (2,768 sequences) incorporate ribonuclease H-like kinases. These enzymes include hexokinase (EC 2.7.1.1); glucokinase (EC 2.7.1.2); fructokinase (EC 2.7.1.4); rhamnulokinase (EC 2.7.1.5); mannokinase (EC 2.7.1.7); gluconokinase (EC 2.7.1.12); L-ribulokinase (EC 2.7.1.16); xylulokinase (EC 2.7.1.17); erythritol kinase (EC 2.7.1.27); glycerol kinase (EC 2.7.1.30); pantothenate kinase (EC 2.7.1.33); D-ribulokinase (EC 2.7.1.47); L-fucolokinase (EC 2.7.1.51); L-xylulokinase (EC 2.7.1.53); allose kinase (EC 2.7.1.55); 2-dehydro-3-deoxygalactonokinase (EC 2.7.1.58); N-acetylglucosamine kinase (EC 2.7.1.59); N-acylmannosamine kinase (EC 2.7.1.60); polyphosphate-glucose phosphotransferase (EC 2.7.1.63); beta-glucoside kinase (EC 2.7.1.85); acetate kinase (EC 2.7.2.1); butyrate kinase (EC 2.7.2.7); branched-chain-fatty-acid kinase (EC 2.7.2.14); and propionate kinase (EC 2.7.2.15).

Group V kinases (1,119 sequences) incorporate TIM β-barrel kinases. These enzymes include pyruvate kinase (EC 2.7.1.40).

Group VI kinases (885 sequences) incorporate GHMP kinases. These enzymes include galactokinase (EC 2.7.1.6); mevalonate kinase (EC 2.7.1.36); homoserine kinase (EC 2.7.1.39); L-arabinokinase (EC 2.7.1.46); fucokinase (EC 2.7.1.52); shikimate kinase (EC 2.7.1.71); 4-(cytidine 5′-diphospho)-2-C-methyl-D-erythriol kinase (EC 2.7.1.148); and phosphomevalonate kinase (EC 2.7.4.2).

Group VII kinases (1,843 sequences) incorporate AIR synthetase-like kinases. These enzymes include thiamine-phosphate kinase (EC 2.7.4.16) and selenide, water dikinase (EC 2.7.9.3).

Group VIII kinases (565 sequences) incorporate riboflavin kinases (565 sequences). These enzymes include riboflavin kinase (EC 2.7.1.26).

Group IX kinases (197 sequences) incorporate dihydroxyacetone kinases. These enzymes include glycerone kinase (EC 2.7.1.29).

Group X kinases (148 sequences) incorporate putative glycerate kinases. These enzymes include glycerate kinase (EC 2.7.1.31).

Group XI kinases (446 sequences) incorporate polyphosphate kinases. These enzymes include polyphosphate kinases (EC 2.7.4.1).

Group XII kinases (263 sequences) incorporate integral membrane kinases. Group XII comprises the dolichol kinase family. These enzymes include dolichol kinases (EC 2.7.1.108). Group XII further comprises the undecaprenol kinase family. These enzymes include undecaprenol kinases (EC 2.7.1.66).

Kinases play indispensable roles in numerous cellular metabolic and signaling pathways, and are among the best-studied enzymes at the structural, biochemical, and cellular level. Despite the fact that all kinases use the same phosphate donor (in most cases, ATP) and catalyze apparently the same phosphoryl transfer reaction, they display remarkable diversity in their structural folds and substrate recognition mechanisms. This probably is due largely to the diverse nature of the structures and properties of their substrates.

### 1.1. Mitogen-Activated Protein Kinase (MAPK)-Activated Protein Kinases (MK2 and MK3)

Different groups of MAPK-activated protein kinases (MAP-KAPKs) have been defined downstream of mitogen-activated protein kinases (MAPKs). These enzymes transduce signals to target proteins that are not direct substrates of the MAPKs and, therefore, serve to relay phosphorylation-dependent signaling with MAPK cascades to diverse cellular functions. One of these groups is formed by the three MAPKAPKs: MK2, MK3 (also known as 3pK), and MK5 (also designated PRAK). Mitogen-activated protein kinase-activated protein kinase 2 (also referred to as “MAPKAPK2”, “MAPKAP-K2”, “MK2”) is a kinase of the serine/threonine (Ser/Thr) protein kinase family. MK2 is highly homologous to MK3 (approximately 75% amino acid identity). The kinase domains of MK2 and MK3 are most similar (approximately 35% to 40% identity) to calcium/calmodulin-dependent protein kinase (CaMK), phosphorylase b kinase, and the C-terminal kinase domain (CTKD) of the ribosomal S6 kinase (RSK) isoforms. The MK2 gene encodes two alternatively spliced transcripts of 370 amino acids (MK2A) and 400 amino acids (MK2B). The MK3 gene encodes one transcript of 382 amino acids. The MK2- and MK3 proteins are highly homologous, yet MK2A possesses a shorter C-terminal region. The C-terminus of MK2B contains a functional bipartite nuclear localization sequence (NLS) (Lys-Lys-Xaa-Xaa-Xaa-Xaa-Xaa-Xaa-Xaa-Xaa-Xaa-Xaa-Lys-Arg-Arg-Lys-Lys; SEQ ID NO: 21) that is not present in the shorter MK2A isoform, indicating that alternative splicing determines the cellular localization of the MK2 isoforms. MK3 possesses a similar nuclear localization sequence. The nuclear localization sequence found in both MK2B and MK3 encompasses a D domain (Leu-Leu-Lys-Arg-Arg-Lys-Lys; SEQ ID NO: 22), which was shown to mediate the specific interaction of MK2B and MK3 with p38α and p38β. MK2B and MK3 also possess a functional nuclear export signal (NES) located N-terminal to the NLS and D domain. The NES in MK2B is sufficient to trigger nuclear export following stimulation, a process which may be inhibited by leptomycin B. The sequence N-terminal to the catalytic domain in MK2 and MK3 is proline rich and contains one (MK3) or two (MK2) putative Src homology 3 (SH3) domain-binding sites, which studies have shown, for MK2, to mediate binding to the SH3 domain of c-Ab1 in vitro. Recent studies suggest that this domain is involved in MK2-mediated cell migration.

MK2B and MK3 are located predominantly in the nucleus of quiescent cells while MK2A is present in the cytoplasm. Both MK2B and MK3 are rapidly exported to the cytoplasm via a chromosome region maintenance protein (CRM1)-dependent mechanism upon stress stimulation. Nuclear export of MK2B appears to be mediated by kinase activation, as phosphomimetic mutation of Thr334 within the activation loop of the kinase enhances the cytoplasmic localization of MK2B. Without being limited by theory, it is thought that MK2B and MK3 may contain a constitutively active nuclear localization signal (NLS) and a phosphorylation-regulated nuclear export signal (NES).

MK2 and MK3 appear to be expressed ubiquitously, with increased relative expression in the heart, lungs, kidney, reproductive organs (mammary and testis), skin and skeletal muscle tissues, as well as in immune-related cells such as white blood cells/leukocytes and dendritic cells.

### 1.1.1. Activation

Various activators of p38α and p38β potently stimulate MK2 and MK3 activity. p38 mediates the in vitro and in vivo phosphorylation of MK2 on four proline-directed sites: Thr25, Thr222, Ser272, and Thr334. Of these sites, only Thr25 is not conserved in MK3. Without being limited by theory, while the function of phosphorylated Thr25 is unknown, its location between the two SH3 domain-binding sites suggests that it may regulate protein-protein interactions. Thr222 in MK2 (Thr201 in MK3) is located in the activation loop of the kinase domain and has been shown to be essential for MK2 and MK3 kinase activity. Thr334 in MK2 (Thr313 in MK3) is located C-terminal to the catalytic domain and is essential for kinase activity. The crystal structure of MK2 has been resolved and, without being limited by theory, suggests that Thr334 phosphorylation may serve as a switch for MK2 nuclear import and export. Phosphorylation of Thr334 also may weaken or interrupt binding of the C terminus of MK2 to the catalytic domain, exposing the NES and promoting nuclear export.

Studies have shown that while p38 is capable of activating MK2 and MK3 in the nucleus, experimental evidence suggests that activation and nuclear export of MK2 and MK3 are coupled by a phosphorylation-dependent conformational switch that also dictates p38 stabilization and localization, and the cellular location of p38 itself is controlled by MK2 and possibly MK3. Additional studies have shown that nuclear p38 is exported to the cytoplasm in a complex with MK2 following phosphorylation and activation of MK2. The interaction between p38 and MK2 may be important for p38 stabilization since studies indicate that p38 levels are low in MK2-deficient cells and expression of a catalytically inactive MK2 protein restores p38 levels.

### 1.1.2. Substrates and Functions

MK2 shares many substrates with MK3. Both enzymes have comparable substrate preferences and phosphorylate peptide substrates with similar kinetic constants. The minimum sequence required for efficient phosphorylation by MK2 was found to be Hyd-Xaa-Arg-Xaa-Xaa-pSer/pThr (SEQ ID NO: 22), where Hyd is a bulky, hydrophobic residue.

Accumulating studies have shown that MK2 phosphorylates a variety of proteins, which include, but are not limited to, 5-Lipooxygenase (ALOX5), Cell Division Cycle 25 Homolog B (CDC25B), Cell Division Cycle 25 Homolog C(CDC25C), Embryonic Lethal, Abnormal Vision, Drosophila-Like 1 (ELAVL1), Heterogeneous Nuclear Ribonucleoprotein A0 (HNRNPA0), Heat Shock Factor protein 1 (HSF1), Heat Shock Protein Beta-1 (HSPB1), Keratin 18 (KRT18), Keratin 20 (KRT20), LIM domain kinase 1 (LIMK1), Lymphocyte-specific protein 1 (LSP1), Polyadenylate-Binding Protein 1 (PABPC1), Poly(A)-specific Ribonuclease (PARN), CAMP-specific 3′,5′-cyclic Phosphodiesterase 4A (PDE4A), RCSD domain containing 1 (RCSD1), Ribosomal protein S6 kinase, 90 kDa, polypeptide 3 (RPS6KA3), TGF-beta activated kinase 1/MAP3K7 binding protein 3 (TAB3), and Tristetraprolin (TTP/ZFP36).

Heat-Shock Protein Beta-1 (also termed HSPB1 or HSP27) is a stress-inducible cytosolic protein that is ubiquitously present in normal cells and is a member of the small heat-shock protein family. The synthesis of HSPB1 is induced by heat shock and other environmental or pathophysiologic stresses, such as UV radiation, hypoxia and ischemia. Besides its putative role in thermoresistance, HSPB1 is involved in the survival and recovery of cells exposed to stressful conditions.

Experimental evidence supports a role for p38 in the regulation of cytokine biosynthesis and cell migration. The targeted deletion of the mk2 gene in mice suggested that although p38 mediates the activation of many similar kinases, MK2 seems to be the key kinase responsible for these p38-dependent biological processes. Loss of MK2 leads (i) to a defect in lipopolysaccharide (LPS)-induced synthesis of cytokines such as tumor necrosis factor alpha (TNF-α), interleukin-6 (IL-6), and gamma interferon (IFN-γ) and (ii) to changes in the migration of mouse embryonic fibroblasts, smooth muscle cells, and neutrophils.

Consistent with a role for MK2 in inflammatory and immune responses, MK2-deficient mice showed increased susceptibility to Listeria monocytogenes infection and reduced inflammation-mediated neuronal death following focal ischemia. Since the levels of p38 protein also are reduced significantly in MK2-deficient cells, it was necessary to distinguish whether these phenotypes were due solely to the loss of MK2. To achieve this, MK2 mutants were expressed in MK2-deficient cells, and the results indicated that the catalytic activity of MK2 was not necessary to restore p38 levels but was required to regulate cytokine biosynthesis.

Knockout or knockdown studies of MK2 provide strong support that activated MK2 enhances stability of IL-6 mRNA through phosphorylation of proteins interacting with the AU-rich 3′ untranslated region of IL-6 mRNA. In particular, it has been shown that MK2 is principally responsible for phosphorylation of hnRNPA0, an mRNA-binding protein that stabilizes IL-6 RNA. In addition, several additional studies investigating diverse inflammatory diseases have found that levels of pro-inflammatory cytokines, such as IL-6, IL-1β, TNF-α and IL-8, are increased in induced sputum from patients with stable chronic obstructive pulmonary disease (COPD) or from the alveolar macrophages of cigarette smokers (Keatings V. et al, Am J Resp Crit. Care Med, 1996, 153:530-534; Lim, S. et al., J Respir Crit. Care Med, 2000, 162:1355-1360).

1.1.3. Regulation of mRNA Translation.

Previous studies using MK2 knockout mice or MK2-deficient cells have shown that MK2 increases the production of inflammatory cytokines, including TNF-α, IL-1, and IL-6, by increasing the rate of translation of its mRNA. No significant reductions in the transcription, processing, and shedding of TNF-α could be detected in MK2-deficient mice. The p38 pathway is known to play an important role in regulating mRNA stability, and MK2 represents a likely target by which p38 mediates this function. Studies utilizing MK2-deficient mice indicated that the catalytic activity of MK2 is necessary for its effects on cytokine production and migration, suggesting that, without being limited by theory, MK2 phosphorylates targets involved in mRNA stability. Consistent with this, MK2 has been shown to bind and/or phosphorylate the heterogeneous nuclear ribonucleoprotein (hnRNP) A0, tristetraprolin (TTP), the poly(A)-binding protein PABP1, and HuR, a ubiquitously expressed member of the ELAV (Embryonic-Lethal Abnormal Visual in Drosophila melanogaster) family of RNA-binding protein. These substrates are known to bind or copurify with mRNAs that contain AU-rich elements in the 3′ untranslated region, suggesting that MK2 may regulate the stability of AU-rich mRNAs such as TNF-α. It currently is unknown whether MK3 plays a similar role, but LPS treatment of MK2-deficient fibroblasts completely abolished hnRNP A0 phosphorylation, suggesting that MK3 is not able to compensate for the loss of MK2.

MK3 participates with MK2 in phosphorylation of the eukaryotic elongation factor 2 (eEF2) kinase. eEF2 kinase phosphorylates and inactivates eEF2. eEF2 activity is critical for the elongation of mRNA during translation, and phosphorylation of eEF2 on Thr56 results in the termination of mRNA translation. MK2 and MK3 phosphorylation of eEF2 kinase on Ser377 suggests that these enzymes may modulate eEF2 kinase activity and thereby regulate mRNA translation elongation.

### 1.1.4. Transcriptional Regulation by MK2 and MK3

Nuclear MK2, similar to many MKs, contributes to the phosphorylation of cAMP response element binding (CREB), Activating Transcription Factor-1 (ATF-1), serum response factor (SRF), and transcription factor ER81. Comparison of wild-type and MK2-deficient cells revealed that MK2 is the major SRF kinase induced by stress, suggesting a role for MK2 in the stress-mediated immediate-early response. Both MK2 and MK3 interact with basic helix-loop-helix transcription factor E47 in vivo and phosphorylate E47 in vitro. MK2-mediated phosphorylation of E47 was found to repress the transcriptional activity of E47 and thereby inhibit E47-dependent gene expression, suggesting that MK2 and MK3 may regulate tissue-specific gene expression and cell differentiation.

### 1.1.5. Other Targets of MK2 and MK3

Several other MK2 and MK3 substrates also have been identified, reflective of the diverse functions of MK2 and MK3 in several biological processes. The scaffolding protein 14-3-3ζ is a physiological MK2 substrate. Studies indicate that 14-3-3ζ interacts with a number of components of cell signaling pathways, including protein kinases, phosphatases, and transcription factors. Additional studies have shown that MK2-mediated phosphorylation of 14-3-3ζ on Ser58 compromises its binding activity, suggesting that MK2 may affect the regulation of several signaling molecules normally regulated by 14-3-3ζ.

Additional studies have shown that MK2 also interacts with and phosphorylates the p16 subunit of the seven-member Arp2 and Arp3 complex (p16-Arc) on Ser77. p16-Arc has roles in regulating the actin cytoskeleton, suggesting that MK2 may be involved in this process. Further studies have shown that the small heat shock protein HSPB1, lymphocyte-specific protein LSP-1, and vimentin are phosphorylated by MK2. HSPB1 is of particular interest because it forms large oligomers which may act as molecular chaperones and protect cells from heat shock and oxidative stress. Upon phosphorylation, HSPB1 loses its ability to form large oligomers and is unable to block actin polymerization, suggesting that MK2-mediated phosphorylation of HSPB1 serves a homeostatic function aimed at regulating actin dynamics that otherwise would be destabilized during stress. MK3 also was shown to phosphorylate HSPB1 in vitro and in vivo, but its role during stressful conditions has not yet been elucidated.

It was also shown that HSPB1 binds to polyubiquitin chains and to the 26S proteasome in vitro and in vivo. The ubiquitin-proteasome pathway is involved in the activation of transcription factor NF-kappa B (NF-κB) by degrading its main inhibitor, I kappa B-alpha (IκB-alpha), and it was shown that overexpresion of HSPB1 increases NF-kappaB (NF-κB) nuclear relocalization, DNA binding, and transcriptional activity induced by etoposide, TNF-alpha, and Interleukin-1 beta (IL-1β). Additionally, previous studies have suggested that HSPB1, under stress conditions, favors the degradation of ubiquitinated proteins, such as phosphorylated I kappa B-alpha (IκB-alpha); and that this function of HSPB1 accounts for its anti-apoptotic properties through the enhancement of NF-kappa B (NF-κB) activity (Parcellier, A. et al., Mol Cell Biol, 23(16): 5790-5802, 2003).

MK2 and MK3 also may phosphorylate 5-lipoxygenase. 5-lipoxygenase catalyzes the initial steps in the formation of the inflammatory mediators, leukotrienes. Tyrosine hydroxylase, glycogen synthase, and Akt also were shown to be phosphorylated by MK2. Finally, MK2 phosphorylates the tumor suppressor protein tuberin on Ser1210, creating a docking site for 14-3-3ζ. Tuberin and hamartin normally form a functional complex that negatively regulates cell growth by antagonizing mTOR-dependent signaling, suggesting that p38-mediated activation of MK2 may regulate cell growth by increasing 14-3-3ζ binding to tuberin.

Accumulating studies have suggested that the reciprocal crosstalk between the p38 MAPK-pathway and signal transducer and activator of transcription 3 (STAT3)-mediated signal-transduction forms a critical axis successively activated in lipopolysaccharide (LPS) challenge models. It was shown that the balanced activation of this axis is essential for both induction and propagation of the inflammatory macrophage response as well as for the control of the resolution phase, which is largely driven by IL-10 and sustained STAT3 activation (Bode, J. et al., Cellular Signalling, 24: 1185-1194, 2012). In addition, another study has shown that MK2 controls LPS-inducible IFNβ gene expression and subsequent IFNβ-mediated activation of STAT3 by neutralizing negative regulatory effects of MK3 on LPS-induced p65 and IRF3-mediated signaling. The study further showed that in mk2/3 knockout macrophages, IFNβ-dependent STAT3 activation occurs independently from IL-10, because, in contrast to IFNβ-, impaired IL-10 expression is not restored upon additional deletion of MK3 in mk2/3 knockout macrophages (Ehlting, C. et al., J. Biol. Chem., 285(27): 24113-24124).

### 1.2. Kinase Inhibition

The eukaryotic protein kinases constitute one of the largest superfamilies of homologous proteins that are related by virtue of their catalytic domains. Most related protein kinases are specific for either serine/threonine or tyrosine phosphorylation. Protein kinases play an integral role in the cellular response to extracellular stimuli. Thus, stimulation of protein kinases is considered to be one of the most common activation mechanisms in signal transduction systems. Many substrates are known to undergo phosphorylation by multiple protein kinases, and a considerable amount of information on primary sequence of the catalytic domains of various protein kinases has been published. These sequences share a large number of residues involved in ATP binding, catalysis, and maintenance of structural integrity. Most protein kinases possess a well conserved 30-32 kDa catalytic domain.

Studies have attempted to identify and utilize regulatory elements of protein kinases. These regulatory elements include inhibitors, antibodies, and blocking peptides.

### 1.2.1. Inhibitors

Enzyme inhibitors are molecules that bind to enzymes thereby decreasing enzyme activity. The binding of an inhibitor may stop a substrate from entering the active site of the enzyme and/or hinder the enzyme from catalyzing its reaction (as in inhibitors directed at the ATP biding site of the kinase). Inhibitor binding is either reversible or irreversible. Irreversible inhibitors usually react with the enzyme and change it chemically (e.g., by modifying key amino acid residues needed for enzymatic activity) so that it no longer is capable of catalyzing its reaction. In contrast, reversible inhibitors bind non-covalently and different types of inhibition are produced depending on whether these inhibitors bind the enzyme, the enzyme-substrate complex, or both.

Enzyme inhibitors often are evaluated by their specificity and potency. The term “specificity” as used in this context refers to the selective attachment of an inhibitor or its lack of binding to other proteins. The term “potency” as used herein refers to an inhibitor's dissociation constant, which indicates the concentration of inhibitor needed to inhibit an enzyme.

Inhibitors of protein kinases have been studied for use as a tool in protein kinase activity regulation Inhibitors have been studied for use with, for example, cyclin-dependent (Cdk) kinase, MAP kinase, serine/threonine kinase, Src Family protein tyrosine kinase, tyrosine kinase, calmodulin (CaM) kinase, casein kinase, checkpoint kinase (Chk1), glycogen synthase kinase 3 (GSK-3), c-Jun N-terminal kinase (JNK), mitogen-activated protein kinase 1 (MEK), myosin light chain kinase (MLCK), protein kinase A, Akt (protein kinase B), protein kinase C, protein kinase G, protein tyrosine kinase, Raf kinase, and Rho kinase.

### 1.2.2. Small-Molecule MK2 Inhibitors

While individual inhibitors that target MK2 with at least modest selectivity with respect to other kinases have been designed, it has been difficult to create compounds with favorable solubility and permeability. As a result, there are relatively few biochemically efficient MK2 inhibitors that have advanced to in vivo pre-clinical studies (Edmunds, J. and Talanian, MAPKAP Kinase 2 (MK2) as a Target for Anti-inflammatory Drug Discovery. In Levin, J and Laufer, S (Ed.), RSC Drug Discovery Series No. 26, p 158-175, the Royal Society of Chemistry, 2012; incorporated by reference in its entirety).

The majority of disclosed MK2 inhibitors are classical type I inhibitors as revealed by crystallographic or biochemical studies. As such, they bind to the ATP site of the kinase and thus compete with intra-cellular ATP (estimated concentration 1 mM-5 mM) to inhibit phosphorylation and activation of the kinase. Representative examples of small-molecule MK2 inhibitors include, but are not limited to,

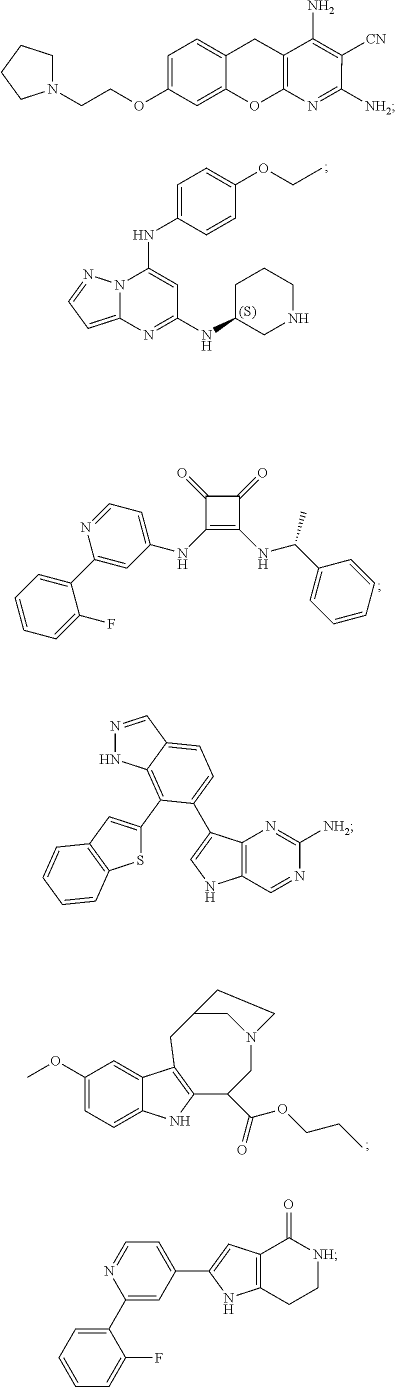

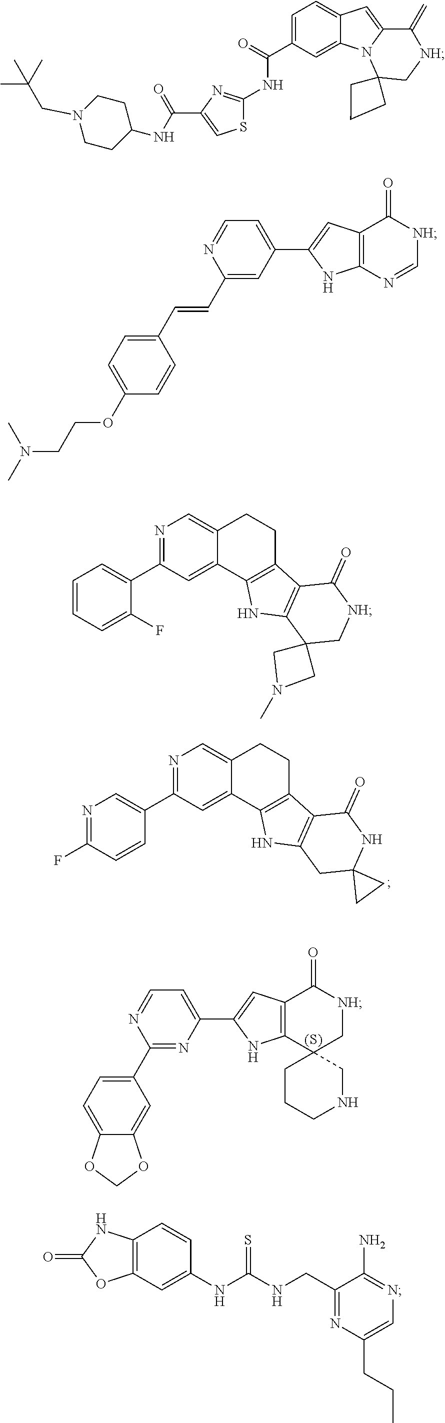

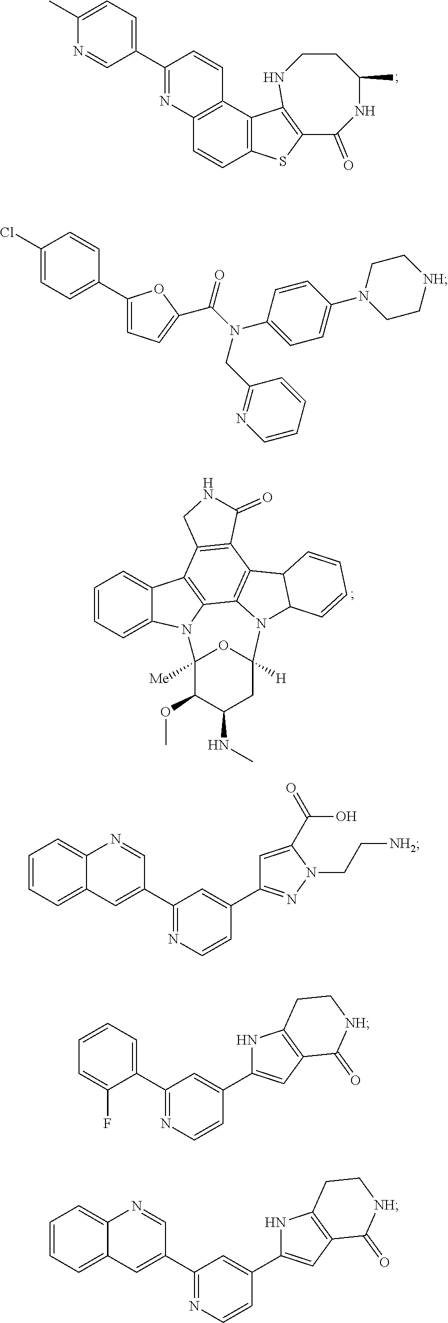

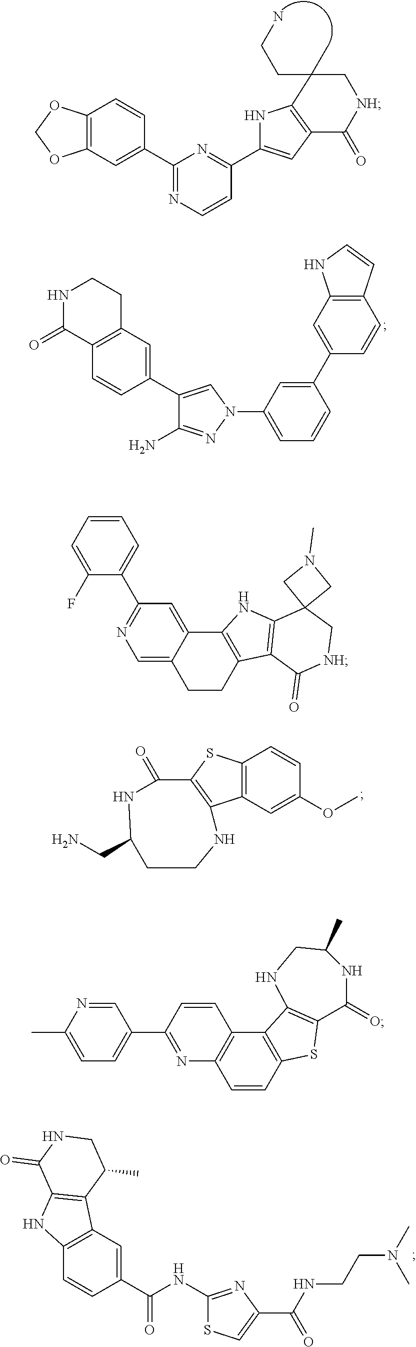

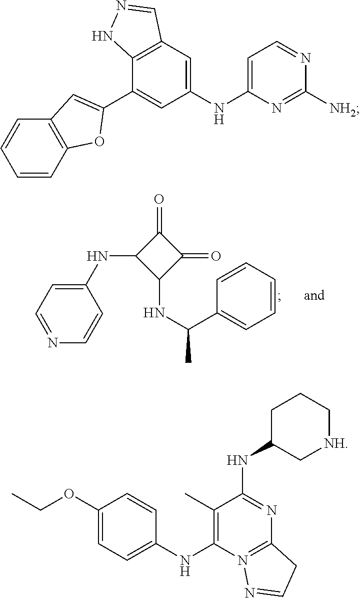

### 1.2.3. Blocking Peptides

A peptide is a chemical compound that is composed of a chain of two or more amino acids whereby the carboxyl group of one amino acid in the chain is linked to the amino group of the other via a peptide bond. Peptides have been used inter alia in the study of protein structure and function. Synthetic peptides may be used inter alia as probes to see where protein-peptide interactions occur Inhibitory peptides may be used inter alia in clinical research to examine the effects of peptides on the inhibition of protein kinases, cancer proteins and other disorders.

The use of several blocking peptides has been studied. For example, extracellular signal-regulated kinase (ERK), a MAPK protein kinase, is essential for cellular proliferation and differentiation. The activation of MAPKs requires a cascade mechanism whereby MAPK is phosphorylated by an upstream MAPKK (MEK) which then, in turn, is phosphorylated by a third kinase MAPKKK (MEKK). The ERK inhibitory peptide functions as a MEK decoy by binding to ERK.

Other blocking peptides include autocamtide-2 related inhibitory peptide (AIP). This synthetic peptide is a highly specific and potent inhibitor of Ca²⁺/calmodulin-dependent protein kinase II (CaMKII). AIP is a non-phosphorylatable analog of autocamtide-2, a highly selective peptide substrate for CaMKII. AIP inhibits CaMKII with an IC50 of 100 nM (IC50 is the concentration of an inhibitor required to obtain 50% inhibition). The AIP inhibition is non-competitive with respect to syntide-2 (CaMKII peptide substrate) and ATP but competitive with respect to autocamtide-2. The inhibition is unaffected by the presence or absence of Ca²⁺/calmodulin. CaMKII activity is inhibited completely by AIP (1 μM) while PKA, PKC and CaMKIV are not affected.

Other blocking peptides include cell division protein kinase 5 (Cdk5) inhibitory peptide (CIP). Cdk5 phosphorylates the microtubule protein tau at Alzheimer's Disease-specific phospho-epitopes when it associates with p25. p25 is a truncated activator, which is produced from the physiological Cdk5 activator p35 upon exposure to amyloid 0 peptides. Upon neuronal infections with CIP, CIPs selectively inhibit p25/Cdk5 activity and suppress the aberrant tau phosphorylation in cortical neurons. The reasons for the specificity demonstrated by CIP are not fully understood.

Additional blocking peptides have been studied for extracellular-regulated kinase 2 (ERK2), ERK3, p38/HOG1, protein kinase C, casein kinase II, Ca²⁺/calmodulin kinase IV, casein kinase II, Cdk4, Cdk5, DNA-dependent protein kinase (DNA-PK), serine/threonine-protein kinase PAK3, phosphoinositide (PI)-3 kinase, PI-5 kinase, PSTAIRE (the cdk highly conserved sequence), ribosomal S6 kinase, GSK-4, germinal center kinase (GCK), SAPK (stress-activated protein kinase), SEK1 (stress signaling kinase), and focal adhesion kinase (FAK).

### 1.2.4. Protein Substrate-Competitive Inhibitors

Most of the protein kinase inhibitors developed to date are ATP competitors. This type of molecule competes for the ATP binding site of the kinase and often shows off-target effects due to serious limitations in its specificity. The low specificity of these inhibitors is due to the fact that the ATP binding site is highly conserved among diverse protein kinases. Non-ATP competitive inhibitors, on the other hand, such as substrate competitive inhibitors, are expected to be more specific as the substrate binding sites have a certain degree of variability among the various protein kinases.

Although substrate competitive inhibitors usually have a weak binding interaction with the target enzyme in vitro, studies have shown that chemical modifications can improve the specific biding affinity and the in vivo efficacy of substrate inhibitors (Eldar-Finkelman, H. et al., Biochim, Biophys. Acta, 1804(3):598-603, 2010). In addition, substrate competitive inhibitors show better efficacy in cells than in cell-free conditions in many cases (van Es, J. et al., Curr. Opin. Gent. Dev. 13:28-33, 2003).

In an effort to enhance specificity and potency in protein kinase inhibition, bisubstrate inhibitors also have been developed. Bisubstrate inhibitors, which consist of two conjugated fragments, each targeted to a different binding site of a bisubstrate enzyme, form a special group of protein kinase inhibitors that mimic two natural substrates/ligands and that simultaneously associate with two regions of given kinases. The principle advantage of bisubstrate inhibitors is their ability to generate more interactions with the target enzyme that could result in improved affinity and selectivity of the conjugates, when compared with single-site inhibitors. Examples of bisubstrate inhibitors include, but are not limited to, nucleotide-peptide conjugates, adenosine derivative-peptide conjugates, and conjugates of peptides with potent ATP-competitive inhibitors.

### 1.2.5. Protein Transduction Domains

The plasma membrane presents a formidable barrier to the introduction of macromolecules into cells. For nearly all therapeutics to exert their effects, at least one cellular membrane must be traversed. Traditional small molecule pharmaceutical development relies on the chance discovery of membrane permeable molecules with the ability to modulate protein function. Although small molecules remain the dominant therapeutic paradigm, many of these molecules suffer from lack of specificity, side effects, and toxicity. Information-rich macromolecules, which have protein modulatory functions far superior to those of small molecules, can be created using rational drug design based on molecular, cellular, and structural data. However, the plasma membrane is impermeable to most molecules of size greater than 500 Da. Therefore, the ability of cell penetrating peptides, such as the basic domain of Trans-Activator of Transcription (Tat), to cross the cell membrane and deliver macromolecular cargo in vivo, can greatly facilitate the rational design of therapeutic proteins, peptides, and nucleic acids.

Protein transduction domains (PTDs) are a class of peptides capable of penetrating the plasma membrane of mammalian cells and of transporting compounds of many types and molecular weights across the membrane. These compounds include effector molecules, such as proteins, DNA, conjugated peptides, oligonucleotides, and small particles such as liposomes. When PTDs are chemically linked or fused to other proteins, the resulting fusion peptides still are able to enter cells. Although the exact mechanism of transduction is unknown, internalization of these proteins is not believed to be receptor-mediated or transporter-mediated. PTDs are generally 10-16 amino acids in length and may be grouped according to their composition, such as, for example, peptides rich in arginine and/or lysine.

The use of PTDs capable of transporting effector molecules into cells has become increasingly attractive in the design of drugs as they promote the cellular uptake of cargo molecules. These cell-penetrating peptides, generally categorized as amphipathic (meaning having both a polar and a nonpolar end) or cationic (meaning of or relating to containing net positively charged atoms) depending on their sequence, provide a non-invasive delivery technology for macromolecules. PTDs often are referred to as “Trojan peptides”, “membrane translocating sequences”, or “cell permeable proteins” (CPPs). PTDs also may be used to assist novel HSPB1 kinase inhibitors to penetrate cell membranes. (see U.S. application Ser. No. 11/972,459, entitled “Polypeptide Inhibitors of HSPB1 Kinase and Uses Therefor,” filed Jan. 10, 2008, and Ser. No. 12/188,109, entitled “Kinase Inhibitors and Uses Thereof,” filed Aug. 7, 2008, the contents of each application are incorporated by reference in their entirety herein).

### 1.2.5.1. Viral PTD Containing Proteins

The first proteins to be described as having transduction properties were of viral origin. These proteins still are the most commonly accepted models for PTD action. The HIV-1 Transactivator of Transcription (Tat) and HSV-1 VP 22 protein are the best characterized viral PTD containing proteins.

Tat (HIV-1 trans-activator gene product) is an 86-amino acid polypeptide, which acts as a powerful transcription factor of the integrated HIV-1 genome. Tat acts on the viral genome, stimulating viral replication in latently infected cells. The translocation properties of the Tat protein enable it to activate quiescent infected cells, and it may be involved in priming of uninfected cells for subsequent infection by regulating many cellular genes, including cytokines The minimal PTD of Tat is the 9 amino acid protein sequence RKKRRQRRR (TAT49-57; SEQ ID NO: 20). Studies utilizing a longer fragment of Tat demonstrated successful transduction of fusion proteins up to 120 kDa. The addition of multiple Tat-PTDs as well as synthetic Tat derivatives has been demonstrated to mediate membrane translocation. Tat PTD containing fusion proteins have been used as therapeutic moieties in experiments involving cancer, transporting a death-protein into cells, and disease models of neurodegenerative disorders.

The mechanism used by transducing peptides to permeate cell membranes has been the subject of considerable interest in recent years, as researchers have sought to understand the biology behind transduction. Early reports that Tat transduction occurred by a nonendocytic mechanism have largely been dismissed as artifactual though other cell-penetrating peptides might be taken up by way of direct membrane disruption. The recent findings that transduction of Tat and other PTDs occurs by way of macropinocytosis, a specialized form of endocytosis, has created a new paradigm in the study of these peptides. Enhanced knowledge of the mechanism of transduction helped improve transduction efficiency with the ultimate goal of clinical success (Snyder E. and Dowdy, S., Pharm Res., 21(3):389-393, 2004).

The current model for Tat-mediated protein transduction is a multistep process that involves binding of Tat to the cell surface, stimulation of macropinocytosis, uptake of Tat and cargo into macropinosomes, and endosomal escape into the cytoplasm. The first step, binding to the cell surface, is thought to be through ubiquitous glycan chains on the cell surface. Stimulation of macropinocytosis by Tat occurs by an unknown mechanism that might include binding to a cell surface protein or occur by way of proteoglycans or glycolipids. Uptake by way of macropinocytosis, a form of fluid phase endocytosis used by all cell types, is required for Tat and polyarginine transduction. The final step in Tat transduction is escape from macropinosomes into the cytoplasm; this process is likely to be dependent on the pH drop in endosomes that, along with other factors, facilitates a perturbation of the membrane by Tat and release of Tat and its cargo (i.e. peptide, protein or drug etc.) to the cytoplasm (Snyder E. and Dowdy, S., Pharm Res., 21(3):389-393, 2004).

VP22 is the HSV-1 tegument protein, a structural part of the HSV virion. VP22 is capable of receptor independent translocation and accumulates in the nucleus. This property of VP22 classifies the protein as a PTD containing peptide. Fusion proteins comprising full length VP22 have been translocated efficiently across the plasma membrane.

1.2.5.2. Homeoproteins with Intercellular Translocation Properties

Homeoproteins are highly conserved, transactivating transcription factors involved in morphological processes. They bind to DNA through a specific sequence of 60 amino acids. The DNA-binding homeodomain is the most highly conserved sequence of the homeoprotein. Several homeoproteins have been described as exhibiting PTD-like activity; they are capable of efficient translocation across cell membranes in an energy-independent and endocytosis-independent manner without cell type specificity.

The Antennapedia protein (Antp) is a trans-activating factor capable of translocation across cell membranes; the minimal sequence capable of translocation is a 16 amino acid peptide corresponding to the third helix of the protein's homeodomain (HD). The internalization of this helix occurs at 4° C., suggesting that this process is not endocytosis dependent. Peptides of up to 100 amino acids produced as fusion proteins with AntpHD penetrate cell membranes.

Other homeodomains capable of translocation include Fushi tarazu (Ftz) and Engrailed (En) homeodomain. Many homeodomains share a highly conserved third helix.

### 1.2.5.3. Human PTDs

Human PTDs may circumvent potential immunogenicity issues upon introduction into a human patient. Peptides with PTD sequences include: Hoxa-5, Hox-A4, Hox-B5, Hox-B6, Hox-B7, HOX-D3, GAX, MOX-2, and FtzPTD. These proteins all share the sequence found in AntpPTD. Other PTDs include Islet-1, Interleukin-1 (IL-1), Tumor Necrosis Factor (TNF), and the hydrophobic sequence from Kaposi-fibroblast growth factor or Fibroblast Growth Factor-4 (FGF-4) signal peptide, which is capable of energy-, receptor-, and endocytosis-independent translocation. Unconfirmed PTDs include members of the Fibroblast Growth Factor (FGF) family. FGFs are polypeptide growth factors that regulate proliferation and differentiation of a wide variety of cells. Several publications have reported that basic fibroblast growth factor (FGF-2) exhibits an unconventional internalization similar to that of VP-22, Tat, and homeodomains. It has also been reported that acidic FGF (FGF-1) translocated cell membranes at temperatures as low as 4° C. However, no conclusive evidence exists about the domain responsible for internalization or the translocation properties of fusion proteins (Beerens, A. et al., Curr Gene Ther., 3(5):486-494, 2003).

### 1.2.5.4. Synthetic PTDs

Several peptides have been synthesized in an attempt to create more potent PTDs and to elucidate the mechanisms by which PTDs transport proteins across cell membranes. Many of these synthetic PTDs are based on existing and well documented peptides, while others are selected for their basic residues and/or positive charges, which are thought to be crucial for PTD function. A few of these synthetic PTDs showed better translocation properties than the existing ones (Beerens, A. et al., Curr Gene Ther., 3(5):486-494, 2003). Exemplary Tat-derived synthetic PTDs include, for example, but are not limited to, WLRRIKAWLRRIKA (SEQ ID NO: 12); WLRRIKA (SEQ ID NO: 13); YGRKKRRQRRR (SEQ ID NO: 14); WLRRIKAWLRRI (SEQ ID NO: 15); FAKLAARLYR (SEQ ID NO: 16); KAFAKLAARLYR (SEQ ID NO: 17); and HRRIKAWLKKI (SEQ ID NO: 18).

### 2. Anatomy and Physiology of the Skin

The skin is the largest organ in the body consisting of several layers and plays an important role in biologic homeostasis. Its reapproximation over the surface of the wound has long been a primary sign of the completion of a significant portion of wound healing. This reclosure of the defect restores the protective function of the skin, which includes protection from bacteria, toxins, and mechanical forces, as well as providing the barrier to retain essential body fluids. The epidermis, which is composed of several layers beginning with the stratum corneum, is the outermost layer of the skin. The innermost skin layer is the deep dermis. The skin has multiple functions, including thermal regulation, metabolic function (vitamin D metabolism), and immune functions. FIG. 1 presents a diagram of skin anatomy.

Epidermis

Closing the wound quickly and efficiently is a function of the epidermis. The epidermis provides body's buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically. The basal-cell layer, or germinative layer, is of importance because it is the primary source of regenerative cells. In the process of wound healing, this is the area that undergoes mitosis in most instances. The upper epidermis, including stratum and granular layer, is the other area of formation of the normal epidermal-barrier function.

When epidermis is injured, the body is subject to invasion by outside agents and loss of body fluids. Epidermal wounds heal primarily by cell migration. Clusters of epidermal cells migrate into the area of damage and cover the defect. These cells are phagocytic and clear the surface of debris and plasma clots. Repair cells originate from local sources that are primarily the dermal appendages and from adjacent intact skin areas. Healing occurs rapidly, and the skin is regenerated and is left unscarred. Blisters are examples of epidermal wounds. They may be small vesicles or larger bullae (blisters greater than 1 cm in diameter).

Stratum Corneum and the Acid Mantle

Stratum corneum is an avascular, multilayer structure that functions as a barrier to the environment and prevents transepidermal water loss. Recent studies have shown that enzymatic activity is involved in the formation of an acid mantle in the stratum corneum. Together, the acid mantle and stratum corneum make the skin less permeable to water and other polar compounds, and indirectly protect the skin from invasion by microorganisms. Normal surface skin pH is between 4 and 6.5 in healthy people; it varies according to area of skin on the body. This low pH forms an acid mantle that enhances the skin barrier function. Damage of the stratum corneum increases the skin pH and, thus, the susceptibility of the skin to bacterial skin infections.

Other Layers of the Epidermis

Other layers of the epidermis below the stratum corneum include the stratum lucidum, stratum granulosum, stratum germinativum, and stratum basale. Each contains living cells with specialized functions (FIG. 2). For example melanin, which is produced by melanocytes in the epidermis, is responsible for the color of the skin. Langerhans cells are involved in immune processing.

Dermal Appendages

Dermal appendages, which include hair follicles, sebaceous and sweat glands, fingernails, and toenails, originate in the epidermis and protrude into the dermis hair follicles and sebaceous and sweat glands contribute epithelial cells for rapid reepithelialization of wounds that do not penetrate through the dermis (termed partial-thickness wounds). The sebaceous glands are responsible for secretions that lubricate the skin, keeping it soft and flexible. They are most numerous in the face and sparse in the palm of the hands and soles of the feet. Sweat gland secretions control skin pH to prevent dermal infections. The sweat glands, dermal blood vessels, and small muscles in the skin (responsible for goose pimples) control temperature on the surface of the body. Nerve endings in the skin include receptors for pain, touch, heat, and cold. Loss of these nerve endings increases the risk for skin breakdown by decreasing the tolerance of the tissue to external forces.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

Dermis

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts.

The deep dermis is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness. Dermal wounds, e.g., cracks or pustules, involve the epidermis, basal membrane, and dermis. Typically, dermal injuries heal rapidly. Cracks in the dermis can exude serum, blood, or pus, and lead to formation of clots or crusts. Pustules are pus-filled vesicles that often represent infected hair follicle.

### 3. Biology of Wound Healing

Wound healing is a dynamic, interactive process involving soluble mediators, blood cells, extracellular matrix, and parenchymal cells. Wound healing generally proceeds through three overlapping dynamic phases: (1) an inflammatory phase, (2) a proliferative phase, and (3) remodeling phase.

The inflammatory phase is triggered by capillary damage, which leads to the formation of a blood clot/provisional matrix composed of fibrin and fibronectin. This provisional matrix fills the tissue defect and enables effector cell influx. Platelets present in the clot release multiple cytokines that participate in the recruitment of inflammatory cells (such as neutrophils, monocytes, and macrophages, amonst others), fibroblasts, and endothelial cells (ECs) (FIG. 5).

The inflammatory phase is followed by a proliferative phase, in which active angiogenesis creates new capillaries, allowing nutrient delivery to the wound site, notably to support fibroblast proliferation. Fibroblasts present in granulation tissue are activated and acquire a smooth muscle cell-like phenotype, then being referred to as myofibroblasts. Myofibroblasts synthesize and deposit extracellular matrix (ECM) components that replace the provisional matrix. They also have contractile properties mediated by α-smooth muscle actin organized in microfilament bundles or stress fibers. Myofibroblastic differentiation of fibroblastic cells begins with the appearance of the protomyofibroblast, whose stress fibers contain only β- and γ-cytoplasmic actins. Protomyofibroblasts can evolve into differentiated myofibroblasts whose stress fibers contain α-smooth muscle actin.

The third healing phase involves gradual remodeling of the granulation tissue and reepithelialization. This remodeling process is mediated largely by proteolytic enzymes, especially matrix metalloproteinases (MMPs) and their inhibitors (TIMPs, tissue inhibitors of metalloproteinases). During the reepithalialization, Type III collagen, the main component of granulation tissue, is replaced gradually by type I collagen, the main structural component of the dermis. Elastin, which contributes to skin elasticity and is absent from granulation tissue, also reappears. Cell density normalizes through apoptosis of vascular cells and myofibroblasts (resolution).

3.1. Inflammation

Tissue injury causes the disruption of blood vessels and extravasation of blood constituents. The blood clot re-establishes hemostasis and provides a provisional extracellular matrix for cell migration. Platelets not only facilitate the formation of a hemostatic plug but also secrete several mediators of wound healing, such as platelet-derived growth factor, which attract and activate macrophages and fibroblasts (Heldin, C. and Westermark B., In: Clark R., ed. The molecular and cellular biology of wound repair, 2^{nd} Ed. New York, Plenum Press, pp. 249-273, (1996)). It was suggested, however, that, in the absence of hemorrhage, platelets are not essential to wound healing; numerous vasoactive mediators and chemotactic factors are generated by the coagulation and activated-complement pathways and by injured or activated parenchymal cells that were shown to recruit inflammatory leukocytes to the site of injury (Id.).

Infiltrating neutrophils cleanse the wounded area of foreign particles and bacteria and then are extruded with the eschar (a dead tissue that falls off (sheds) from healthy skin or is phagocytosed by macrophages). In response to specific chemoattractants, such as fragments of extracellular-matrix protein, transforming growth factor β (TGF-β), and monocyte chemoattractant protein-1 (MCP-1), monocytes also infiltrate the wound site and become activated macrophages that release growth factors (such as platelet-derived growth factor and vascular endothelial growth factor), which initiate the formation of granulation tissue. Macrophages bind to specific proteins of the extracellular matrix by their integrin receptors, an action that stimulates phagocytosis of microorganisms and fragments of extracellular matrix by the macrophages (Brown, E. Phagocytosis, Bioessays, 17:109-117 (1995)). Studies have reported that adherence to the extracellular matrix also stimulates monocytes to undergo metamorphosis into inflammatory or reparative macrophages. These macrophages play an important role in the transition between inflammation and repair (Riches, D., In Clark R., Ed. The molecular and cellular biology of wound repair, 2^{nd} Ed. New York, Plenum Press, pp. 95-141). For example, adherence induces monocytes and macrophages to express Colony-Stimulating Factor-1 (CSF-1), a cytokine necessary for the survival of monocytes and macrophages; Tumor Necrosis Factor-α (TNF-α), a potent inflammatory cytokine; and Platelet-Derived Growth Factor (PDGF), a potent chemoattractant and mitogen for fibroblasts. Other cytokines shown to be expressed by monocytes and macrophages include Transforming Growth Factor (TGF-α), Interleukin-1 (IL-1), Transforming Growth Factor β (TGF-β), and Insulin-like Growth Factor-I (IGF-I) (Rappolee, D. et al., Science, 241, pp. 708-712 (1988)). The monocyte- and macrophage-derived growth factors have been suggested to be necessary for the initiation and propagation of new tissue formation in wounds, because macrophage depleted animals have defective wound repair (Leibovich, S, and Ross, R., Am J Pathol, 78, pp 1-100 (1975)).

Wound healing is a complex biological process that is regulated by numerous growth factors, cytokines, and chemokines MK2 is a major regulator of cytokine and chemokine expression, which can recruit local and circulating immunomodulatory cells at wound sites. A recent study with cultured keratinocytes has shown that depleting MK2 through the use of small interfering RNAs severely impairs the ability of the keratinocytes to produce several cytokines, including Tumor Necrosis Factor (TNF) and Interleukin-8 (IL-8) (Johansen et al., J Immunol, 176:1431-1438, 2006). Similarly, in vivo studies have shown that expression levels of several cytokines and chemokines, such as Interleukin-6 (IL-6), Regulated on Activation Normal T cell Expressed and Secreted (RANTES), Tumor Necrosis Factor-alpha (TNF-α), and Interleukin-1 beta (IL-1β), are significantly reduced in the wounds of MK2 knockout mice. These data suggest that MK2 signaling represents an important biochemical pathway that controls the ability of wound infiltrating immunomodulatory cells to produce cytokines and chemokines

3.2. Epithelialization

Reepithelialization of wounds begins within hours after injury. Epidermal cells from skin appendages, such as hair follicles, quickly remove clotted blood and damaged stroma from the wound space. At the same time, the cells undergo phenotypic alteration that includes retraction of intracellular tonofilaments (Paladini, R. et al., J. Cell Biol, 132, pp. 381-397 (1996)); dissolution of most inter-cellular desmosomes, which provide physical connections between the cells; and formation of peripheral cytoplasmic actin filaments, which allow cell movement and migration (Goliger, J. and Paul, D. Mol Biol Cell, 6, pp. 1491-1501 (1995); Gabbiani, G. et al., J Cell Biol, 76, PP. 561-568 (1978)). Furthermore, epidermal and dermal cells no longer adhere to one another, because of the dissolution of hemidesmosomal links between the epidermis and the basement membrane, which allows the lateral movement of epidermal cells. The expression of integrin receptors on epidermal cells allows them to interact with a variety of extracellular-matrix proteins (e.g., fibronectin and vitronectin) that are interspersed with stromal type I collagen at the margin of the wound and interwoven with the fibrin clot in the wound space (Clark, R., J Invest Dermatol, 94, Suppl, pp. 128S-134S (1990)). The migrating epidermal cells dissect the wound, separating desiccated eschar (a dead tissue that falls off (sheds) from healthy skin) from viable tissue. The path of dissection appears to be determined by the array of integrins that the migrating epidermal cells express on their cell membranes.

The degradation of the extracellular matrix, which is required if the epidermal cells are to migrate between the collagenous dermis and the fibrin eschar, depends on the production of collagenase by epidermal cells (Pilcher, B. et al., J Cell Biol, 137, pp. 1445-1457 (1997)), as well as the activation of plasmin by plasminogen activator produced by the epidermal cells (Bugge, T. et al., Cell, 87, 709-719 (1996)). Plasminogen activator also activates collagenase (matrix metalloproteinase-1) (Mignatti, P. et al., Proteinases and Tissue Remodeling. In Clark, R. Ed. The molecular and cellular biology of wound repair. 2^{nd} Ed. New York, Plenum Press, 427-474 (1996)) and facilitates the degradation of collagen and extracellular-matrix proteins.

One to two days after injury, epidermal cells at the wound margin begin to proliferate behind the actively migrating cells. The stimuli for the migration and proliferation of epidermal cells during reepithelialization have not been determined, but several possibilities have been suggested. The absence of neighbor cells at the margin of the wound (the “free edge” effect) may signal both migration and proliferation of epidermal cells. Local release of growth factors and increased expression of growth-factor receptors may also stimulate these processes. Leading contenders include Epidermal Growth Factor (EGF), Transforming Growth Factor-α (TGF-α), and Keratinocyte Growth Factor (KGF) (Nanney, L. and King, L. Epidermal Growth Factor and Transforming Growth Factor-α. In Clark, R. Ed. The molecular and cellular biology of wound repair. 2^{nd} Ed. New York, Plenum Press, pp. 171-194 (1996); Werner, S. et al., Science, 266, pp. 819-822 (1994); Abraham, J. and Klagsburn, M. Modulation of Wound Repair by Members of the Fiborblast Growth Factor family. In Clark, R. Ed. The molecular and cellular biology of wound repair. 2^{nd} Ed. New York, Plenum Press, pp. 195-248 (1996)). As re-epithelialization ensues, basement-membrane proteins reappear in a very ordered sequence from the margin of the wound inward, in a zipper-like fashion (Clark R. et al., J. Invest Dermatol, 79, pp. 264-269 (1982)). Epidermal cells revert to their normal phenotype, once again firmly attaching to the reestablished basement membrane and underlying dermis.

3.3. Formation of Granulation Tissue

New stroma, often called granulation tissue, begins to invade the wound space approximately four days after injury. Numerous new capillaries endow the new stroma with its granular appearance. Macrophages, fibroblasts, and blood vessels move into the wound space at the same time (Hunt, T. ed. Wound Healing and Wound Infection: Theory and Surgical Practice. New York, Appleton-Century-Crofts (1980)). The macrophages provide a continuing source of growth factors necessary to stimulate fibroplasia and angiogenesis; the fibroblasts produce the new extracellular matrix necessary to support cell ingrowth; and blood vessels carry oxygen and nutrients necessary to sustain cell metabolism.

Growth factors, especially Platelet-Derived Growth Factor-4 (PDGF-4) and Transforming Growth Factor β-1 (TGF-β1) (Roberts, A. and Sporn, M, Transforming Growth Factor-1, In Clark, R. ed. The molecular and cellular biology of wound repair. 2^{nd} Ed. New York, Plenum Press, pp. 275-308 (1996)) in concert with the extracellular-matrix molecules (Gray, A. et al., J Cell Sci, 104, pp. 409-413 (1993); Xu, J. and Clark, R., J Cell Biol, 132, pp. 239-149 (1996)), were shown to stimulate fibroblasts of the tissue around the wound to proliferate, express appropriate integrin receptors, and migrate into the wound space. It was reported that platelet-derived growth factor accelerates the healing of chronic pressure sores (Robson, M. et al., Lancet, 339, pp. 23-25 (1992) and diabetic ulcers (Steed, D., J Vasc Surg, 21, pp. 71-78 (1995)). In some other cases, basic Fibroblast Growth Factor (bFGF) was effective for treating chronic pressure sores (Robson, M. et al., Ann Surg, 216, pp. 401-406 (1992).

The structural molecules of newly formed extracellular matrix, termed the provisional matrix (Clark, R. et al., J. Invest Dermatol, 79, pp. 264-269, 1982), contribute to the formation of granulation tissue by providing a scaffold or conduit for cell migration. These molecules include fibrin, fibronectin, and hyaluronic acid (Greiling, D. and Clark R., J. Cell Sci, 110, pp. 861-870 (1997)). The appearance of fibronectin and the appropriate integrin receptors that bind fibronectin, fibrin, or both on fibroblasts was suggested to be the rate-limiting step in the formation of granulation tissue. While the fibroblasts are responsible for the synthesis, deposition, and remodeling of the extracellular matrix, the extracellular matrix itself can have a positive or negative effect on the ability of fibroblasts to perform these tasks, and to generally interact with their environment (Xu, J. and Clark, R., J Cell Sci, 132, pp. 239-249 (1996); Clark, R. et al., J Cell Sci, 108, pp. 1251-1261).

Cell movement into a blood clot of cross-linked fibrin or into tightly woven extracellular matrix requires an active proteolytic system that can cleave a path for cell migration. A variety of fibroblast-derived enzymes, in addition to serum-derived plasmin, are suggested to be potential candidates for this task, including plasminogen activator, collagenases, gelatinase A, and stromelysin (Mignatti, P. et al., Proteinases and Tissue Remodeling. In Clark, R. Ed. The molecular and cellular biology of wound repair. 2^{nd} Ed. New York, Plenum Press, 427-474 (1996); Vaalamo, M. et al., J Invest Dermatol, 109, pp. 96-101 (1997)). After migrating into wounds, fibroblasts commence the synthesis of extracellular matrix. The provisional extracellular matrix is replaced gradually with a collagenous matrix, perhaps in response to Transforming Growth Factor-β1 (TGF-β1) signaling (Clark, R. et al., J Cell Sci, 108, pp. 1251-1261 (1995); Welch, M. et al., J. Cell Biol, 110, pp. 133-145 (1990))

Once an abundant collagen matrix has been deposited in the wound, the fibroblasts stop producing collagen, and the fibroblast-rich granulation tissue is replaced by a relatively acellular scar. Cells in the wound undergo apoptosis triggered by unknown signals. It was reported that dysregulation of these processes occurs in fibrotic disorders, such as keloid formation, hypertrophic scars, morphea, and scleroderma.

3.4. Neovascularization

The formation of new blood vessels (neovascularization) is necessary to sustain the newly formed granulation tissue. Angiogenesis is a complex process that relies on extracellular matrix in the wound bed as well as migration and mitogenic stimulation of endothelial cells (Madri, J. et al., Angiogenesis in Clark, R. Ed. The molecular and cellular biology of wound repair. 2^{nd} Ed. New York, Plenum Press, pp. 355-371 (1996)). The induction of angiogenesis was initially attributed to acidic or basic Fibroblast Growth Factor. Subsequently, many other molecules have also been found to have angiogenic activity, including vascular endothelial growth factor (VEGF), Transforming Growth Factor-β (TGF-β), angiogenin, angiotropin, angiopoietin-1, and thrombospondin (Folkman, J. and D'Amore, P, Cell, 87, pp. 1153-1155 (1996)).

Low oxygen tension and elevated lactic acid were suggested also to stimulate angiogenesis. These molecules induce angiogenesis by stimulating the production of basic Fibroblast Growth Factor (FGF) and Vascular Endothelial Growth Factor (VEGF) by macrophages and endothelial cells. For example, it was reported that activated epidermal cells of the wound secrete large quantities of Vascular Endothelial cell Growth Factor (VEGF) (Brown, L. et al., J Exp Med, 176, 1375-1379 (1992)).

Basic fibroblast growth factor was hypothesized to set the stage for angiogenesis during the first three days of wound repair, whereas vascular endothelial-cell growth factor is critical for angiogenesis during the formation of granulation tissue on days 4 through 7 (Nissen, N. et al., Am J Pathol, 152, 1445-1552 (1998)).

In addition to angiogenesis factors, it was shown that appropriate extracellular matrix and endothelial receptors for the provisional matrix are necessary for angiogenesis. Proliferating microvascular endothelial cells adjacent to and within wounds transiently deposit increased amounts of fibronectin within the vessel wall (Clark, R. et al., J. Exp Med, 156, 646-651 (1982)). Since angiogenesis requires the expression of functional fibronectin receptors by endothelial cells (Brooks, P. et al., Science, 264, 569-571 (1994)), it was suggested that perivascular fibronectin acts as a conduit for the movement of endothelial cells into the wound. In addition, protease expression and activity were shown to also be necessary for angiogenesis (Pintucci, G. et al., Semin Thromb Hemost, 22, 517-524 (1996)).

The series of events leading to angiogenesis has been proposed as follows. Injury causes destruction of tissue and hypoxia. Angiogenesis factors, such as acidic and basic Fibroblast Growth Factor (FGF), are released immediately from macrophages after cell disruption, and the production of vascular endothelial-cell growth factor by epidermal cells is stimulated by hypoxia. Proteolytic enzymes released into the connective tissue degrade extracellular-matrix proteins. Fragments of these proteins recruit peripheral-blood monocytes to the site of injury, where they become activated macrophages and release angiogenesis factors. Certain macrophage angiogenesis factors, such as basic fibroblast growth factor (bFGF), stimulate endothelial cells to release plasminogen activator and procollagenase. Plasminogen activator converts plasminogen to plasmin and procollagenase to active collagenase, and in concert these two proteases digest basement membranes. The fragmentation of the basement membrane allows endothelial cells stimulated by angiogenesis factors to migrate and form new blood vessels at the injured site. Once the wound is filled with new granulation tissue, angiogenesis ceases and many of the new blood vessels disintegrate as a result of apoptosis (Ilan, N. et al., J Cell Sci, 111, 3621-3631 (1998)). This programmed cell death has been suggested to be regulated by a variety of matrix molecules, such as thrombospondins 1 and 2, and anti-angiogenesis factors, such as angiostatin, endostatin, and angiopoietin 2 (Folkman, J., Angiogenesis and angiogenesis inhibition: an overview, EXS, 79, 1-8, (1997)).

3.5. Wound Contraction and Extracellular Matrix Reorganization

Wound contraction involves a complex and orchestrated interaction of cells, extracellular matrix, and cytokines During the second week of healing, fibroblasts assume a myofibroblast phenotype characterized by large bundles of actin-containing microfilaments disposed along the cytoplasmic face of the plasma membrane of the cells and by cell-cell and cell-matrix linkages (Welch, M. et al., J Cell Biol, 110, 133-145 (1990); Desmouliere, A. and Gabbiani, G. The role of the myofibroblast in wound healing and fibrocontractive diseases. In Clark, R. Ed. The molecular and cellular biology of wound repair. 2^{nd} Ed. New York, Plenum Press, pp. 391-423 (1996)). The appearance of the myofibroblasts corresponds to the commencement of connective-tissue compaction and the contraction of the wound. This contraction was suggested to require stimulation by Transforming Growth Factor (TGF)-β1 or β2 and Platelet-Derived Growth Factor (PDGF), attachment of fibroblasts to the collagen matrix through integrin receptors, and cross-links between individual bundles of collagen. (Montesano, R. and Orci, Proc Natl Acad Sci USA, 85, 4894-4897 (1988); Clark, R. et al., J Clin Invest, 84, 1036-1040 (1989); Schiro, J. et al., Cell, 67, 403-410 (1991); Woodley, D. et al., J Invest Dermatol, 97, 580-585 (1991)).

Collagen remodeling during the transition from granulation tissue to scar is dependent on continued synthesis and catabolism of collagen at a low rate. The degradation of collagen in the wound is controlled by several proteolytic enzymes, termed matrix metalloproteinases (MMP), which are secreted by macrophages, epidermal cells, and endothelial cells, as well as fibroblasts (Mignatti, P. et al., Proteinases and Tissue Remodeling. In Clark, R. Ed. The molecular and cellular biology of wound repair. 2^{nd} Ed. New York, Plenum Press, 427-474 (1996)). Various phases of wound repair have been suggested to rely on distinct combinations of matrix metalloproteinases and tissue inhibitors of metalloproteinases (Madlener, M. et al, Exp Cell Res, 242, 201-210 (1998)).

Wounds gain only about 20 percent of their final strength in the first three weeks, during which fibrillar collagen has accumulated relatively rapidly and has been remodeled by contraction of the wound. Thereafter, the rate at which wounds gain tensile strength is slow, reflecting a much slower rate of accumulation of collagen and collagen remodeling with the formation of larger collagen bundles and an increase in the number of intermolecular cross-links. Nevertheless, it was suggested that wounds never attain the same breaking strength (the tension at which skin breaks) as uninjured skin, and that, at maximal strength, a scar is only 70 percent as strong as normal skin (Levenson, S. et al., Ann Surg, 161. 293-308 (1965)).

### 4. Wound Closure Techniques

Wound closure techniques have evolved from the earliest development of suturing materials to include such resources as synthetic sutures, absorbables, staples, tapes, and adhesive compounds. The engineering of sutures in synthetic material along with standardization of traditional materials (e.g., catgut, silk) has made for superior aesthetic results. Similarly, the creation of natural glues, surgical staples, tapes, and more recently, the cyanoacrylate tissue adhesives to substitute for sutures has supplemented the armamentarium of wound closure techniques. The cyanoacrylate tissue adhesives are liquid monomers that polymerize on contact with tissue surfaces in an exothermic reaction creating a strong yet flexible film that bonds the apposed wound edges.

Surgical wound closure facilitates the biological event of healing by joining the wound edges and directly apposes the tissue layers, which serves to minimize new tissue formation within the wound. However, remodeling of the wound occurs, and tensile strength is achieved between the newly apposed edges. Closure can serve both functional and aesthetic purposes, which include elimination of dead space by approximating the subcutaneous tissues, minimization of scar formation by careful epidermal alignment, and avoidance of a depressed scar by precise eversion of skin edges. If dead space is limited with opposed wound edges, then new tissue has limited room for growth. Correspondingly, atraumatic handling of tissues combined with avoidance of tight closures and undue tension contribute to a better result.

Sutures

A general classification of sutures includes natural and synthetic materials, absorbable and nonabsorbable materials, and monofilament and multifilament materials.

Natural materials are more traditional and are still used in suturing today. Examples of natural materials include gut, silk, and even cotton. Gut is absorbable, but cotton and silk are not. Gut is considered a monofilament, whereas silk and cotton are braided multifilaments.

Synthetic materials cause less reaction, and the resultant inflammatory reaction around the suture material is minimized. Various absorbable and nonabsorbable synthetic materials are available for suturing.

Absorbable sutures are applicable to a wound that heals quickly and needs minimal temporary support and are used for alleviating tension on wound edges. The newer synthetic absorbable sutures were shown to retain their strength until the absorption process starts. Examples of absorbable sutures include the monofilamentous Monocryl® (poliglecaprone), Maxon® (polyglycolide-trimethylene carbonate), and PDS® (polydioxanone). Braided absorbable sutures include Vicryl® (polyglactin), and Dexon® (polyglycolic acid).

Nonabsorbable sutures offer longer mechanical support, compared to absorbable suture materials, which lose their tensile strength before complete absorption. Gut can last 4-5 days in terms of tensile strength. In the chromic form (i.e., treated in chromic acid salts), gut can last up to 3 weeks. Vicryl® and Dexon® maintain tensile strength for 7-14 days, although complete absorption takes several months. Polytrimethylene Carbonate Sutures (Maxon®) and Polydioxanone (PDS®) are considered long-term absorbable sutures, lasting several weeks and likewise requiring several months for complete absorption. Nonabsorbable sutures have varying tensile strengths and may be subject to some degree of degradation. Silk has the lowest strength and nylon has the highest, although Prolene® is comparable. Both nylon and Prolene® require extra throws to secure knots in place. Polyester has a high degree of tensile strength, and Novafil® is appreciated for its elastic properties. Nonabsorbable sutures comprise nylon, Prolene® (polypropylene), Novafil® (polybutester), PTFE (polytetrafluoroethylene), steel, and polyester. Nylon and steel sutures can be monofilaments or multifilaments. Prolene®, Novafil®, and PTFE are monofilaments. Polyester suture is braided.

Monofilaments (single strand of suture material) have less drag through the tissues but are susceptible to instrumentation damage. Infection is avoided with the monofilament, unlike the braided multifilament, which can potentially sustain bacterial inocula.

Adhesives

Problems (e.g., reactivity, premature reabsorption) can occur with sutures and lead to an undesirable result, both cosmetically and functionally. Use of surgical adhesives can simplify skin closure. Several adhesives have been developed to alleviate this problem and to facilitate wound closure. One substance, cyanoacrylate, easily forms a strong flexible bond.

Octyl-2-cyanoacrylate (Dermabond®, Ethicon, Somerville, N.J.) is the only cyanoacrylate tissue adhesive approved by the U.S. Food and Drug Administration (FDA) for superficial skin closure. Octyl-2-cyanoacrylate should only be used for superficial skin closure and should not be implanted subcutaneously. Subcutaneous sutures are used to take the tension off the skin edges prior to applying the octyl-2-cyanoacrylate. Subcutaneous suture placement aids in averting the skin edges and minimizing the chances of deposition of cyanoacrylate into the subcutaneous tissues.

Fibrin-based tissue adhesives can be created from autologous sources or pooled blood. They are typically used for hemostasis and can seal tissues. Although they do not have adequate tensile strength to close skin, fibrin tissue adhesives can be used to fixate skin grafts or seal cerebrospinal fluid leaks. Commercial preparations such as Tisseel® (Baxter) and Hemaseel® (Haemacure) are FDA-approved fibrin tissue adhesives made from pooled blood sources. These fibrin tissue adhesives are relatively strong and can be used to fixate tissues. Autologous forms of fibrin tissue adhesives can be made from patient's plasma. The concentration of fibrinogen in the autologous preparations is less than the pooled forms; therefore, these forms have a lower tensile strength.

Other Materials

Staples provide a fast method for wound closure and have been associated with decreased wound infection rates. Staples are composed of stainless steel, which has been shown to be less reactive than traditional suturing material. The act of stapling requires minimal skin penetration, and, thus, fewer microorganisms are carried into the lower skin layers. Staples are more expensive than traditional sutures and also require great care in placement, especially in ensuring the eversion of wound edges. However, with proper placement, resultant scar formation is cosmetically equivalent to that of other techniques.

Closure using adhesive tapes or strips was first described in France in the 1500s. This method allowed the wound edges to be joined and splinted. The porous paper tapes (e.g., Steri-Strips®) in use today are reminiscent of these earlier splints and are used to ensure proper wound apposition and to provide additional suture reinforcement. These tapes can be used either with sutures or alone. Often, skin adhesives (e.g., Mastisol®, tincture of Benzoin) aid in tape adherence.

Newer products, such as the ClozeX® (Wellesley, Mass.) adhesive strip, allow for rapid and effective wound closure that results in adequate cosmesis. Additionally, wound closure with adhesive strips can be significantly cheaper than suturing or using a tissue adhesive. However, adhesive strips are not appropriate for many types of lacerations.

### 5. Cutaneous Scar

A scar is a fibrous tissue that replaces normal tissues destroyed by injury or disease. Damage to the outer layer of skin is healed by rebuilding the tissue, and in these instances, scarring is slight. When the thick layer of tissue beneath the skin is damaged, however, rebuilding is more complicated. The body lays down collagen fibers (a protein which is naturally produced by the body), and this usually results in a noticeable scar. After the wound has healed, the scar continues to alter as new collagen is formed and the blood vessels return to normal, allowing most scars to fade and improve in appearance over the two years following an injury. However, there is some visible evidence of the injury, and hair follicles and sweat glands do not grow back. A wound does not become a scar until the skin has healed completely. Skin conditions such as eczema and psoriasis and injuries, such as minor burns or sunburn, are not scars because the skin is broken or still being repaired. However, these conditions could lead to a minor scar if scratched before the outer layer of skin is healed.

A cutaneous scar is a dermal fibrous replacement tissue, which results from a wound that had healed by resolution rather than regeneration. Final appearance is influenced largely by the interval between wounding and complete healing 2 to 3 weeks later. Once the scar has formed, it undergoes several distinct macro- and microscopic changes during the maturation process and is completed on average after one year (Bond, J. et al., Plastic and Reconstructive Surgery, vol. 121, No. 5, pp. 1650-1658, (2008)). Patients under 30 years exhibit a slower rate of scar maturation and poorer final appearance than patients over 55 years. The redness of a scar fades after 7 months and, in contrast with rubor (redness) elsewhere, does not reflect an inflammatory process (after the first month) (Bond J. et al, Plastic and Reconstructive Surgery, vol. 121, no. 2., pp. 487-496, 2008). The scar is devoid of dermal appendages and never reaches the same tensile strength as the surrounding skin (Beanes, S. et al., Expert Reviews in Molecular Medicine, vol. 5, no. 8, pp. 1-22, (2003)).

Scar tissue consists mainly of disorganized collagenous extracellular matrix. This is produced by myofibroblasts, which differentiate from dermal fibroblasts in response to wounding, which causes a rise in the local concentration of Transforming Growth Factor-β, a secreted protein that exists in at least three isoforms called TGF-β1, TGF-β2 and TGF-β3 (referred to collectively as TGF-β). TGF-β is an important cytokine associated with fibrosis in many tissue types (Beanes, S. et al., Expert Reviews in Molecular Medicine, vol. 5, no. 8, pp. 1-22 (2003)).

Myofibroblasts are characterized by the presence of a contractile apparatus that contains bundles of actin microfilaments with associated contractile proteins, such as non-muscle myosin, which is analogous to stress fibers that have been described in cultured fibroblasts. These actin bundles terminate at the myoblast surface in the fibronexus, a specialized adhesion complex that uses transmembrane integrins to link intracellular actin with extracellular fibronectin fibrils. Most myofibroblasts express Alpha-Actin-2 (ACTA2; also known as alpha-smooth muscle actin or α-SMA), and the expression of ACTA2 and collagen type I in myofibroblasts is coordinated and regulated by Transforming Growth Factor-β1 (TGF-β1) (Tomasek, J. et al., Nat. Rev. Mol. Cell. Biol., 3: 349-363). Additionally, previous studies have shown that integrins play an important role in TGF-β-induced myofibroblast differentiation (Lygoe, K. et al., Wound Repair Regen, 12(4):461-470, 2004). ACTA2, TGF-β, and Integrins are currently the principal targets to suppress scarring (Beausang, E. et al., Plastic and Reconstructive Surgery, vol. 102, no. 6, pp. 1954-1961 (1998); Niessen, F. et al., Plastic and Reconstructive Surgery, vol. 102, no. 6, pp. 1962-1972 (1998)).

### 6. Types of Cutaneous Scars

Skin tissue repair results in a broad spectrum of scar types, ranging from a “normal” fine line to a variety of abnormal scars, including wide spread scars, atrophic scars, scar contractures, hypertrophic scars, and keloid scars.

6.1. Wide Spread (Stretched) Scars

Wide spread (stretched) scars appear when the fine lines of surgical scars gradually become stretched and widened, which usually happens in the three weeks after surgery. They are typically flat, pale, soft, symptomless scars often seen after knee or shoulder surgery. Stretch marks (abdominal striae) after pregnancy are variants of widespread scars in which there has been injury to the dermis and subcutaneous tissues but the epidermis is unbreached. There is no elevation, thickening, or nodularity in mature wide-spread scars, which distinguishes them from hypertrophic scars.

6.2. Atrophic Scars

Atrophic scars are flat and depressed below the surrounding skin. They are generally small and often round with an indented or inverted center. Atrophic scarring can be a result of surgery, trauma, and such common conditions as acne vulgaris and varicellar (chickenpox).

6.3. Scar Contractures

Scars that cross joints or skin creases at right angles are prone to develop shortening or contracture. Scar contractures occur when the scar is not fully matured, often tend to be hypertrophic, and are typically disabling and dysfunctional. They are common after burn injury across joints or skin concavities.

6.4. Pathological Scars

Pathological scars are thought to be caused by disordered regulation of wound cellularity and collagen synthesis (M. Sharad, Indian Journal of Dermatology, Venereology and Leprology, vol. 71, no. 1, pp. 3-8, 2005). Pathological scars are hyper-responsive to Transforming Growth Factor-beta1 (TGF-β1); connective tissue growth factor (CTGF) expression increases 150-fold and 100-fold in hypertrophic and keloid scars, respectively, in response to TGF-β1 compared with normal fibroblasts (Colwell, A et al., Plastic and Reconstructive Surgery, vol. 116, no. 5, pp. 1387-1390 (2005)). Failure of apoptosis also plays a role. Keloid fibroblasts in particular are highly resistant to fatty acid synthase-mediated apoptosis and the tumor suppressor genes, p53 and p63, which are involved in the induction of apoptosis (Nedelec, B. et al., Surgery, vol. 130, no. 5, pp. 798-808 (2001); Chodon, T. et al., American Journal of Pathology, vol. 157, no. 5, pp. 1661-1669 (2000); Tanaka, A. et al., Journal of Dermatological Science, vol. 34, no. 1, pp. 17-24, (2004); De Felice, B. et al., Molecular Genetics and Genomics, vol. 272, no. 1, pp. 28-34 (2004)). In addition, there are also predisposing systemic traits. Burn patients who subsequently develop hypertrophic scars have higher Interleukin-10 (IL-10), TGF-β1 serum levels, and elevated numbers of Interleukin-4 (IL-4)-positive Th2 cells early after burn injury, compared with those that develop normal scars (Tredget, E. et al., Journal of Interferon and Cytokine Research, vol. 26, no. 3, pp. 179-189 (2006)). Familial clustering and the markedly higher predisposition of patients of Afro-Carribean origin to developing keloids has suggested that there is a major genetic contribution with keloid susceptibility loci having been found on chromosomes 2q23 and 7p11 (Bayat, A. et al, British Journal of Plastic Surgery, vol. 58, no. 7, pp. 914-921 (2005); Marneros, A. et al., Journal of Investigative Dermatology, vol. 122, no. 5, pp. 1126-1132 (2004)).

Hypertrophic Scars (Red or Dark and Raised)

Hypertrophic scars are raised scars that remain within the boundaries of the original lesion, generally regressing spontaneously after the initial injury. Hypertrophic scars are hard, raised, red, itchy, tender, and contracted. They typically occur after burn injury on the trunk and extremities. Clinically and histologically, hypertrophic scars and keloid scars are very similar but, unlike keloids, hypertrophic scars enlarge by pushing on the scar's boundaries, whereas keloids invade the surrounding tissue. Hypertrophic scars mature and flatten over time. Keloids usually persist indefinitely without treatment.

Hypertrophic scars show the same whorled, hyalinized bundles of collagen as keloids, and are more vascular and cellular than normal scars. Two types of fibroblasts appear in hypertrophic scars. One is noncycling and does not proliferate; the other type, present in smaller numbers, rapidly proliferates and displays active synthesis.

Keloid Scars (Red or Dark and Raised)

Keloid scars are benign fibrous proliferations in the dermis that arise after dermal trauma. They are raised above the surface of the skin and extend beyond the boundaries of the original wound. These scars are permanent and do not regress with the passage of time. Keloids are often cosmetically disfiguring and can be painful. The extent of scarring is not directly proportional to the severity of the original wound (Datubo-Brown, D., Br J Plast Surg, 43:70-77, (1990); Murray, J. Demartol Clin, 11:697-708 (1993)). Excessive scarring in keloid tissue is related to exuberant deposition and insufficient degradation of collagen and other extracellular proteins, including, chondroitin-4-sulfate (C4S), fibronectin, and elastin. Histologically, keloid tissue is distinctive because of the chaotic orientation of collagen fibers. The individual collagen fibers are thickened, hyalinized, and highly eosinophilic. The fibers are arranged usually in nodules or “whorls.” (Murray, J. Demartol Clin, 11:697-708 (1993)). The etiology of keloid formation remains poorly understood. The wound healing sequence does not differ markedly from that seen in normal scars. The main distinction between keloids and normal scars lies in the degree of fibroplasia, the amount of intercellular ground substance, and the time frame of active cellular metabolism.

Keloids may be inflamed, itchy, and painful, especially during their growth phase. Common presentations are in the ear lobe after ear piercing, the deltoid after vaccination, the sternum after acne, chickenpox, trauma, or surgery. It was reported that some people are predisposed genetically to Keloids, with dark skinned races being more prone to them, though there are few large epidemiological studies.

Intermediate Scars

Scars that are difficult to categorize have been termed intermediate scars. However, if a raised scar is still emerging after a year, a true keloid is a potential diagnosis, whereas hypertrophic scars should show some evidence of regression within this time.

### 7. Mechanisms of Pathological Scarring

7.1. Role of Myofibroblasts

Various cytokines and growth factors have been studied for their role in wound healing. TGF-β1, a potent inducer of myofibroblastic differentiation, acts directly on granulation tissue formation and fibrogenic cell activation. In addition to its specific induction of Alpha-Actin-2 (ACTA2) expression, TGF-β promotes extracellular matrix (ECM) deposition. TGF-β1 not only induces the synthesis of ECM (particularly fibrillar collagens and fibronectin) but also reduces metalloproteinase (MMP) activity by promoting Tissue Inhibitors of Metalloproteinase (TIMP) expression. The effect of TGF-β1 on myofibroblastic differentiation requires ED-A fibronectin, illustrating the important role of ECM components in the activity of soluble mediators. It was shown recently that ED-A fibronectin induces lung fibroblast differentiation by binding to the α4β7 integrin receptor and by MAPK/Erk 1/2-dependent signaling; however, some other studies have shown that this integrin is not expressed by dermal fibroblast suggesting that specific mechanisms are involved in the different fibroblast populations.

7.2. Role of Mechanical Stress

The activity of myofibroblastic cells depends on the mechanical environment, which is modulated by these cells' contractile properties and their intimate relationship with the extracellular matrix (ECM). Features of myofibroblastic differentiation, such as stress fibers, ED-A fibronectin, and ACTA2 expression, appear earlier in granulation tissue subjected to increased mechanical tension exerted by splinting of a full-thickness wound with a plastic frame. Likewise, fibroblasts cultured on substrates of variable stiffness adopt different phenotypes, soft surfaces being associated with a lack of stress fibers. Moreover, shear forces exerted by fluid flow can induce Transforming Growth Factor-β1 (TGF-β1) production and differentiation of fibroblasts cultured in collagen gels, in the absence of other external stimuli, such as cytokine treatment. All these processes were shown to involve a dialogue between epidermal cells and connective cells, which determines the normal or the pathological nature of tissue repair.

7.3. Myofibroblast Origins

Myofibroblasts can originate from various cell types, including, but not limited to, locally recruited connective tissue fibroblasts. Marked phenotypic heterogeneity of fibroblastic cells has been observed in connective tissue. Different subpopulations reside in different locations within the organ and exhibit specific activation and deactivation properties. At least three subpopulations have been identified in the dermis, namely superficial dermal fibroblasts, reticular fibroblasts (which reside in the deep dermis), and fibroblasts associated with hair follicles.

These subpopulations exhibit marked differences when cultured separately. Pericytes have also been implicated in both normal and pathological tissue repair. In diffuse cutaneous systemic sclerosis, microvascular pericytes represent a link between microvascular damage and fibrosis by transdifferentiating into myofibroblasts. Endothelical cells (ECs) were identified recently as a possible source of tumoral (myo) fibroblasts. Many studies suggested that epithelial-mesenchymal transdifferentiation of nonmalignant epithelial or epithelial-derived carcinoma cells is a major source of fibrosis- and tumor-associated myofibroblasts. Moreover, local mesenchymal stem cells are likely involved in tissue repair. These mesenchymal stem cells have been described in the dermal sheath that surrounds the hair follicle facing epithelial stem cells. They are involved in dermal papilla regeneration and can become myofibroblasts in response to insults. Foci containing both epithelial stem cells and mesenchymal stem cells may constitute a cooperative niche. Recent studies suggested that mesenchymal stem cells from subcutaneous fat are responsible for collagen accumulation in scars. Bone marrow-derived mesenchymal stem cells that are nonhematopoietic precursor cells also were shown to contribute to the maintenance and regeneration of connective tissues through engraftment and differentiation into wound-healing myofibroblasts. Engraftment in injured organs is modulated by the severity of the damage. Intravenously administered mesenchymal stem cells, however, show very poor engraftment in healthy organs.

Studies have shown that circulating cells called fibrocytes also are involved in the tissue repair process. Fibrocytes enter damaged skin along with inflammatory cells and acquire a myofibroblastic phenotype. Fibrocytes are recruited to sites of burn injury, where they stimulate the local inflammatory response and produce extracellular matrix proteins, thus contributing to hypertrophic scar (HS) formation. Pericytes, endothelial cells, epithelial cells, local mesenchymal stem cells, bone marrow-derived mesenchymal stem cells, and fibrocytes may represent alternative sources of myofibroblasts when local resources are overwhelmed, particularly after severe acute insult (e.g., extensive burns) or in chronic situations such as fibrosis. These diverse cell types were suggested to generate myofibroblast subpopulations whose phenotype can be modulated by their interactions with neighboring cells and extracellular matrix.

7.4. Hypertrophic Scars and Keloids

Abnormal wound repair results from impaired granulation tissue remodeling, leading, for example, to hypertrophic or keloid scars. Contrary to hypertrophic scars, keloid scars do not contain ACTA2, possibly owing to the presence of protomyofibroblasts that can deposit large amounts of extracellular matrix (ECM) but are not able to develop enough forces to contract the lesion. Numerous myofibroblasts express ACTA2 in hypertrophic scars, explaining why contractures are observed only with hypertrophic scars but not keloids. However, the use of ACTA2 to distinguish hypertrophic scars and keloids has been rediscussed recently, suggesting that this protein can be expressed in both pathological situations. Keloids contain thick collagen fibers, whereas hypertrophic scars contain thin fibers organized into nodules. Thus, collagen maturation and the MMP/TIMP system play an important role in excessive scar formation. For example, the expression of lysyl hydroxylase (LH)-2b, a splice variant of LH-2, an enzyme involved in collagen fibril cross-linking, has been linked to pathological fibrosis.

In hypertrophic scars and keloids, the granulation tissue continues to grow, due to the excessive secretion of growth factors and/or a lack of molecules required for apoptosis or extracellular matrix remodeling. Hypertrophic scars contain an excess of microvessels, most of which are partially or totally occluded due to overproliferation and the functional regression of endothelial cells induced by (myo)fibroblast hyperactivity and excessive collagen production. Focal up-regulation of p53 expression, which inhibits apoptosis, has been observed in situations of excessive scarring. For example, it has been suggested that mechanical loading early in the proliferative phase of wound healing produces hypertrophic scars by inhibiting apoptosis through an Akt-dependent mechanism.

Extracellular matrix changes also seem to be important in the apoptotic process: in vivo, covering granulation tissue with a vascularized skin flap induces metalloproteinase up-regulation and a decline in Tissue Inhibitors of Metalloproteinases (TIMPs), leading to rapid loss of granulation tissue cells by apoptosis. The matrix environment can also modulate fibroblast apoptosis in vitro. Hic-5, a focal adhesion protein that is upregulated by TGF-β1, is an essential component of the mechanisms regulating autocrine TGF-β1 production and resulting in a pathogenic myofibroblast phenotype. Furthermore, mechanical compression of hypertrophic scars can restore the organization observed in normal scar tissue and trigger myofibroblast apoptosis. The epithelium may also be involved in excessive scarring. For example, a study has shown that, in hypertrophic scars, keratinocytes express an activated CD36-positive phenotype (the expression of CD36 in normal keratinocytes is absent, occurring only in response to specific stimuli). It was suggested that hypertrophic scar formation is not only due to dermis dysfunction but results from perturbation of dermal-epidermal interactions involving neurohormonal factors. Mechanical stress stimulates mechanosensitive nociceptors in skin sensory fibers that release neuropeptides involved in vessel modifications and fibroblast activation. It was shown recently that occlusive therapy reduces dermal fibrosis by hydrating the epidermis and altering the pro- and antifibrotic signals produced following injury.

### 8. Mechanobiology of Scarring

During the growth and development of the human body, the skin expands to cover the growing skeleton and soft tissues and it is subjected constantly to extrinsic and intrinsic mechanical forces. These extrinsic forces include skin-stretching tensions (e.g., due to body movement) and external stimuli (e.g., scratch). Intrinsic forces include extracellular matrix (ECM) tension by the underlying skeletal growth, and fluid shear force and hydrostatic and osmotic pressures by the extracellular fluid (ECF). Following skin injury, the mechanophysiological conditions are changed by wound healing, granulation tissue formation, wound contraction, and epithelialization. Coagulation and inflammation cause edema and blood circulatory alterations in the skin and wound, thereby impacting the ECF-based mechanophysiology. Moreover, the proliferative and remodeling phases, which start within 1 week of injury and can continue for months, cause granulation tissue formation and wound contraction by myofibroblast activity. These mechanophysiological alterations of the injured skin considerably influence the degree of scarring (Ogawa, R., Wound Rep Reg, 19, S2-S9, 2011).

8.1. Cellular and Tissue Responses to Mechanical Forces on Cutaneous Wounds

Mechanical forces, including stretching tension, shear force, scratch, compression, and hydrostatic and osmotic pressures, can be perceived by cellular mechanoreceptors/mechanosensors and/or nerve fiber receptors (including mechanosensitive (MS) nociceptors) that produce the somatic sensation of mechanical force. Cellular mechanoreceptors include the mechanosensitive ion channels (e.g., Ca²⁺, K⁺, Na⁺, and Mg²⁺), cytoskeleton (e.g., actin filaments), and cell adhesion molecules (CAMs) (e.g., integrins). Skin resident cells are attached to the extracellular matrix via cell adhesion molecules, and the cytoskeleton is connected to mechanosensitive ion channels and cell adhesion molecules. When the extracellular matrix is distorted by mechanical forces such as skin tension, the cytoskeleton is altered and mechanosensitive ion channels are activated. In contrast, extracellular fluid (ECF)-based pressure cannot activate mechanosensitive ion channels through cytoskeletal alteration, as hydrostatic pressure impacts ion inflow but not cell shape. Cells convert mechanical stimuli into electrical signals through mechanoreceptors, thereby accelerating cell proliferation, angiogenesis, and epithelialization through various mechanotransduction pathways. In particular, transforming growth factor (TGF-β)/Smad, integrin, mitogen-activated protein kinase G protein, Tumor Necrosis Factor(TNF)/NF-kB, Wnt/β-catenin, interleukin, and calcium ion pathways have been the subject of extensive research in cutaneous scarring. TGF-β is involved in the way scar tissue reacts to mechanical forces.

For example, keloid-derived fibroblasts subjected to mechanical force in the form of equibiaxial strain have shown to produce more TGF-β1 and -β2 than normal skin-derived fibroblasts. Another study has shown that stretching a myofibroblast-derived extracellular matrix in the presence of mechanically apposing stress fibers immediately activates latent TGF-β1, compared with relaxed tissues; and that the stressed tissues exhibit increased activation of Smad2/3, which are the downstream targets of TGF-β1 signaling.

G proteins are additional membrane proteins that modulate mechanotransduction pathways. Mechanical stimulation alters the G protein conformation, leading to growth factor-like changes that initiate secondary messenger cascades and initiate cell growth. Calcium ion mechanosensitive channels are involved in phospholipase C activation, which can lead to protein kinase C activation and subsequent epidermal growth factor (EGF) activation. These mechanotransduction pathways are suggested to be associated with cutaneous scarring as a cellular response.

At the tissue level, sensory fibers act as mechanical stimuli receptors in the skin. Mechanical stimuli are received by mechanosensitive nociceptors, and signals are transmitted to dorsal root ganglia that contain neuronal cell bodies in the afferent spinal nerves. This results in neuropeptide release from the peripheral terminals of primary afferent sensory neurons, which innervate the skin and often contact epidermal and dermal cells. These neuropeptides can directly modulate the functions of keratinocytes, fibroblasts, Langerhans cells, mast cells, dermal microvascular endothelial cells, and infiltrating immune cells. Substance P (SP), calcitonin gene-based peptide (CGRP), neurokinin A, vasoactive intestinal peptide, and somatostatin are neuropeptides that effectively modulate skin and immune cell functions, including cell proliferation, cytokine production, antigen presentation, sensory neurotransmission, mast cell degradation, and vasodilation, and increase vascular permeability under physiological or pathophysiological conditions.

These proinflammatory responses are termed neurogenic inflammation. Substance P and calcitonin gene-based peptide (CGRP) act through the neurokinin 1 receptor and CGRP1 receptor, respectively, and are synthesized during nerve growth factor (NGF) regulation. Some have also suggested a relationship between burn and abnormal scars (e.g., keloids and hypertrophic scars) and neurogenic inflammation/neuropeptide activities.

8.2. Clinical Evidence of the Relationship between Mechanical Forces and Scarring

While appropriate amounts of intrinsic tension are necessary for wound closure, extrinsic mechanical force also contributes to scarring after wounding. Studies have shown that mechanical forces promote the growth of fibroproliferative skin disorders such as hypertrophic scars and keloids. Therefore, striking the balance of these forces is important to prevent scar production.

Keloids and hypertrophic scars may constitute two stages of a continuous disease, with only the chronic inflammation strength being different between them. Although distinguishing between a keloid and a hypertrophic scar remains imprecise, with respect to hyalinizing collagen bundle formation, the inflammation of a keloid was shown to be much greater than that of a hypertrophic scar, and the inflammation of either was shown to be greater than that of a mature scar. The inflammation strength reflects the degree of angiogenesis in and around the scar, including the redness of the scar itself and of the skin adjacent to the scar. Keloids display scar and adjacent skin redness; in contrast, redness on adjacent skin is not observed in hypertrophic scars. It has been suggested that these inflammatory features are closely related to the mechanical force sensitivity, although many other chronic inflammation triggers may be involved.

Hypertrophic scars can occur anywhere in the body, especially when a scar is long, wide, and located on a frequently moved joint. Long and wide scars can produce an imbalance of the skin stretching forces on adjacent scars and can sometimes cause scar contracture. Plastic surgeons divide scars and release contractures using geometrical plasties (e.g., z- and w-plasties) and small-wave incisions for scar and scar contracture treatments. In contrast, heavy scars rarely occur on the scalp or the anterior lower leg. Even in patients with keloids or hypertrophic scars covering the entire body, heavy scars on the scalp or the anterior lower leg are rare. The commonality in these sites is that the bones lie directly under the skin; consequently, the skin at these sites is rarely subjected to tension. Based on the site specificity of scar development, it has been suggested that mechanical forces may not only promote keloid/hypertrophic scars growth, but may also be a primary trigger for their generation.

8.3. Relationship Between Scar Growth and the Direction of the Stretching Tension

Hypertrophic scars do not grow beyond the boundaries of the original wound, and thus only grow vertically. In contrast, keloids grow and spread both vertically and horizontally, similar in many respects to slowly growing malignant tumors. The direction of their horizontal growth results in characteristic shapes that depend on their location. For example, keloids on the anterior chest grow in a “crab's claw”-like pattern, whereas shoulder keloids grow in a “butterfly” shape. These patterns reflect the predominant directions of skin tension at these sites. Finite element analysis of the mechanical force distribution around keloids revealed high skin tension at the keloid edges and lower tension at the keloid centers. The result indicates why keloids generally stop growing in their central regions. Keloid expansion occurred in the direction of skin pulling, and the skin stiffness at the keloid circumference directly correlated with the degree of skin tension. These observations suggested that skin tension is closely associated with the pattern and degree of keloid growth, and that the differences in growth pattern between hypertrophic scars and normal scars of those of keloids may reflect differences in their responsiveness to skin tension.

### 9. Clinical Mechanobiology Strategies for Scar Prevention and Treatment

To limit skin stretching and external mechanical stimuli during wound healing/scarring, wounds or scars a wound should be covered by fixable materials, such as tape, bandages, garments, or silicone gel sheets. A randomized-controlled trial (RCT) showed that tape fixation helped to prevent hypertrophic scar formation after a cesarean section in 70 subjects, with significantly less scar volume when paper tape was used. Other RCTs have shown that silicone gel sheeting significantly reduces the incidence of hypertrophic scars or keloids. It was shown also that silicone gel sheeting reduces tension at the scar edges, suggesting an important mechanism for hypertrophic scar formation.

Fluid control may also help prevent and treat scars by inducing hydrostatic pressure gradients and shear forces that alter genomic expression through mechanosensitive ion channels. Therefore, the control of extracellular fluid (ECF)-based mechanical forces (fluid shear forces, hydrostatic pressure, and osmotic pressure) may be achieved through various devices or materials (e.g., vacuum-assisted closure, wound dressings).

Based on the described relationships between scar formation and mechanobiology, several potential scar therapeutic approaches have been suggested. With respect to neurogenic inflammation, neuropeptide blockade using continuous local anesthesia was suggested to be effective for abnormal scar treatment. Peripheral nerve activity, including neuropeptide release, can be controlled via the central nervous system. Mechanoreceptors and neuropeptides can be inhibited, such as through ion channel, integrin, or neuropeptide receptor blockers. For example, calcium channel blockers are already in use for scar treatments, where they have been shown to decrease extracellular matrix formation and inhibit fibroblast and vascular smooth muscle cell proliferation (Ogawa, R., Wound Repair and Regeneration, 19(S1), S2-S9, (2011))

### 10. In Vitro Scratch Wound Healing Assay

The in vitro scratch wound healing assay is a straightforward and economical method to study wound healing in vitro. This method mimics cell migration during wound healing in vivo and is based on the observation that, upon creation of a new artificial gap, a so-called “scratch,” on a confluent cell monolayer, the cells on the edge of the newly created gap will move toward the opening to close the “scratch” until new cell-cell contacts are established again. The basic steps involve creating a “scratch” on monolayer cells, capturing images at the beginning and regular intervals during cell migration to close the scratch, and comparing the images to determine the rate of cell migration (Rodriquez, L. et al., Methods Mol. Biol., 294:23-29, 2005; Liang, C-C et al., Nature Protocols, 2:329-333, 2007).

One of the major advantages of this simple method is that it mimics to some extent migration of cells in vivo. For example, removal of part of the endothelium in the blood vessels will induce migration of endothelial cells (ECs) into the denuded area to close the wound. Furthermore, the patterns of migration either as loosely connected populations (e.g., fibroblasts) or as sheets of cells (e.g., epithelial and ECs) also mimic the behavior of these cells during migration in vivo. Another advantage of the in vitro scratch assay is its particular suitability to study the regulation of cell migration by cell interaction with extracellular matrix (ECM) and cell-cell interactions. In other popular methods, such as Boyden chamber assays, preparation of cells in suspension before the assays disrupts cell-cell and cell-ECM interactions. In addition, the in vitro scratch assay is also compatible with microscopy, including live cell imaging, allowing analysis of intracellular signaling events (e.g., by visualization of green fluorescent protein (GFP)-tagged proteins for subcellular localization or fluorescent resonance energy transfer for protein-protein interactions) during cell migration (Liang, C-C et al., Nature Protocols, 2:329-333, 2007).

The migration path of individual cells in the leading edge of the scratch can be tracked with the aid of time-lapse microscopy and image analysis software. Capturing of an image at the beginning of the experiment with fluorescence microscopy can mark the cells with expression of exogenous gene or downregulation of endogenous genes by RNA interference (e.g., using a GFP marker). By comparing the tracks of these cells with surrounding control cells under the same experimental conditions, determination of the role of a particular gene in the regulation of directional cell migration using the assay is possible (Liang, C-C et al., Nature Protocols, 2:329-333, 2007).

Although developed and more suitable for measuring migration of population of cells, the in vitro scratch assay has also been combined with other techniques, such as microinjection or gene transfection, to assess the effects of expression of exogenous genes on migration of individual cells (Etienne-Manneville, S. et al., Cell, 106, 489-498, 2001; Fukata, Y. et al., J. Cell Biol., 145, 347-361, 1999; Abbi, S. et al., Mol. Biol. Cell., 13:3178-3191, 2002).

### 11. Animal Models of Hypertrophic Scar and Keloid Scarring

Although cell culture can be used to verify the mechanism of action of a new therapy and to establish a safe human dose range, a predictive in vivo model is needed to assess the safety and efficacy of a treatment in humans. Attempts have been made to construct suitable animal models of heavy scars using mice, rats, and rabbits; however, these models, especially for keloids, are driven more by an acute inflammatory response than by chronic inflammation, leading to immature scar formation. A hypertrophic mouse model based on mechanical force loading showed that scars subjected to tension exhibit less apoptosis, and that inflammatory cells and mechanical forces promote fibrosis. These findings suggested that mechanical forces strongly modulate cellular behavior in the scar.

There are several animal models of hypertrophic and keloid scarring: (1) heterologous hypertrophic scarring or keloid implant in immunodeficient animals (athymic mice and rats) (Kischer, C. et al., J Trauma 29:672-677 (1989); Kischer, C. et al., Anat Rec; 225:189-196 (1989)); (2) heterologous hypertrophic scarring or keloid implant in immune privileged site (hamster cheek pouch) (Hochman, B. et al., Acta Cir Bras, 20:200-212 (2005)); (3) hypertrophic scarring or keloid induction via chemically mediated injury (guinea pigs) (Aksoy, M. et al., Aesthetic Plast Surg, 26:388-396 (2002)); (4) hypertrophic scarring or keloid induction in specific anatomic sites (rabbit ear) (Morris, D. et al., Plast Reconstr Surg 100:674-81 (1997); and (5) hypertrophic scarring or keloid induction in deep dermal wounds in a porcine model (Silverstein, P. et al., Ann Res Progress Report of the US Army Institute of Surgical Research (section 37) (1972); Silverstein, P. et al., Hypertrophic scar in the experimental animal. In: The ultrastructure of collagen. Springfield, Ill.: Thomas (1976); Zhu, K. et al., Burns, 29: 649-64 (2003); Zhu, K. et al, Burns, 30:518-30 (2004)).

TABLE 1
Animal Models of Hypertrophic Scarring or Keloid

| Vulgar Name | Genus | Species | Lineage |
|---|---|---|---|
| Rat | Rattus sp. | R. novergicus | Wistar (athymic) |
| Mouse | Mus sp. | M. musculus | Nude (athymic) |
| Hamster | Mesocricetus sp. | M. auratus | |
| Guinea pig | Cavia sp. | M. porcellus | |
| Rabbit | Oryctolagus sp. | O. cuniculus | White New Zealand |
| Pig | Sus sp. | S. scrofa | Duroc |
| | | | Yorkshire |
| | | | Large White |

Scar scales have been devised to quantify scar appearance in response to treatment. There are currently at least five scar scales that were originally designed to assess subjective parameters in an objective way (Table 2): The Vancouver Scar Scale (VSS), Manchester Scar Scale (MSS), Patient and Observer Scar Assessment Scale (POSAS), Visual Analog Scale (VAS), and Stony Brook Scar Evaluation Scale (SBSES). These observer-dependent scales consider factors such as scar height or thickness, pliability, surface area, texture, pigmentation, and vascularity (Nedelec, B. et al. J Burn Care Rehabil. 21:205-12 (2000)). The measurements range across a continuum of values. Thus, the scales are used to determine change within an individual rather than between individuals. Scar scales are used frequently in research settings and are beneficial to study small, linear scars. Scar scales are only minimally useful for studying large scars and for assessing the functional affects of scarring (Fearmonti, R. et al., Eplasty, 10:e43, 2010). It is not unusual for individual studies of disfiguring scars to create their own clinical scar scales in agreement with regulators, as in the Juvista® study by Renovo Group PLC, which used a proprietary Global Scar Comparison Scale after EMA agreement as its primary endpoint, which had the benefit of photographic based assessment amenable to an independent clinical expert consensus panel (Renovo Corporate Presentation, December 2010).

TABLE 2
Comparison of Scar Assessment Scales

| | Scoring | Attributes | | |
|---|---|---|---|---|
| Scale | System | analyzed | Deficiencies | Advantages |
| Vancouver Scar | 0 to 13 | Vascularity, | Lacks patient | Used widely in |
| Scale | | height/thickness, | perception | literature for |
| | | pliability, and | Pigmentation | outcome |
| | | pigmentation | subscale less | measure |
| | | | applicable to | in burn studies |
| | | | large, heterogeneous | |
| | | | scars Operator- | |
| | | | dependent errors | |
| | | | Excludes pain and | |
| | | | pruritis | |
| Visual Analog | 0 to 100 | Vascularity, | Photo-based scale | Simpler than |
| Scale with scar | “excellent” to | pigmentation, | does not include | VSS |
| ranking | “poor” | acceptability, | patient assessment | Assessments of |
| | | observer comfort | | intra- and |
| | | plus contour and | | interrater |
| | | summing the | | reliability easier |
| | | individual scores | | to conduct |
| Patient and | 5 to 50 | VSS plus surface | Items represented | Focuses on scar |
| Observer Scar | | area; patient | may not adequately | severity from |
| Assessment | | assessments | express patient's | clinician's and |
| Scale | | of pain, itching, | perceptions and | patient's points |
| | | color, stiffness, | concerns | of view |
| | | thickness, relief | | |
| Manchester | 5 (best) to 18 | VAS plus scar | Arbitrary | Applicable to a |
| Scar | (worse) | color, | assessment and | wider range of |
| Scale | | skin texture, | weighting of | scars |
| | | relationship to | items | Uses descriptors |
| | | surrounding skin, | | related to clinical |
| | | texture, margins, | | significance |
| | | size, multiplicity | | instead of |
| | | | | physical |
| | | | | measurement |
| | | | | alone |
| The Stony | 0 (worst) to 5 | VAS plus width, | Photo-based scale | Specifically |
| Brook | (best) | height, color, | does not include | developed to |
| Scar | | presence of | patient assessment | assess short-term |
| Evaluation | | suture/staple | Not designed for | appearance of |
| Scale | | marks | long-term scar | repaired |
| | | | assessment | lacerations |

12.1 Vancouver Scar Scale (VSS)

The Vancouver Scar Scale (VSS), first described by Sullivan in 1990, is perhaps the most recognized burn scar assessment method (Nedelec, B. et al., J Burn Care Rehabil. 21:205-12 (2000); Sullivan, T. et al., J Burn Care Rehabil. 11:256-60 (1990). It assesses four variables: vascularity, height/thickness, pliability, and pigmentation. Patient perception of his or her respective scars is not factored into the overall score. The VSS remains widely applicable to evaluate therapy and as a measure of outcome in burn studies.

12.2. Visual Analog Scale (VAS)

The multidimensional Visual Analog Scale (VAS) is a photograph-based scale derived from evaluating standardized digital photographs in 4 dimensions (pigmentation, vascularity, acceptability, and observer comfort) plus contour. It sums the individual scores to get a single overall score ranging from “excellent” to “poor.” It has shown high observer reliability and internal consistency when compared to expert panel evaluation, but it has shown only moderate reliability when used among lay panels (Duncan, J. et al. PRS. 118(4):909-18 (2006); Durani, P. et al., J Plastic Reconstr Aesth Surg, 62:713-20 (2009); Micomonaco, D. et al., J Otolaryngol Head Neck Surg 38(1):77-89 (2009)).

12.3. Patient and Observer Scar Assessment Scale (POSAS)

The Patient and Observer Scar Assessment Scale (POSAS) includes subjective symptoms of pain and pruritus and expands on the objective data captured in the VSS. It consists of two numerical numeric scales: The Patient Scar Assessment Scale and the Observer Scar Assessment Scale. It assesses vascularity, pigmentation, thickness, relief, pliability, and surface area, and it incorporates patient assessments of pain, itching, color, stiffness, thickness, and relief. The POSAS is the only scale that considers subjective symptoms of pain and pruritus, but like other scales, it also lacks functional measurements as to whether the pain or pruritus interferes with quality of life. The POSAS has been applied to postsurgical scars and is used in the evaluation of linear scars following breast cancer surgery. Reportedly, it shows internal consistency and interobserver reliability when compared to the VSS with the added benefit of capturing the patients' ratings.

12.4. Manchester Scar Scale (MSS)

The Manchester Scar Scale (MSS) (Beausang, E. et al. Plast Reconstr Surg. 102:1954 (1998)) differs from the POSAS in that it includes an overall VAS that is added to the individual attribute scores. It assesses and rates seven scar parameters: scar color (perfect, slight, obvious, or gross mismatch to surrounding skin), skin texture (matte or shiny), relationship to surrounding skin (range from flush to keloid), texture (range normal to hard), margins (distinct or indistinct), size (<1 cm, 1-5 cm, >5 cm), and single or multiple. Two Scores from the two scales are added together to give an overall score for the scar, with higher scores representing clinically worse scars. These data are analyzed in conjunction with details regarding race, ethnic background, history, cause, symptoms, treatments, and responses. Unlike the VSS, the MSS groups together vascularity and pigmentation under the heading of “color mismatch” relative to the surrounding tissue, allowing it to achieve better interrater agreement as compared to the VSS. It is thus applicable to a wider range of scars and well-suited for postoperative scars. The MSS has not been used in research, however, perhaps because of the wide applicability of the VSS and POSAS.

12.5. The Stony Brook Scar Evaluation Scale (SBSES)

The Stony Brook Scar Evaluation Scale (SBSES) was proposed in 2007 by Singer et al (Singer, A. et al., Plast Reconstr Surg. 120(7):1892-7 (2007)) and is a 6-item ordinal wound evaluation scale developed to measure short-term cosmetic outcome of wounds 5 to 10 days after injury up to the time of suture removal. It incorporates assessments of individual attributes with a binary response (1 or 0) for each, as well as overall appearance, to yield a score ranging from 0 (worst) to 5 (best). The SBSES has only recently been proposed for use in research, as it was designed to measure short-term rather than long-term wound outcomes. It thus has limited applicability to pathologic scar assessment.

### 13. Therapeutic Strategies for the Treatment of Cutaneous Scarring

Table 3 lists examples of currently available therapeutic strategies for the treatment of hypertrophic scarring.

TABLE 3
Selection of Currently Available Therapeutics for the Treatment of Hypertrophic
Scarring (Arabi, S. et al, PLoS Medicine, 4(9), e234, pp. 0001-0007, 2007)

| Therapy (Manufacture) | Category | Active Principle |
|---|---|---|
| Rose hip oil (various) | Natural remedies | Unknown |
| Vitamin E (various) | Natural remedies | Unknown |
| Corticosteroids (various) | Pharmaceutical | Unknown may be anti- |
| | | inflammatory |
| Juvista ® (Renovo) | Pharmaceutical | Anti-inflammatory |
| Neosporin ® (Johnson & Johnson) | Pharmaceutical | Antibiotic |
| Compression garment (various) | Wound Dressing | Unknown; may interfere with |
| | | mechanotransduction pathways |
| | | and tissue perfusion |
| Hydrogel sheeting (Avogel) | Wound Dressing | Unknown; may be anti- |
| | | inflammatory |
| Silicone sheeting (various) | Wound Dressing | Unknown; may interfere with |
| | | tissue perfusion |
| Smooth beam laser (Candela) | Non-ablative laser | Unknown; may stimulate |
| | | collagen remodeling |
| Erbium laser (various) | Ablative laser | Removes surface of scar |
| Chemical peel (N/A) | Surgical | Remove surface of scar |
| Revision surgery (N/A) | Surgical | Remove scar |

### 13.1. Targeting Inflammatory Mediators

The inflammatory response is a normal component of the wound healing process, serving both as an immunological barrier to infection and as a stimulus for fibrosis to close the site of injury. Observations from human pathological specimens and from healing fetal wounds have suggested that a robust inflammatory response may underlie the excessive fibrosis seen in hypertrophic scar formation. Mast cells, macrophages, and lymphocytes have all been implicated in this process. For example, mast cells have been shown to directly regulate stromal cell activity in vitro as well as to be strongly associated with the induction of fibrosis in vivo. Mechanical activity, age-specific changes, and delayed epithelialization have all been implicated as inciting factors for this intense inflammatory response.

Following cutaneous injury, endothelial damage and platelet aggregation occur resulting in the secretion of cytokines including the transforming growth factor (TGF)-β family, platelet-derived growth factors (PDGFs), and epidermal growth factors (EGFs). These cytokines stimulate fibroblast proliferation and matrix secretion, and induce leukocyte recruitment. Leukocytes, in turn, reinforce fibroblast activity, fight infection, and increase vascular permeability and ingrowth. They do this acting through the Transforming Growth Factor-beta (TGF-β) family, Fibroblast Growth Factors (FGFs), vascular endothelial growth factors (VEGFs), and other factors. Prostaglandins and Sma and Mad related protein (SMAD) activation also increase inflammatory cell proliferation and impair matrix breakdown.

Sma and Mad related protein (SMAD) is a family of evolutionarily conserved intracellular mediators that regulate the activity of particular genes as well as cell growth and proliferation. SMADs carry out their functions as part of the Transforming Growth Factor beta (TGF-β) signaling pathway, which transmits signals from the outside of the cell to the nucleus. The name “SMAD” was coined with the identification of human SMAD1 in reference to its sequence similarity to the SMA and MAD (Mothers Against Decapentaplegic homology) proteins.

Signaling by TGF-β1 is initiated by type I and II receptor-mediated phosphorylation. Activated TGF-β1 receptor I phosphorylates SMAD2 and SMAD3 (R-Smads) at their C terminus, which is antagonized by inhibitory SMAD6 and SMAD-7 (1-Smads). Following phosphorylation, R-SMADs form complexes with SMAD4 (Co-SMAD), translocate to the nucleus, and activate extracellular gene transcription. R-Smads are also phosphorylated by MAPK, particularly on the linker region that bridges the N-terminal MH1 and C-terminal MH2 domains. BMPs utilize a specific intracellular signaling cascade to target genes via R-SMADS (SMAD1,5,8), Co-SMAD (SMAD4) and I-SMADS (SMAD6,7)

SMAD7 is a known intracellular antagonist of TGF-β signaling, it inhibits TGF-β-induced transcriptional responses, whereas SMAD6 is a known inhibitor of TGF-β and BMP (bone morphogenic protein, a member of the TGF-β super family). The signaling axis of TGF-β/Smad2/3 and BMP (4/7)/Smad1 have been implicated in clinical IPF (Neininger, A. et al., J Biol Chem 277:3065-3068, 2002; Broekelmann, T. et al., Proc Natl Acad Sci USA, 88:6642-6646, 1991; Fernandez, I. E. and Eickelberg, O., Proc Am Thorac Soc 9:111-116, 2012; Murray, L. A., PLoS One 3:e4039, 2008; Aad, G., et al., Phys Rev Lett, 105:161801, 2010; Jonigk, D. et al., Virchows Arch 457:369-380, 2010; Zhang, K. et al., Am J Pathol 147:352-361, 1995; Kim, K. et al., J Clin Invest 119:213-224, 2009; Cutroneo, K. R. and Phan, S. H, J Cell Biochem 89:474-483, 2003; Flechsig, P. et al., Clin Cancer Res 18:3616-3627, 2012).

Increased levels of TGF-β1 and β2 as well as decreased levels of TGF-β3 have been associated with hypertrophic scarring through inflammatory cell stimulation, fibroblast proliferation, adhesion, matrix production, and contraction. Consistent with these observations, anti-inflammatory agents (cytokine inhibitors, corticosteroids, interferon α and β, and methotrexate) have been used with some success to reduce scar formation. For example, it was shown that function-blocking anti-TGF-β1 and β2 antibodies can reduce wound scarring in rat incision wounds (Shah, M. et al., J Cell Sci 107:1137-57, 1994). This experimentally confirmed approach has also been translated into the development of a recombinant TGF-β3 (Avotermin; Juvista®), whose early clinical trial results showed some potential to provide an accelerated and permanent improvement in scarring (Ferguson, M. W. et al., Lancet, 373: 1264-74, 2009), but failed in pivotal trials.

Increased vascular density, extensive microvascular obstruction, and malformed vessels have been observed also in hypertrophic scars, suggesting that structural changes may account for the persistent high inflammatory cell density observed in hypertrophic scars.

Other examples of anti-scarring agents, which have been used in the treatment of hypertrophic scars and keloids, include EXC001 (an anti-sense RNA against Connective Tissue Growth Factor (CTGF); Excaliard Pharmaceuticals), AZX100 (a phosphopeptide analog of Heat Shock Protein 20 (HSP20); Capstone Therapeutics Corp), PRM-151 (recombinant human serum amyloid P/Pentaxin 2; Promedior), PXL01 (a synthetic peptide derived from human Lactoferrin; PharaSurgics AB), DSC127 (an angiotensin analog; Derma Sciences, Inc), RXI-109 (a self-delivering RNAi compound that targets Connective Tissue Growth Factor (CTGF); Galena Biopharma), TCA (trichloroacetic acid; Isfahan University of Medical Sciences), Botox® (Capital District Health Authority and Allergan); Botulium toxin type A (Chang Gung Memorial Hospital), 5-fluorouracil, bleomycin, onion extract, pentoxifylline, prolyl-4-hydroxylase, verapamil, tacrolimus, and anti-TNF-α agents, tamoxifen, tretinoin, colchicine, calcium antagonists, tranilast, zinc, and vitamin E (Koc, E. et al., Dermatologic Surgery, vol. 34, no. 11, pp. 1507-1514 (2008); Lope, L. et al., Journal of Investigative Dermatology, vol. 129, no. 3, pp. 590-598 (2009); Rawlins, J. et al., Burns, vol. 32, no. 1, pp. 42-45 (2006); Kim I. et al, Wound Repair and Regeneration, vol. 11, no. 5, pp. 368-372 (2003); Copcu, E. et al., Journal of Burn Care and Rehabilitation, vol. 25, no. 1, pp. 1-7 (2004); Kim, A. et al., Journal of the American Academy of Dermatology, vol. 45, no. 5, pp. 707-711 (2001); LaDuca, J. and Gaspari, A., Dermatologic Clinics, vol. 19, no. 4, pp. 617-635, (2001)).

13.2. Targeting Epithelial-Mesenchymal Interaction

Epithelial cells play a number of important roles in normal skin physiology, which includes acting as stem cell niches and participating in complex signaling pathways to regulate mesenchymal cell function. The net result is the constant renewal of skin layers and the regulation of matrix deposition and remodeling. Cell-based skin substitutes take advantage of the regenerative nature of skin and are used clinically to cover wounds, but their utility in subsequent scar formation remains unknown. Epidermal stem cells have been suggested to act in concert with mesenchymal cells in the dermal papillae, functioning to recruit new cells to sites of skin regeneration, but large traumatic skin defects (such as those following burn injuries) were shown to destroy the resident epidermal stem cell population and cannot be spontaneously regenerated.

In addition to their regenerative function, epithelial cells have been shown to modulate mesenchymal cell proliferation and activity in normal skin and during wound healing and scar formation. In healing wounds, epithelial cells promote fibrosis and scarring through multiple pathways, such as, without limitation, those involving SMADs, including the signaling pathways through which TGF-β family members signal, phosphoinositide-3 kinase (PI3K), and Connective Tissue Growth Factor (CTGF). Epithelial cells stimulate fibroblasts during hypertrophic scar formation and fibroblasts themselves undergo intrinsic changes during the process of scarring. Subsequently, fibroblasts remain in an activated state, participating in cytokine autocrine loops that maintain fibrosis. Hypertrophic scar fibroblasts also have fundamentally altered profiles of cellular apoptosis, matrix production, and matrix degradation. It is unclear, however, whether these altered, profibrotic properties are due to genetic predisposition or are secondary to unique conditions present in the wound environment.

13.3. Targeting the Physical Environment

Following injury, the wound is a complex and mechanically unique environment with multiple levels of interaction between cells and the surrounding milieu. Fibroblasts and keratinocytes respond to the density and orientation of collagen and other matrix components. As a result, cells near the wound margin proliferate, while those further away from the edge of the wound are less active. At the same time, these cells actively produce and remodel the surrounding matrix. It was proposed that this delicate balance, which is responsible for a rapid and healthy response to injury, when disturbed, leads to aberrant wound healing.

Studies have shown that cells in the skin are also able to respond to their mechanical environment. Specifically, cell surface molecules, such as the integrin family, are activated by mechanical forces, leading to increased fibroblast survival as well as to the remodeling of deposited collagen and fibrin. While the intracellular signaling involved in this process is complex and incompletely understood, transcriptional regulators such as AKT/protein kinase B and focal adhesion kinase (FAK) have been found to be important elements. Keratinocyte proliferation and migration have been shown to be regulated similarly by mechanical stress. Following tissue injury, mechanotransduction serves a biological function to signal the presence of a tissue defect. Cells experience the highest levels of mechanical stress on the edge of a monolayer and, in the same way, the wound margin experiences high levels of mechanical stress. It was proposed that these stresses may have evolved to stimulate components of wound healing and initiate repair. Differences in exogenous forces may act to change cellular activation in the wound healing milieu and, when overactivated, lead to hypertrophic scar formation. A skin subjected to high level of stress (secondary to trauma or joint movement) usually shows robust hypertrophic scar formation.

Oxygen tension is another component of the physical environment that may be responsible for scar formation. Changes in levels of the transcription factor Hypoxia-Inducible Factor (HIF)-1α during fetal skin development were suggested to be partly responsible for the transition from scarless to scarred healing. Varying levels of HIF-1α in turn result in changes in a number of downstream proteins including Transforming Growth Factor-beta3 (TGF-β3) and Vascular Endothelial Growth Factor (VEGF). Changes in hypoxia signaling pathways contribute to the maturation of fetal skin and the development of a scarring phenotype following wounding. Changes in oxygen tension and increases in reactive oxygen species have also been shown to mediate early scar formation in tissues such as the lung and heart.

13.4. Surgical Anti-Scarring Therapies

Surgical excision is followed usually by recurrence unless adjunct therapies are employed since the new surgical wound is subject to the same mechanical and biochemical forces of the original lesion. The recurrence rate has been reported to range from 45-100% when surgical excision is performed as monotherapy (Mathangi-Ramakrishnan K et al., Plast Reconstr Surg 1974; 53, pp. 276-80 (1974); Cosman, B. and Wolff, M. Plast Reconstr Surg, 50, pp. 163-6 (1972); Lawrence, W., Ann Plast Surg, 27, pp. 164-78 (1991)). Furthermore, keloids that have recurred after excision were suggested to be more likely to recur if excised again (Cosman, B. et al., Plast Reconstr Surg, 27, pp. 335-58 (1961); Kovalic, J. and Perez, C., Int J Radiat Oncol Biol Phys, 17, pp. 77-80 (1989)).

13.5. Dermal Substitutes

The quality of skin wound healing was shown to be improved by the application of scaffolds as skin replacement materials. The skin replacement materials should protect wounds from infection and fluid loss; should be stable enough to function as a provisional matrix; should not elicit immunogenic reactions; and composition, pore size, and degradability of the substitute should support cell migration and function (Arabi, S. et al., PLoS Medicine, 4(9), e234, 1-7). Table 4 shows examples of currently available skin replacement materials.

TABLE 4
Selection of Currently Available Therapeutics for the
Prevention of Hypertrophic Scarring (Arabi, S. et al.,
PLoS Medicine, 4(9), e234, 1-7)

| Therapy | Category | Active Principle |
|---|---|---|
| Alloderm (LifeCell) | Skin Substitute | Transplantation (decellularized |
| | | human allograft) |
| Integra dermal | Skin Substitute | Transplantation (artificially |
| regeneration template | | manufacture matrix) |
| Epicel (Genzyme) | Skin Substitute | Transplantation (cultured |
| | | autograft kerationocytes) |

13.5.1. Natural Biological Materials

Natural biological materials, such as human or porcine cadaver skin or porcine small intestine submucosa (Oasis®), can be used as dermal substitutes because they provide a structurally intact natural three-dimensional (3D) extracellular matrix (ECM) of collagen and elastin. To improve materials for dermal substitution, several attempts have been made to remove cell remnants. Harsh methods can remove the cell remnants very effectively but often destroy the extracellular matrix structure, whereas milder methods are less efficient in removing all cell remnants. The removal of cell remnants can be achieved using different procedures. In the production of Alloderm®, for example, donor skin is treated with NaCl-SDS, which results in the retention of the basement membrane and in good immunogenic properties both in in vitro and in animal studies. The use of natural human or animal issues also requires extensive sterilization procedures to prevent potential disease transmission. Aggressive sterilization methods like ethylene oxide or gamma-irradiation were shown to induce structural changes in the dermis, whereas treatment with glycerol has shown little effect on the dermal structure.

13.5.2. Constructive Biological Materials

Dermal substitutes can be produced from purified biological molecules by means of lyophilization. Collagen is used often as the main component. To control aspects, such as pore size and interconnectivity, different freeze-drying (FD) procedures have been used during scaffold production. The properties can be adjusted by supplementing the substitutes with glycosaminoglycans (GAGs) and by cross-linking. The use of purified biological components allows the selection of materials with low or no antigenic potential. The precisely controlled production results in products with well-defined composition and properties. Many different molecules, such as growth factors and matrix components, can be added to the product. Examples of constructed dermal substitutes used for treatment include, but are not limited to, bilayer noncellularized dermal regeneration templates (e.g., Integra® (Integra™ Life Sciences), Renoskin® (Perouse Platie), and Hyalomatrix® (Anika Therapeutics)) and single layer cellularized dermal regeneration templates (e.g., Pelnac® (Gunze Ltd.), Mamiderm® (Dr. Suwelack Skin & Health Care AG), Single-layer Integra® (Integra™ Life Sciences), Alloderm™ (LifeCell), Strattice® (LifeCell), Permacol® (Tissue Science Laboratories), and Glyaderm® (Euro Skin Bank)).

Collagen, a major component of dermal substitutes, contains telopeptides located on the ends of the trihelical collagen molecule, which may induce an immune response. However, substitutes based on telopeptide-containing collagen were not rejected, suggesting that the possible immunogenicity of telopeptides in collagen does not interfere with the application of collagen in wound healing. Alternatively, collagen degradation can be reduced by the addition of extracellular components to protect the collagen from metalloproteinase degradation. For example, it was shown that the resistance of collagen scaffolds to collagenases could be increased by the addition of glycosaminoglycans such as chondroitin 6-sulfate, chondroitin 4-sulfate, dermatan sulfate, heparin, and heparan sulfate. The use of glycosaminoglycans also provides the possibility of controlling certain mechanical properties and pore sizes of the scaffolds. It has been hypothesized that coating of collagen fibers with fibronectin, hyaluronic acid, or elastin could stabilize dermal substitutes in a porcine full thickness wound model.

Studies have suggested that vascularization of dermal substitutes is important for high take rates and can be affected by the addition of extracellular components. The application of collagen/chondroitin-6-sulfate scaffolds like Integra® requires up to 3 weeks for the dermal substitute to become fully vascularized. The simultaneous application of the matrix and a split-skin graft generally results in graft loss due to the antiangiogenic properties of chondroitin-6-sulfate as showed in a chorioallantoic membrane (CAM) assay. In contrast, elastin and elastin-derived peptides promoted angiogenesis in a CAM assay and elastin-derived peptides were shown to function as chemoattractants for vascular smooth muscle cells. The application of collagen/elastin scaffolds showed increased vascularization one week post-wounding in a porcine excisional wound model.

13.5.3 Synthetic Substitutes

Dermal substitutes can also be constructed from non-biological molecules, which are not present in normal skin. Several synthetic substitutes have been tested in vitro or in animal experiments to assess their potential as dermal substitutes. Materials designed for this purpose should provide a provisional three dimensional support and interact with cells to control their function and to guide the complex processes of tissue formation and regeneration.

Fibroblasts and other cells require binding sites and chemotactic signals in the material for migration and proliferation. Interactions on synthetic materials such as tissue culture plastic, however, are distinctly different from those in natural extracellular matrix. The architecture and composition of the substrates, which affect adherence, migration, signaling, and cell function, therefore, can hamper the biological functioning of synthetic materials as dermal substitutes. In order to improve the use of synthetic matrices in tissue engineering applications, biomimetic protein sequences, such as the RGD (Arginine-Glycine-Aspartate) sequences, can be incorporated. The incorporation of these RGD sequences into self-assembling hydrogels have been shown to facilitate the migration and persistence of fibroblasts and results in more natural cell morphology but also in increased cell-matrix interactions such as contraction.

Hydrogels (Self-Assembling Peptides (SAP))

Self-assembly allows optimal control over the scaffold structure and composition. Techniques use specially designed peptides that automatically assemble into three dimensional structures under appropriate conditions through the formation of numerous noncovalent weak chemical bonds. The resulting peptide hydrogels present a fibrous nanoenvironment to the cells that is similar to skin. Self-Assembling Peptides can be produced cheaply in bulk and reduce the risk of disease transmission as no natural materials are used in the production of the scaffold.

Solid Freeform Fabrication (SFF)

Solid Freeform Fabrication (SFF), also known as rapid prototyping, is a collection of techniques whereby a three-dimensional scaffold is created by depositing individual layers through one of many different computers controlled spraying or printing techniques. SFF techniques allow for the creation of practically limitless shapes of scaffolds, from ears, to miniature houses. Furthermore, these techniques also allow for the deposition of viable cells with a level of control and precision that is impossible to achieve with approaches like ES.

13.6. Radiation

Radiation therapy is used infrequently as a monotherapy. When combined with surgical excision, the recurrence rate following radiation treatment has been reported between 10-20% (Sclafani, A. et al., Dermatol Surg, 22, pp. 569-74 (1996); Ragoowansi, R et al., Br J Plast Surg, 54, pp. 504-8 (2001)). A dose of at least 1500GY, delivered in fractions within 10 days of surgery, is recommended by some investigators (Doombos, J. et al., Int J Radiat Oncol Biol Phys, 18, pp. 833-839 (1990)) Inhibition of fibroblast proliferation and angiogenesis during the exaggerated wound-healing process is the proposed mechanism of action.

13.7. Pressure Therapy

Compression therapy for keloids was reported initially in the 1960s. The mechanism by which continuous pressure decreases the size and thickness of hypertrophic scars and keloids is not completely understood. Some studies have suggested that continuous pressure exerts its effect by producing tissue ischemia, decreasing tissue metabolism and increasing collagenase activity. Other theories include pressure-induced release of metalloproteinase-9 or prostaglandin E2 that may effect scar softening by the induction of extracellular matrix remodeling.

13.8. Cryotherapy

Cryotherapy has been used as a monotherapy and in combination with other techniques to treat keloid and hypertrophic scars. The mechanism by which cryotherapy exerts its therapeutic effect depends upon freezing-induced ischemic damage to the microcirculation. Freezing induces vascular damage and circulatory stasis leading to anoxia with eventual necrosis (Shaffer J. et al., J Am Acad Dermatol, 46, S63-97 (2002); Alster, T and West, T Ann Plast Surg, 39, pp. 418-32 (1997)). Therapy typically involves treating the entire scar with two or three freeze-thaw cycles of 30 seconds each. Cryotherapy was suggested to be more effective when combined with other procedures such as intralesional corticosteroids (Lahiri, A. et al., Br J Plast Surg, 54, pp. 633-635 (2001)).

13.9. Silicone Gel Sheeting and Other Dressings

Multiple studies have reported significant scar softening and decreased pruritus following application of topical silicone gel sheeting or cushions for at least 12 hours daily for 2-4 months. Silicone sheeting also has been used to prevent hypertrophic scarring (Gold, M. et al., Dermatol Surg, 27, pp. 641-642 (2001)). While the mechanism of action is not completely understood, it has been suggested that hydration, not pressure or silicone, may lead to fibroblast modification (Beranak, J., Dermatol Surg, 23, pp. 401-405 (1997)).

13.10. Laser

New lasers such as the nonablative fractional laser, has been employed for the treatment of scarring, although evidence of its efficacy is largely anecdotal (Mustoe, T., British Medical Journal, vol. 328, no. 7452, pp. 1329-1330 (2004)). It was shown that pulsed-dye lasers (PDL) can be used for treating resistant keloids in combination with intralesional steroids (Mustoe, T. et al., Plastic and Reconstructive Surgery, vol. 110, no. 2, pp. 560-571 (2002); Kuo, Y. et al., Lasers in Surgery and Medicine, vol. 36, No. 1, pp. 31-37 (2005)). Laser treatment has also been shown to be able to flatten hypertrophic scars and reduce erythema (redness of the skin), although with conflicting reports of success (Smit, J. et al., British Journal of Plastic Surgery, vol. 58, no. 7, pp. 981-987 (2005)). Some studies reported that that so called “laser welding” of skin wounds produce better scars in rats (Gulsoy, Z et al., Lasers in Medical Science, vol. 21, no. 1, pp. 5-10 (2006)).

Cutaneous scarring is an enormous medical problem with approximately 100 million patients acquiring scars each year (Bush, J. et al., Wound Rep Reg, 19: S32-S37, 2011). People with abnormal skin scarring may face physical, aesthetic, psychological, and social consequences that may be associated with substantial emotional and financial costs. However, scar reduction and elimination remain an unmet medical need because of the difficulty in their treatment.

### 14. The Role of the p38 MAPK-MK2 Signaling Pathway in Cutaneous Wound Healing

p38 mitogen-activated protein kinase (MAPK) and its upstream and downstream signaling molecules have been shown to play an important role in the response to cellular stress from stimuli (Saklatvala, Curr Opin Pharmacol, 4:372-377, 2004; Edmunds, J. and Talanian, MAPKAP Kinase 2 (MK2) as a Target for Anti-inflammatory Drug Discovery. In Levin, J and Laufer, S (Ed.), RSC Drug Discovery Series No. 26, p 158-175, the Royal Society of Chemistry, 2012).

There are four isoforms of p38 (i.e., p38α, p38β, p38γ, and p38δ) with p38α being most clearly associated with inflammation. Cytokines and other extracellular stimuli (such as growth factors, DNA damage, and oxidative stress) signal through multiple receptors and other mechanisms to activate a cascade of kinases starting with a MAP3K (e.g., MEKK3 or TAK1), then a MAP2K (e.g., MKK3 or MKK6), and then a MAPK (such as p38α) (FIG. 3). By direct and indirect effects, including the stabilization, translocation, and translation of mRNAs, p38 plays a major role in the production of proinflammatory cytokines, such as TNF-α, IL-6, and IFN-γ, as well as the induction of other pro-inflammatory cytokines, such as COX-2.

Generally, in resting cells, p38 MAPK and MK2 are physically bound together in the nucleus. Cellular stress causes the phosphorylation of p38 MAPK by an upstream kinase, such as MKK3 (Kim et al., Am J Physiol Renal Physiol, 292:F1471-1478, 2007). The activated p38 MAPK then phosphorylates MK2 at residues Thr-222, Ser-272, and/or Thr-334 (Engel et al., EMBO J, 17: 3363-3371, 1998). The activated MK2 and p38, still physically bound together, translocate to cytoplasm, where they phosphorylate their respective target protein (Ben-Levy et al., Curr Biol, 8:1049-1057, 1998).

In turn, activated MK2 mediates phosphorylation of HSPB1 in response to stress, leading to dissociation of HSPB1 from large small heat-shock protein (sHsps) oligomers, thereby impairing their chaperone activities and ability to protect against oxidative stress effectively.

MK2 is also involved in inflammatory and immune responses by regulating Tumor Necrosis Factor (TNF) and IL-6 production post-transcriptionally. This activity is mediated by phosphorylation of Adenine- and Uridine (AU)-Rich Elements (ARES)-binding proteins, such as Embryonic Lethal, Abnormal Vision, Drosophila-Like 1 (ELAVL1), Heterogeneous Nuclear Ribonucleoprotein A0 (HNRNPA0), Polyadenylate-Binding Protein 1 (PABPC1), and Tristetraprolin (TTP/ZFP36), which, in turn, regulate the stability and translation of TNF-α and IL-6 mRNAs. Phosphorylation of TTP/ZFP36, a major post-transcriptional regulator of TNF-α, promotes its binding to 14-3-3 proteins and reduces its affinity to ARE mRNA, thereby inhibits degradation of ARE-containing transcript (FIG. 4).

In addition, MK2 also plays an important role in the late G2/M checkpoint following DNA damage through a process of post-transcriptional mRNA stabilization. Following DNA damage, MK2 relocalizes from nucleus to cytoplasm and phosphorylates Heterogeneous Nuclear Ribonucleoprotein A0 (HNRNPA0) and Poly(A)-specific Ribonuclease (PARN), leading to stabilization of Growth arrest and DNA-damage-inducible protein 45A (GADD45A) mRNA. Additionally, studies have shown that MK2 is involved in the toll-like receptor signaling pathway (TLR) in dendritic cells and in acute TLR-induced macropinocytosis by phosphorylating and activating Ribosomal protein S6 kinase, 90 kDa, polypeptide 3 (RPS6KA3).

Although enzymes at each level of the aforementioned p38 MAPK signaling cascade have been explored for anti-cytokine drug discovery, it is difficult to generalize how upstream or downstream targets in such a pathway might vary in their potential for efficacy. For example, upstream targets might have multiple effects, enhancing efficacy, but might be bypassed by other signaling mechanisms, limiting the impact of inhibition. Undesirable side-effects are similarly difficult to predict. Therefore, specific properties of signaling mechanisms like that of the p38 pathway must be considered case by case to select the best targets based on empirical experience. (Edmunds, J. and Talanian, MAPKAP Kinase 2 (MK2) as a Target for Anti-inflammatory Drug Discovery. In Levin, J and Laufer, S (Ed.), RSC Drug Discovery Series No. 26, p 158-175, the Royal Society of Chemistry, 2012).

Indeed, while there have been many reports of p38 inhibitors with promising properties in vitro and in animal models of disease, none have achieved clinical success (Edmunds, J. and Talanian, MAPKAP Kinase 2 (MK2) as a Target for Anti-inflammatory Drug Discovery. In Levin, J and Laufer, S (Ed.), RSC Drug Discovery Series No. 26, p 158-175, the Royal Society of Chemistry, 2012). Many targets beyond those related to cytokine production are regulated by p38, consistent with observed pleiotropic consequences of its inhibition and suggesting multiple mechanisms of toxicity and even proinflammatory effects. For example, in hepatocytes, p38 directly and indirectly down-regulates JNK, thereby modulating hepatocyte sensitivity to lipopolysaccharide (LPS) and TNF-α induced cell death; this may be an important mechanism of p38 inhibition-induced liver toxicity. In addition, activation of MSK1 and MSK2 by p38 may induce expression of anti-inflammatory cytokine IL-10, and therefore inhibition of p38 may have a proinflammatory effect that contributes to the observed transient suppression of inflammatory markers by p38 inhibitors. Thus, there are significant concerns that, as an anti-inflammatory strategy, p38 inhibition will not result in adequate efficacy or acceptable safety.

On the other hand, MK2 attracted wide attention as a potential drug discovery target when it was reported that MK2-deficient knockout mice are viable and fertile, and are defective in TNF-α production. Splenocytes derived from these animals are defective in the production of several pro-inflammatory cytokines, including TNF-α, IL-6 and IFN-γ and the animals themselves are resistant to collagen-induced arthritis, a mouse model of rheumatoid arthritis (RA), as well as in ovalbumin-induced airway inflammation, a mouse model of asthma. Dosed orally, inhibitors of MK2 can block acute systemic induction of TNF-α by LPS in rats and can reduce paw swelling in the rat streptococcal cell wall (SCW)-induced arthritis model. These results suggested that MK2 mediates most or all inflammatory signals of the p38 cascade while other p38 substrates regulate the pathways responsible for toxicity or attenuated efficacy; and that MK2 inhibition might deliver on the promise of p38 inhibition for anti-inflammatory efficacy while also giving a more favorable safety profile.

Recent MK2 knockout studies suggested that MK2 may be involved in cutaneous wound healing. For example, it was shown that the kinetics of wound healing are significantly affected by the absence of MK2 in excisional wounds. Histological examination showed a higher level of acanthosis (meaning an increase in the thickness of the prickle cell layer of the epidermis) of the migrating wound keratinocyte layer as well as a higher level of collagen deposition in the granulation tissue of the wounds from wild type mice than those from MK2 knockout mice. The study further showed that the expression of many cytokines and chemokines was significantly affected at different days post wounding; and that the delayed healing rate of wounds in MK2 knockout mice can be significantly improved by passive transfer of macrophages with intact MK2. These results suggested an important role of MK2 gene expression in macrophages participating in the process of cutaneous wound healing (Thuraisingam et al., J Invest Dermatol., 130(1):278-286, 2010).

Given that abnormalities in cell migration, proliferation, inflammation, and the synthesis and secretion of extracellular matrix proteins and cytokines, and remodeling during wound healing processes are associated with the formation of pathological scars in cutaneous tissues, these previous studies suggest that targeting an aberrant activity of MK2 in cutaneous wounds may be an effective means for treating, reducing or preventing scar formation in cutaneous tissue.

While MK2 drug discovery efforts have combined simultaneous consideration of in-vitro potency, solubility, cell permeability and clearance to produce potentially low-dose compounds, in vivo activity of small-molecule MK2 inhibitors has been hampered by limited inhibition of TNF-α production in whole blood due, presumably, to the difficulty in achieving unbound plasma levels in excess of the cell-based assay EC₅₀ values. In addition to the difficulties posed by the high ATP affinity of nonphosphorylated MK2, poor correlations have been observed between the inhibition of recombinant MK2 and cell assay potency within series of compounds, suggesting further complexities, such as variations in analogue-specific properties that affect cell potency, e.g., membrane penetration (Edmunds, J. and Talanian, MAPKAP Kinase 2 (MK2) as a Target for Anti-inflammatory Drug Discovery. In Levin, J and Laufer, S (Ed.), RSC Drug Discovery Series No. 26, p 158-175, the Royal Society of Chemistry, 2012).

The described invention offers approaches to intervene in the process of cutaneous scar formation by utilizing a cell-penetrating, peptide-based inhibitor of MK2.

### SUMMARY OF THE INVENTION

According to one aspect, the described invention provides, a pharmaceutical composition for use in treating a cutaneous scar in a subject in need thereof, comprising a therapeutic amount of a Mitogen-Activated Protein Kinase-Activated Protein Kinase 2 (MK2) inhibitor comprising an MK2 polypeptide inhibitor of amino acid sequence YARAAARQARAKALARQLGVAA (SEQ ID NO: 1) or a functional equivalent thereof, and a pharmaceutically acceptable carrier, wherein the subject in need thereof has suffered a wound, and the therapeutic amount is effective (a) to reduce incidence, severity, or both, of the cutaneous scar without impairing normal wound healing and (b) to treat the cutaneous scar in the subject, such that at least one of the wound size, scar area, and collagen whorl formation in the wound is reduced compared to the control.

According to one embodiment, the wound is an abrasion, a laceration, a crush, a contusion, a puncture, an avulsion, a burn, an ulcer, an incisional wound, a high-tension wound, or a combination thereof. According to another embodiment, the cutaneous scar is a pathological scar, an incisional scar, or a combination thereof. According to another embodiment, the pathological scar is selected from the group consisting of a hypertrophic scar, a keloid, an atrophic scar, a scar contracture, or a combination thereof. According to another embodiment, the pathological scar results from a high-tension wound located in close proximity to a joint comprising a knee, an elbow, a wrist, a shoulder, a hip, a spine, or a combination thereof. According to another embodiment, the pathological scar results from an abrasion, a laceration, an incision, a crush, a contusion, a puncture, an avulsion, a burn, an ulcer, an autoimmune skin disorder, or a combination thereof. According to another embodiment, the autoimmune skin disorder is selected from the group consisting of systemic lupus erythematosus (SLE), systemic sclerosis (scleroderma), pemphigus, vitiligo, dermatitis herpetiformis, psoriasis, or a combination thereof.

According to another embodiment, the therapeutic amount is effective to inhibit at least 65% of a kinase activity of at least one kinase selected from the group consisting of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof without substantially inhibiting an off-target protein. According to another embodiment, the therapeutic amount is effective to reduce either a level of transforming growth factor-□ (TGF-β) expression in the wound; or number of at least one immunomodulatory cell or a progenitor cell infiltrating into the wound, or both. According to another embodiment, the immunomodulatory cell is selected from the group consisting of a monocyte, a mast cell, a dendritic cell, a macrophage, a T-lymphocyte, a fibrocyte, or a combination thereof. According to another embodiment, the progenitor cell is selected from the group consisting of a hematopoitic stem cell, a mesenchymal stem cell, or a combination thereof.

According to another embodiment, the pharmaceutical composition further comprises at least one additional therapeutic agent selected from the group consisting of an anti-inflammatory agent, an analgesic agent, an anti-infective agent, or a combination thereof. According to another embodiment, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof. According to another embodiment, the additional therapeutic agent is selected from the group consisting of rose hip oil, vitamin E, 5-fluorouracil, bleomycin, onion extract, pentoxifylline, prolyl-4-hydroxylase, verapamil, tacrolimus, tamoxifen, tretinoin, colchicine, tranilst, zinc, an antibiotic, and a combination thereof.

According to another embodiment, the functional equivalent of the MK2 polypeptide inhibitor of amino acid sequence YARAAARQARAKALARQLGVAA (SEQ ID NO: 1) has at least 80 percent sequence identity to amino acid sequence YARAAARQARAKALARQLGVAA (SEQ ID NO: 1); and is a polypeptide of amino acid sequence selected from the group consisting of YARAAARQARAKALNRQLGVA (SEQ ID NO: 19), FAKLAARLYRKALARQLGVAA (SEQ ID NO: 3), KAFAKLAARLYRKALARQLGVAA (SEQ ID NO: 4), YARAAARQARAKALARQLAVA (SEQ ID NO: 5), YARAAARQARAKALARQLGVA (SEQ ID NO: 6), or HRRIKAWLKKIKALARQLGVAA (SEQ ID NO: 7). According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide is of amino acid sequence YARAAARQARA (SEQ ID NO: 11), and the second polypeptide comprises a therapeutic domain whose sequence has at least 70 percent sequence identity to amino acid sequence KALARQLGVAA (SEQ ID NO: 2) and is selected from the group consisting of a polypeptide of amino acid sequence KALARQLAVA (SEQ ID NO: 8), a polypeptide of amino acid sequence KALARQLGVA (SEQ ID NO: 9), a polypeptide of amino acid sequence KALARQLGVAA (SEQ ID NO: 10). According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11) and is a polypeptide of amino acid sequence selected from the group consisting of WLRRIKAWLRRIKA (SEQ ID NO: 12), WLRRIKA (SEQ ID NO: 13), YGRKKRRQRRR (SEQ ID NO: 14), WLRRIKAWLRRI (SEQ ID NO: 15), FAKLAARLYR (SEQ ID NO: 16), KAFAKLAARLYR (SEQ ID NO: 17), and HRRIKAWLKKI (SEQ ID NO: 18); and the second polypeptide is of amino acid sequence KALARQLGVAA (SEQ ID NO: 2).

According to another embodiment, the pharmaceutically acceptable carrier is a controlled release carrier. According to another embodiment, the pharmaceutically acceptable carrier comprises particles. According to another embodiment, the therapeutic amount is effective to modulate an expression level of at least one scar-related gene or scar-related protein in a wound selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD).

According to another embodiment, the pharmaceutical composition further comprises a small molecule MK2 inhibitor, wherein the small molecule MK2 inhibitor is a pyrrolopyridone analogue or a multicyclic lactam analogue. According to another embodiment, the therapeutic amount of the MK2 polypeptide inhibitor of the pharmaceutical composition is of an amount from about 0.000001 mg/kg body weight to about 100 mg/kg body weight.

According to another aspect, the described invention provides a method for treating a cutaneous scar in a subject in need thereof, wherein the subject in need thereof has suffered a wound, wherein the method comprises administering to the subject a pharmaceutical composition comprising a therapeutic amount of a Mitogen-Activated Protein Kinase-Activated Protein Kinase 2 (MK2) inhibitor comprising an MK2 polypeptide inhibitor of amino acid sequence YARAAARQARAKALARQLGVAA (SEQ ID NO: 1) or a functional equivalent thereof, and a pharmaceutically acceptable carrier, wherein the therapeutic amount is effective (a) to reduce incidence, severity, or both, of the cutaneous scar without impairing normal wound healing and (b) to treat the cutaneous scar in the subject, such that at least one of the wound size, scar area, and collagen whorl formation in the wound is reduced compared to the control.

According to one embodiment, the wound is an abrasion, a laceration, a crush, a contusion, a puncture, an avulsion, a burn, an ulcer, an incisional wound, a high-tension wound, or a combination thereof. According to another embodiment, the cutaneous scar is a pathological scar, an incisional scar, or a combination thereof. According to another embodiment, the pathological scar is selected from the group consisting of a hypertrophic scar, a keloid, an atrophic scar, a scar contracture, or a combination thereof. According to another embodiment, the pathological scar results from a high-tension wound located in close proximity to a joint comprising a knee, an elbow, a wrist, a shoulder, a hip, a spine, or a combination thereof. According to another embodiment, the pathological scar results from an abrasion, a laceration, an incision, a crush, a contusion, a puncture, an avulsion, a burn, an ulcer, an autoimmune skin disorder, or a combination thereof. According to another embodiment, the autoimmune skin disorder is selected from the group consisting of systemic lupus erythematosus (SLE), systemic sclerosis (scleroderma), pemphigus, vitiligo, dermatitis herpetiformis, psoriasis, or a combination thereof.

According to another embodiment, the administering is topically. According to another embodiment, the administering is by means of a dressing comprising the pharmaceutical composition. According to another embodiment, at least one surface of the dressing is impregnated with the pharmaceutical composition. According to another embodiment, the dressing is selected from the group consisting of a gauze dressing, a tulle dressing, an alginate dressing, a polyurethane dressing, a silicone foam dressing, a synthetic polymer scaffold dressing, or a combination thereof. According to another embodiment, the dressing is an occlusive dressing selected from the group consisting of a film dressing, a semi-permeable film dressing, a hydrogel dressing, a hydrocolloid dressing, and a combination thereof. According to another embodiment, the administering is by means of a dermal substitute, wherein the pharmaceutical composition is embedded in a dermal substitute that provides a three dimensional scaffold. According to another embodiment, the dermal substitute is made of a natural biological material, a constructive biological material, or a synthetic material. According to another embodiment, the natural biological material comprises human cadaver skin, porcine cadaver skin, or porcine small intestine submucosa. According to another embodiment, the natural biological material comprises a matrix. According to another embodiment, the natural biological material consists essentially of a matrix that is sufficiently devoid of cell remnants. According to another embodiment, the constructive biological material comprises collagen, glycosaminoglycan, fibronectin, hyaluonic acid, elastine, or a combination thereof. According to another embodiment, the constructive biological material is a bilayer, non-cellularized dermal regeneration template or a single layer, cellularized dermal regeneration template. According to another embodiment, the synthetic dermal substitute comprises a hydrogel. According to another embodiment, the synthetic dermal substitute further comprises an RGD peptide with amino acid sequence Arginine-Glycine-Aspartate.

According to another embodiment, the administering is intraperitoneally, intravenously, intradermally, intramuscularly, or a combination thereof. According to another embodiment, the administering is via an injection device, wherein the injection device is soaked with the pharmaceutical composition prior to administration. According to another embodiment, the injection device is selected from the group consisting of a needle, a cannula, a catheter, a suture, or a combination thereof.

According to another embodiment, the therapeutic amount of the MK2 polypeptide inhibitor of the amino acid sequence YARAAARQARAKALARQLGVAA (SEQ ID NO: 1) or a functional equivalent thereof for intradermal injection ranges from 50 ng/100 μl/linear centimeter of wound margin to 500 ng/100 μl/linear centimeter of wound margin. According to another embodiment, the therapeutic amount of the MK2 polypeptide inhibitor of the amino acid sequence YARAAARQARAKALARQLGVAA (SEQ ID NO: 1) or a functional equivalent thereof for intraperitoneal administration ranges from 70 μg/kg to 80 μg/kg.

According to another embodiment, the administering is in a single dose at one time. According to another embodiment, the administering is in a plurality of doses for a period of at least one day, at least one week, at least one month, at least one year, or a combination thereof. According to another embodiment, the administering is at least once daily, at least once weekly, or at least once monthly.

According to another embodiment, the pharmaceutical composition further comprises at least one additional therapeutic agent selected from the group consisting of an anti-inflammatory agent, an analgesic agent, an anti-infective agent, or a combination thereof. According to another embodiment, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof. According to another embodiment, the additional therapeutic agent is selected from the group consisting of rose hip oil, vitamin E, 5-fluorouracil, bleomycin, onion extract, pentoxifylline, prolyl-4-hydroxylase, verapamil, tacrolimus, tamoxifen, tretinoin, colchicine, tranilst, zinc, an antibiotic, and a combination thereof.

According to another embodiment, the administering is before, during, or after closing of the wound. According to another embodiment, the closing of the wound is by means of at least one subcutaneous suture, at least one staple, at least one adhesive tape, a surgical adhesive, or a combination thereof. According to another embodiment, the surgical adhesive comprises octyl-2-cyanoacrylate or fibrin tissue adhesive.

According to another embodiment, the functional equivalent of the MK2 polypeptide inhibitor of amino acid sequence YARAAARQARAKALARQLGVAA (SEQ ID NO: 1) has at least 80 percent sequence identity to amino acid sequence YARAAARQARAKALARQLGVAA (SEQ ID NO: 1); and is a polypeptide of amino acid sequence selected from the group consisting of YARAAARQARAKALNRQLGVA (SEQ ID NO: 19), FAKLAARLYRKALARQLGVAA (SEQ ID NO: 3), KAFAKLAARLYRKALARQLGVAA (SEQ ID NO: 4), YARAAARQARAKALARQLAVA (SEQ ID NO: 5), YARAAARQARAKALARQLGVA (SEQ ID NO: 6), or HRRIKAWLKKIKALARQLGVAA (SEQ ID NO: 7). According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide is of amino acid sequence YARAAARQARA (SEQ ID NO: 11), and the second polypeptide comprises a therapeutic domain whose sequence has at least 70 percent sequence identity to amino acid sequence KALARQLGVAA (SEQ ID NO: 2) and is selected from the group consisting of a polypeptide of amino acid sequence KALARQLAVA (SEQ ID NO: 8), a polypeptide of amino acid sequence KALARQLGVA (SEQ ID NO: 9), a polypeptide of amino acid sequence KALARQLGVAA (SEQ ID NO: 10). According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11) and is a polypeptide of amino acid sequence selected from the group consisting of WLRRIKAWLRRIKA (SEQ ID NO: 12), WLRRIKA (SEQ ID NO: 13), YGRKKRRQRRR (SEQ ID NO: 14), WLRRIKAWLRRI (SEQ ID NO: 15), FAKLAARLYR (SEQ ID NO: 16), KAFAKLAARLYR (SEQ ID NO: 17), and HRRIKAWLKKI (SEQ ID NO: 18); and the second polypeptide is of amino acid sequence KALARQLGVAA (SEQ ID NO: 2).

According to another embodiment, the therapeutic amount is effective to inhibit at least 65% of a kinase activity of at least one kinase selected from the group consisting of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof without substantially inhibiting an off-target protein. According to another embodiment, the therapeutic amount is effective to modulate an expression level of at least one scar-related gene or scar-related protein in a wound selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD).

According to another embodiment, the therapeutic amount is effective to reduce either a level of transforming growth factor-β (TGF-β) expression in the wound; or number of at least one immunomodulatory cell or a progenitor cell infiltrating into the wound, or both. According to another embodiment, the immunomodulatory cell is selected from the group consisting of a monocyte, a mast cell, a dendritic cell, a macrophage, a T-lymphocyte, a fibrocyte, or a combination thereof. According to another embodiment, the progenitor cell is selected from the group consisting of a hematopoitic stem cell, a mesenchymal stem cell, or a combination thereof.

According to another embodiment, the pharmaceutical composition further comprises a small molecule MK2 inhibitor, wherein the small molecule MK2 inhibitor is a pyrrolopyridone analogue or a multicyclic lactam analogue.

According to another aspect, the described invention provides a dressing for use in treating a cutaneous scar in a subject in need thereof, wherein the subject in need thereof has suffered a wound, wherein the dressing comprises a pharmaceutical composition comprising a therapeutic amount of a Mitogen-Activated Protein Kinase-Activated Protein Kinase 2 (MK2) inhibitor comprising an MK2 polypeptide inhibitor of the amino acid sequence YARAAARQARAKALARQLGVAA (SEQ ID NO: 1) or a functional equivalent thereof, and a pharmaceutically acceptable carrier, wherein the therapeutic amount is effective (a) to reduce incidence, severity, or both, of the cutaneous scar without impairing normal wound healing and (b) to treat the cutaneous scar in the subject, such that at least one of the wound size, scar area, and collagen whorl formation in the wound is reduced compared to the control.

According to one embodiment, the dressing is selected from the group consisting of a gauze dressing, a tulle dressing, an alginate dressing, a polyurethane dressing, a silicone foam dressing, a collagen dressing, a synthetic polymer scaffold, peptide-soaked sutures or a combination thereof. According to another embodiment, the dressing is an occlusive dressing selected from the group consisting of a film dressing, a semi-permeable film dressing, a hydrogel dressing, a hydrocolloid dressing, and a combination thereof. According to another embodiment, the dressing further comprises a dermal substitute embedded in or on a surface of the dressing with the pharmaceutical composition, and wherein the dermal substitute provides a three-dimensional extracellular scaffold. According to another embodiment, the dermal substitute is made of a natural biological material, a constructive biological material, or a synthetic material. According to another embodiment, the natural biological material comprises human cadaver skin, porcine cadaver skin, or porcine small intestine submucosa. According to another embodiment, the natural biological material comprises a matrix. According to another embodiment, the natural biological material consists essentially of a matrix that is sufficiently devoid of cell remnants. According to another embodiment, the constructive biological material comprises collagen, glycosaminoglycan, fibronectin, hyaluonic acid, elastine, or a combination thereof. According to another embodiment, the constructive biological material is a bilayer, non-cellularized dermal regeneration template or a single layer, cellularized dermal regeneration template. According to another embodiment, the synthetic dermal substitute comprises a hydrogel. According to another embodiment, the synthetic dermal substitute further comprises an RGD peptide with amino acid sequence Arginine-Glycine-Aspartate.

According to another embodiment, the wound is an abrasion, a laceration, a crush, a contusion, a puncture, an avulsion, a burn, an ulcer, an incisional wound, a high-tension wound, or a combination thereof. According to another embodiment, the cutaneous scar is a pathological scar, an incisional scar, or a combination thereof. According to another embodiment, the pathological scar is selected from the group consisting of a hypertrophic scar, a keloid, an atrophic scar, a scar contracture, or a combination thereof. According to another embodiment, the pathological scar results from a high-tension wound located in close proximity to a joint comprising a knee, an elbow, a wrist, a shoulder, a hip, a spine, or a combination thereof. According to another embodiment, the pathological scar results from an abrasion, a laceration, an incision, a crush, a contusion, a puncture, an avulsion, a burn, an ulcer, an autoimmune skin disorder, or a combination thereof. According to another embodiment, the autoimmune skin disorder is selected from the group consisting of systemic lupus erythematosus (SLE), systemic sclerosis (scleroderma), pemphigus, vitiligo, dermatitis herpetiformis, psoriasis, or a combination thereof. According to another embodiment, the dressing is a mechano-active dressing further comprising an anti-infective agent, a growth factor, a vitamin, a clotting agent, or a combination thereof.

According to another embodiment, the pharmaceutical composition further comprises at least one additional therapeutic agent selected from the group consisting of an anti-inflammatory agent, an analgesic agent, an anti-infective agent, or a combination thereof. According to another embodiment, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof. According to another embodiment, the additional therapeutic agent is selected from the group consisting of rose hip oil, vitamin E, 5-fluorouracil, bleomycin, onion extract, pentoxifylline, prolyl-4-hydroxylase, verapamil, tacrolimus, tamoxifen, tretinoin, colchicine, tranilst, zinc, an antibiotic, and a combination thereof.

According to another embodiment, the functional equivalent of the MK2 polypeptide inhibitor of amino acid sequence YARAAARQARAKALARQLGVAA (SEQ ID NO: 1) has at least 80 percent sequence identity to amino acid sequence YARAAARQARAKALARQLGVAA (SEQ ID NO: 1); and is a polypeptide of amino acid sequence selected from the group consisting of YARAAARQARAKALNRQLGVA (SEQ ID NO: 19), FAKLAARLYRKALARQLGVAA (SEQ ID NO: 3), KAFAKLAARLYRKALARQLGVAA (SEQ ID NO: 4), YARAAARQARAKALARQLAVA (SEQ ID NO: 5), YARAAARQARAKALARQLGVA (SEQ ID NO: 6), or HRRIKAWLKKIKALARQLGVAA (SEQ ID NO: 7). According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide is of amino acid sequence YARAAARQARA (SEQ ID NO: 11), and the second polypeptide comprises a therapeutic domain whose sequence has at least 70 percent sequence identity to amino acid sequence KALARQLGVAA (SEQ ID NO: 2) and is selected from the group consisting of a polypeptide of amino acid sequence KALARQLAVA (SEQ ID NO: 8), a polypeptide of amino acid sequence KALARQLGVA (SEQ ID NO: 9), a polypeptide of amino acid sequence KALARQLGVAA (SEQ ID NO: 10). According to another embodiment, the functional equivalent of the polypeptide YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11) and is a polypeptide of amino acid sequence selected from the group consisting of WLRRIKAWLRRIKA (SEQ ID NO: 12), WLRRIKA (SEQ ID NO: 13), YGRKKRRQRRR (SEQ ID NO: 14), WLRRIKAWLRRI (SEQ ID NO: 15), FAKLAARLYR (SEQ ID NO: 16), KAFAKLAARLYR (SEQ ID NO: 17), and HRRIKAWLKKI (SEQ ID NO: 18); and the second polypeptide is of amino acid sequence KALARQLGVAA (SEQ ID NO: 2).

According to another embodiment, the pharmaceutically acceptable carrier is a controlled release carrier. According to another embodiment, the pharmaceutically acceptable carrier comprises particles.

According to another embodiment, the therapeutic amount is effective to inhibit at least 65% of a kinase activity of at least one kinase selected from the group consisting of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof without substantially inhibiting an off-target protein. According to another embodiment, the therapeutic amount is effective to modulate an expression level of at least one scar-related gene or scar-related protein in a wound selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD).

According to another embodiment, the therapeutic amount is effective to reduce either a level of transforming growth factor-β (TGF-β) expression in the wound; or number of at least one immunomodulatory cell or a progenitor cell infiltrating into the wound, or both. According to another embodiment, the immunomodulatory cell is selected from the group consisting of a monocyte, a mast cell, a dendritic cell, a macrophage, a T-lymphocyte, a fibrocyte, or a combination thereof. According to another embodiment, the progenitor cell is selected from the group consisting of a hematopoitic stem cell, a mesenchymal stem cell, or a combination thereof.

According to another embodiment, the pharmaceutical composition further comprises a small molecule MK2 inhibitor, wherein the small molecule MK2 inhibitor is a pyrrolopyridone analogue or a multicyclic lactam analogue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows anatomy of the skin.

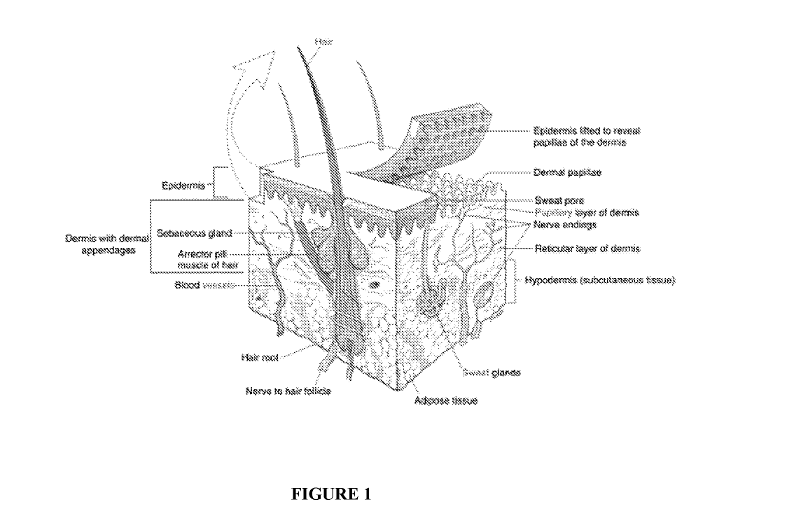

FIG. 2 shows layers of the epidermis.

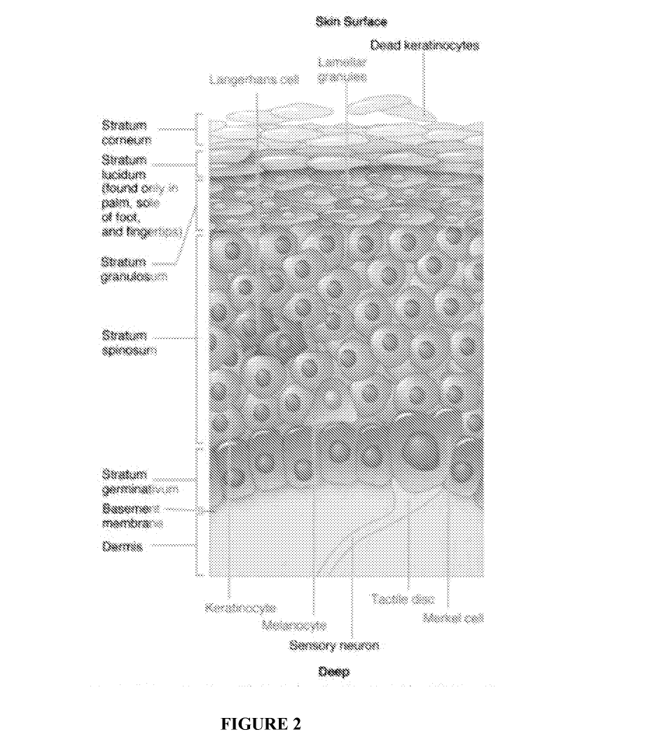

FIG. 3 shows p38 MAPK-MK2 signaling cascade.

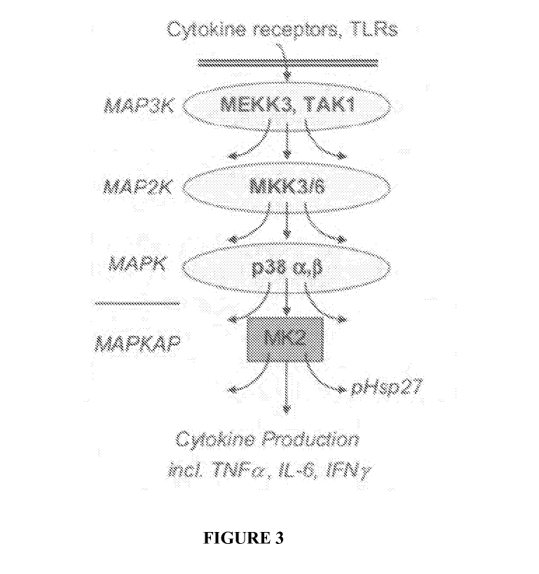

FIG. 4 shows a model for anti-TNF-α consequences of MK2 inhibition.

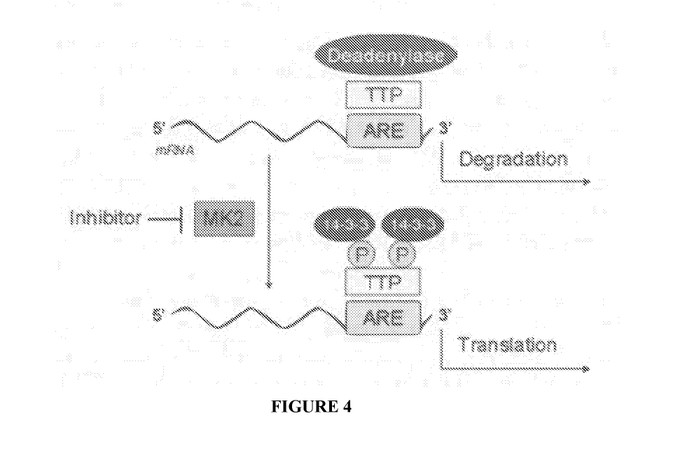

FIG. 5 shows overview of wound repair and fibrosis.

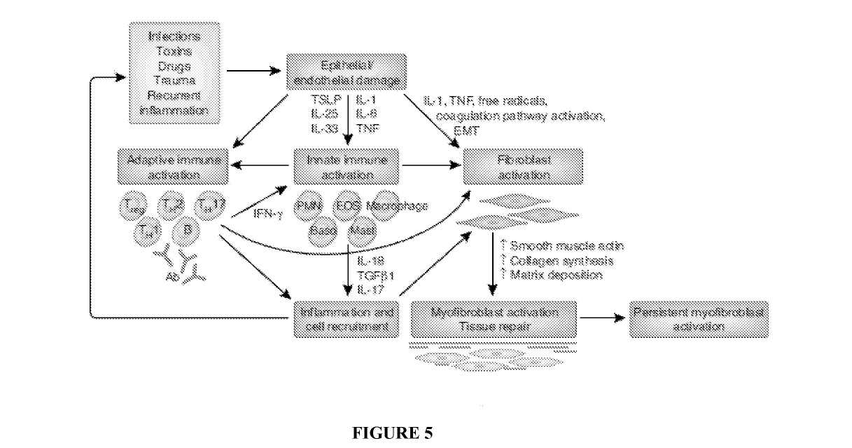

FIG. 6 shows gross comparison of scar appearance in a PBS treated mouse and a MMI-0100-treated mouse. Scale bar=2 mm.

FIG. 7 shows scar area comparison between control and MMI-100 (SEQ ID NO: 1)-treated mice. Scar edges were identified and scar areas were quantified using Image J software. Scar areas were compared using student's t-test; n=5; *, P=0.011.

FIG. 8 shows histological comparison of scars in MMI-0100 (SEQ ID NO: 1)-treated mice with PBS treated group. Scar areas were outlined, and areas were measured by using Image J software. Scale bar=100 μm.

FIG. 9 shows comparison of cross sectional areas of scars treated with a control (PBS) or MMI-0100 (SEQ ID NO: 1). n=5, *, P=0.015.

FIG. 10 shows quantitative reverse transcription polymerase chain reaction (qRT-PCR) comparison of scar related gene transcripts with the PBS-treated group (n=3) and the MMI-0100 (SEQ ID NO: 1)-treated group (n=4). *, P=0.016.

FIG. 11 shows comparison of cell population in scar areas with the PBS-treated group (n=5) and the MMI-0100 (SEQ ID NO: 1)-treated group (n=4). *, P=0.02.

FIG. 12 shows gross comparison of scar appearance in a PBS treated mouse and a MMI-0300-treated mouse on day 4 and day 14. The scale bar ruler=2.2 cm.

FIG. 13 shows histological comparison of scars in MMI-0300 (SEQ ID NO: 3)-treated mice with PBS treated group. Scar areas were outlined, and areas were measured by using Image J software.

FIG. 14 shows quantitative reverse transcription polymerase chain reaction (qRT-PCR) comparison of scar related gene transcripts with the PBS-treated group (n=3) and the MMI-0300-treated group (n=6).

FIG. 15 shows comparison of cell population in scar areas with the young and old PBS-treated and the MMI-0100 (SEQ ID NO: 1)-treated groups.

FIG. 16 shows wound size as a percentage (pct) of wound size in Red Duroc pigs at Day 0 for wound sites treated with MMI-100 (300 μM) (1^{st} bar), MMI-100 (30 μM) (2^{nd} bar), and PBS control (3^{rd} bar). Asterisk indicates statistical significance (p<0.05).

### DETAILED DESCRIPTION OF THE INVENTION

### Glossary

The term “abrasion” as used herein refers to a scraping or rubbing away of a body surface by friction.

The term “administer” as used herein means to give or to apply. The term “administering” as used herein includes in vivo administration, as well as administration directly to tissue ex vivo. Generally, administration may be systemic (i.e., affecting the entire body), e.g., orally, buccally, parenterally (e.g., intravenous, intraarterial, subcutaneous, intraperitoneal (i.e., into a body cavity), etc.), topically, by inhalation or insufflation (i.e., through the mouth or through the nose), or rectally in dosage unit formulations containing conventional nontoxic pharmaceutically acceptable carriers, adjuvants, and vehicles as desired, or may be local by means such as, but not limited to, injection, implantation, grafting, topical application, or parenterally.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

Antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary. For example, in the mouse and rat, λ chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ than κc.

In mammals, there are five classes of antibodies, IgA, IgD, IgE, IgG, and IgM, each with its own class of heavy chain—α (for IgA), δ (for IgD), ε (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (VK and VX) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-fungal agent” as used herein means any of a group of chemical substances having the capacity to inhibit the growth of or to destroy fungi. Anti-fungal agents include but are not limited to Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, PyrroInitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconawle, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “anti-infective agent” as used herein refers to an agent that is capable of inhibiting the spread of an infectious agent such as an infectious microorganism, e.g., a bacteria, a virus, a nematode, a parasite, etc. Exemplary anti-infective agents may include antibiotic agent, antifungal agent, anti-viral agent, anti-protozoal agent, etc.

The term “anti-inflammatory agent” as used herein refers to an agent that reduces inflammation. The term “steroidal anti-inflammatory agent”, as used herein, refer to any one of numerous compounds containing a 17-carbon 4-ring system and includes the sterols, various hormones (as anabolic steroids), and glycosides. The term “non-steroidal anti-inflammatory agents” refers to a large group of agents that are aspirin-like in their action, including ibuprofen (Advil)®, naproxen sodium (Aleve)®, and acetaminophen (Tylenol)®.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation include pyrimethamine (Daraprim®) sulfadiazine, and Leucovorin.

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferon-□, Interferon-□, Interferon-□, Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

The term “atrophic scar” as used herein refers to a scar, which is flat and depressed below the surrounding skin. They are generally small and often round with an indented or inverted center. Atrophic scarring can be a result of surgery, trauma, and such common conditions as acne vulgaris and varicellar (chickenpox).

The term “autoimmune disorder” as used herein refers to disease, disorders or conditions in which the body's immune system, which normally fights infections and viruses, is misdirected and attacks the body's own normal, healthy tissue.

The term “avulsion” as used herein refers to a forcible tearing away or separation of a bodily structure or part, either as the result of injury or as an intentional surgical procedure.

The term “biodegradable”, as used herein, refers to material that will break down actively or passively over time by simple chemical processes, by action of body enzymes or by other similar biological activity mechanisms.

The term “biomimetic” as used herein refers to materials, substances, devices, processes, or systems that imitate or “mimic” natural materials made by living organisms.

The term “burn” as used herein refers to an injury to tissues caused by the contact with heat, flame, chemicals, electricity, or radiation. First degree burns show redness; second degree burns show vesication (a blistered spot); third degree burns show necrosis (cell death) through the entire skin. Burns of the first and second degree are partial-thickness burns, those of the third degree are full-thickness burns.

The term “carrier” as used herein describes a material that does not cause significant irritation to an organism and does not abrogate the biological activity and properties of the peptide of the composition of the described invention. Carriers must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “clotting agent” as used herein refers to an agent that promotes the clotting of blood. Exemplary clotting agents include but are not limited to thrombin, prothrombin, fibrinogen, etc.

The term “component” as used herein refers to a constituent part, element or ingredient.

The term “condition”, as used herein, refers to a variety of health states and is meant to include disorders or diseases caused by any underlying mechanism or disorder, injury, and the promotion of healthy tissues and organs.

The term “contact” and all its grammatical forms as used herein refers to a state or condition of touching or of immediate or local proximity.

The term “controlled release” as used herein refers to any drug-containing formulation in which the manner and profile of drug release from the formulation are regulated. This refers to immediate as well as non-immediate release formulations, with non-immediate release formulations including, but not limited to, sustained release and delayed release formulations.

The term “contusion” as used herein refers to an injury in which the skin is not broken, a contusion is caused when blood vessels are damaged or broken as the result of a blow to the skin. The raised area of a bump or bruise results from blood leaking from these injured blood vessels into the tissues as well as from the body's response to the injury.

The term “crush” as used herein refers to a bruise or contusion from pressure between two solid bodies.

The term “cutaneous scar” as used herein refers to a dermal fibrous replacement tissue, which results from a wound that healed by resolution rather than regeneration.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally, but, to use hormone terminology, may have autocrine, paracrine or even endocrine effects. They include type I cytokines, which encompass many of the interleukins, as well as several hematopoietic growth factors; type II cytokines, including the interferons and interleukin-10; tumor necrosis factor (“TNF”)-related molecules, including TNF-α and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell; inflammatory cytokines may be produced by virtually all nucleated cells, for example, endo/epithelial cells and macrophages. Cytokines often regulate the expression of, and trigger cascades of, other cytokines

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “immunomodulatory cell(s)” as used herein refer(s) to cell(s) that are capable of augmenting or diminishing immune responses by expressing chemokines, cytokines and other mediators of immune responses.

The term “inflammatory cytokines” or “inflammatory mediators” as used herein refers to the molecular mediators of the inflammatory process, which may modulate being either pro- or anti-inflamatory in their effect. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, pro-inflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

Among the pro-inflammatory mediators, IL-1, IL-6, and TNF-α are known to activate hepatocytes in an acute phase response to synthesize acute-phase proteins that activate complement. Complement is a system of plasma proteins that interact with pathogens to mark them for destruction by phagocytes. Complement proteins can be activated directly by pathogens or indirectly by pathogen-bound antibody, leading to a cascade of reactions that occurs on the surface of pathogens and generates active components with various effector functions. IL-1, IL-6, and TNF-α also activate bone marrow endothelium to mobilize neutrophils, and function as endogenous pyrogens, raising body temperature, which helps eliminating infections from the body. A major effect of the cytokines is to act on the hypothalamus, altering the body's temperature regulation, and on muscle and fat cells, stimulating the catabolism of the muscle and fat cells to elevate body temperature. At elevated temperatures, bacterial and viral replications are decreased, while the adaptive immune system operates more efficiently.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include interleukin-1 (IL-1), interleukin-1β (IL-1β), interleukin-6 (IL-6), interleukin-8 (IL-8), and interleukin-12 (IL-12).

The term “Tumor Necrosis Factor” or “TNF” as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The term “delayed release” is used herein in its conventional sense to refer to a formulation in which there is a time delay between administration of the formulation and the release of the therapeutic agent therefrom. “Delayed release” may or may not involve gradual release of the therapeutic agent over an extended period of time, and thus may or may not be “sustained release.”

The term “disease” or “disorder”, as used herein, refers to an impairment of health or a condition of abnormal functioning.

The terms “dose” and “dosage” are used interchangeably to refer to the quantity of a drug or other remedy to be taken or applied all at one time or in fractional amounts within a given period.

The term “drug” as used herein refers to a therapeutic agent or any substance, other than food, used in the prevention, diagnosis, alleviation, treatment, or cure of disease.

The term “effective amount” refers to the amount necessary or sufficient to realize a desired biologic effect.

The term “excisional wound” as used herein refers to a wound resulting from surgical removal or cutting away of tissue. The term “excisional wound” includes, but is not limited to, tears, abrasions, cuts, punctures, or lacerations in the epithelial layer of the skin that may extend into the dermal layer and even into subcutaneous fat and beyond.

The term “extracellular matrix” as used herein refers to a tissue derived or bio-synthetic material that is capable of supporting the growth of a cell or culture of cells.

The term “extracellular matrix deposition” as used herein refers to the secretion of fibrous elements (e.g., collagen, elastin, and reticulin), link proteins (e.g., fibronectin, laminin), and space filling molecules (e.g., glycosaminoglycans) by cells.

The term “formulation” as used herein refers to a mixture prepared according to a formula, recipe or procedure.

The term “functional equivalent” as used herein refers to a peptide having similar or identical effects or use. For example, functionally equivalents of the polypeptide MMI-0100 (YARAAARQARAKALARQLGVAA; SEQ ID NO: 1) of the describe invention possess kinase inhibition activities or kinetic parameters, which are similar or identical to those of the polypeptide MMI-0100 (SEQ ID NO: 1) in vitro, ex vivo, or in vivo. Likewise, the “functional equivalent” of YARAAARQARA (SEQ ID NO: 11) possesses an ability to penetrate the plasma membrane of mammalian cells and to transport compounds of many types across the membrane, which is similar or identical to that of YARAAARQARA (SEQ ID NO: 11).

The term “gene delivery vehicle” as used herein refers to a component that facilitates delivery to a cell of a coding sequence for expression of a polypeptide in the cell. The gene delivery vehicle can be any component or vehicle capable of accomplishing the delivery of a gene or cDNA to a cell, for example, a liposome, a virus particle, or an expression vector.

The term “granulation” as used herein refers to a process whereby small red, grain-like prominences form on a raw surface in the process of healing.

The term “granulomatous inflammation” as used herein refers to an inflammation reaction characterized by a predominance of regular to epithelioid macrophages with or without multinucleated giant cells and connective tissue.

The term “hydrophilic” as used herein refers to a material or substance having an affinity for polar substances, such as water. The term “lipophilic” as used herein refers to preferring or possessing an affinity for a non-polar environment compared to a polar or aqueous environment.

The term “high tension wound” as used herein refers to a wound occurred in areas at or near a joint, including areas at or near elbow or knee. Other areas of the “high tension wound” include midsternal chest and post cesarean section wound.

The term “hypertrophic scar” as used herein refers to a cutaneous condition characterized by the formation of excess, raised scar tissue, but not growing beyond the boundary of the original wound.

The term “IC₅₀ value” as used herein refers to the concentration of an inhibitor that is needed to inhibit 50% of a given biological process or component of a process (i.e., an enzyme, cell, or cell receptor).

The term “in close proximity” as used herein refers to a distance very near.

The term “incisional wound” as used herein refers to a wound made by a clean cut, as with a sharp instrument.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, soluble inflammatory mediators of the inflammatory response work together with cellular components in a systemic fashion in the attempt to contain and eliminate the agents causing physical distress. The term “inflammatory mediators” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1, interleukin-4, interleukin-6, interleukin-8, tumor necrosis factor (TNF), interferon-gamma, and interleukin 12.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%. The terms “further inhibiting”, “further inhibit” or “further inhibition are used herein to refer to reducing the amount or rate of a second process, to stopping the second process entirely, or to decreasing, limiting, or blocking the action or function thereof in addition to reducing the amount or rate of a first process, to stopping the first process entirely, or to decreasing, limiting, or blocking the action or function thereof. The term “inhibitory profile” as used herein refers to the characteristic pattern of reduction of the amount or rate or decrease, blocking or limiting of the action of more than one protein or enzyme. The terms “substantially inhibiting”, “substantially inhibit”, “substantially inhibited”, or “substantially inhibition” are used here to refer to inhibition of kinase activity by at least 65%.

The term “inhibitor” as used herein refers to a second molecule that binds to a first molecule thereby decreasing the first molecule's activity. Enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “injection”, as used herein, refers to introduction into subcutaneous tissue, muscular tissue, a vein, an artery, or other canals or cavities in the body by force.

The term “injury,” as used herein, refers to damage or harm to a structure or function of the body caused by an outside agent or force, which may be physical or chemical.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, or more than about 99% free of. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “keloid” or “keloid scar” as used herein refers to a benign fibrous proliferation in the dermis that arise after dermal trauma. Keloid scars are raised above the surface of the skin and extend beyond the boundaries of the original wound.

The term “kinase” as used herein refers to an enzyme that catalyzes the phosphorylation of a substrate by adenosine triphosphate (ATP).

The term “laceration” as used herein refers to a torn and ragged wound or an accidental cut wound.

The term “long-term” release, as used herein, means that an implant is constructed and arranged to deliver therapeutic levels of the active ingredient for at least about 5, 6, 7, 8, 9, 10, 11, 12, 13, 14, 15, 16, 17, 18, 19, 20, 21, 22, 23, 24, 25, 26, 27, 29, 29, 30, 31, 32, 33, 34, 35, 36, 37, 38, 39, 40, 41, 42, 43, 44, 45, 46, 47, 48, 48, 50, 51, 52, 53, 54, 55, 56, 57, 58, 59, or 60 days.

The term “manifestation” as used herein refers to the display or disclosure of characteristic signs or symptoms of an illness. Thus, the term “skin manifestation” as used herein refers to the display or disclosure of characteristic signs or symptoms of an illness on the skin.

The term “mechano-active dressing” as used herein refers to a medical or surgical covering for a wound that is configured to be removably secured to a skin surface near a wound in order to apply tension to the wound.

The term “modulate” as used herein means to regulate, alter, adapt, or adjust to a certain measure or proportion.

The term “neovascularization” as used herein refers to the new growth of blood vessels with the result that the oxygen and nutrient supply is improved. Similarly, the term “angiogenesis” refers to the vascularization process involving the development of new capillary blood vessels.

The term “nucleic acid” is used herein to refer to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The term “off-target protein” as used herein refers to a protein, which can be affected by a pharmaceutical composition but whose effect is not the primary therapeutic effect of the composition.

The term “operatively linked” as used herein refers to a linkage in which two or more protein domains or peptides are ligated or combined via recombinant DNA technology or chemical reaction such that each protein domain or polypeptide of the resulting fusion peptide retains its original function. For example, SEQ ID NO: 1 is constructed by operatively linking a protein transduction domain (SEQ ID NO: 26) with a therapeutic domain (SEQ ID NO: 2), thereby creating a fusion peptide that possesses both the cell penetrating function of SEQ ID NO: 26 and the MK2 kinase inhibitor function of SEQ ID NO: 2.

The term “parenteral” as used herein refers to introduction into the body by way of an injection (i.e., administration by injection) outside the gastrointestinal tract, including, for example, subcutaneously (i.e., an injection beneath the skin), intramuscularly (i.e., an injection into a muscle); intravenously (i.e., an injection into a vein), or by infusion techniques. A parenterally administered composition is delivered using a needle, e.g., a surgical needle. The term “surgical needle” as used herein, refers to any needle adapted for delivery of fluid (i.e., those capable of flow) compositions into a selected anatomical structure. Injectable preparations, such as sterile injectable aqueous or oleaginous suspensions, may be formulated according to the known art using suitable dispersing or wetting agents and suspending agents.

The terms “particles”, as used herein, refer to extremely small constituents, e.g., femoparticles (10⁻¹⁵ m), picoparticles (10⁻¹²), nanoparticles (10⁻⁹ m), microparticles (10⁻⁶ m), milliparticles (10⁻³ m)) that may contain in whole or in part the MK2 inhibitor as described herein.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity.”

(a) The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

(b) The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination.

(c) The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e) The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

The term “pathological scar” as used herein refers to a scar arising as a result of a disease, disorder, condition, or injury.

The term “pharmaceutically acceptable carrier” as used herein refers to one or more compatible solid or liquid filler, diluent or encapsulating substance which is/are suitable for administration to a human or other vertebrate animal. The components of the pharmaceutical compositions also are capable of being commingled in a manner such that there is no interaction which would substantially impair the desired pharmaceutical efficiency.

The term “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

The term “pharmaceutical composition” is used herein to refer to a composition that is employed to prevent, reduce in intensity, cure or otherwise treat a target condition or disease.

The term “prevent” as used herein refers to the keeping, hindering or averting of an event, act or action from happening, occurring, or arising.

The term “prodrug” as used herein means a peptide or derivative which is in an inactive form and which is converted to an active form by biological conversion following administration to a subject.

The term “puncture” as used herein refers to a wound in which the opening is relatively small as compared to the depth, as produced by a narrow pointed object.

The term “recombinant” as used herein refers to a substance produced by genetic engineering.

The term “reduced” or “to reduce” as used herein refer to a diminution, a decrease, an attenuation or abatement of the degree, intensity, extent, size, amount, density or number.

The term “reepithelialization” as used herein refers to the reformation of epithelium over a denuded surface (e.g., wound).

The term “release” and its various grammatical forms, refers to dissolution of an active drug component and diffusion of the dissolved or solubilized species by a combination of the following processes: (1) hydration of a matrix, (2) diffusion of a solution into the matrix; (3) dissolution of the drug; and (4) diffusion of the dissolved drug out of the matrix.

The term “reduce” or “reducing” as used herein refers to a diminution, a decrease, an attenuation, limitation or abatement of the degree, intensity, extent, size, amount, density, number or occurrence of disorder in individuals at risk of developing the disorder.

The term “remodeling” as used herein refers to the replacement of and/or devascularization of granulation tissue.

The term “scaffold” as used herein refers to a substance or structure used to enhance or promote the growth of cells and/or the formation of tissue. A scaffold is typically a three dimensional porous structure that provides a template for cell growth.

The term “scar” as used herein refers to a fibrous tissue replacing normal tissue destroyed by injury or disease. The term “scarring” as used herein refers to the condition when fibrous tissue replaces normal tissue destroyed by injury or disease. The term “scar area” as used herein refers to the extent of normal tissue that is destroyed by injury or disease and is replaced by fibrous tissue.

The term “scar contracture” or “contracture scar” as used herein refers to a permanent tightening of skin that may affect the underlying muscles and tendons that limit mobility and possible damage or degeneration of nerves. Contractures develop when normal elastic connective tissues are replaced with inelastic fibrous tissue, which makes the tissues resistant to stretching and prevents normal movement of the affected area.

The term “scar-related gene” as used herein refers to a piece of DNA encoding a protein that is activated in response to scarring as part of the normal wound healing process. The term “scar-related gene product” as used herein refers to the protein that is expressed in response to scarring as part of the normal wound healing process.

The term “similar” is used interchangeably with the terms analogous, comparable, or resembling, meaning having traits or characteristics in common.

The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The phrase “subject in need of such treatment” is used to refer to a patient who has or will suffer a wound that can result in cutaneous scarring unless the context and usage of the phrase indicates otherwise.

The term “substantially pure”, as used herein, refers to a condition of a therapeutic agent such that it has been substantially separated from the substances with which it may be associated in living systems or during synthesis. According to some embodiments, a substantially pure therapeutic agent is at least 70% pure, at least 75% pure, at least 80% pure, at least 85% pure, at least 90% pure, at least 95% pure, at least 96% pure, at least 97% pure, at least 98% pure, or at least 99% pure.

The term “susceptible” as used herein refers to a member of a population at risk.

The term “sustained release” (also referred to as “extended release”) is used herein in its conventional sense to refer to a drug formulation that provides for gradual release of a therapeutic agent over an extended period of time, and that preferably, although not necessarily, results in substantially constant levels of the agent over an extended time period.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “moiety” as used herein refers to a functional group of a molecule. The term “targeting moiety” as used herein refers to a functional group attached to a molecule that directs the molecule to a specific target, cell type or tissue.

The term “therapeutic agent” as used herein refers to a drug, molecule, nucleic acid, protein, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The terms “therapeutic amount”, “therapeutic effective amount” or an “amount effective” of one or more of the active agents is an amount that is sufficient to provide the intended benefit of treatment. An effective amount of the active agents that can be employed ranges from generally 0.1 mg/kg body weight and about 50 mg/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a surgeon using standard methods.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation.

The term “topical” as used herein refers to administration of a composition at, or immediately beneath, the point of application. The phrase “topically applying” describes application onto one or more surfaces(s) including epithelial surfaces. Topical administration also may involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation. Patches suitable for use in the described invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated by reference in its entirety. These patches are well known in the art and generally available commercially.

The term “traumatic wound” as used herein refers to a wound that is the result of an injury.

The term “treat” or “treating” includes abrogating, substantially inhibiting, slowing or reversing the progression of a disease, condition, disorder or injury, substantially ameliorating clinical or esthetical symptoms of a disease, condition, disorder or injury, substantially preventing the appearance of clinical or esthetical symptoms of a disease, condition, disorder or injury, and protecting from harmful or annoying symptoms. The term “treat” or “treating” as used herein further refers to accomplishing one or more of the following: (a) reducing the severity of the disease, condition, disorder or injury; (b) limiting development of symptoms characteristic of the disease, condition, disorder or injury being treated; (c) limiting worsening of symptoms characteristic of the disease, condition, disorder or injury being treated; (d) limiting recurrence of the disease, condition, disorder or injury in patients that have previously had the disease, condition, disorder or injury; and (e) limiting recurrence of symptoms in patients that were previously symptomatic for the disease, condition, disorder or injury.

The term “ulcer” as used herein refers to a lesion of the skin that is characterized by the formation of pus and necrosis (death of surrounding tissue) usually resulting from inflammation or ischemia.

The term “wound” as used herein refers to a disruption of the normal continuity of structures caused by a physical (e.g., mechanical) force, a biological or a chemical means. The term “wound” includes, but is not limited to, incisional wounds, excisional wounds, traumatic wounds, lacerations, punctures, cuts, and the like. The term “wound size” as used herein refers to a physical measure of disruption of the normal continuity of structures caused by a physical (e.g., mechanical) force, a biological or a chemical means.

The term “full-thickness wound” as used herein refers to destruction of tissue extending through the second layer of skin (dermis) to involve subcutaneous tissue under and possibly muscle or bone; the tissue can appear snowy white, gray, or brown, with a firm leathery texture.

The term “partial-thickness wound” as used herein refers to destruction of tissue through the first layer of skin (epidermis), extending into, but not through, the dermis.

The term “vitamin” as used herein, refers to any of various organic substances essential in minute quantities to the nutrition of most animals act especially as coenzymes and precursors of coenzymes in the regulation of metabolic processes. Non-limiting examples of vitamins usable in context of the present invention include vitamin A and its analogs and derivatives: retinol, retinal, retinol palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

The term “wound closure” as used herein refers to the healing of a wound such that the edges of the wound are rejoined to form a continuous barrier.

The term “wound healing” as used herein refers to a regenerative process with the induction of a temporal and spatial healing program, including, but not limited to, the processes of inflammation, granulation, neovascularization, migration of fibroblast, endothelial and epithelial cells, extracellular matrix deposition, reepithealization, and remodeling.

### I. Compositions for Treating, Reducing or Preventing a Cutaneous Scar

According to one aspect, the described invention provides a pharmaceutical composition for use in treating a cutaneous scar in a subject who has suffered or is suffering from a wound, the pharmaceutical composition comprising a therapeutic amount of a Mitogen-Activated Protein Kinase-Activated Protein Kinase 2 (MK2) inhibitor comprising an MK2 polypeptide inhibitor or a functional equivalent thereof, and a pharmaceutically acceptable carrier, wherein the therapeutic amount is effective to reduce scar areas in the subject.

MK2 Inhibitor

According to one embodiment, the Mitogen-Activated Protein Kinase-Activated Protein Kinase 2 (MK2) inhibitor is an MK2 polypeptide inhibitor or a functional equivalent thereof. According to some embodiments, the MK2 polypeptide inhibitor is selected from the group consisting of a polypeptide MMI-0100 of amino acid sequence YARAAARQARAKALARQLGVAA (SEQ ID NO: 1), a polypeptide MMI-0200 of amino acid sequence YARAAARQARAKALNRQLGVA (SEQ ID NO: 19), a polypeptide MMI-0300 of amino acid sequence FAKLAARLYRKALARQLGVAA (SEQ ID NO: 3), a polypeptide MMI-0400 of amino acid sequence KAFAKLAARLYRKALARQLGVAA (SEQ ID NO: 4), and a polypeptide MMI-0500 of amino acid sequence HRRIKAWLKKIKALARQLGVAA (SEQ ID NO: 7). According to one embodiment, the MK2 polypeptide inhibitor is a polypeptide MMI-0100 of amino acid sequence YARAAARQARAKALARQLGVAA (SEQ ID NO: 1). According to another embodiment, the MK2 polypeptide inhibitor is a polypeptide MMI-0200 of amino acid sequence YARAAARQARAKALNRQLGVA (SEQ ID NO: 19). According to another embodiment, the MK2 polypeptide inhibitor is a polypeptide MMI-0300 of amino acid sequence FAKLAARLYRKALARQLGVAA (SEQ ID NO: 3). According to another embodiment, the MK2 polypeptide inhibitor is a polypeptide MMI-0400 of amino acid sequence KAFAKLAARLYRKALARQLGVAA (SEQ ID NO: 4). According to another embodiment, the MK2 polypeptide inhibitor is a polypeptide MMI-0500 of amino acid sequence HRRIKAWLKKIKALARQLGVAA (SEQ ID NO: 7).

According to another embodiment, the functional equivalent of the MK2 polypeptide inhibitor MMI-0100 of amino acid sequence YARAAARQARAKALARQLGVAA (SEQ ID NO: 1) has a substantial sequence identity to the amino acid sequence YARAAARQARAKALARQLGVAA (SEQ ID NO: 1).

According to another embodiment, the functional equivalent of the MK2 polypeptide inhibitor MMI-0100 of amino acid sequence YARAAARQARAKALARQLGVAA (SEQ ID NO: 1) has at least 80 percent sequence identity to the amino acid sequence YARAAARQARAKALARQLGVAA (SEQ ID NO: 1). According to another embodiment, the functional equivalent of the MK2 polypeptide inhibitor MMI-0100 of amino acid sequence YARAAARQARAKALARQLGVAA (SEQ ID NO: 1) has at least 90 percent sequence identity to the amino acid sequence YARAAARQARAKALARQLGVAA (SEQ ID NO: 1). According to another embodiment, the functional equivalent of the MK2 polypeptide inhibitor MMI-0100 of amino acid sequence YARAAARQARAKALARQLGVAA (SEQ ID NO: 1) has at least 95 percent sequence identity to the amino acid sequence YARAAARQARAKALARQLGVAA (SEQ ID NO: 1).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0200 of amino acid sequence YARAAARQARAKALNRQLGVA (SEQ ID NO: 19)

According to another embodiment, the functional equivalent of the MK2 polypeptide inhibitor MMI-0100 of amino acid sequence YARAAARQARAKALARQLGVAA (SEQ ID NO: 1) is a polypeptide MMI-0300 of amino acid sequence FAKLAARLYRKALARQLGVAA (SEQ ID NO: 3).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0400 of amino acid sequence KAFAKLAARLYRKALARQLGVAA (SEQ ID NO: 4).

According to another embodiment, the functional equivalent of the MK2 polypeptide inhibitor MMI-0100 of amino acid sequence YARAAARQARAKALARQLGVAA (SEQ ID NO: 1) is a polypeptide of amino acid sequence YARAAARQARAKALARQLAVA (SEQ ID NO: 5).

According to another embodiment, the functional equivalent of the MK2 polypeptide inhibitor MMI-0100 of amino acid sequence YARAAARQARAKALARQLGVAA (SEQ ID NO: 1) is a polypeptide of amino acid sequence YARAAARQARAKALARQLGVA (SEQ ID NO: 6).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0500 of amino acid sequence HRRIKAWLKKIKALARQLGVAA (SEQ ID NO: 7).

According to another embodiment, the Mitogen-Activated Protein Kinase-Activated Protein Kinase 2 (MK2) inhibitor further comprises a small molecule MK2 inhibitor. Exemplary small molecule MK2 inhibitors have been described in Anderson, D. R. et al., Bioorg. Med. Chem. Lett., 15: 1587 (2005); Wu, J.-P. et al., Bioorg. Med. Chem. Lett., 17: 4664 (2007); Trujillo, J. I. et al., Bioorg. Med. Chem. Lett., 17: 4657 (2007); Goldberg, D. R. et al., Bioorg. Med. Chem. Lett., 18: 938 (2008); Xiong, Z. et al., Bioorg. Med. Chem. Lett., 18: 1994 (2008); Anderson, D. R. et al., J. Med. Chem., 50: 2647 (2007); Lin, S. et al., Bioorg. Med. Chem. Lett., 19: 3238 (2009); Anderson, D. R. et al., Bioorg. Med. Chem. Lett., 19: 4878 (2009); Anderson, D. R. et al., Bioorg. Med. Chem. Lett., 19: 4882 (2009); Harris, C. M. et al., Bioorg. Med. Chem. Lett., 20: 334 (2010); Schlapbach, A. et al., Bioorg. Med. Chem. Lett., 18: 6142 (2008); and Velcicky, J. et al., Bioorg. Med. Chem. Lett., 20: 1293 (2010), the entire disclosure of each of which is incorporated herein by reference.

According to some such embodiments, the small molecule MK2 inhibitor includes, but is not limited to:

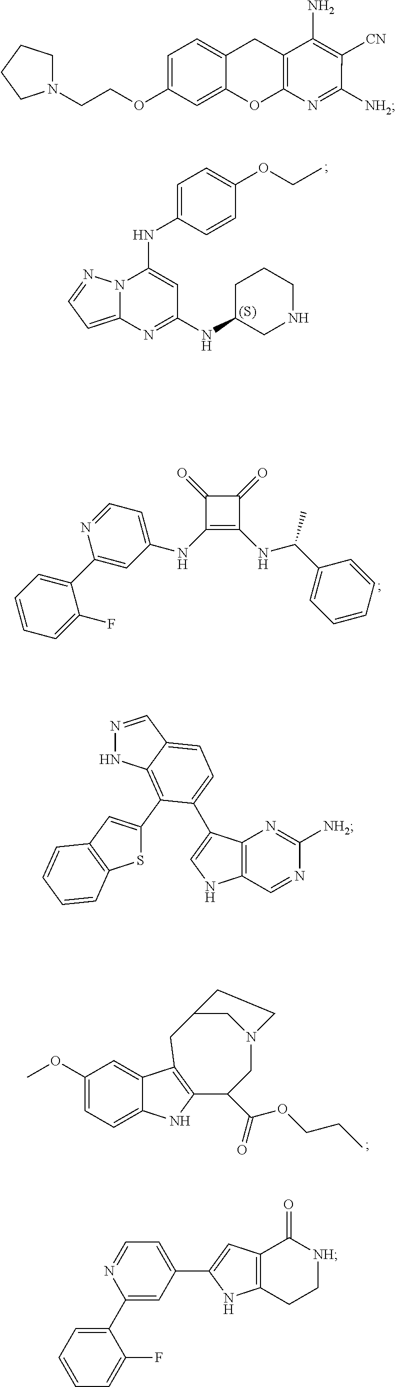

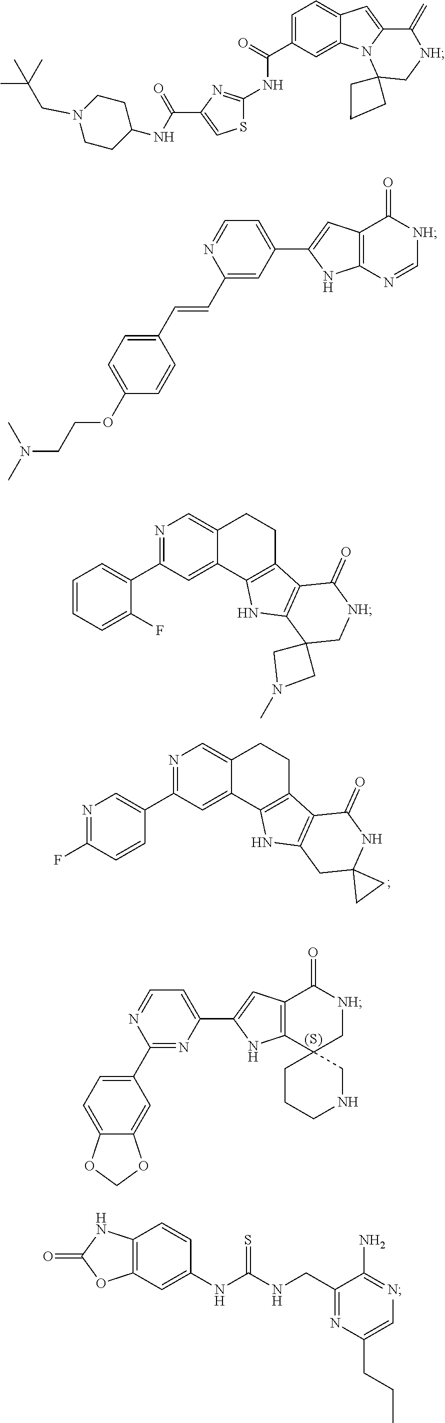

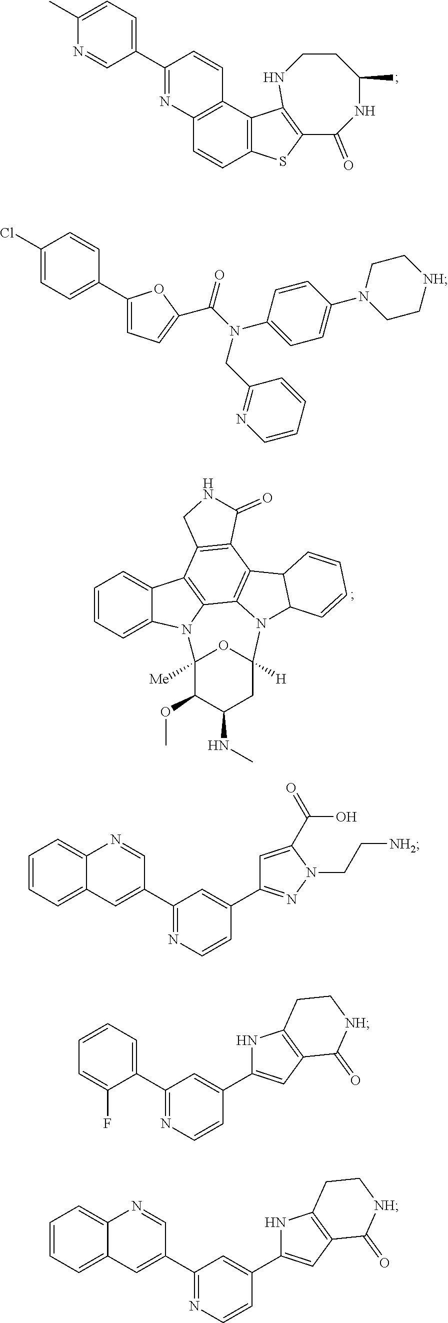

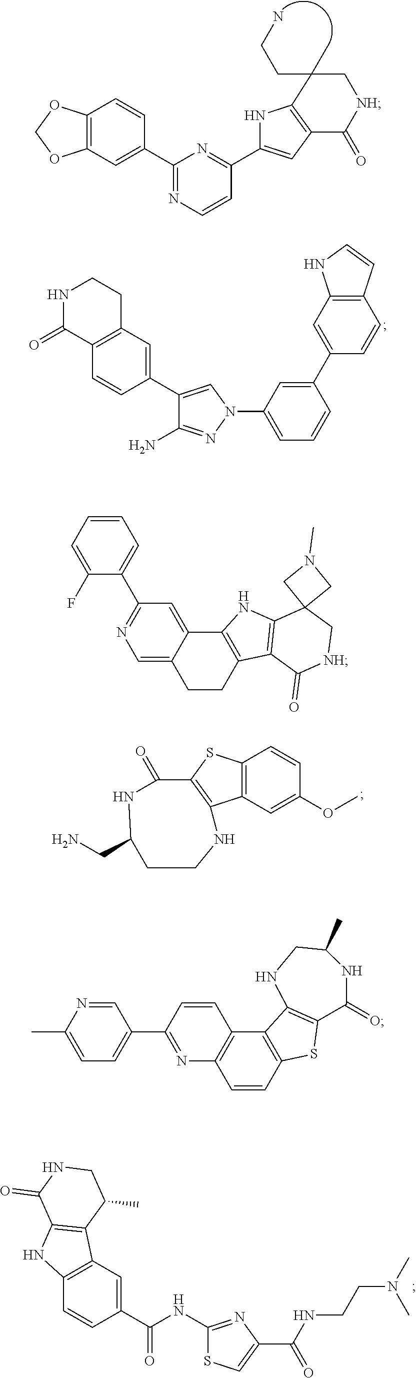

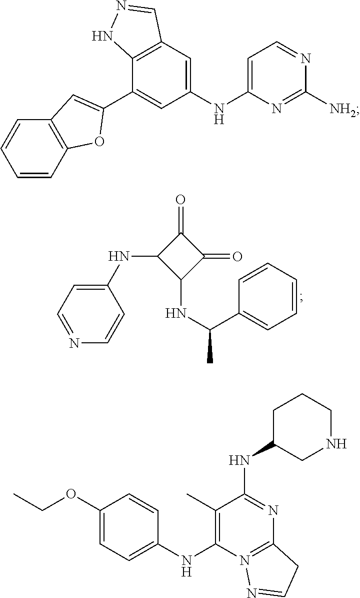

or a combination thereof.

According to another embodiment, the small molecule MK2 inhibitor competes with ATP for binding to MK2. According to some embodiments, the small molecule MK2 inhibitor is a pyrrolopyridine analogue or a multi-cyclic lactam analogue.

According to another embodiment, the small molecule MK2 inhibitor is a pyrrolopyridine analogue. Exemplary pyrrolopyridine analogues are described in Anderson, D. R. et al., “Pyrrolopyridine inhibitors of mitogen-activated protein kinase-activated protein kinase 2 (MK-2),” J. Med. Chem., 50: 2647-2654 (2007), the entire disclosure of which is incorporated herein by reference. According to another embodiment, the pyrrolopyridine analogue is a 2-aryl pyridine compound of formula I:

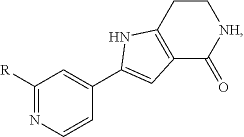

wherein R is H, Cl, phenyl, pyridine, pyrimidine, thienyl, naphthyl, benzothienyl, or quinoline. According to another embodiment, the pyrrolopyridine analogue is a 2-aryl pyridine compound of formula II:

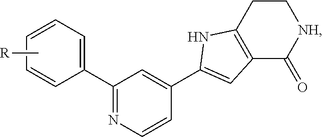

wherein R is OH, Cl, F, CF₃, CN, acetyl, methoxy, NH₂, CO₂H, CONH-cyclopropyl, CONH-cyclopentyl, CONH-cyclohexyl, CONHCH₂-phenyl, CONH(CH₂)₂-phenyl, or CON(methyl)CH₂-phenyl.

According to another embodiment, the small molecule MK2 inhibitor is a multi-cyclic lactam analogue. Exemplary multicyclic lactam analogues are described in Recesz, L. et al., “In vivo and in vitro SAR of tetracyclic MAPKAP-K₂ (MK2) inhibitors: Part I,” Bioorg. Med. Chem. Lett., 20: 4715-4718 (2010); and Recesz, L. et al., “In vivo and in vitro SAR of tetracyclic MAPKAP-K2 (MK2) inhibitors: Part II,” Bioorg. Med. Chem. Lett., 20: 4719-4723 (2010), the entire disclosure of each of which is incorporated herein by reference.

Cutaneous Scar

According to one embodiment, the cutaneous scar can result from healing of a wound. According to another embodiment, the wound is characterized by aberrant activity of Mitogen-Activated Protein Kinase-Activated Protein Kinase 2 (MK2) in a tissue compared to the activity of Mitogen-Activated Protein Kinase-Activated Protein Kinase 2 (MK2) in the tissue of a normal control subject.

According to another embodiment, the therapeutic amount is effective to reduce incidence, severity, or both, of the cutaneous scar without impairing normal wound healing.

According to another embodiment, the pharmaceutical composition is capable of improving alignment of collagen fibers in the wound. According to another embodiment, the therapeutic amount is effective to reduce collagen whorl formation in the wound.

According to one embodiment, the therapeutic amount is effective to accelerate wound healing compared to a control. According to another embodiment, the therapeutic amount is effective to decrease wound size compared to a control. According to some such embodiments, the therapeutic amount is effective to decrease wound size compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 1 day of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 2 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 3 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 4 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 5 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 6 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 7 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 8 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 9 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 10 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 11 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 12 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 13 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 14 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 21 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 30 days of the administration.

According to another embodiment, the therapeutic amount is effective to reduce scarring compared to a control. According to another embodiment, the therapeutic amount is effective to reduce scarring compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration. According to another embodiment, the therapeutic amount is effective to reduce scarring compared to a control as measured by visual analog scale (VAS) score, color matching (CM), matte/shiny (M/S) assessment, contour (C) assessment, distortion (D) assessment, texture (T) assessment, or a combination thereof.

According to another embodiment, the therapeutic amount is effective to decrease scar area compared to a control. According to some such embodiments, the therapeutic amount is effective to decrease scar area compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 1 day of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 2 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 3 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 4 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 5 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 6 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 7 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 8 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 9 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 10 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 11 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 12 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 13 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 14 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 21 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 30 days of the administration.

According to some embodiments, the pharmaceutical composition is capable of modulating expression of a scar-related gene or production of a scar-related gene product. According to one embodiment, the therapeutic amount is effective to modulate the expression of a scar-related gene. According to another embodiment, the therapeutic amount is effective to modulate messenger RNA (mRNA) level expressed from a scar-related gene. According to another embodiment, the therapeutic amount is effective to modulate level of a scar-related gene product expressed from a scar-related gene.

According to some such embodiments, the scar-related gene encodes one or more of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD). According to one embodiment, the scar-related gene encodes Transforming Growth Factor-β1 (TGF-β1). According to another embodiment, the scar-related gene encodes Tumor Necrosis Factor-α (TNF-α). According to another embodiment, the scar-related gene encodes a collagen. According to another embodiment, the collagen is collagen type 1α2 (col1α2) or collagen type 3α1 (col 3α1). According to another embodiment, the scar-related gene encodes Interleukin-6 (IL-6). According to another embodiment, the scar-related gene encodes chemokine (C-C motif) ligand 2 (CCL2) (or monocyte chemotactic protein-1 (MCP-1)). According to another embodiment, the scar-related gene encodes chemokine (C-C motif) receptor 2 (CCR2). According to another embodiment, the scar-related gene encodes EGF-like module-containing mucin-like hormone receptor-like 1 (EMR1). According to another embodiment, the scar-related gene encodes a sma/mad-related protein (SMAD).

According to some such embodiments, the scar-related gene product is selected from the group consisting of Transforming Growth Factor-β1 (TGF-β1), Tumor Necrosis Factor-α(TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD). According to another embodiment, the scar-related gene product is Tumor Necrosis Factor-α (TNF-α). According to another embodiment, the scar-related gene product is a collagen. According to another embodiment, the collagen is collagen type 1α2 (col1α2) or collagen type 3α1 (col 3α1). According to another embodiment, the scar-related gene product is Interleukin-6 (IL-6). According to another embodiment, the scar-related gene product is chemokine (C-C motif) ligand 2 (CCL2) (or monocyte chemotactic protein-1 (MCP-1)). According to another embodiment, the scar-related gene product is chemokine (C-C motif) receptor 2 (CCR2). According to another embodiment, the scar-related gene product is EGF-like module-containing mucin-like hormone receptor-like 1 (EMR1). According to another embodiment, the scar-related gene product is a sma/mad-related protein (SMAD).

According to another embodiment, the pharmaceutical composition is capable of reducing infiltration of one or more types of inflammatory or stem cells, including, without limitation, monocytes, fibrocytes, macrophages, lymphocytes, and mast or dendritic cells, into the wound.

According to another embodiment, the therapeutic amount is effective to reduce infiltration of at least one immunomodulatory cell into the wound. According to some such embodiments, the immunomodulatory cell is selected from the group consisting of a monocyte, a mast cell, a dendritic cell, a macrophage, a T-lymphocyte, or a fibrocyte. According to one embodiment, the immunomodulatory cell is a mast cell. According to another embodiment, the mast cell is characterized by expression of cell surface marker(s) including without limitation CD45 and CD117. According to another embodiment, the immunomodulatory cell is a monocyte. According to another embodiment, the monocyte is characterized by expression of cell surface marker(s) including without limitation CD11b. According to another embodiment, the immunomodulatory cell is a macrophage. According to another embodiment, the macrophage is characterized by expression of cell surface marker(s) including without limitation F4/80. According to another embodiment, the immunomodulatory cell is a T-lymphoyte. According to another embodiment, the T-lymphocyte is a helper T-lymphocyte or a cytotoxic T lymphocyte. According to another embodiment, the T-lymphocyte is characterized by expression of cell surface marker(s) including without limitation CD4, CD8, or a combination thereof.

According to another embodiment, the therapeutic amount is effective to reduce infiltration of at least one progenitor cell into the wound. According to some such embodiments, the progenitor cell is selected from the group consisting of a hematopoitic stem cell, a mesenchymal stem cell, or a combination thereof. According to one embodiment, the progenitor cell is a hematopoietic stem cell. According to another embodiment, the hematopoietic stem cell is characterized by expression of cell surface marker(s) including without limitation CD45 and Sca1. According to another embodiment, the progenitor cell is a mesenchymal stem cell. According to another embodiment, the mesenchymal stem cell is characterized by expression of cell surface marker(s) including without limitation Sca1 and not CD45.

According to another embodiment, the therapeutic amount is effective to reduce a level of transforming growth factor-β (TGF-β) expression in the wound. According to another embodiment, the therapeutic amount is effective to reduce messenger RNA (mRNA) level of transforming growth factor-β (TGF-β) in the wound. According to another embodiment, the therapeutic amount is effective to reduce protein level of transforming growth factor-β (TGF-β) in the wound.

According to another embodiment, the therapeutic amount is effective to modulate a level of an inflammatory mediator in the wound. According to some embodiments, the inflammatory mediator thus modulated can be without limitation interleukin-1 (IL-1), interleukin-4 (IL-4), interleukin-6 (IL-6), interleukin-8 (IL-8), tumor necrosis factor (TNF), interferon-gamma (IFN-γ), interleukin 12 (IL-12), or a combination thereof.

According to some embodiments, the wound is an abrasion, a laceration, a crush, a contusion, a puncture, an avulsion, a burn, an ulcer, or a combination thereof. According to one embodiment, the wound is an abrasion. According to another embodiment, the wound is a laceration. According to another embodiment, the wound is a crush. According to another embodiment, the wound is a contusion. According to another embodiment, the wound is a puncture. According to another embodiment, the wound is an avulsion. According to another embodiment, the wound is a burn. According to another embodiment, the wound is an ulcer.

According to another embodiment, the wound is an incisional wound.

According to another embodiment, the cutaneous scar is a pathological scar, meaning a scar arising as a result of a disease, disorder, condition, or injury.

According to another embodiment, the pathological scar is a hypertrophic scar.

According to another embodiment, the pathological scar is a keloid.

According to another embodiment, the pathological scar is an atrophic scar.

According to another embodiment, the pathological scar is a scar contracture.

According to another embodiment, the cutaneous scar is an incisional scar.

According to another embodiment, the hypertrophic scar results from a high-tension wound. According to another embodiment, the high-tension wound is located in close proximity to a joint. According to another embodiment, the joint is a knee, an elbow, a wrist, a shoulder, a hip, a spine, across a finger, or a combination thereof. The term “in close proximity” as used herein refers to a distance very near. According to one embodiment, the distance is from about 0.001 mm to about 15 cm. According to another embodiment, the distance is from about 0.001 mm to about 0.005 mm. According to another embodiment, the distance is from about 0.005 mm to about 0.01 mm. According to another embodiment, the distance is from about 0.01 mm to about 0.05 mm. According to another embodiment, the distance is from about 0.05 mm to about 0.1 mm. According to another embodiment, the distance is from about 0.1 mm to about 0.5 mm. According to another embodiment, the distance is from about 0.5 mm to about 1 mm. According to another embodiment, the distance is from about 1 mm to about 2 mm. According to another embodiment, the distance is from about 2 mm to about 3 mm. According to another embodiment, the distance is from about 3 mm to about 4 mm. According to another embodiment, the distance is from about 4 mm to about 5 mm. According to another embodiment, the distance is from about 5 mm to about 6 mm. According to another embodiment, the distance is from about 6 mm to about 7 mm. According to another embodiment, the distance is from about 7 mm to about 8 mm. According to another embodiment, the distance is from about 8 mm to about 9 mm. According to another embodiment, the distance is from about 9 mm to about 1 cm. According to another embodiment, the distance is from about 1 cm to about 2 cm. According to another embodiment, the distance is from about 2 cm to about 3 cm. According to another embodiment, the distance is from about 3 cm to about 4 cm. According to another embodiment, the distance is from about 4 cm to about 5 cm. According to another embodiment, the distance is from about 5 cm to about 6 cm. According to another embodiment, the distance is from about 6 cm to about 7 cm. According to another embodiment, the distance is from about 7 cm to about 8 cm. According to another embodiment, the distance is from about 8 cm to about 9 cm. According to another embodiment, the distance is from about 9 cm to about 10 cm. According to another embodiment, the distance is from about 10 cm to about 11 cm. According to another embodiment, the distance is from about 11 cm to about 12 cm. According to another embodiment, the distance is from about 12 cm to about 13 cm. According to another embodiment, the distance is from about 14 cm to about 15 cm.

According to some embodiments, the pathological scar results from an abrasion, a laceration, an incision, a crush, a contusion, a puncture, an avulsion, a burn, an ulcer, or a combination thereof. According to one embodiment, the pathological scar results from an abrasion. According to another embodiment, the pathological scar results from a laceration. According to another embodiment, the pathological scar results from an incision. According to another embodiment, the pathological scar results from a crush. According to another embodiment, the pathological scar results from a contusion. According to another embodiment, the pathological scar results from a puncture. According to another embodiment, the pathological scar results from an avulsion. According to another embodiment, the pathological scar results from a burn. According to another embodiment, the pathological scar results from an ulcer.

Cutaneous Scar Associated with an Autoimmune Skin Disorder

The term “autoimmune disorder” as used herein refers to disease, disorders or conditions in which the body's immune system, which normally fights infections and viruses, is misdirected and attacks the body's own normal, healthy tissue. In higher organisms, multiple mechanisms of immunological tolerance eliminate or inactivate lymphocytes that bear receptors specific for autoantigens. However, some autoreactive lymphcytes can escape from such mechanisms and present themselves within the peripheral lymphocyte pool.

Autoimmunity is caused by a complex interaction of multiple gene products, unlike immunodeficiency diseases, where a single dominant genetic trait is often the main disease determinant. (Reviewed in Fathman, C. G. et al., “An array of possibilities for the study of autoimmunity” Nature, 435(7042): 605-611 (2005); Anaya, J.-M., “Common mechanisms of autoimmune diseases (the autoimmune tautology),” Autoimmunity Reviews, 11(11): 781-784 (2012)). Autoimmune diseases are major causes of morbidity and mortality throughout the world and are difficult to treat. (Reviewed in for example in Hayter, S. M. et al., “Updated assessment of the prevalence, spectrum and case definition of autoimmune disease,” Autoimmunity Reviews, 11(10): 754-765 (2012); and Rioux, J. D. et al., “Paths to understanding the genetic basis of autoimmune disease,” Nature, 435(7042): 584-589 (2005)).

One mechanism by which the pathogenic potential of such autoreactive lymphocytes is kept in check is through a dedicated lineage of regulatory T (T_{R}) cells. These have been targeted for therapeutic intervention in a wide variety of autoimmune disorders (Reviewed in Kronenberg, M. et al., “Regulation of immunity by self-reactive T cells,” Nature, 435(7042): 598-604 (2005)).

Other components of the pathological cascade in autoimmune disorders that have received attention include, for example, factors involved in lymphocyte homing to target tissues; enzymes that are critical for the penetration of blood vessels and the extracellular matrix by immune cells; cytokines that mediate pathology within the tissues; various cell types that mediate damage at the site of disease, cell antigens; specific adaptive receptors, including the T-cell receptor (TCR) and immunoglobulin; and toxic mediators, such as complement components and nitric oxide. (Reviewed in Feldmann, M. et al., “Design of effective immunotherapy for human autoimmunity,” Nature, 435(7042): 612-619 (2005)).

Although mutations in a single gene can cause autoimmunity, most autoimmune diseases are associated with multiple sequence variants. (Reviewed in Rioux, J. D. et al., “Paths to understanding the genetic basis of autoimmune disease,” Nature, 435(7042): 584-589 (2005); and Goodnow, C. C. et al., “Cellular and genetic mechanisms of self-tolerance and autoimmunity,” Nature, 435(7042): 590-596 (2005)). Autoimmune disorders can be associated with chronic inflammation. Such autoimmune disorders are known as “autoinflammatory conditions”. (Reviewed in Hashkes, P. J. et al., “Autoinflammatory syndromes,” Pediatr. Clin. North Am., 59(2): 447-470 (2012)).

Systemic autoimmunity encompasses autoimmune conditions in which autoreactivity is not limited to a single organ or organ system. This definition includes, but is not limited to, autoimmune diseases including autoimuune skin disease manifestations such as systemic lupus erythematosus (SLE), systemic sclerosis (scleroderma), pemphigus, vitiligo, dermatitis herpetiformis, psoriasis, etc. Cutaneous SLE is a common systemic autoimmune disorder that includes specific skin manifestations such as “butterfly” rash, photosensitive rash dermatitis, and discoid lesions as well as vasculitis and alopecia. SLE is characterized by the presence of antinuclear antibodies (ANAs) and is associated with chronic inflammation. Scleroderma (or systemic sclerosis) is marked by inflammation, followed by deposition of ANAs in skin and viscera. Scleroderma is characterized by a marked reduction in circulation in peripheral arteries of distal fingertips (often stimulated by cold temperatures) known as Reynauld's phenomenon. Pemphigus comprises a group of autoimmune blistering diseases characterized by autoantibody induced epidermal cell-cell detachment (acantholysis). Pemphigus manifests clinically with flaccid blisters and skin erosions. Vitiligo is a skin depigmentation disorder that may be associated with other autoimmune disorders such as the autoimmune polyendocrine syndrome type I. Vitiligo is characterized by the presence of anti-melanocyte autoantibodies, skin infiltration of CD4+ and CD8+ T lymphocytes and overexpression of type I cytokine profiles. Dermatitis herpetiformis (DH) is a life long very pruritic, polymorphic blisteric skin disease associated with gluten sensitivity. The predominiant autoantigen in DH is tissue transglutaminase, found in the intestine and the skin. Psoriasis is a common autoimmune skin disease with a genetic basis affecting 1-3% of the Caucasian population. Psoriasis is characterized by hyperkeratosis, epidermal hyperplasia (acanthosis) and inflammation and dilation of dermal capillaries. (Paul, W. E., “Chapter 1: The immune system: an introduction,” Fundamental Immunology, 4^{th} Edition, Ed. Paul, W. E., Lippicott-Raven Publishers, Philadelphia (1999); Nancy, A.-L. and Yehuda, S., “Prediction and prevention of autoimmune skin disorders,” Arch. Dermatol. Res., 301: 57-64 (2009)).

According to some other embodiments, the pharmaceutical composition is capable of treating a cutaneous scar associated with an autoimmune skin disorder. According to some such embodiments, the autoimmune skin disorder is selected from the group consisting of systemic lupus erythematosus (SLE), systemic sclerosis (scleroderma), pemphigus, vitiligo, dermatitis herpetiformis, psoriasis, or a combination thereof. According to one embodiment, the autoimmune skin disorder is systemic lupus erythematosus (SLE). According to another embodiment, the autoimmune skin disorder is systemic sclerosis (scleroderma). According to another embodiment, the autoimmune skin disorder is pemphigus. According to another embodiment, the autoimmune skin disorder is vitiligo. According to another embodiment, the autoimmune skin disorder is dermatitis herpetiformis. According to another embodiment, the autoimmune skin disorder is psoriasis.

Effect on Kinase Activity

According to some other embodiments, the pharmaceutical composition may be chosen based on its ability to inhibit, or not to inhibit, one or more selected kinases selected from the group consisting of Abelson murine leukemia viral oncogene homolog 1 (Ab1), Abelson murine leukemia viral oncogene homolog 1 (T₃₁₅₁) (Ab1 (T3151)), Abelson murine leukemia viral oncogene homolog 1 (Y253F) (Ab1 (Y253F)), Anaplastic lymphoma kinase (ALK), Abelson-related gene (Arg), 5′-AMP-activated protein kinase catalytic subunit alpha-1 (AMPKα1), 5′-AMP-activated protein kinase catalytic subunit alpha-2 (AMPKα2), AMPK-related protein kinase 5 (ARKS), Apoptosis signal regulating kinase 1 (ASK1), Aurora kinase B (Aurora-B), AXL receptor tyrosine kinase (Ax1), Bone marrow tyrosine kinase gene in chromosome X protein (Bmx), Breast tumor kinase (BRK), Bruton's tyrosine kinase (BTK), Bruton's tyrosine kinase (R28H) (BTK (R28H)), Ca²⁺/calmodulin-dependent protein kinase I (CaMKI), Ca²⁺/calmodulin-dependent protein kinase IIβ (CaMIIβ), Ca²⁺/calmodulin-dependent protein kinase IIγ (CaMKIIγ), Ca²⁺/calmodulin-dependent protein kinase δ (CaMKIδ), Ca2⁺/calmodulin-dependent protein kinase IIδ (CaMKIIδ), Ca²⁺/calmodulin-dependent protein kinase IIγ (CaMKIIγ), Cell devision kinase 2 (CDK2/cyclinE), Cell devision kinase 3 (CDK3/cyclinE), Cell devision kinase 6 (CDK6/cyclinD3), Cell devision kinase 7 (CDK7/cyclinH/MAT1), Cell devision kinase 9 (CDK9/cyclin T1), Checkpoint kinase 2 (CHK2), Checkpoint kinase 2 (1157T) (CHK2 (1157T)), Checkpoint kinase 2 (R145W) (CHK2 (R145W)), Proto-oncogene tyrosine-protein kinase cKit (D816V) (cKit (D816V)), C-src tyrosine kinase (CSK), Raf proto-oncogene serine/threonine protein kinase (c-RAF), Proto-oncogene tyrosine-protein kinase (cSRC), Death-associated protein kinase 1 (DAPK1), Death-associated protein kinase 2 (DAPK2), Dystrophia myotonica-protein kinase (DMPK), DAP kinase-related apoptosis-inducing protein kinase 1 (DRAK1), Epidermal growth factor receptor (EGFR), Epidermal growth factor receptor (EGFR L858R), Epidermal growth factor receptor L861Q (EGFR (L861Q)), Eph receptor A2 (EphA2) (EphA2), Eph receptor A3 (EphA3), Eph receptor A5 (EphA5), Eph receptor B2 (EphB2), Eph receptor B4 (EphB4), Erythroblastic leukemia viral oncogene homolog 4 (ErbB4), c-Fes protein tyrosine kinase (Fes), Fibroblast growth factor receptor 2 (FGFR2), Fibroblast growth factor receptor 3 (FGFR3), Fibroblast growth factor receptor 4 (FGFR4), Fms-like tyrosine kinase receptor-3 (Flt3), FMS proto-oncogene (Fms), Haploid germ cell-specific nuclear protein kinase (Haspin), Insulin receptor-related receptor (IRR), Interleukin-1 receptor-associated kinase 1 (IRAK1), Interleukin-1 receptor-associated kinase 4 (IRAK4), IL2-inducible T-cell kinase (Itk), Kinase insert domain receptor (KDR), Lymphocyte cell-specific protein-tyrosine kinase (Lck), Lymphocyte-oriented kinase (LOK), Lyn tyrosine protein kinase (Lyn), MAP kinase-activated protein kinase 2 (MK2), MAP kinase-activated protein kinase 3 (MK3), MEK1, Maternal embryonic leucine zipper kinase (MELK), c-Mer proto-oncogene tyrosine kinase (Mer), c-Met proto-oncogene tyrosine kinase (Met), c-Met proto-oncogene tyrosine kinase D1246N (Met (D1246N)), c-Met proto-oncogene tyrosine kinase Y1248D (Met Y1248D), Misshapen/NIK-related kinase (MINK), MAP kinase kinase 6 (MKK6), Myosin light-chain kinase (MLCK), Mixed lineage kinase 1 (MLK1), MAP kinase signal-integrating kinase 2 (MnK2), Myotonic dystrophy kinase-related CDC42-binding kinase alpha (MRCKα), Myotonic dystrophy kinase-related CDC42-binding kinase beta (MRCKβ), Mitogen- and stress-activated protein kinase 1 (MSK1), Mitogen- and stress-activated protein kinase 2 (MSK2), Muscle-specific serine kinase 1 (MSSK1), Mammalian STE20-like protein kinase 1 (MST1), Mammalian STE20-like protein kinase 2 (MST2), Mammalian STE20-like protein kinase 3 (MST3), Muscle, skeletal receptor tyrosine-protein kinase (MuSK), Never in mitosis A-related kinase 2 (NEK2), Never in mitosis A-related kinase 3 (NEK3), Never in mitosis A-related kinase 11 (NEK11), 70 kDa ribosomal protein S6 kinase 1 (p70S6K), PAS domain containing serine/threonine kinase (PASK), Phosphorylase kinase subunit gamma-2 (PhKγ2), Pim-1 kinase (Pim-1), Protein kinase B alpha (PKBα), Protein kinase B beta (PKBβ), Protein kinase B gamma (PKBγ), Protein kinase C, alpha (PKCα), Protein kinase C, beta1 (PKCβ1), Protein kinase C, beta II (PKCβII), Protein kinase C, gamma (PKCγ), Protein kinase C, epsilon (PKCε), Protein kinase C, iota (PCKι), Protein kinase C, mu (PKCμ), Protein kinase C, zeta (PKCζ), protein kinase D2 (PKD2), cGMP-dependent protein kinase 1 alpha (PKG1α), cGMP-dependent protein kinase 1 beta (PKG1β), Protein-kinase C-related kinase 2 (PRK2), Proline-rich tyrosine kinase 2 (Pyk2), Proto-oncogene tyrosine-protein kinase receptor Ret V804L (Ret (V804L)), Receptor-interacting serine-threonine kinase 2 (RIPK2), Rho-associated protein kinase I (ROCK-I), Rho-associated protein kinase II (ROCK-II), Ribosomal protein S6 kinase 1 (Rsk1), Ribosomal protein S6 kinase 2 (Rsk2), Ribosomal protein S6 kinase 3 (Rsk3), Ribosomal protein S6 kinase 4 (Rsk4), Stress-activated protein kinase 2A T106M (SAPK2a, T106M), Stress-activated protein kinase 3 (SAPK3), Serum/glucocorticoid regulated kinase (SGK), Serum/glucocorticoid regulated kinase 2 (SGK2), Serum/glucocorticoid-regulated kinase 3 (SGK3), Proto-oncogene tyrosine-protein kinase Src 1-530 (Src, 1-530), Serine/threonine-protein kinase 33 (STK33), Spleen tyrosine kinase (Syk), Thousand and one amino acid protein 1 (TAO1), Thousand and one amino acid protein 2 (TAO2), Thousand and one amino acid protein 3 (TAO3), TANK-binding kinase 1 (TBK1), Tec protein tyrosine kinase (Tec), Tunica interna endothelial cell kinase 2 (Tie2), Tyrosine kinase receptor A (TrkA), BDNF/NT-3 growth factors receptor (TrkB), TXK tyrosine kinase (Txk), WNK lysine deficient protein kinase 2 (WNK2), WNK lysine deficient protein kinase 3 (WNK3), Yamaguchi sarcoma viral oncogene homolog 1 (Yes), Zeta-chain (TCR) Associated Protein kinase 70 kDa (ZAP-70), and ZIP kinase (ZIPK).

According to some other embodiments, the pharmaceutical composition is capable of inhibiting a kinase activity of Mitogen-Activated Protein Kinase-Activated Protein Kinase 2 (MK2). According to some such embodiments, the therapeutic amount is effective to inhibit the kinase activity of Mitogen-Activated Protein Kinase-Activated Protein Kinase 2 (MK2). According to one embodiment, the therapeutic amount is effective to inhibit at least 50% of the kinase activity of MK2 kinase. According to another embodiment, the therapeutic amount is effective to inhibit at least 65% of the kinase activity of MK2 kinase. According to another embodiment, the therapeutic amount is effective to inhibit at least 75% of the kinase activity of MK2 kinase. According to another embodiment, the therapeutic amount is effective to inhibit at least 80% of the kinase activity of MK2 kinase. According to another embodiment, the therapeutic amount is effective to inhibit at least 85% of the kinase activity of MK2 kinase. According to another embodiment, the therapeutic amount is effective to inhibit at least 90% of the kinase activity of MK2 kinase. According to another embodiment, the therapeutic amount is effective to inhibit at least 95% of the kinase activity of MK2 kinase.

According to another embodiment, the MK2 polypeptide inhibitor inhibits the kinase activity of Mitogen-Activated Protein Kinase-Activated Protein Kinase 2 (MK2) with an inhibition activity of IC₅₀ of at least about 12 μM.

According to another embodiment, the pharmaceutical composition is capable of inhibiting a kinase activity of Mitogen-Activated Protein Kinase-Activated Protein Kinase 3 (MK3). According to some such embodiments, the therapeutic amount is effective to inhibit the kinase activity of Mitogen-Activated Protein Kinase-Activated Protein Kinase 3 (MK3). According to one embodiment, the therapeutic amount is effective to inhibit at least 50% of the kinase activity of MK3 kinase. According to another embodiment, the therapeutic amount is effective to inhibit at least 65% of the kinase activity of MK3 kinase. According to another embodiment, the therapeutic amount is effective to inhibit at least 70% of the kinase activity of MK3 kinase. According to another embodiment, the therapeutic amount is effective to inhibit at least 75% of the kinase activity of MK3 kinase. According to another embodiment, the therapeutic amount is effective to inhibit at least 80% of the kinase activity of MK3 kinase. According to another embodiment, the therapeutic amount is effective to inhibit at least 85% of the kinase activity of MK3 kinase. According to another embodiment, the therapeutic amount is effective to inhibit at least 90% of the kinase activity of MK3 kinase. According to another embodiment, the therapeutic amount is effective to inhibit at least 95% of the kinase activity of MK3 kinase.

According to another embodiment, the MK2 polypeptide inhibitor inhibits the kinase activity of Mitogen-Activated Protein Kinase-Activated Protein Kinase 3 (MK3) with an inhibition activity of IC₅₀ of at least about 16 μM.

According to another embodiment, the pharmaceutical composition is capable of inhibiting a kinase activity of Ca²⁺/calmodulin-dependent protein kinase I (CaMKI). According to some such embodiments, the therapeutic amount is effective to inhibit the kinase activity of Ca²⁺/calmodulin-dependent protein kinase I (CaMKI). According to one embodiment, the therapeutic amount is effective to inhibit at least 50% of the kinase activity of Ca²⁺/calmodulin-dependent protein kinase I (CaMKI). According to another embodiment, the therapeutic amount is effective to inhibit at least 65% of the kinase activity of Ca²⁺/calmodulin-dependent protein kinase I (CaMKI). According to another embodiment, the therapeutic amount is effective to inhibit at least 70% of the kinase activity of Ca²⁺/calmodulin-dependent protein kinase I (CaMKI). According to another embodiment, the therapeutic amount is effective to inhibit at least 75% of the kinase activity of Ca²⁺/calmodulin-dependent protein kinase I (CaMKI). According to another embodiment, the therapeutic amount is effective to inhibit at least 80% of the kinase activity of Ca²⁺/calmodulin-dependent protein kinase I (CaMKI). According to another embodiment, the therapeutic amount is effective to inhibit at least 85% of the kinase activity of Ca²⁺/calmodulin-dependent protein kinase I (CaMKI). According to another embodiment, the therapeutic amount is effective to inhibit at least 90% of the kinase activity of Ca²⁺/calmodulin-dependent protein kinase I (CaMKI). According to another embodiment, the therapeutic amount is effective to inhibit at least 95% of the kinase activity of Ca²⁺/calmodulin-dependent protein kinase I (CaMKI).

According to another embodiment, the MK2 polypeptide inhibitor inhibits the kinase activity of Ca²⁺/calmodulin-dependent protein kinase I (CaMKI) with an inhibition activity of IC₅₀ of at least about 12 μM.

According to another embodiment, the pharmaceutical composition is capable of inhibiting a kinase activity of BDNF/NT-3 growth factors receptor (TrkB). According to some such embodiments, the therapeutic amount is effective to inhibit the kinase activity of BDNF/NT-3 growth factors receptor (TrkB). According to one embodiment, the therapeutic amount is effective to inhibit at least 50% of the kinase activity of BDNF/NT-3 growth factors receptor (TrkB). According to another embodiment, the therapeutic amount is effective to inhibit at least 65% of the kinase activity of BDNF/NT-3 growth factors receptor (TrkB). According to another embodiment, the therapeutic amount is effective to inhibit at least 70% of the kinase activity of BDNF/NT-3 growth factors receptor (TrkB). According to another embodiment, the therapeutic amount is effective to inhibit at least 75% of the kinase activity of BDNF/NT-3 growth factors receptor (TrkB). According to another embodiment, the therapeutic amount is effective to inhibit at least 80% of the kinase activity of BDNF/NT-3 growth factors receptor (TrkB). According to another embodiment, the therapeutic amount is effective to inhibit at least 85% of the kinase activity of BDNF/NT-3 growth factors receptor (TrkB). According to another embodiment, the therapeutic amount is effective to inhibit at least 90% of the kinase activity of BDNF/NT-3 growth factors receptor (TrkB). According to another embodiment, the therapeutic amount is effective to inhibit at least 95% of the kinase activity of BDNF/NT-3 growth factors receptor (TrkB).

According to another embodiment, the MK2 polypeptide inhibitor inhibits the kinase activity of BDNF/NT-3 growth factors receptor (TrkB) with an inhibition activity of IC₅₀ of at least about 5 μM.

According to another embodiment, the pharmaceutical composition is capable of inhibiting a kinase activity of Mitogen-Activated Protein Kinase-Activated Protein Kinase 2 (MK2) and a kinase activity of calcium/calmodulin-dependent protein kinase I (CaMKI). According to one embodiment, the therapeutic amount is effective to inhibit the kinase activity of Mitogen-Activated Protein Kinase-Activated Protein Kinase 2 (MK2) and the kinase activity of calcium/calmodulin-dependent protein kinase I (CaMKI). According to another embodiment, the therapeutic amount is effective to: (1) inhibit the kinase activity of Mitogen-Activated Protein Kinase-Activated Protein Kinase 2 (MK2); and (2) further inhibit the kinase activity of calcium/calmodulin-dependent protein kinase I (CaMKI).

According to one embodiment, the therapeutic amount is effective to: (1) inhibit at least 50% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 50% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 50% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 50% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 50% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 50% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 50% of the kinase activity of MK2 kinase; and (2) further inhibit at least 95% of the kinase activity of Ca²⁺/calmodulin-dependent protein kinase I (CaMKI).

According to one embodiment, the therapeutic amount is effective to: (1) inhibit at least 65% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 65% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 65% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 65% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 65% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 65% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 65% of the kinase activity of MK2 kinase; and (2) further inhibit at least 95% of the kinase activity of Ca²⁺/calmodulin-dependent protein kinase I (CaMKI).

According to one embodiment, the therapeutic amount is effective to: (1) inhibit at least 75% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 75% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 75% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 75% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 75% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 75% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 75% of the kinase activity of MK2 kinase; and (2) further inhibit at least 95% of the kinase activity of Ca²⁺/calmodulin-dependent protein kinase I (CaMKI).

According to one embodiment, the therapeutic amount is effective to: (1) inhibit at least 80% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 80% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 80% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 80% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 80% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 80% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 80% of the kinase activity of MK2 kinase; and (2) further inhibit at least 95% of the kinase activity of Ca²⁺/calmodulin-dependent protein kinase I (CaMKI).

According to one embodiment, the therapeutic amount is effective to: (1) inhibit at least 85% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 85% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 85% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 85% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 85% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 85% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 85% of the kinase activity of MK2 kinase; and (2) further inhibit at least 95% of the kinase activity of Ca²⁺/calmodulin-dependent protein kinase I (CaMKI).

According to one embodiment, the therapeutic amount is effective to: (1) inhibit at least 90% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 90% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 90% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 90% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 90% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 90% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 90% of the kinase activity of MK2 kinase; and (2) further inhibit at least 95% of the kinase activity of Ca²⁺/calmodulin-dependent protein kinase I (CaMKI).

According to one embodiment, the therapeutic amount is effective to: (1) inhibit at least 95% of the kinase activity of MK2 kinase; and (2) further inhibit at least 65% of the kinase activity of Ca²⁺/calmodulin-dependent protein kinase I (CaMKI). According to another embodiment, the therapeutic amount is effective to: (1) inhibit at least 95% of the kinase activity of MK2 kinase; and (2) further inhibit at least 70% of the kinase activity of Ca²⁺/calmodulin-dependent protein kinase I (CaMKI). According to another embodiment, the therapeutic amount is effective to: (1) inhibit at least 95% of the kinase activity of MK2 kinase; and (2) further inhibit at least 75% of the kinase activity of Ca²⁺/calmodulin-dependent protein kinase I (CaMKI). According to another embodiment, the therapeutic amount is effective to: (1) inhibit at least 95% of the kinase activity of MK2 kinase; and (2) further inhibit at least 80% of the kinase activity of Ca²⁺/calmodulin-dependent protein kinase I (CaMKI). According to another embodiment, the therapeutic amount is effective to: (1) inhibit at least 95% of the kinase activity of MK2 kinase; and (2) further inhibit at least 85% of the kinase activity of Ca²⁺/calmodulin-dependent protein kinase I (CaMKI). According to another embodiment, the therapeutic amount is effective to: (1) inhibit at least 95% of the kinase activity of MK2 kinase; and (2) further inhibit at least 90% of the kinase activity of Ca²⁺/calmodulin-dependent protein kinase I (CaMKI). According to another embodiment, the therapeutic amount is effective to: (1) inhibit at least 95% of the kinase activity of MK2 kinase; and (2) further inhibit at least 95% of the kinase activity of Ca²⁺/calmodulin-dependent protein kinase I (CaMKI).

According to another embodiment, the pharmaceutical composition is capable of inhibiting a kinase activity of Mitogen-Activated Protein Kinase-Activated Protein Kinase 2 (MK2) and a kinase activity of BDNF/NT-3 growth factors receptor (TrkB). According to some such embodiments, the therapeutic amount is effective to inhibit the kinase activity of Mitogen-Activated Protein Kinase-Activated Protein Kinase 2 (MK2) and the kinase activity of BDNF/NT-3 growth factors receptor (TrkB). According to another embodiment, the therapeutic amount is effective to: (1) inhibit the kinase activity of Mitogen-Activated Protein Kinase-Activated Protein Kinase 2 (MK2); and (2) further inhibit the kinase activity of BDNF/NT-3 growth factors receptor (TrkB).

According to one embodiment, the therapeutic amount is effective to: (1) inhibit at least 50% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 50% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 65% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 65% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 70% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 70% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 75% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 75% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 80% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 80% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 85% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 85% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 90% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 90% of the kinase activity of BDNF/NT-3 growth factors receptor (TrkB). According to another embodiment, the therapeutic amount is effective to: (1) inhibit at least 90% of the kinase activity of MK2 kinase; and (2) further inhibit at least 95% of the kinase activity of BDNF/NT-3 growth factors receptor (TrkB).

According to one embodiment, the therapeutic amount is effective to: (1) inhibit at least 95% of the kinase activity of MK2 kinase; and (2) further inhibit at least 50% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 65% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 70% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 75% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 80% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 85% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 92% of the kinase activity of BDNF/NT-3 growth factors receptor (TrkB). According to another embodiment, the therapeutic amount is effective to: (1) inhibit at least 95% of the kinase activity of MK2 kinase; and (2) further inhibit at least 95% of the kinase activity of BDNF/NT-3 growth factors receptor (TrkB).

According to another embodiment, the pharmaceutical composition is capable of inhibiting a kinase activity of Mitogen-Activated Protein Kinase-Activated Protein Kinase 2 (MK2), a kinase activity of calcium/calmodulin-dependent protein kinase I (CaMKI), and a kinase activity of BDNF/NT-3 growth factors receptor (TrkB). According to some such embodiments, the therapeutic amount is effective to inhibit the kinase activity of Mitogen-Activated Protein Kinase-Activated Protein Kinase 2 (MK2), the kinase activity of calcium/calmodulin-dependent protein kinase I (CaMKI), and the kinase activity of BDNF/NT-3 growth factors receptor (TrkB). According to another embodiment, the therapeutic amount is effective to: (1) inhibit the kinase activity of Mitogen-Activated Protein Kinase-Activated Protein Kinase 2 (MK2); (2) further inhibit the kinase activity of calcium/calmodulin-dependent protein kinase I (CaMKI); (3) further inhibit the kinase activity of BDNF/NT-3 growth factors receptor (TrkB). According to some embodiments, the therapeutic amount is effective to: (1) inhibit at least 50%, at least 65%, at least 70%, at least 75%, at least 80%, at least 85%, at least 90%, or at least 95% of the kinase activity of Mitogen-Activated Protein Kinase-Activated Protein Kinase 2 (MK2); (2) further inhibit at least 50%, at least 65%, at least 70%, at least 75%, at least 80%, at least 85%, at least 90%, or at least 95% of the kinase activity of calcium/calmodulin-dependent protein kinase I (CaMKI); (3) further inhibit at least 50%, at least 65%, at least 70%, at least 75%, at least 80%, at least 85%, at least 90%, or at least 95% of the kinase activity of BDNF/NT-3 growth factors receptor (TrkB).

According to some embodiments, an inhibitory profile of the polypeptide of amino acid YARAAARQARAKALARQLGVAA (SEQ ID NO: 1) or a functional equivalent thereof depends on dosage, route of administration, cell type, or a combination thereof.

According to some embodiments, at least one Mitogen-Activated Protein Kinase-Activated Protein Kinase 2 (MK2) inhibitor or a functional equivalent thereof of the described invention substantially inhibits at least one kinase selected from the group consisting of: Abelson murine leukemia viral oncogene homolog 1 (Ab1), Abelson murine leukemia viral oncogene homolog 1 (T3151) (Ab1 (T3151)), Abelson murine leukemia viral oncogene homolog 1 (Y253F) (Ab1 (Y253F)), Anaplastic lymphoma kinase (ALK), Abelson-related gene (Arg), 5′-AMP-activated protein kinase catalytic subunit alpha-1 (AMPKα1), 5′-AMP-activated protein kinase catalytic subunit alpha-2 (AMPKα2), AMPK-related protein kinase 5 (ARKS), Apoptosis signal regulating kinase 1 (ASK1), Aurora kinase B (Aurora-B), AXL receptor tyrosine kinase (Ax1), Bone marrow tyrosine kinase gene in chromosome X protein (Bmx), Breast tumor kinase (BRK), Bruton's tyrosine kinase (BTK), Bruton's tyrosine kinase (R28H) (BTK (R28H)), Ca²⁺/calmodulin-dependent protein kinase I (CaMKI), Ca²⁺/calmodulin-dependent protein kinase IIβ (CaMIIβ), Ca²⁺/calmodulin-dependent protein kinase IIγ (CaMKIIγ), Ca²⁺/calmodulin-dependent protein kinase δ (CaMKIδ), Ca²⁺/calmodulin-dependent protein kinase IIδ (CaMKIIδ), Ca2⁺/calmodulin-dependent protein kinase IV (CaMKIV), Cell devision kinase 2 (CDK2/cyclinE), Cell devision kinase 3 (CDK3/cyclinE), Cell devision kinase 6 (CDK6/cyclinD3), Cell devision kinase 7 (CDK7/cyclinH/MAT1), Cell devision kinase 9 (CDK9/cyclin T1), Checkpoint kinase 2 (CHK2), Checkpoint kinase 2 (1157T) (CHK2 (1157T)), Checkpoint kinase 2 (R145W) (CHK2 (R145W)), Proto-oncogene tyrosine-protein kinase cKit (D816V) (cKit (D816V)), C-src tyrosine kinase (CSK), Raf proto-oncogene serine/threonine protein kinase (c-RAF), Proto-oncogene tyrosine-protein kinase (cSRC), Death-associated protein kinase 1 (DAPK1), Death-associated protein kinase 2 (DAPK2), Dystrophia myotonica-protein kinase (DMPK), DAP kinase-related apoptosis-inducing protein kinase 1 (DRAK1), Epidermal growth factor receptor (EGFR), Epidermal growth factor receptor (EGFR L858R), Epidermal growth factor receptor L861Q (EGFR (L861Q)), Eph receptor A2 (EphA2) (EphA2), Eph receptor A3 (EphA3), Eph receptor A5 (EphA5), Eph receptor B2 (EphB2), Eph receptor B4 (EphB4), Erythroblastic leukemia viral oncogene homolog 4 (ErbB4), c-Fes protein tyrosine kinase (Fes), Fibroblast growth factor receptor 2 (FGFR2), Fibroblast growth factor receptor 3 (FGFR3), Fibroblast growth factor receptor 4 (FGFR4), Fms-like tyrosine kinase receptor-3 (Flt3), FMS proto-oncogene (Fms), Haploid germ cell-specific nuclear protein kinase (Haspin), Insulin receptor-related receptor (IRR), Interleukin-1 receptor-associated kinase 1 (IRAK1), Interleukin-1 receptor-associated kinase 4 (IRAK4), IL2-inducible T-cell kinase (Itk), Kinase insert domain receptor (KDR), Lymphocyte cell-specific protein-tyrosine kinase (Lck), Lymphocyte-oriented kinase (LOK), Lyn tyrosine protein kinase (Lyn), MAP kinase-activated protein kinase 2 (MK2), MAP kinase-activated protein kinase 3 (MK3), MEK1, Maternal embryonic leucine zipper kinase (MELK), c-Mer proto-oncogene tyrosine kinase (Mer), c-Met proto-oncogene tyrosine kinase (Met), c-Met proto-oncogene tyrosine kinase D1246N (Met (D1246N)), c-Met proto-oncogene tyrosine kinase Y1248D (Met Y1248D), Misshapen/NIK-related kinase (MINK), MAP kinase kinase 6 (MKK6), Myosin light-chain kinase (MLCK), Mixed lineage kinase 1 (MLK1), MAP kinase signal-integrating kinase 2 (MnK2), Myotonic dystrophy kinase-related CDC42-binding kinase alpha (MRCKα), Myotonic dystrophy kinase-related CDC42-binding kinase beta (MRCKβ), Mitogen- and stress-activated protein kinase 1 (MSK1), Mitogen- and stress-activated protein kinase 2 (MSK2), Muscle-specific serine kinase 1 (MSSK1), Mammalian STE20-like protein kinase 1 (MST1), Mammalian STE20-like protein kinase 2 (MST2), Mammalian STE20-like protein kinase 3 (MST3), Muscle, skeletal receptor tyrosine-protein kinase (MuSK), Never in mitosis A-related kinase 2 (NEK2), Never in mitosis A-related kinase 3 (NEK3), Never in mitosis A-related kinase 11 (NEK11), 70 kDa ribosomal protein S6 kinase 1 (p70S6K), PAS domain containing serine/threonine kinase (PASK), Phosphorylase kinase subunit gamma-2 (PhKγ2), Pim-1 kinase (Pim-1), Protein kinase B alpha (PKBα), Protein kinase B beta (PKBβ), Protein kinase B gamma (PKBγ), Protein kinase C, alpha (PKCα), Protein kinase C, beta1 (PKCβ1), Protein kinase C, beta II (PKCβII), Protein kinase C, gamma (PKCγ), Protein kinase C, epsilon (PKCε), Protein kinase C, iota (PCKι), Protein kinase C, mu (PKCμ), Protein kinase C, zeta (PKCζ), protein kinase D2 (PKD2), cGMP-dependent protein kinase 1 alpha (PKG1α), cGMP-dependent protein kinase 1 beta (PKG1β), Protein-kinase C-related kinase 2 (PRK2), Proline-rich tyrosine kinase 2 (Pyk2), Proto-oncogene tyrosine-protein kinase receptor Ret V804L (Ret (V804L)), Receptor-interacting serine-threonine kinase 2 (RIPK2), Rho-associated protein kinase I (ROCK-I), Rho-associated protein kinase II (ROCK-II), Ribosomal protein S6 kinase 1 (Rsk1), Ribosomal protein S6 kinase 2 (Rsk2), Ribosomal protein S6 kinase 3 (Rsk3), Ribosomal protein S6 kinase 4 (Rsk4), Stress-activated protein kinase 2A T106M (SAPK2a, T106M), Stress-activated protein kinase 3 (SAPK3), Serum/glucocorticoid regulated kinase (SGK), Serum/glucocorticoid regulated kinase 2 (SGK2), Serum/glucocorticoid-regulated kinase 3 (SGK3), Proto-oncogene tyrosine-protein kinase Src 1-530 (Src, 1-530), Serine/threonine-protein kinase 33 (STK33), Spleen tyrosine kinase (Syk), Thousand and one amino acid protein 1 (TAO1), Thousand and one amino acid protein 2 (TAO2), Thousand and one amino acid protein 3 (TAO3), TANK-binding kinase 1 (TBK1), Tec protein tyrosine kinase (Tec), Tunica interna endothelial cell kinase 2 (Tie2), Tyrosine kinase receptor A (TrkA), BDNF/NT-3 growth factors receptor (TrkB), TXK tyrosine kinase (Txk), WNK lysine deficient protein kinase 2 (WNK2), WNK lysine deficient protein kinase 3 (WNK3), Yamaguchi sarcoma viral oncogene homolog 1 (Yes), Zeta-chain (TCR) Associated Protein kinase 70 kDa (ZAP-70), and ZIP kinase (ZIPK). According to some embodiments, the Mitogen-Activated Protein Kinase-Activated Protein Kinase 2 (MK2) inhibitor is selected from the group consisting of a polypeptide MMI-0100 of amino acid sequence YARAAARQARAKALARQLGVAA (SEQ ID NO: 1), a polypeptide MMI-0200 of amino acid sequence YARAAARQARAKALNRQLGVA (SEQ ID NO: 19), a polypeptide MMI-0300 of amino acid sequence FAKLAARLYRKALARQLGVAA (SEQ ID NO: 3), a polypeptide MMI-0400 of amino acid sequence KAFAKLAARLYRKALARQLGVAA (SEQ ID NO: 4), and a polypeptide MMI-0500 of amino acid sequence HRRIKAWLKKIKALARQLGVAA (SEQ ID NO: 7).

According to some other embodiments, at least two Mitogen-Activated Protein Kinase-Activated Protein Kinase 2 (MK2) inhibitors selected from the group consisting of a polypeptide MMI-0100 of amino acid sequence YARAAARQARAKALARQLGVAA (SEQ ID NO: 1), a polypeptide MMI-0200 of amino acid sequence YARAAARQARAKALNRQLGVA (SEQ ID NO: 19), a polypeptide MMI-0300 of amino acid sequence FAKLAARLYRKALARQLGVAA (SEQ ID NO: 3), a polypeptide MMI-0400 of amino acid sequence KAFAKLAARLYRKALARQLGVAA (SEQ ID NO: 4), and a polypeptide MMI-0500 of amino acid sequence HRRIKAWLKKIKALARQLGVAA (SEQ ID NO: 7) substantially inhibits a kinase selected from the group consisting of: Anaplastic lymphoma kinase (ALK), Breast tumor kinase (BRK), Bruton's tyrosine kinase (BTK), Ca²⁺/calmodulin-dependent protein kinase I (including CaMKIδ), Ca²⁺/calmodulin-dependent protein kinase II (CaMKII, including CaMKIIβ, CaMKIIδ and CaMKIIγ), Ca²⁺/calmodulin-dependent protein kinase IV (CaMKIV), Checkpoint kinase 2 (CHK2 (R145W)), Proto-oncogene tyrosine-protein kinase cKit (D816V) (cKit (D816V)), C-src tyrosine kinase (CSK), Proto-oncogene tyrosine-protein kinase (cSRC), Death-associated protein kinase 1 (DAPK1), Death-associated protein kinase 2 (DAPK2), DAP kinase-related apoptosis-inducing protein kinase 1 (DRAK1), Epidermal growth factor receptor (EGFR), Epidermal growth factor receptor L861Q (EGFR (L861Q)), Eph receptor A2 (EphA2), Eph receptor A3 (EphA3), Eph receptor A5 (EphA5), Eph receptor B2 (EphB2), Erythroblastic leukemia viral oncogene homolog 4 (ErbB4), c-Fes protein tyrosine kinase (Fes), Fibroblast growth factor receptor 2 (FGFR2), Fibroblast growth factor receptor 3 (FGFR3), and Fibroblast growth factor receptor 4 (FGFR4), Fms-like tyrosine kinase receptor-3 (Flt3), Insulin receptor-related receptor (IRR), Lymphocyte-oriented kinase (LOK), Lyn tyrosine protein kinase (Lyn), MAP kinase-activated protein kinase 2 (MK2), MAP kinase-activated protein kinase 3 (MK3), Maternal embryonic leucine zipper kinase (MELK), Myosin light-chain kinase (MLCK), Mitogen- and stress-activated protein kinase (MSK1), Mammalian STE20-like protein kinase 1 (MST1), Mammalian STE20-like protein kinase 2 (MST2), Never in mitosis A-related kinase 11(NEK11), 70 kDa ribosomal protein S6 kinase 1 (p70S6K), PAS domain containing serine/threonine kinase (PASK), Pim-1 kinase (Pim-1), Protein kinase B, gamma (PKBγ), Protein kinase C, mu (PKCμ), protein kinase D2 (PKD2), Protein-kinase C-related kinase 2 (PRK2), Serum/glucocorticoid-regulated kinase 3 (SGK3), Proto-oncogene tyrosine-protein kinase Src (Src), Spleen tyrosine kinase (Syk), Tec protein tyrosine kinase (Tec), Tunica interna endothelial cell kinase 2 (Tie2), Tyrosine kinase receptor A (TrkA), BDNF/NT-3 growth factors receptor (TrkB), Zeta-chain (TCR) Associated Protein kinase 70 kDa (ZAP-70), and ZIP kinase (ZIPK).

Combination Therapy

According to some embodiments, the pharmaceutical composition further comprises at least one additional therapeutic agent.

According to some such embodiments, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

According to another embodiment, the additional therapeutic agent is an anti-inflammatory agent.

According to some embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to another embodiment, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

According to another embodiment, the anti-inflammatory agent includes, without limitation, Transforming Growth Factor-beta3 (TGF-β3), an anti-Tumor Necrosis Factor-alpha (TNF-α) agent, or a combination thereof.

According to some embodiments, the therapeutic peptide of the present invention has no effect on normal wound healing. According to some other embodiments, the therapeutic peptide of the present invention is capable of exerting an antibacterial effect on a wound.

According to some embodiments, the additional agent is an analgesic agent. According to some embodiments, the analgesic agent relieves pain by elevating the pain threshold without disturbing consciousness or altering other sensory modalities. According to some such embodiments, the analgesic agent is a non-opioid analgesic. “Non-opioid analgesics” are natural or synthetic substances that reduce pain but are not opioid analgesics. Examples of non-opioid analgesics include, but are not limited to, etodolac, indomethacin, sulindac, tolmetin, nabumetone, piroxicam, acetaminophen, fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, but are not limited to, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to another embodiment, the additional agent is an anti-infective agent. According to another embodiment, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Other examples of at least one additional therapeutic agent include, but are not limited to, rose hip oil, vitamin E, 5-fluorouracil, bleomycin, onion extract, pentoxifylline, prolyl-4-hydroxylase, verapamil, tacrolimus, tamoxifen, tretinoin, colchicine, a calcium antagonist, tranilst, zinc, an antibiotic, and a combination thereof.

Reducing Off-Target Affects

According to another embodiment, the pharmaceutical composition inhibits the kinase activity of at least one kinase selected from the group of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof, without substantially inhibiting the activity of one or more off-target proteins. According to some such embodiments, the off-target protein is an off-target kinase or an off-target receptor.

According to some embodiments, the off-target protein is selected from the group consisting of Abelson murine leukemia viral oncogene homolog 1 (Ab1), Abelson murine leukemia viral oncogene homolog 1 (T3151) (Ab1 (T3151)), Abelson murine leukemia viral oncogene homolog 1 (Y253F) (Ab1 (Y253F)), Anaplastic lymphoma kinase (ALK), Abelson-related gene (Arg), 5′-AMP-activated protein kinase catalytic subunit alpha-1 (AMPKα1), 5′-AMP-activated protein kinase catalytic subunit alpha-2 (AMPKα2), AMPK-related protein kinase 5 (ARKS), Apoptosis signal regulating kinase 1 (ASK1), Aurora kinase B (Aurora-B), AXL receptor tyrosine kinase (Ax1), Bone marrow tyrosine kinase gene in chromosome X protein (Bmx), Breast tumor kinase (BRK), Bruton's tyrosine kinase (BTK), Bruton's tyrosine kinase (R28H) (BTK (R28H)), Ca2⁺/calmodulin-dependent protein kinase IIβ (CaMIIβ), Ca²⁺/calmodulin-dependent protein kinase IIγ (CaMKIIγ), Ca²⁺/calmodulin-dependent protein kinase δ (CaMKIδ), Ca²⁺/calmodulin-dependent protein kinase IIδ (CaMKIIδ), Ca²⁺/calmodulin-dependent protein kinase IV (CaMKIV), Cell devision kinase 2 (CDK2/cyclinE), Cell devision kinase 3 (CDK3/cyclinE), Cell devision kinase 6 (CDK6/cyclinD3), Cell devision kinase 7 (CDK7/cyclinH/MAT1), Cell devision kinase 9 (CDK9/cyclin T1), Checkpoint kinase 2 (CHK2), Checkpoint kinase 2 (1157T) (CHK2 (1157T)), Checkpoint kinase 2 (R145W) (CHK2 (R145W)), Proto-oncogene tyrosine-protein kinase cKit (D816V) (cKit (D816V)), C-src tyrosine kinase (CSK), Raf proto-oncogene serine/threonine protein kinase (c-RAF), Proto-oncogene tyrosine-protein kinase (cSRC), Death-associated protein kinase 1 (DAPK1), Death-associated protein kinase 2 (DAPK2), Dystrophia myotonica-protein kinase (DMPK), DAP kinase-related apoptosis-inducing protein kinase 1 (DRAK1), Epidermal growth factor receptor (EGFR), Epidermal growth factor receptor (EGFR L858R), Epidermal growth factor receptor L861Q (EGFR (L861Q)), Eph receptor A2 (EphA2) (EphA2), Eph receptor A3 (EphA3), Eph receptor A5 (EphA5), Eph receptor B2 (EphB2), Eph receptor B4 (EphB4), Erythroblastic leukemia viral oncogene homolog 4 (ErbB4), c-Fes protein tyrosine kinase (Fes), Fibroblast growth factor receptor 2 (FGFR2), Fibroblast growth factor receptor 3 (FGFR3), Fibroblast growth factor receptor 4 (FGFR4), Fms-like tyrosine kinase receptor-3 (Flt3), FMS proto-oncogene (Fms), Haploid germ cell-specific nuclear protein kinase (Haspin), Insulin receptor-related receptor (IRR), Interleukin-1 receptor-associated kinase 1 (IRAK1), Interleukin-1 receptor-associated kinase 4 (IRAK4), IL2-inducible T-cell kinase (Itk), Kinase insert domain receptor (KDR), Lymphocyte cell-specific protein-tyrosine kinase (Lck), Lymphocyte-oriented kinase (LOK), Lyn tyrosine protein kinase (Lyn), MEK1, Maternal embryonic leucine zipper kinase (MELK), c-Mer proto-oncogene tyrosine kinase (Mer), c-Met proto-oncogene tyrosine kinase (Met), c-Met proto-oncogene tyrosine kinase D1246N (Met (D1246N)), c-Met proto-oncogene tyrosine kinase Y1248D (Met Y1248D), Misshapen/NIK-related kinase (MINK), MAP kinase kinase 6 (MKK6), Myosin light-chain kinase (MLCK), Mixed lineage kinase 1 (MLK1), MAP kinase signal-integrating kinase 2 (MnK2), Myotonic dystrophy kinase-related CDC42-binding kinase alpha (MRCKα), Myotonic dystrophy kinase-related CDC42-binding kinase beta (MRCKβ), Mitogen- and stress-activated protein kinase 1 (MSK1), Mitogen- and stress-activated protein kinase 2 (MSK2), Muscle-specific serine kinase 1 (MSSK1), Mammalian STE20-like protein kinase 1 (MST1), Mammalian STE20-like protein kinase 2 (MST2), Mammalian STE20-like protein kinase 3 (MST3), Muscle, skeletal receptor tyrosine-protein kinase (MuSK), Never in mitosis A-related kinase 2 (NEK2), Never in mitosis A-related kinase 3 (NEK3), Never in mitosis A-related kinase 11 (NEK11), 70 kDa ribosomal protein S6 kinase 1 (p70S6K), PAS domain containing serine/threonine kinase (PASK), Phosphorylase kinase subunit gamma-2 (PhKγ2), Pim-1 kinase (Pim-1), Protein kinase B alpha (PKBα), Protein kinase B beta (PKBβ), Protein kinase B gamma (PKBγ), Protein kinase C, alpha (PKCα), Protein kinase C, beta1 (PKCγ1), Protein kinase C, beta II (PKCβII), Protein kinase C, gamma (PKCγ), Protein kinase C, epsilon (PKCε), Protein kinase C, iota (PCKι), Protein kinase C, mu (PKCμ), Protein kinase C, zeta (PKCζ), protein kinase D2 (PKD2), cGMP-dependent protein kinase 1 alpha (PKG1α), cGMP-dependent protein kinase 1 beta (PKG1β), Protein-kinase C-related kinase 2 (PRK2), Proline-rich tyrosine kinase 2 (Pyk2), Proto-oncogene tyrosine-protein kinase receptor Ret V804L (Ret (V804L)), Receptor-interacting serine-threonine kinase 2 (RIPK2), Rho-associated protein kinase I (ROCK-I), Rho-associated protein kinase II (ROCK-II), Ribosomal protein S6 kinase 1 (Rsk1), Ribosomal protein S6 kinase 2 (Rsk2), Ribosomal protein S6 kinase 3 (Rsk3), Ribosomal protein S6 kinase 4 (Rsk4), Stress-activated protein kinase 2A T106M (SAPK2a, T106M), Stress-activated protein kinase 3 (SAPK3), Serum/glucocorticoid regulated kinase (SGK), Serum/glucocorticoid regulated kinase 2 (SGK2), Serum/glucocorticoid-regulated kinase 3 (SGK3), Proto-oncogene tyrosine-protein kinase Src 1-530 (Src, 1-530), Serine/threonine-protein kinase 33 (STK33), Spleen tyrosine kinase (Syk), Thousand and one amino acid protein 1 (TAO1), Thousand and one amino acid protein 2 (TAO2), Thousand and one amino acid protein 3 (TAO3), TANK-binding kinase 1 (TBK1), Tec protein tyrosine kinase (Tec), Tunica interna endothelial cell kinase 2 (Tie2), Tyrosine kinase receptor A (TrkA), TXK tyrosine kinase (Txk), WNK lysine deficient protein kinase 2 (WNK2), WNK lysine deficient protein kinase 3 (WNK3), Yamaguchi sarcoma viral oncogene homolog 1 (Yes), Zeta-chain (TCR) Associated Protein kinase 70 kDa (ZAP-70), and ZIP kinase (ZIPK).

According to some embodiments, the MK2 polypeptide inhibitor inhibits the binding activity of an off-target protein with an inhibition activity of IC₅₀ value of at least about 30 μM.

According to some embodiments, the off-target kinase is selected from the group consisting of Anaplastic lymphoma kinase (ALK), 5′-AMP-activated protein kinase catalytic subunit alpha-1 (AMPKα1), 5′-AMP-activated protein kinase catalytic subunit alpha-2 (AMPKα2), AMPK-related protein kinase 5 (ARKS), Apoptosis signal regulating kinase 1 (ASK1), Aurora kinase B (Aurora-B), AXL receptor tyrosine kinase (Ax1), Bone marrow tyrosine kinase gene in chromosome X protein (Bmx), Breast tumor kinase (BRK), Bruton's tyrosine kinase (BTK), Bruton's tyrosine kinase (R28H) (BTK (R28H)), Ca²⁺/calmodulin-dependent protein kinase IIβ (CaMIIβ), Ca²⁺/calmodulin-dependent protein kinase IIγ (CaMKIIγ), Ca²⁺/calmodulin-dependent protein kinase δ (CaMKIδ), Ca²⁺/calmodulin-dependent protein kinase IIδ (CaMKIIδ), Ca²⁺/calmodulin-dependent protein kinase IV (CaMKIV), Cell devision kinase 2 (CDK2/cyclinE), Cell devision kinase 3 (CDK3/cyclinE), Cell devision kinase 6 (CDK6/cyclinD3), Cell devision kinase 7 (CDK7/cyclinH/MAT1), Cell devision kinase 9 (CDK9/cyclin T1), Checkpoint kinase 2 (CHK2), Checkpoint kinase 2 (1157T) (CHK2 (1157T)), Checkpoint kinase 2 (R145W) (CHK2 (R145W)), Proto-oncogene tyrosine-protein kinase cKit (D816V) (cKit (D816V)), C-src tyrosine kinase (CSK), Raf proto-oncogene serine/threonine protein kinase (c-RAF), Proto-oncogene tyrosine-protein kinase (cSRC), Death-associated protein kinase 1 (DAPK1), Death-associated protein kinase 2 (DAPK2), Dystrophia myotonica-protein kinase (DMPK), DAP kinase-related apoptosis-inducing protein kinase 1 (DRAK1), Fms-like tyrosine kinase receptor-3 (Flt3), Haploid germ cell-specific nuclear protein kinase (Haspin), Insulin receptor-related receptor (IRR), Interleukin-1 receptor-associated kinase 1 (IRAK1), Interleukin-1 receptor-associated kinase 4 (IRAK4), IL2-inducible T-cell kinase (Itk), Lymphocyte cell-specific protein-tyrosine kinase (Lck), Lymphocyte-oriented kinase (LOK), Lyn tyrosine protein kinase (Lyn), MEK1, Maternal embryonic leucine zipper kinase (MELK), c-Mer proto-oncogene tyrosine kinase (Mer), c-Met proto-oncogene tyrosine kinase (Met), c-Met proto-oncogene tyrosine kinase D1246N (Met (D1246N)), c-Met proto-oncogene tyrosine kinase Y1248D (Met Y1248D), Misshapen/NIK-related kinase (MINK), MAP kinase kinase 6 (MKK6), Myosin light-chain kinase (MLCK), Mixed lineage kinase 1 (MLK1), MAP kinase signal-integrating kinase 2 (MnK2), Myotonic dystrophy kinase-related CDC42-binding kinase alpha (MRCKα), Myotonic dystrophy kinase-related CDC42-binding kinase beta (MRCKβ), Mitogen- and stress-activated protein kinase 1 (MSK1), Mitogen- and stress-activated protein kinase 2 (MSK2), Muscle-specific serine kinase 1 (MSSK1), Mammalian STE20-like protein kinase 1 (MST1), Mammalian STE20-like protein kinase 2 (MST2), Mammalian STE20-like protein kinase 3 (MST3), Muscle, skeletal receptor tyrosine-protein kinase (MuSK), Never in mitosis A-related kinase 2 (NEK2), Never in mitosis A-related kinase 3 (NEK3), Never in mitosis A-related kinase 11 (NEK11), 70 kDa ribosomal protein S6 kinase 1 (p70S6K), PAS domain containing serine/threonine kinase (PASK), Phosphorylase kinase subunit gamma-2 (PhKγ2), Pim-1 kinase (Pim-1), Protein kinase B alpha (PKBα), Protein kinase B beta (PKBβ), Protein kinase B gamma (PKBγ), Protein kinase C, alpha (PKCα), Protein kinase C, beta 1 (PKCβ1), Protein kinase C, beta II (PKCβII), Protein kinase C, gamma (PKCγ), Protein kinase C, epsilon (PKCε), Protein kinase C, iota (PCKι), Protein kinase C, mu (PKCμ), Protein kinase C, zeta (PKCζ), protein kinase D2 (PKD2), cGMP-dependent protein kinase 1 alpha (PKG1α), cGMP-dependent protein kinase 1 beta (PKG1β), Protein-kinase C-related kinase 2 (PRK2), Proline-rich tyrosine kinase 2 (Pyk2), Proto-oncogene tyrosine-protein kinase receptor Ret V804L (Ret (V804L)), Receptor-interacting serine-threonine kinase 2 (RIPK2), Rho-associated protein kinase I (ROCK-I), Rho-associated protein kinase II (ROCK-II), Ribosomal protein S6 kinase 1 (Rsk1), Ribosomal protein S6 kinase 2 (Rsk2), Ribosomal protein S6 kinase 3 (Rsk3), Ribosomal protein S6 kinase 4 (Rsk4), Stress-activated protein kinase 2A T106M (SAPK2a, T106M), Stress-activated protein kinase 3 (SAPK3), Serum/glucocorticoid regulated kinase (SGK), Serum/glucocorticoid regulated kinase 2 (SGK2), Serum/glucocorticoid-regulated kinase 3 (SGK3), Proto-oncogene tyrosine-protein kinase Src 1-530 (Src, 1-530), Serine/threonine-protein kinase 33 (STK33), Spleen tyrosine kinase (Syk), Thousand and one amino acid protein 1 (TAO0), Thousand and one amino acid protein 2 (TAO2), Thousand and one amino acid protein 3 (TAO3), TANK-binding kinase 1 (TBK1), Tec protein tyrosine kinase (Tec), Tunica interna endothelial cell kinase 2 (Tie2), Tyrosine kinase receptor A (TrkA), TXK tyrosine kinase (Txk), WNK lysine deficient protein kinase 2 (WNK2), WNK lysine deficient protein kinase 3 (WNK3), Yamaguchi sarcoma viral oncogene homolog 1 (Yes), Zeta-chain (TCR) Associated Protein kinase 70 kDa (ZAP-70), and ZIP kinase (ZIPK).

According to some embodiments, the off-target protein is an off-target kinase. According to some such embodiments, the pharmaceutical composition inhibits less than 50% of the kinase activity of the off-target kinase. According to such embodiment, the pharmaceutical composition inhibits less than 65% of the kinase activity of the off-target kinase. According to another embodiment, the pharmaceutical composition inhibits less than 50% of the kinase activity of the off-target kinase. According to another embodiment, the pharmaceutical composition inhibits less than 40% of the kinase activity of the off-target kinase. According to another embodiment, the pharmaceutical composition inhibits less than 20% of the kinase activity of the off-target kinase. According to another embodiment, the pharmaceutical composition inhibits less than 15% of the kinase activity of the off-target kinase. According to another embodiment, the pharmaceutical composition inhibits less than 10% of the kinase activity of the off-target kinase. According to another embodiment, the pharmaceutical composition inhibits less than 9% of the kinase activity of the off-target kinase. According to another embodiment, the pharmaceutical composition inhibits less than 8% of the kinase activity of the off-target kinase. According to another embodiment, the pharmaceutical composition inhibits less than 7% of the kinase activity of the off-target kinase. According to another embodiment, the pharmaceutical composition inhibits less than 6% of the kinase activity of the off-target kinase. According to another embodiment, the pharmaceutical composition inhibits less than 5% of the kinase activity of the off-target kinase. According to another embodiment, the pharmaceutical composition inhibits less than 4% of the kinase activity of the off-target kinase. According to another embodiment, the pharmaceutical composition inhibits less than 3% of the kinase activity of the off-target kinase. According to another embodiment, the pharmaceutical composition inhibits less than 2% of the kinase activity of the off-target kinase. According to another embodiment, the pharmaceutical composition inhibits less than 1% of the kinase activity of the off-target kinase. According to another embodiment, the pharmaceutical composition increases the kinase activity of the off-target kinase.

According to some embodiments, the off-target protein is an off-target receptor. According to some such embodiments, the pharmaceutical composition inhibits less than 50% of the binding activity of the off-target receptor. According to such embodiment, the pharmaceutical composition inhibits less than 65% of the binding activity of the off-target receptor. According to another embodiment, the pharmaceutical composition inhibits less than 50% of the binding activity of the off-target receptor. According to another embodiment, the pharmaceutical composition inhibits less than 40% of the binding activity of the off-target receptor. According to another embodiment, the pharmaceutical composition inhibits less than 20% of the binding activity of the off-target receptor. According to another embodiment, the pharmaceutical composition inhibits less than 15% of the binding activity of the off-target receptor. According to another embodiment, the pharmaceutical composition inhibits less than 10% of the binding activity of the off-target receptor. According to another embodiment, the pharmaceutical composition inhibits less than 9% of the binding activity of the off-target receptor. According to another embodiment, the pharmaceutical composition inhibits less than 8% of the binding activity of the off-target receptor. According to another embodiment, the pharmaceutical composition inhibits less than 7% of the binding activity of the off-target receptor. According to another embodiment, the pharmaceutical composition inhibits less than 6% of the binding activity of the off-target receptor. According to another embodiment, the pharmaceutical composition inhibits less than 5% of the binding activity of the off-target receptor. According to another embodiment, the pharmaceutical composition inhibits less than 4% of the binding activity of the off-target receptor. According to another embodiment, the pharmaceutical composition inhibits less than 3% of the binding activity of the off-target receptor. According to another embodiment, the pharmaceutical composition inhibits less than 2% of the binding activity of the off-target receptor. According to another embodiment, the pharmaceutical composition inhibits less than 1% of the binding activity of the off-target receptor. According to another embodiment, the pharmaceutical composition increases the binding activity of the off-target receptor.

According to some embodiments, the off-target receptor is selected from the group consisting of angiotensin 2, bombesin, melanocortin 4, neurokinin 2, neuropeptide Y, serotonin 2A, vasoactive intestinal peptide, and small conductance calcium-activated K+ channel. According to one embodiment, the off-target receptor is angiotensin 2. According to one embodiment, the off-target receptor is bombesin. According to one embodiment, the off-target receptor is melanocortin 4. According to one embodiment, the off-target receptor is neurokinin 2. According to one embodiment, the off-target receptor is neuropeptide Y. According to one embodiment, the off-target receptor is serotonin 2A. According to one embodiment, the off-target receptor is vasoactive intestinal peptide. According to one embodiment, the off-target receptor is small conductance calcium-activated K+ channel.

According to some embodiments, the one or more other selected kinase that is not substantially inhibited is selected from the group of Ca²⁺/calmodulin-dependent protein kinase II (CaMKII, including its subunit CaMKIIδ), Proto-oncogene serine/threonine-protein kinase (PIM-1), cellular-Sarcoma (c-SRC), Spleen Tyrosine Kinase (SYK), c-Src Tyrosine Kinase (CSK), and Insulin-like Growth Factor 1 Receptor (IGF-1R).

According to some embodiments, in order to enhance drug efficacy and to reduce accumulation of the polypeptide MMI-0100 of amino acid sequence YARAAARQARAKALARQLGVAA (SEQ ID NO: 1) or its functional equivalent in non-target tissues, the polypeptide of the present invention of amino acid sequence YARAAARQARAKALARQLGVAA (SEQ ID NO: 1) or its functional equivalent can be linked or associated with a targeting moiety, which directs the polypeptide to a specific cell type or tissue. Examples of the targeting moiety include, but are not limited to, (i) a ligand for a known or unknown receptor or (ii) a compound, a peptide, or a monoclonal antibody that binds to a specific molecular target, e.g., a peptide or carbohydrate, expressed on the surface of a specific cell type.

According to another embodiment, the functional equivalent of the polypeptide MMI-0100 of amino acid sequence YARAAARQARAKALARQLGVAA (SEQ ID NO: 1) is a fusion peptide comprising a first polypeptide operatively linked to a second polypeptide, wherein the first polypeptide is of amino acid sequence YARAAARQARA (SEQ ID NO: 11), and the second polypeptide comprises a therapeutic domain whose sequence has a substantial identity to amino acid sequence KALARQLGVAA (SEQ ID NO: 2).

According to another embodiment, the second polypeptide has at least 70 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2). According to another embodiment, the second polypeptide has at least 80 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2). According to another embodiment, the second polypeptide has at least 90 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2). According to another embodiment, the second polypeptide has at least 95 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2).

According to another embodiment, the second polypeptide is a polypeptide of amino acid sequence KALARQLAVA (SEQ ID NO: 8). According to another embodiment, the second polypeptide is a polypeptide of amino acid sequence KALARQLGVA (SEQ ID NO: 9). According to another embodiment, the second polypeptide is a polypeptide of amino acid sequence KALARQLGVAA (SEQ ID NO: 10); see, e.g., U.S. Published Application No. 2009-0196927, U.S. Published Application No. 2009-0149389, and U.S. Published Application No2010-0158968, each of which is incorporated herein by reference in its entirety.

According to another embodiment, the functional equivalent of the polypeptide MMI-0100 of amino acid sequence YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11), and the second polypeptide is of amino acid sequence KALARQLGVAA (SEQ ID NO: 2).

According to another embodiment, the first polypeptide is a polypeptide of amino acid sequence WLRRIKAWLRRIKA (SEQ ID NO: 12). According to another embodiment, first polypeptide is a polypeptide of amino acid sequence WLRRIKA (SEQ ID NO: 13). According to another embodiment, the first polypeptide is a polypeptide of amino acid sequence YGRKKRRQRRR (SEQ ID NO: 14). According to another embodiment, the first polypeptide is a polypeptide of amino acid sequence WLRRIKAWLRRI (SEQ ID NO: 15). According to another embodiment, the first polypeptide is a polypeptide of amino acid sequence FAKLAARLYR (SEQ ID NO: 16). According to another embodiment, the first polypeptide is a polypeptide of amino acid sequence KAFAKLAARLYR (SEQ ID NO: 17). According to another embodiment, the first polypeptide is a polypeptide of amino acid sequence HRRIKAWLKKI (SEQ ID NO: 18).

Therapeutic Amount/Dose

According to some embodiments, the therapeutic amount of the MK2 polypeptide inhibitor of the pharmaceutical composition is of an amount from about 0.000001 mg/kg body weight to about 100 mg/kg body weight. According to another embodiment, the therapeutic amount of the MK2 polypeptide inhibitor of the pharmaceutical composition is of an amount from about 0.00001 mg/kg body weight to about 100 mg/kg body weight. According to another embodiment, the therapeutic amount of the MK2 polypeptide inhibitor of the pharmaceutical composition is of an amount from about 0.0001 mg/kg body weight to about 100 mg/kg body weight. According to another embodiment, the therapeutic amount of the MK2 polypeptide inhibitor of the pharmaceutical composition is of an amount from about 0.001 mg/kg body weight to about 10 mg/kg body weight. According to another embodiment, the therapeutic amount of the MK2 polypeptide inhibitor of the pharmaceutical composition is of an amount from about 0.01 mg/kg body weight to about 10 mg/kg body weight. According to another embodiment, the therapeutic amount of the MK2 polypeptide inhibitor of the pharmaceutical composition is of an amount from about 0.1 mg/kg (or 100 μg/kg) body weight to about 10 mg/kg body weight. According to another embodiment, the therapeutic amount of the MK2 polypeptide inhibitor of the pharmaceutical composition is of an amount from about 1 mg/kg body weight to about 10 mg/kg body weight. According to another embodiment, the therapeutic amount of the MK2 polypeptide inhibitor of the pharmaceutical composition is of an amount from about 10 mg/kg body weight to about 100 mg/kg body weight. According to another embodiment, the therapeutic amount of the MK2 polypeptide inhibitor of the pharmaceutical composition is of an amount from about 2 mg/kg body weight to about 10 mg/kg body weight. According to another embodiment, the therapeutic amount of the MK2 polypeptide inhibitor of the pharmaceutical composition is of an amount from about 3 mg/kg body weight to about 10 mg/kg body weight. According to another embodiment, the therapeutic amount of the MK2 polypeptide inhibitor of the pharmaceutical composition is of an amount from about 4 mg/kg body weight to about 10 mg/kg body weight. According to another embodiment, the therapeutic amount of the MK2 polypeptide inhibitor of the pharmaceutical composition is of an amount from about 5 mg/kg body weight to about 10 mg/kg body weight. According to another embodiment, the therapeutic amount of the MK2 polypeptide inhibitor of the pharmaceutical composition is of an amount from about 60 mg/kg body weight to about 100 mg/kg body weight. According to another embodiment, the therapeutic amount of the MK2 polypeptide inhibitor of the pharmaceutical composition is of an amount from about 70 mg/kg body weight to about 100 mg/kg body weight. According to another embodiment, the therapeutic amount of the MK2 polypeptide inhibitor of the pharmaceutical composition is of an amount from about 80 mg/kg body weight to about 100 mg/kg body weight. According to another embodiment, the therapeutic amount of the MK2 polypeptide inhibitor of the pharmaceutical composition is of an amount from about 90 mg/kg body weight to about 100 mg/kg body weight. According to another embodiment, the therapeutic amount of the MK2 polypeptide inhibitor of the pharmaceutical composition is of an amount from about 0.000001 mg/kg body weight to about 90 mg/kg body weight. According to another embodiment, the therapeutic amount of the MK2 polypeptide inhibitor of the pharmaceutical composition is of an amount from about 0.000001 mg/kg body weight to about 80 mg/kg body weight. According to another embodiment, the therapeutic amount of the MK2 polypeptide inhibitor of the pharmaceutical composition is of an amount from about 0.000001 mg/kg body weight to about 70 mg/kg body weight. According to another embodiment, the therapeutic amount of the MK2 polypeptide inhibitor of the pharmaceutical composition is of an amount from about 0.000001 mg/kg body weight to about 60 mg/kg body weight. According to another embodiment, the therapeutic amount of the MK2 polypeptide inhibitor of the pharmaceutical composition is of an amount from about 0.000001 mg/kg body weight to about 50 mg/kg body weight. According to another embodiment, the therapeutic amount of the MK2 polypeptide inhibitor of the pharmaceutical composition is of an amount from about 0.000001 mg/kg body weight to about 40 mg/kg body weight. According to another embodiment, the therapeutic amount of the MK2 polypeptide inhibitor is of an amount from about 0.000001 mg/kg body weight to about 30 mg/kg body weight. According to another embodiment, the therapeutic amount of the MK2 polypeptide inhibitor of the pharmaceutical composition is of an amount from about 0.000001 mg/kg body weight to about 20 mg/kg body weight. According to another embodiment, the therapeutic amount of the MK2 polypeptide inhibitor of the pharmaceutical composition is of an amount from about 0.000001 mg/kg body weight to about 10 mg/kg body weight. According to another embodiment, the therapeutic amount of the MK2 polypeptide inhibitor of the pharmaceutical composition is of an amount from about 0.000001 mg/kg body weight to about 1 mg/kg body weight. According to another embodiment, the therapeutic amount of the MK2 polypeptide inhibitor of the pharmaceutical composition is of an amount from about 0.000001 mg/kg body weight to about 0.1 mg/kg body weight. According to another embodiment, the therapeutic amount of the MK2 polypeptide inhibitor of the pharmaceutical composition is of an amount from about 0.000001 mg/kg body weight to about 0.1 mg/kg body weight. According to another embodiment, the therapeutic amount of the MK2 polypeptide inhibitor of the pharmaceutical composition is of an amount from about 0.000001 mg/kg body weight to about 0.01 mg/kg body weight. According to another embodiment, the therapeutic amount of the MK2 polypeptide inhibitor of the pharmaceutical composition is of an amount from about 0.000001 mg/kg body weight to about 0.001 mg/kg body weight. According to another embodiment, the therapeutic amount of the MK2 polypeptide inhibitor of the pharmaceutical composition is of an amount from about 0.000001 mg/kg body weight to about 0.0001 mg/kg body weight. According to another embodiment, the therapeutic amount of the MK2 polypeptide inhibitor of the pharmaceutical composition is of an amount from about 0.000001 mg/kg body weight to about 0.00001 mg/kg body weight.

According to some other embodiments, the therapeutic dose of the MK2 polypeptide inhibitor of the pharmaceutical composition ranges from 1 μg/kg/day to 25 μg/kg/day. According to some other embodiments, the therapeutic dose of the MK2 polypeptide inhibitor of the pharmaceutical composition ranges from 1 μg/kg/day to 2 μg/kg/day. According to some other embodiments, the therapeutic dose of the MK2 polypeptide inhibitor of the pharmaceutical composition ranges from 2 μg/kg/day to 3 μg/kg/day. According to some other embodiments, the therapeutic dose of the MK2 polypeptide inhibitor of the pharmaceutical composition ranges from 3 μg/kg/day to 4 μg/kg/day. According to some other embodiments, the therapeutic dose of the MK2 polypeptide inhibitor of the pharmaceutical ranges from 4 μg/kg/day to 5 μg/kg/day. According to some other embodiments, the therapeutic dose of the polypeptide inhibitor of the pharmaceutical composition ranges from 5 μg/kg/day to 6 μg/kg/day. According to some other embodiments, the therapeutic dose of the MK2 polypeptide inhibitor of the pharmaceutical composition ranges from 6 μg/kg/day to 7 μg/kg/day. According to some other embodiments, the therapeutic dose of the MK2 polypeptide inhibitor of the pharmaceutical composition ranges from 7 μg/kg/day to 8 μg/kg/day. According to some other embodiments, the therapeutic dose of the MK2 polypeptide inhibitor of the pharmaceutical composition ranges from 8 μg/kg/day to 9 μg/kg/day. According to some other embodiments, the therapeutic dose of the MK2 polypeptide inhibitor of the pharmaceutical composition ranges from 9 μg/kg/day to 10 μg/kg/day. According to some other embodiments, the therapeutic dose of the MK2 polypeptide inhibitor of the pharmaceutical composition ranges from 1 μg/kg/day to 5 μg/kg/day. According to some other embodiments, the therapeutic dose of the MK2 polypeptide inhibitor of the pharmaceutical composition ranges from 5 μg/kg/day to 10 μg/kg/day. According to some other embodiments, the therapeutic dose of the MK2 polypeptide inhibitor of the pharmaceutical composition ranges from 10 μg/kg/day to 15 μg/kg/day. According to some other embodiments, the therapeutic dose of the MK2 polypeptide inhibitor of the pharmaceutical composition ranges from 15 μg/kg/day to 20 μg/kg/day. According to some other embodiments, the therapeutic dose of the MK2 polypeptide inhibitor of the pharmaceutical composition ranges from 25 μg/kg/day to 30 μg/kg/day. According to some other embodiments, the therapeutic dose of the MK2 polypeptide inhibitor of the pharmaceutical composition ranges from 30 μg/kg/day to 35 μg/kg/day. According to some other embodiments, the therapeutic dose of the MK2 polypeptide inhibitor of the pharmaceutical composition ranges from 35 μg/kg/day to 40 μg/kg/day. According to some other embodiments, the therapeutic dose of the MK2 polypeptide inhibitor of the pharmaceutical composition ranges from 40 μg/kg/day to 45 μg/kg/day. According to some other embodiments, the therapeutic dose of the MK2 polypeptide inhibitor of the pharmaceutical composition ranges from 45 μg/kg/day to 50 μg/kg/day. According to some other embodiments, the therapeutic dose of the MK2 polypeptide inhibitor of the pharmaceutical composition ranges from 50 μg/kg/day to 55 μg/kg/day. According to some other embodiments, the therapeutic dose of the MK2 polypeptide inhibitor of the pharmaceutical composition ranges from 55 μg/kg/day to 60 μg/kg/day. According to some other embodiments, the therapeutic dose of the MK2 polypeptide inhibitor of the pharmaceutical composition ranges from 60 μg/kg/day to 65 μg/kg/day. According to some other embodiments, the therapeutic dose of the MK2 polypeptide inhibitor of the pharmaceutical composition ranges from 65 μg/kg/day to 70 μg/kg/day. According to some other embodiments, the therapeutic dose of the MK2 polypeptide inhibitor of the pharmaceutical composition ranges from 70 μg/kg/day to 75 μg/kg/day. According to some other embodiments, the therapeutic dose of the MK2 polypeptide inhibitor of the pharmaceutical composition ranges from 80 μg/kg/day to 85 μg/kg/day. According to some other embodiments, the therapeutic dose of the MK2 polypeptide inhibitor of the pharmaceutical composition ranges from 85 μg/kg/day to 90 μg/kg/day. According to some other embodiments, the therapeutic dose of the MK2 polypeptide inhibitor of the pharmaceutical composition ranges from 90 μg/kg/day to 95 μg/kg/day. According to some other embodiments, the therapeutic dose of the MK2 polypeptide inhibitor of the pharmaceutical composition ranges from 95 μg/kg/day to 100 μg/kg/day.

According to another embodiment, the therapeutic dose of the MK2 polypeptide inhibitor of the pharmaceutical composition is 1 μg/kg/day. According to another embodiment, the therapeutic dose of the MK2 polypeptide inhibitor of the pharmaceutical composition is 2 μg/kg/day. According to another embodiment, the therapeutic dose of the MK2 polypeptide inhibitor of the pharmaceutical composition is 5 μg/kg/day. According to another embodiment, the therapeutic dose of the MK2 polypeptide inhibitor of the pharmaceutical composition is 10 μg/kg/day.

Formulation

The MK2 polypeptide inhibitor or a functional equivalent thereof may be administered in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the described invention or may be prepared by separately reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The formulations may be presented conveniently in unit dosage form and may be prepared by any of the methods well known in the art of pharmacy. All methods include the step of bringing into association a therapeutic agent(s), or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

According to some embodiments, the carrier is a controlled release carrier. The term “controlled release” is intended to refer to any drug-containing formulation in which the manner and profile of drug release from the formulation are controlled. This includes immediate as well as non-immediate release formulations, with non-immediate release formulations including, but not limited to, sustained release and delayed release formulations. According to some embodiments, the controlled release of the pharmaceutical composition is mediated by changes in temperature. According to some other embodiments, the controlled release of the pharmaceutical composition is mediated by changes in pH.

Injectable depot forms may be made by forming microencapsulated matrices of a therapeutic agent/drug in biodegradable polymers such as, but not limited to, polyesters (polyglycolide, polylactic acid and combinations thereof), polyester polyethylene glycol copolymers, polyamino-derived biopolymers, polyanhydrides, polyorthoesters, polyphosphazenes, sucrose acetate isobutyrate (SAIB), photopolymerizable biopolymers, naturally-occurring biopolymers, protein polymers, collagen, and polysaccharides. Depending upon the ratio of drug to polymer and the nature of the particular polymer employed, the rate of drug release may be controlled. Such long acting formulations may be formulated with suitable polymeric or hydrophobic materials (for example as an emulsion in acceptable oil) or ion exchange resins, or as sparingly soluble derivatives, for example, as a sparingly soluble salt. Depot injectable formulations also are prepared by entrapping the drug in liposomes or microemulsions which are compatible with body tissues.

According to some embodiments, the carrier is a delayed release carrier. According to another embodiment, the delayed release carrier comprises a biodegradable polymer. According to another embodiment, the biodegradable polymer is a synthetic polymer. According to another embodiment, the biodegradable polymer is a naturally occurring polymer.

According to some embodiments, the carrier is a sustained release carrier. According to another embodiment, the sustained-release carrier comprises a biodegradable polymer. According to another embodiment, the biodegradable polymer is a synthetic polymer. According to another embodiment, the biodegradable polymer is a naturally occurring polymer.

According to some embodiments, the carrier is a short-term release carrier. The term “short-term” release, as used herein, means that an implant is constructed and arranged to deliver therapeutic levels of the active ingredient for about 1, 2, 3, 4, 5, 6, 7, 8, 9, 10, 11, 12, 13, 14, 15, 16, 17, 18, 19, 20, 21, 22, or 23 hours. According to some other embodiments, the short term release carrier delivers therapeutic levels of the active ingredient for about 1, 2, 3, or 4 days.

According to some embodiments, the carrier is a long-term release carrier. According to another embodiment, the long-term-release carrier comprises a biodegradable polymer. According to another embodiment, the biodegradable polymer is a synthetic polymer.

According to some embodiments, the carrier comprises particles. The term “particles” as used herein refers to refers to an extremely small constituent (e.g., nanoparticles, microparticles, or in some instances larger) in or on which is contained the composition as described herein.

The compositions also may contain appropriate adjuvants, including, without limitation, preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

According to some embodiments, the polypeptides of the present invention are covalently attached to polyethylene glycol (PEG) polymer chains. According to some other embodiments, the polypeptides of the present invention are stapled with hydrocarbons to generate hydrocarbon-stapled peptides that are capable of forming stable alpha-helical structure (Schafineister, C. et al., J. Am. Chem. Soc., 2000, 122, 5891-5892, incorporated herein by reference in its entirety).

According to some other embodiments, the polypeptides of the present invention are encapsulated or entrapped into microspheres, nanocapsules, liposomes, or microemulsions, or comprises d-amino acids in order to increase stability, to lengthen delivery, or to alter activity of the peptides. These techniques are well known in the art and can lengthen the stability and release simultaneously by hours to days, or delay the uptake of the drug by nearby cells.

### II. Dressings for Treating, Reducing or Preventing a Cutaneous Scar

According to another aspect, the described invention provides a dressing for use in treating a cutaneous scar in a subject in need thereof, comprising a pharmaceutical composition comprising a therapeutic amount of a Mitogen-Activated Protein Kinase-Activated Protein Kinase 2 (MK2) inhibitor comprising a polypeptide inhibitor or a functional equivalent thereof, and a pharmaceutically acceptable carrier, wherein the therapeutic amount is effective to treat, reduce or prevent a cutaneous scar in the subject.

Dressing

According to one embodiment, examples of suitable dressings for the purpose of the invention include, but are not limited to, a gauze dressing, a tulle dressing, an alginate dressing, a polyurethane dressing, a silicone foam dressing, a collagen dressing, a synthetic polymer scaffold, s peptide-soaked suture, or a combination thereof.

Gauze dressings can stick to the wound surface and disrupt the wound bed when removed. As a result gauze dressings are used generally on minor wounds or as secondary dressings.

Tulle dressings do not stick to wound surfaces. They are suitable for use in flat, shallow wounds, and are useful in patients with sensitive skin. Examples of tulle dressings include, but are not limited to, Jelonet® and Paranet®.

Alginate dressings are composed of calcium alginate (a seaweed component). When in contact with a wound, calcium in the dressing is exchanged with sodium from wound fluid and this turns the dressing into a gel that maintains a moist wound environment. These dressings are good for exudating wounds and help in debridement of sloughing wounds. In general, alginate dressings are not used on low exudating wounds as this will cause dryness and scabbing. Alginate dressing are changed daily. Examples of alginate dressings include, but are not limited to, Kaltostat® and Sorbsan®.

Polyurethane or silicone foam dressings are designed to absorb large amounts of exudates. They maintain a moist wound environment but are not as useful as alginates or hydrocolloids for debridement. In general, they are not used on low exudating wounds as this will cause dryness and scabbing. Examples of polyurethane or silicone foam dressings include, but are not limited to, Allevyn® and Lyofoam®.

Collagen dressings are generally provided in the form of pads, gels or particles. They promote the deposit of newly formed collagen in the wound bed, and absorb exudate and provide a moist environment.

Other suitable dressings include occlusive dressings. The term “occlusive dressing” as used herein refers to a dressing that prevents air or bacteria from reaching a wound or lesion and that retains moisture, heat, body fluids, and medication. Traditional dressings such as gauze and telfa pads (non-adhesive pads) promote desiccation of the wound surface and adhere to it as well. When removed, the dressing strips away newly formed epithelium, causing bleeding and prolongation of the healing process. Since the wound is dry and cracked, movement is often painful and inhibited. Studies have shown that occlusive dressings prevent desiccation and eschar formation by trapping moisture next to the wound bed. According to some such embodiments, the occlusive dressings are totally occlusive dressings. According to some other embodiments, the occlusive dressings are semi-permeable dressings. Examples of occlude dressings for the purpose of the invention include, but are not limited to, film dressings (totally occlusive dressings), semi-permeable film dressings, hydrogel dressings, hydrocolloid dressings, or a combination thereof.

Film dressings and semi-permeable film dressings comprise sheets of materials that may be used to cover wounds. Such dressings may comprise sterile materials. Suitable materials, from which such films may be manufactured, include polyurethane and chitin. Film dressing (or semi-permeable film dressings) may be coated with adhesives, such as acrylic adhesives, in order to assist their retention at sites where they are required. Dressings of this type may be transparent, and therefore allow the progress of wound healing to be checked. These dressings are generally suitable for shallow wounds with low exudate. Examples of film or semi-permeable film dressings include, but are not limited to, OpSite® and Tegaderm®

Hydrogel dressings are composed mainly of water in a complex network of fibers that keep the polymer gel intact. Water is released to keep the wound moist. These dressings may be used for necrotic or sloughy wound beds to rehydrate and remove dead tissue. They are not used for moderate to heavily exudating wounds. Examples of hydrogel dressings include, but are not limited to, Tegagel®, Intrasite®.

Hydrocolloid dressings are composed of hydrophilic particles, such as gelatin and pectin, connected together with a hydrophobic adhesive matrix, and are covered by an outer film or foam layer. When hydrocolloids are applied to a wound, any exudate in the wound contact area is absorbed to form a swollen gel, which fills the wound and provides a controlled absorption gradient to the rest of the dressing. This creates a warm, moist environment that promotes debridement and healing. Depending on the hydrocolloid dressing chosen, they may be suitable for use in wounds with light to heavy exudate, sloughing or granulating wounds. Dressings of this sort are available in many forms (adhesive or non-adhesive pad, paste, powder) but most commonly as self-adhesive pads. Examples of hydrocolloid dressings include, but are not limited to, DuoDERM® and Tegasorb®.

The skilled artisan will be aware that a suitable wound healing dressing to be used on a particular wound may be selected with reference to the type of the wound, size of the wound, and healing progression of the wound.

According to some embodiments, the dressing of the present invention comprises a mechano-active dressing. According to some such embodiments, the mechano-active dressing is configured to be removably secured to a skin surface near a wound in order to apply tension to the wound. The mechano-active dressing can shield the wound from endogenous stress originating from the skin itself (e.g., stress transferred to the wound via stratum corneum, epidermal, or dermal tissue), and/or exogenous stress (e.g., stress transferred to the wound via physical body movement or muscle action). In some such embodiments, the mechano-active dressing shields the wound from endogenous stress without affecting exogenous stress on the wound. In some other embodiments, the mechano-active dressing shields the wound from exogenous stress without affecting endogenous stress on the wound.

The mechano-active dressing can be removably secured to the skin surface in a variety of ways. For example, the mechano-active dressing may be removably secured to the skin surface with an adhesive, with a skin piercing device, or both. Suitable adhesives include, but are not limited to, polyacryl-based, polysobutylene-based, and silicone-based pressure sensitive adhesives. Suitable skin-piercing devices include, but are not limited to, micro needles, sutures, anchors staples, microtines, and the like.

According to some embodiments, the mechano-active dressing is a stress-shielding device, which is manufactured using silicone polymer sheets (NuSil, Lafayette, Calif.) and pressure-sensitive adhesive (NuSil) secured to Teflon® extension sheets (DuPont, Wilmington, Del.) (Gurtner, G. et al., Ann Surg, 254: 217-225, 2011, incorporated by reference).

According to some other embodiments, the mechano-active dressing comprises an active agent that can be useful in aiding in some aspect of the wound healing process. Examples of the active agent, include, but are not limited to, an anti-infective agent, a growth factor, vitamin (e.g., vitamin E), a clotting agent that promotes clotting of blood (e.g., a thrombin agent), or a combination thereof.

According to another embodiment, the dressing further comprises a dermal substitute, which is embedded in or on a surface of the dressing with the pharmaceutical composition of the described invention and provides a three-dimensional extracellular scaffold.

According to some embodiments, the dermal substitute is applied to a wound prior to wound closure. According to some other embodiments, the dermal substitute is applied to a wound at the time of wound closure. According to some other embodiments, the dermal substitute is applied to a wound after wound closure.

According to another embodiment, the dermal substitute is made of a natural biological material, including, but not limited to, human cadaver skin, porcine cadaver skin, and porcine small intestine submucosa. According to another embodiment, the natural biological material comprises a matrix. According to another embodiment, the natural biological material consists essentially of a matrix that is substantially devoid of cell remnants.

According to another embodiment, the dermal substitute is a constructive biological material. Examples of suitable constructive biological materials include, but are not limited to, collagen, glycosaminoglycan, fibronectin, hyaluonic acid, elastine, and a combination thereof. According to another embodiment, the constructive biological material is a bilayer, non-cellularized dermal regeneration template. According to another embodiment, the constructive biological material is a single layer, cellularized dermal regeneration template.

According to another embodiment, the dermal substitute is a synthetic dermal substitute. According to another embodiment, the synthetic dermal substitute contains a peptide of amino acid sequence Arginine-Glycine-Aspartate (RGD). According to another embodiment, the peptide of amino acid sequence Arginine-Glycine-Aspartate (RGD) is a biomimetic peptide. According to another embodiment, the synthetic dermal substitute comprises a hydrogel.

MK2 Inhibitor

According to one embodiment, the Mitogen-Activated Protein Kinase-Activated Protein Kinase 2 (MK2) inhibitor is an MK2 polypeptide inhibitor or a functional equivalent thereof. According to some embodiments, the MK2 polypeptide inhibitor is selected from the group consisting of a polypeptide MMI-0100 of amino acid sequence YARAAARQARAKALARQLGVAA (SEQ ID NO: 1), a polypeptide MMI-0200 of amino acid sequence YARAAARQARAKALNRQLGVA (SEQ ID NO: 19), a polypeptide MMI-0300 of amino acid sequence FAKLAARLYRKALARQLGVAA (SEQ ID NO: 3), a polypeptide MMI-0400 of amino acid sequence KAFAKLAARLYRKALARQLGVAA (SEQ ID NO: 4), and a polypeptide MMI-0500 of amino acid sequence HRRIKAWLKKIKALARQLGVAA (SEQ ID NO: 7). According to one embodiment, the MK2 polypeptide inhibitor is a polypeptide MMI-0100 of amino acid sequence YARAAARQARAKALARQLGVAA (SEQ ID NO: 1). According to another embodiment, the MK2 polypeptide inhibitor is a polypeptide MMI-0200 of amino acid sequence YARAAARQARAKALNRQLGVA (SEQ ID NO: 19). According to another embodiment, the MK2 polypeptide inhibitor is a polypeptide MMI-0300 of amino acid sequence FAKLAARLYRKALARQLGVAA (SEQ ID NO: 3). According to another embodiment, the MK2 polypeptide inhibitor is a polypeptide MMI-0400 of amino acid sequence KAFAKLAARLYRKALARQLGVAA (SEQ ID NO: 4). According to another embodiment, the MK2 polypeptide inhibitor is a polypeptide MMI-0500 of amino acid sequence HRRIKAWLKKIKALARQLGVAA (SEQ ID NO: 7).

According to another embodiment, the functional equivalent of the MK2 polypeptide inhibitor MMI-0100 of amino acid sequence YARAAARQARAKALARQLGVAA (SEQ ID NO: 1) has a substantial sequence identity to the amino acid sequence YARAAARQARAKALARQLGVAA (SEQ ID NO: 1).

According to another embodiment, the functional equivalent of the MK2 polypeptide inhibitor MMI-0100 of amino acid sequence YARAAARQARAKALARQLGVAA (SEQ ID NO: 1) has at least 80 percent sequence identity to the amino acid sequence YARAAARQARAKALARQLGVAA (SEQ ID NO: 1). According to another embodiment, the functional equivalent of the MK2 polypeptide inhibitor MMI-0100 of amino acid sequence YARAAARQARAKALARQLGVAA (SEQ ID NO: 1) has at least 90 percent sequence identity to the amino acid sequence YARAAARQARAKALARQLGVAA (SEQ ID NO: 1). According to another embodiment, the functional equivalent of the MK2 polypeptide inhibitor MMI-0100 of amino acid sequence YARAAARQARAKALARQLGVAA (SEQ ID NO: 1) has at least 95 percent sequence identity to the amino acid sequence YARAAARQARAKALARQLGVAA (SEQ ID NO: 1).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0200 of amino acid sequence YARAAARQARAKALNRQLGVA (SEQ ID NO: 19)

According to another embodiment, the functional equivalent of the MK2 polypeptide inhibitor MMI-0100 of amino acid sequence YARAAARQARAKALARQLGVAA (SEQ ID NO: 1) is a polypeptide MMI-0300 of amino acid sequence FAKLAARLYRKALARQLGVAA (SEQ ID NO: 3).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0400 of amino acid sequence KAFAKLAARLYRKALARQLGVAA (SEQ ID NO: 4).

According to another embodiment, the functional equivalent of the MK2 polypeptide inhibitor MMI-0100 of amino acid sequence YARAAARQARAKALARQLGVAA (SEQ ID NO: 1) is a polypeptide of amino acid sequence YARAAARQARAKALARQLAVA (SEQ ID NO: 5).

According to another embodiment, the functional equivalent of the MK2 polypeptide inhibitor MMI-0100 of amino acid sequence YARAAARQARAKALARQLGVAA (SEQ ID NO: 1) is a polypeptide of amino acid sequence YARAAARQARAKALARQLGVA (SEQ ID NO: 6).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0500 of amino acid sequence HRRIKAWLKKIKALARQLGVAA (SEQ ID NO: 7).

According to another embodiment, the Mitogen-Activated Protein Kinase-Activated Protein Kinase 2 (MK2) inhibitor further comprises a small molecule MK2 inhibitor. Exemplary small molecule MK2 inhibitors have been described in Anderson, D. R. et al., Bioorg. Med. Chem. Lett., 15: 1587 (2005); Wu, J.-P. et al., Bioorg. Med. Chem. Lett., 17: 4664 (2007); Trujillo, J. I. et al., Bioorg. Med. Chem. Lett., 17: 4657 (2007); Goldberg, D. R. et al., Bioorg. Med. Chem. Lett., 18: 938 (2008); Xiong, Z. et al., Bioorg. Med. Chem. Lett., 18: 1994 (2008); Anderson, D. R. et al., J. Med. Chem., 50: 2647 (2007); Lin, S. et al., Bioorg. Med. Chem. Lett., 19: 3238 (2009); Anderson, D. R. et al., Bioorg. Med. Chem. Lett., 19: 4878 (2009); Anderson, D. R. et al., Bioorg. Med. Chem. Lett., 19: 4882 (2009); Harris, C. M. et al., Bioorg. Med. Chem. Lett., 20: 334 (2010); Schlapbach, A. et al., Bioorg. Med. Chem. Lett., 18: 6142 (2008); and Velcicky, J. et al., Bioorg. Med. Chem. Lett., 20: 1293 (2010), the entire disclosure of each of which is incorporated herein by reference.

According to some such embodiments, the small molecule MK2 inhibitor includes, but is not limited to:

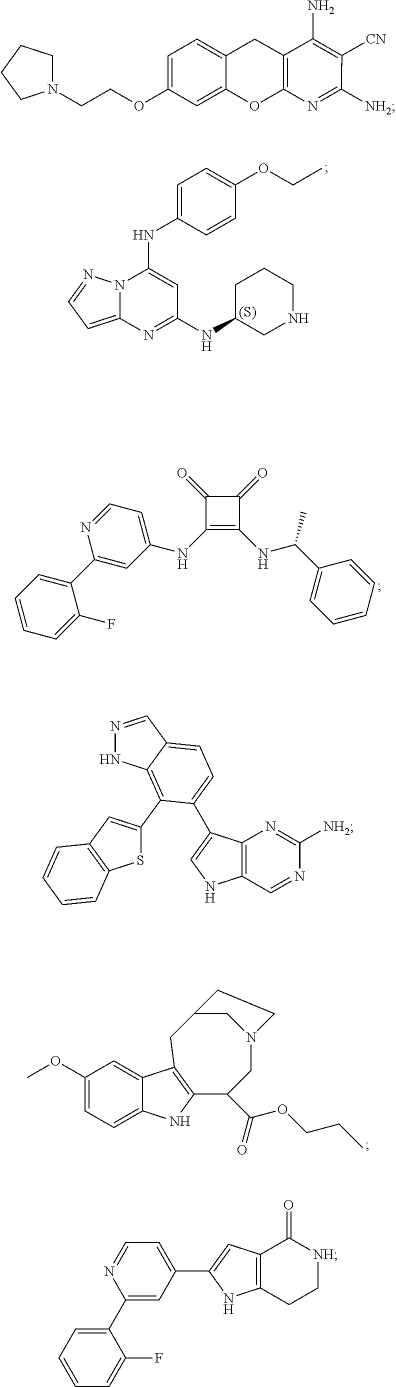

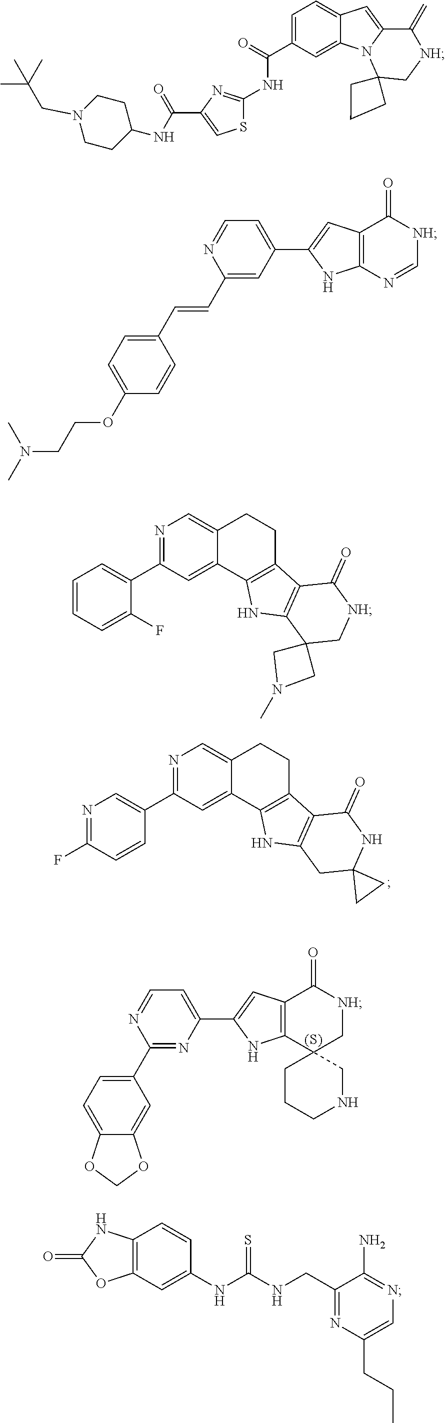

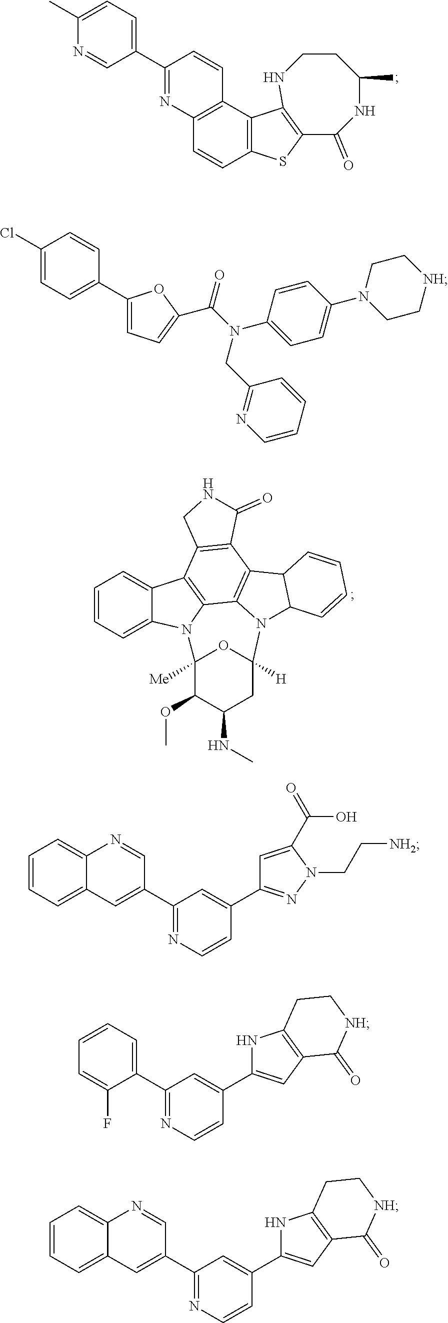

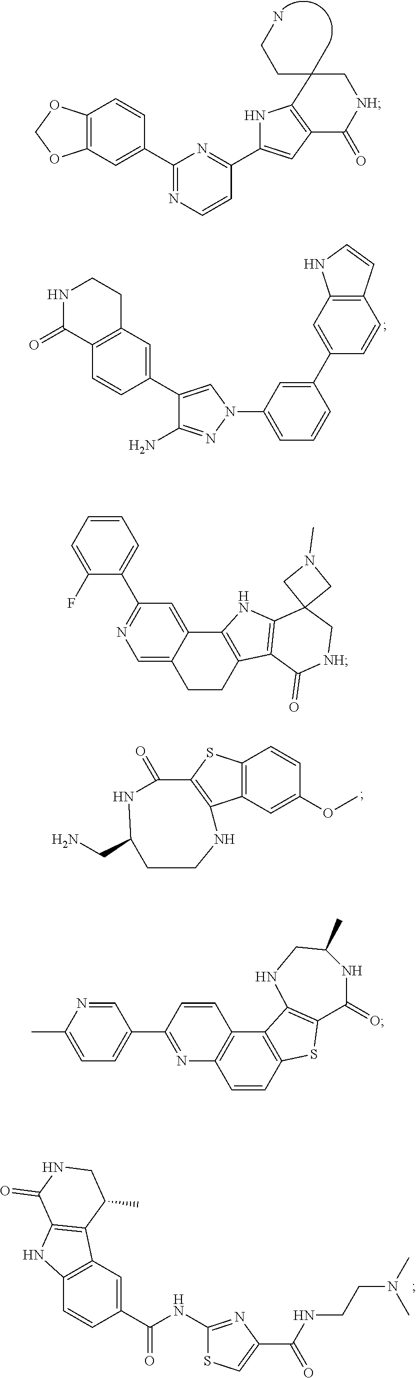

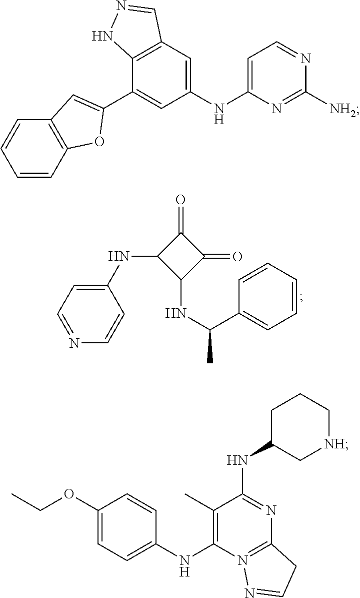

or a combination thereof.

According to another embodiment, the small molecule MK2 inhibitor competes with ATP for binding to MK2. According to some embodiments, the small molecule MK2 inhibitor is a pyrrolopyridine analogue or a multi-cyclic lactam analogue.

According to another embodiment, the small molecule MK2 inhibitor is a pyrrolopyridine analogue. Exemplary pyrrolopyridine analogues are described in Anderson, D. R. et al., “Pyrrolopyridine inhibitors of mitogen-activated protein kinase-activated protein kinase 2 (MK-2),” J. Med. Chem., 50: 2647-2654 (2007), the entire disclosure of which is incorporated herein by reference. According to another embodiment, the pyrrolopyridine analogue is a 2-aryl

pyridine compound of formula I:

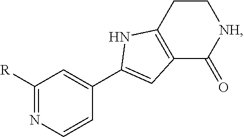

wherein R is H, Cl, phenyl, pyridine, pyrimidine, thienyl, naphthyl, benzothienyl, or quinoline. According to another embodiment, the pyrrolopyridine analogue is a 2-aryl pyridine compound of formula II:

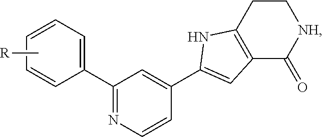

wherein R is OH, Cl, F, CF₃, CN, acetyl, methoxy, NH₂, CO₂H, CONH-cyclopropyl, CONH-cyclopentyl, CONH-cyclohexyl, CONHCH₂-phenyl, CONH(CH₂)₂-phenyl, or CON(methyl)CH₂-phenyl.

According to another embodiment, the small molecule MK2 inhibitor is a multi-cyclic lactam analogue. Exemplary multicyclic lactam analogues are described in Recesz, L. et al., “In vivo and in vitro SAR of tetracyclic MAPKAP-K₂ (MK2) inhibitors: Part I,” Bioorg. Med. Chem. Lett., 20: 4715-4718 (2010); and Recesz, L. et al., “In vivo and in vitro SAR of tetracyclic MAPKAP-K2 (MK2) inhibitors: Part II,” Bioorg. Med. Chem. Lett., 20: 4719-4723 (2010), the entire disclosure of each of which is incorporated herein by reference.

Cutaneous Scar

According to one embodiment, the cutaneous scar can result from healing of a wound. According to another embodiment, the wound is characterized by aberrant activity of Mitogen-Activated Protein Kinase-Activated Protein Kinase 2 (MK2) in a tissue compared to the activity of Mitogen-Activated Protein Kinase-Activated Protein Kinase 2 (MK2) in the tissue of a normal control subject.

According to another embodiment, the therapeutic amount is effective to reduce incidence, severity, or both, of the cutaneous scar without impairing normal wound healing.

According to another embodiment, the pharmaceutical composition is capable of improving alignment of collagen fibers in the wound. According to another embodiment, the therapeutic amount is effective to reduce collagen whorl formation in the wound.

According to one embodiment, the therapeutic amount is effective to accelerate wound healing compared to a control. According to another embodiment, the therapeutic amount is effective to decrease wound size compared to a control. According to some such embodiments, the therapeutic amount is effective to decrease wound size compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 1 day of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 2 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 3 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 4 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 5 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 6 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 7 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 8 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 9 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 10 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 11 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 12 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 13 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 14 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 21 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 30 days of the administration.

According to another embodiment, the therapeutic amount is effective to reduce scarring compared to a control. According to another embodiment, the therapeutic amount is effective to reduce scarring compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration. According to another embodiment, the therapeutic amount is effective to reduce scarring compared to a control as measured by visual analog scale (VAS) score, color matching (CM), matte/shiny (M/S) assessment, contour (C) assessment, distortion (D) assessment, texture (T) assessment, or a combination thereof.

According to another embodiment, the therapeutic amount is effective to decrease scar area compared to a control. According to some such embodiments, the therapeutic amount is effective to decrease scar area compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 1 day of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 2 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 3 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 4 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 5 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 6 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 7 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 8 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 9 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 10 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 11 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 12 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 13 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 14 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 21 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 30 days of the administration.

According to some embodiments, the pharmaceutical composition is capable of modulating expression of a scar-related gene or production of a scar-related gene product. According to one embodiment, the therapeutic amount is effective to modulate the expression of a scar-related gene. According to another embodiment, the therapeutic amount is effective to modulate messenger RNA (mRNA) level expressed from a scar-related gene. According to another embodiment, the therapeutic amount is effective to modulate level of a scar-related gene product expressed from a scar-related gene.

According to some such embodiments, the scar-related gene encodes one or more of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD). According to one embodiment, the scar-related gene encodes Transforming Growth Factor-β1 (TGF-β1). According to another embodiment, the scar-related gene encodes Tumor Necrosis Factor-α (TNF-α). According to another embodiment, the scar-related gene encodes a collagen. According to another embodiment, the collagen is collagen type 1α2 (col1α2) or collagen type 3α1 (col 3α1). According to another embodiment, the scar-related gene encodes Interleukin-6 (IL-6). According to another embodiment, the scar-related gene encodes chemokine (C-C motif) ligand 2 (CCL2) (or monocyte chemotactic protein-1 (MCP-1)). According to another embodiment, the scar-related gene encodes chemokine (C-C motif) receptor 2 (CCR2). According to another embodiment, the scar-related gene encodes EGF-like module-containing mucin-like hormone receptor-like 1 (EMR1). According to another embodiment, the scar-related gene encodes a sma/mad-related protein (SMAD).

According to some such embodiments, the scar-related gene product is selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD). According to another embodiment, the scar-related gene product is Tumor Necrosis Factor-α (TNF-α). According to another embodiment, the scar-related gene product is a collagen. According to another embodiment, the collagen is collagen type 1α2 (col 1α2) or collagen type 3α1 (col 3α1). According to another embodiment, the scar-related gene product is Interleukin-6 (IL-6). According to another embodiment, the scar-related gene product is chemokine (C-C motif) ligand 2 (CCL2) (or monocyte chemotactic protein-1 (MCP-1)). According to another embodiment, the scar-related gene product is chemokine (C-C motif) receptor 2 (CCR2). According to another embodiment, the scar-related gene product is EGF-like module-containing mucin-like hormone receptor-like 1 (EMR1). According to another embodiment, the scar-related gene product is a sma/mad-related protein (SMAD).

According to another embodiment, the pharmaceutical composition is capable of reducing infiltration of one or more types of inflammatory or stem cells, including, without limitation, monocytes, fibrocytes, macrophages, lymphocytes, and mast or dendritic cells, into the wound.

According to another embodiment, the therapeutic amount is effective to reduce infiltration of at least one immunomodulatory cell into the wound. According to some such embodiments, the immunomodulatory cell is selected from the group consisting of a monocyte, a mast cell, a dendritic cell, a macrophage, a T-lymphocyte, or a fibrocyte. According to one embodiment, the immunomodulatory cell is a mast cell. According to another embodiment, the mast cell is characterized by expression of cell surface marker(s) including without limitation CD45 and CD117. According to another embodiment, the immunomodulatory cell is a monocyte. According to another embodiment, the monocyte is characterized by expression of cell surface marker(s) including without limitation CD11b. According to another embodiment, the immunomodulatory cell is a macrophage. According to another embodiment, the macrophage is characterized by expression of cell surface marker(s) including without limitation F4/80. According to another embodiment, the immunomodulatory cell is a T-lymphoyte. According to another embodiment, the T-lymphocyte is a helper T-lymphocyte or a cytotoxic T-lymphocyte. According to another embodiment, the T-lymphocyte is characterized by expression of cell surface marker(s) including without limitation CD4, CD8, or a combination thereof.

According to another embodiment, the therapeutic amount is effective to reduce infiltration of at least one progenitor cell into the wound. According to some such embodiments, the progenitor cell is selected from the group consisting of a hematopoitic stem cell, a mesenchymal stem cell, or a combination thereof. According to one embodiment, the progenitor cell is a hematopoietic stem cell. According to another embodiment, the hematopoietic stem cell is characterized by expression of cell surface marker(s) including without limitation CD45 and Sca1. According to another embodiment, the progenitor cell is a mesenchymal stem cell. According to another embodiment, the mesenchymal stem cell is characterized by expression of cell surface marker(s) including without limitation Sca1 and not CD45.

According to another embodiment, the therapeutic amount is effective to reduce a level of transforming growth factor-β (TGF-β) expression in the wound. According to another embodiment, the therapeutic amount is effective to reduce messenger RNA (mRNA) level of transforming growth factor-β (TGF-β) in the wound. According to another embodiment, the therapeutic amount is effective to reduce protein level of transforming growth factor-β (TGF-β) in the wound.

According to another embodiment, the therapeutic amount is effective to modulate a level of an inflammatory mediator in the wound. According to some embodiments, the inflammatory mediator thus modulated can be without limitation interleukin-1 (IL-1), interleukin-4 (IL-4), interleukin-6 (IL-6), interleukin-8 (IL-8), tumor necrosis factor (TNF), interferon-gamma (IFN-γ), interleukin 12 (IL-12), or a combination thereof.

According to some embodiments, the wound is an abrasion, a laceration, a crush, a contusion, a puncture, an avulsion, a burn, an ulcer, or a combination thereof. According to one embodiment, the wound is an abrasion. According to another embodiment, the wound is a laceration. According to another embodiment, the wound is a crush. According to another embodiment, the wound is a contusion. According to another embodiment, the wound is a puncture. According to another embodiment, the wound is an avulsion. According to another embodiment, the wound is a burn. According to another embodiment, the wound is an ulcer.

According to another embodiment, the wound is an incisional wound.

According to another embodiment, the cutaneous scar is a pathological scar, meaning a scar arising as a result of a disease, disorder, condition, or injury.

According to another embodiment, the pathological scar is a hypertrophic scar.

According to another embodiment, the pathological scar is a keloid.

According to another embodiment, the pathological scar is an atrophic scar.

According to another embodiment, the pathological scar is a scar contracture.

According to another embodiment, the cutaneous scar is an incisional scar.

According to another embodiment, the hypertrophic scar results from a high-tension wound. According to another embodiment, the high-tension wound is located in close proximity to a joint. According to another embodiment, the joint is a knee, an elbow, a wrist, a shoulder, a hip, a spine, across a finger, or a combination thereof. The term “in close proximity” as used herein refers to a distance very near. According to one embodiment, the distance is from about 0.001 mm to about 15 cm. According to another embodiment, the distance is from about 0.001 mm to about 0.005 mm. According to another embodiment, the distance is from about 0.005 mm to about 0.01 mm. According to another embodiment, the distance is from about 0.01 mm to about 0.05 mm. According to another embodiment, the distance is from about 0.05 mm to about 0.1 mm. According to another embodiment, the distance is from about 0.1 mm to about 0.5 mm. According to another embodiment, the distance is from about 0.5 mm to about 1 mm. According to another embodiment, the distance is from about 1 mm to about 2 mm. According to another embodiment, the distance is from about 2 mm to about 3 mm. According to another embodiment, the distance is from about 3 mm to about 4 mm. According to another embodiment, the distance is from about 4 mm to about 5 mm. According to another embodiment, the distance is from about 5 mm to about 6 mm. According to another embodiment, the distance is from about 6 mm to about 7 mm. According to another embodiment, the distance is from about 7 mm to about 8 mm. According to another embodiment, the distance is from about 8 mm to about 9 mm. According to another embodiment, the distance is from about 9 mm to about 1 cm. According to another embodiment, the distance is from about 1 cm to about 2 cm. According to another embodiment, the distance is from about 2 cm to about 3 cm. According to another embodiment, the distance is from about 3 cm to about 4 cm. According to another embodiment, the distance is from about 4 cm to about 5 cm. According to another embodiment, the distance is from about 5 cm to about 6 cm. According to another embodiment, the distance is from about 6 cm to about 7 cm. According to another embodiment, the distance is from about 7 cm to about 8 cm. According to another embodiment, the distance is from about 8 cm to about 9 cm. According to another embodiment, the distance is from about 9 cm to about 10 cm. According to another embodiment, the distance is from about 10 cm to about 11 cm. According to another embodiment, the distance is from about 11 cm to about 12 cm. According to another embodiment, the distance is from about 12 cm to about 13 cm. According to another embodiment, the distance is from about 14 cm to about 15 cm.

According to some embodiments, the pathological scar results from an abrasion, a laceration, an incision, a crush, a contusion, a puncture, an avulsion, a burn, an ulcer, or a combination thereof. According to one embodiment, the pathological scar results from an abrasion. According to another embodiment, the pathological scar results from a laceration. According to another embodiment, the pathological scar results from an incision. According to another embodiment, the pathological scar results from a crush. According to another embodiment, the pathological scar results from a contusion. According to another embodiment, the pathological scar results from a puncture. According to another embodiment, the pathological scar results from an avulsion. According to another embodiment, the pathological scar results from a burn. According to another embodiment, the pathological scar results from an ulcer.

Cutaneous Scar Associated with an Autoimmune Skin Disorder

The term “autoimmune disorder” as used herein refers to disease, disorders or conditions in which the body's immune system, which normally fights infections and viruses, is misdirected and attacks the body's own normal, healthy tissue. In higher organisms, multiple mechanisms of immunological tolerance eliminate or inactivate lymphocytes that bear receptors specific for autoantigens. However, some autoreactive lymphcytes can escape from such mechanisms and present themselves within the peripheral lymphocyte pool.

Autoimmunity is caused by a complex interaction of multiple gene products, unlike immunodeficiency diseases, where a single dominant genetic trait is often the main disease determinant. (Reviewed in Fathman, C. G. et al., “An array of possibilities for the study of autoimmunity” Nature, 435(7042): 605-611 (2005); Anaya, J.-M., “Common mechanisms of autoimmune diseases (the autoimmune tautology),” Autoimmunity Reviews, 11(11): 781-784 (2012)). Autoimmune diseases are major causes of morbidity and mortality throughout the world and are difficult to treat. (Reviewed in for example in Hayter, S. M. et al., “Updated assessment of the prevalence, spectrum and case definition of autoimmune disease,” Autoimmunity Reviews, 11(10): 754-765 (2012); and Rioux, J. D. et al., “Paths to understanding the genetic basis of autoimmune disease,” Nature, 435(7042): 584-589 (2005)).

One mechanism by which the pathogenic potential of such autoreactive lymphocytes is kept in check is through a dedicated lineage of regulatory T (T_{R}) cells. These have been targeted for therapeutic intervention in a wide variety of autoimmune disorders (Reviewed in Kronenberg, M. et al., “Regulation of immunity by self-reactive T cells,” Nature, 435(7042): 598-604 (2005)).

Other components of the pathological cascade in autoimmune disorders that have received attention include, for example, factors involved in lymphocyte homing to target tissues; enzymes that are critical for the penetration of blood vessels and the extracellular matrix by immune cells; cytokines that mediate pathology within the tissues; various cell types that mediate damage at the site of disease, cell antigens; specific adaptive receptors, including the T-cell receptor (TCR) and immunoglobulin; and toxic mediators, such as complement components and nitric oxide. (Reviewed in Feldmann, M. et al., “Design of effective immunotherapy for human autoimmunity,” Nature, 435(7042): 612-619 (2005)).

Although mutations in a single gene can cause autoimmunity, most autoimmune diseases are associated with multiple sequence variants. (Reviewed in Rioux, J. D. et al., “Paths to understanding the genetic basis of autoimmune disease,” Nature, 435(7042): 584-589 (2005); and Goodnow, C. C. et al., “Cellular and genetic mechanisms of self-tolerance and autoimmunity,” Nature, 435(7042): 590-596 (2005)). Autoimmune disorders can be associated with chronic inflammation. Such autoimmune disorders are known as “autoinflammatory conditions”. (Reviewed in Hashkes, P. J. et al., “Autoinflammatory syndromes,” Pediatr. Clin. North Am., 59(2): 447-470 (2012)).

Systemic autoimmunity encompasses autoimmune conditions in which autoreactivity is not limited to a single organ or organ system. This definition includes, but is not limited to, autoimmune diseases including autoimuune skin disease manifestations such as systemic lupus erythematosus (SLE), systemic sclerosis (scleroderma), pemphigus, vitiligo, dermatitis herpetiformis, psoriasis, etc. Cutaneous SLE is a common systemic autoimmune disorder that includes specific skin manifestations such as “butterfly” rash, photosensitive rash dermatitis, and discoid lesions as well as vasculitis and alopecia. SLE is characterized by the presence of antinuclear antibodies (ANAs) and is associated with chronic inflammation. Scleroderma (or systemic sclerosis) is marked by inflammation, followed by deposition of ANAs in skin and viscera. Scleroderma is characterized by a marked reduction in circulation in peripheral arteries of distal fingertips (often stimulated by cold temperatures) known as Reynauld's phenomenon. Pemphigus comprises a group of autoimmune blistering diseases characterized by autoantibody induced epidermal cell-cell detachment (acantholysis). Pemphigus manifests clinically with flaccid blisters and skin erosions. Vitiligo is a skin depigmentation disorder that may be associated with other autoimmune disorders such as the autoimmune polyendocrine syndrome type I. Vitiligo is characterized by the presence of anti-melanocyte autoantibodies, skin infiltration of CD4+ and CD8+ T lymphocytes and overexpression of type I cytokine profiles. Dermatitis herpetiformis (DH) is a life long very pruritic, polymorphic blisteric skin disease associated with gluten sensitivity. The predominiant autoantigen in DH is tissue transglutaminase, found in the intestine and the skin. Psoriasis is a common autoimmune skin disease with a genetic basis affecting 1-3% of the Caucasian population. Psoriasis is characterized by hyperkeratosis, epidermal hyperplasia (acanthosis) and inflammation and dilation of dermal capillaries. (Paul, W. E., “Chapter 1: The immune system: an introduction,” Fundamental Immunology, 4^{th} Edition, Ed. Paul, W. E., Lippicott-Raven Publishers, Philadelphia (1999); Nancy, A.-L. and Yehuda, S., “Prediction and prevention of autoimmune skin disorders,” Arch. Dermatol. Res., 301: 57-64 (2009)).

According to some other embodiments, the pharmaceutical composition is capable of treating a cutaneous scar associated with an autoimmune skin disorder. According to some such embodiments, the autoimmune skin disorder is selected from the group consisting of systemic lupus erythematosus (SLE), systemic sclerosis (scleroderma), pemphigus, vitiligo, dermatitis herpetiformis, psoriasis, or a combination thereof. According to one embodiment, the autoimmune skin disorder is systemic lupus erythematosus (SLE). According to another embodiment, the autoimmune skin disorder is systemic sclerosis (scleroderma). According to another embodiment, the autoimmune skin disorder is pemphigus. According to another embodiment, the autoimmune skin disorder is vitiligo. According to another embodiment, the autoimmune skin disorder is dermatitis herpetiformis. According to another embodiment, the autoimmune skin disorder is psoriasis.

Combination Therapy

According to some embodiments, the pharmaceutical composition further comprises at least one additional therapeutic agent.

According to some such embodiments, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

According to some other embodiments, the additional therapeutic agent is an anti-inflammatory agent.

According to some embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to another embodiment, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

According to another embodiment, the anti-inflammatory agent includes, without limitation, Transforming Growth Factor-beta3 (TGF-β3), an anti-Tumor Necrosis Factor-alpha (TNF-α) agent, or a combination thereof.

According to some embodiments, the additional agent is an analgesic agent. According to some embodiments, the analgesic agent relives pain by elevating the pain threshold without disturbing consciousness or altering other sensory modalities. According to some such embodiments, the analgesic agent is a non-opioid analgesic. According to some such embodiments, the analgesic agent is a non-opioid analgesic. “Non-opioid analgesics” are natural or synthetic substances that reduce pain but are not opioid analgesics. Examples of non-opioid analgesics include, but are not limited to, etodolac, indomethacin, sulindac, tolmetin, nabumetone, piroxicam, acetaminophen, fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, but are not limited to, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to another embodiment, the additional agent is an anti-infective agent. According to another embodiment, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Other examples of at least one additional therapeutic agent include, but are not limited to, rose hip oil, vitamin E, 5-fluorouracil, bleomycin, onion extract, pentoxifylline, prolyl-4-hydroxylase, verapamil, tacrolimus, tamoxifen, tretinoin, colchicine, a calcium antagonist, tranilst, zinc, an antibiotic, or a combination thereof.

### III. Methods for Treating, Reducing or Preventing a Cutaneous Scar

According to another aspect, the described invention provides a method for treating a cutaneous scar in a subject who has suffered or is suffering from a wound, wherein the method comprises administering to the subject a pharmaceutical composition comprising a therapeutic amount of a Mitogen-Activated Protein Kinase-Activated Protein Kinase 2 (MK2) inhibitor comprising a polypeptide inhibitor or a functional equivalent thereof, and a pharmaceutically acceptable carrier, wherein the therapeutic amount is effective to reduce scar area in the subject.

MK2 Inhibitor

According to one embodiment, the Mitogen-Activated Protein Kinase-Activated Protein Kinase 2 (MK2) inhibitor is an MK2 polypeptide inhibitor or a functional equivalent thereof. According to some embodiments, the MK2 polypeptide inhibitor is selected from the group consisting of a polypeptide MMI-0100 of amino acid sequence YARAAARQARAKALARQLGVAA (SEQ ID NO: 1), a polypeptide MMI-0200 of amino acid sequence YARAAARQARAKALNRQLGVA (SEQ ID NO: 19), a polypeptide MMI-0300 of amino acid sequence FAKLAARLYRKALARQLGVAA (SEQ ID NO: 3), a polypeptide MMI-0400 of amino acid sequence KAFAKLAARLYRKALARQLGVAA (SEQ ID NO: 4), and a polypeptide MMI-0500 of amino acid sequence HRRIKAWLKKIKALARQLGVAA (SEQ ID NO: 7). According to one embodiment, the MK2 polypeptide inhibitor is a polypeptide MMI-0100 of amino acid sequence YARAAARQARAKALARQLGVAA (SEQ ID NO: 1). According to another embodiment, the MK2 polypeptide inhibitor is a polypeptide MMI-0200 of amino acid sequence YARAAARQARAKALNRQLGVA (SEQ ID NO: 19). According to another embodiment, the MK2 polypeptide inhibitor is a polypeptide MMI-0300 of amino acid sequence FAKLAARLYRKALARQLGVAA (SEQ ID NO: 3). According to another embodiment, the MK2 polypeptide inhibitor is a polypeptide MMI-0400 of amino acid sequence KAFAKLAARLYRKALARQLGVAA (SEQ ID NO: 4). According to another embodiment, the MK2 polypeptide inhibitor is a polypeptide MMI-0500 of amino acid sequence HRRIKAWLKKIKALARQLGVAA (SEQ ID NO: 7).

According to another embodiment, the functional equivalent of the MK2 polypeptide inhibitor MMI-0100 of amino acid sequence YARAAARQARAKALARQLGVAA (SEQ ID NO: 1) has a substantial sequence identity to the amino acid sequence YARAAARQARAKALARQLGVAA (SEQ ID NO: 1).

According to another embodiment, the functional equivalent of the MK2 polypeptide inhibitor MMI-0100 of amino acid sequence YARAAARQARAKALARQLGVAA (SEQ ID NO: 1) has at least 80 percent sequence identity to the amino acid sequence YARAAARQARAKALARQLGVAA (SEQ ID NO: 1). According to another embodiment, the functional equivalent of the MK2 polypeptide inhibitor MMI-0100 of amino acid sequence YARAAARQARAKALARQLGVAA (SEQ ID NO: 1) has at least 90 percent sequence identity to the amino acid sequence YARAAARQARAKALARQLGVAA (SEQ ID NO: 1). According to another embodiment, the functional equivalent of the MK2 polypeptide inhibitor MMI-0100 of amino acid sequence YARAAARQARAKALARQLGVAA (SEQ ID NO: 1) has at least 95 percent sequence identity to the amino acid sequence YARAAARQARAKALARQLGVAA (SEQ ID NO: 1).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0200 of amino acid sequence YARAAARQARAKALNRQLGVA (SEQ ID NO: 19)

According to another embodiment, the functional equivalent of the MK2 polypeptide inhibitor MMI-0100 of amino acid sequence YARAAARQARAKALARQLGVAA (SEQ ID NO: 1) is a polypeptide MMI-0300 of amino acid sequence FAKLAARLYRKALARQLGVAA (SEQ ID NO: 3).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0400 of amino acid sequence KAFAKLAARLYRKALARQLGVAA (SEQ ID NO: 4).

According to another embodiment, the functional equivalent of the MK2 polypeptide inhibitor MMI-0100 of amino acid sequence YARAAARQARAKALARQLGVAA (SEQ ID NO: 1) is a polypeptide of amino acid sequence YARAAARQARAKALARQLAVA (SEQ ID NO: 5).

According to another embodiment, the functional equivalent of the MK2 polypeptide inhibitor MMI-0100 of amino acid sequence YARAAARQARAKALARQLGVAA (SEQ ID NO: 1) is a polypeptide of amino acid sequence YARAAARQARAKALARQLGVA (SEQ ID NO: 6).

According to another embodiment, the functional equivalent of the MK2 polypeptide inhibitor MMI-0100 of amino acid sequence YARAAARQARAKALARQLGVAA (SEQ ID NO: 1) is a polypeptide MMI-0500 of amino acid sequence HRRIKAWLKKIKALARQLGVAA (SEQ ID NO: 7).

According to another embodiment, the Mitogen-Activated Protein Kinase-Activated Protein Kinase 2 (MK2) inhibitor further comprises a small molecule MK2 inhibitor. Exemplary small molecule MK2 inhibitors have been described in Anderson, D. R. et al., Bioorg. Med. Chem. Lett., 15: 1587 (2005); Wu, J.-P. et al., Bioorg. Med. Chem. Lett., 17: 4664 (2007); Trujillo, J. I. et al., Bioorg. Med. Chem. Lett., 17: 4657 (2007); Goldberg, D. R. et al., Bioorg. Med. Chem. Lett., 18: 938 (2008); Xiong, Z. et al., Bioorg. Med. Chem. Lett., 18: 1994 (2008); Anderson, D. R. et al., J. Med. Chem., 50: 2647 (2007); Lin, S. et al., Bioorg. Med. Chem. Lett., 19: 3238 (2009); Anderson, D. R. et al., Bioorg. Med. Chem. Lett., 19: 4878 (2009); Anderson, D. R. et al., Bioorg. Med. Chem. Lett., 19: 4882 (2009); Harris, C. M. et al., Bioorg. Med. Chem. Lett., 20: 334 (2010); Schlapbach, A. et al., Bioorg. Med. Chem. Lett., 18: 6142 (2008); and Velcicky, J. et al., Bioorg. Med. Chem. Lett., 20: 1293 (2010), the entire disclosure of each of which is incorporated herein by reference.

According to some such embodiments, the small molecule MK2 inhibitor includes, but is not limited to:

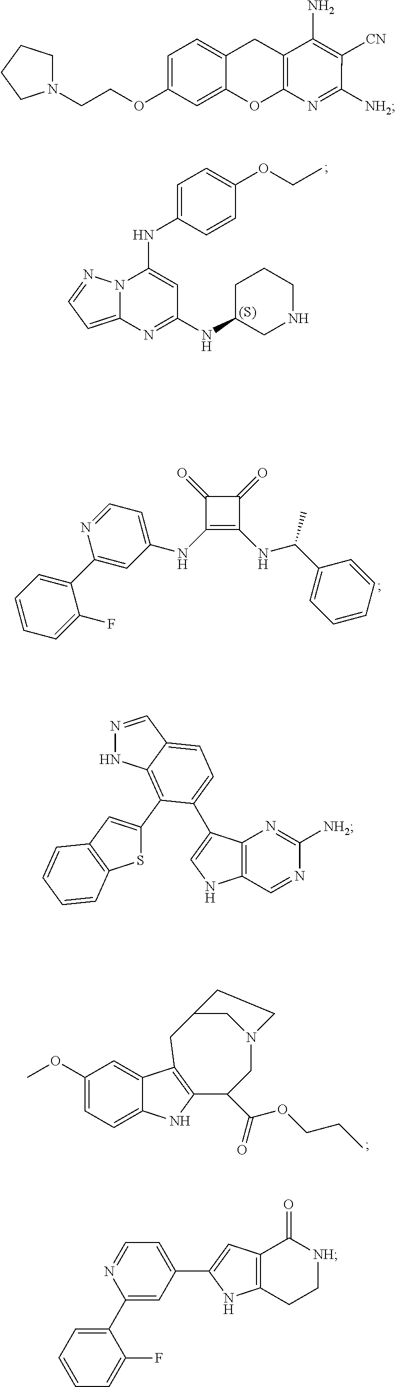

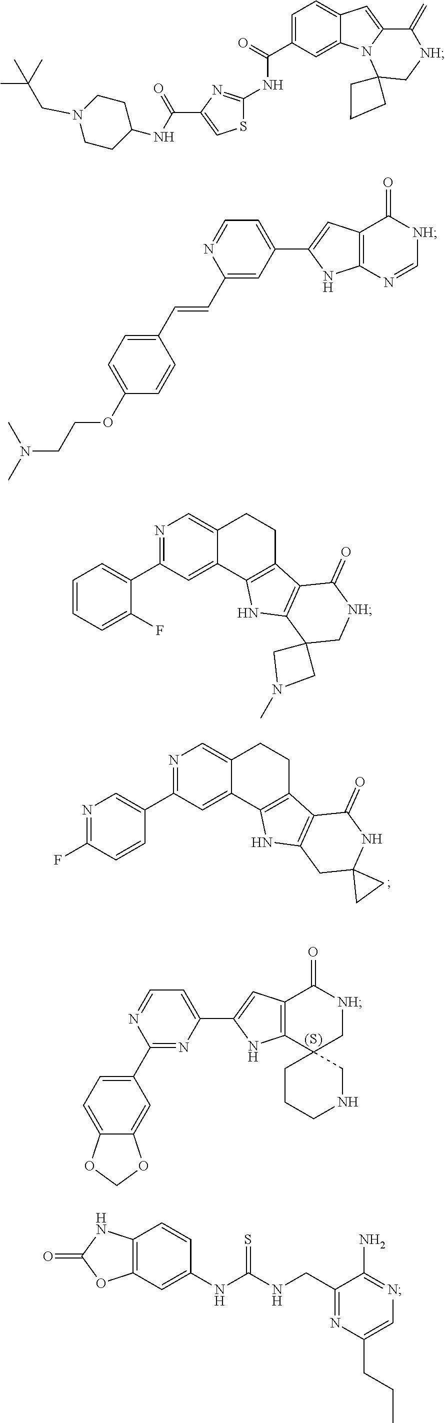

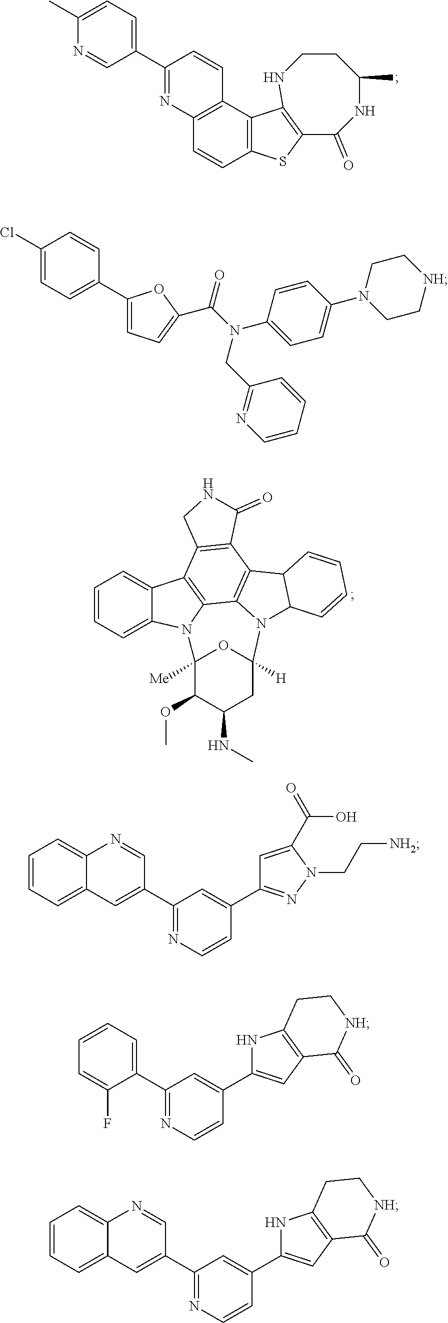

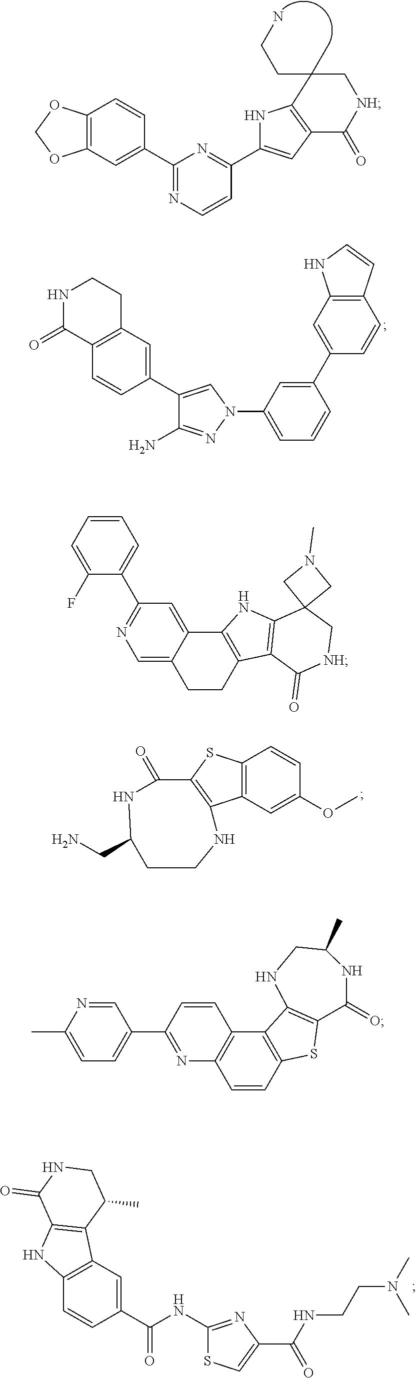

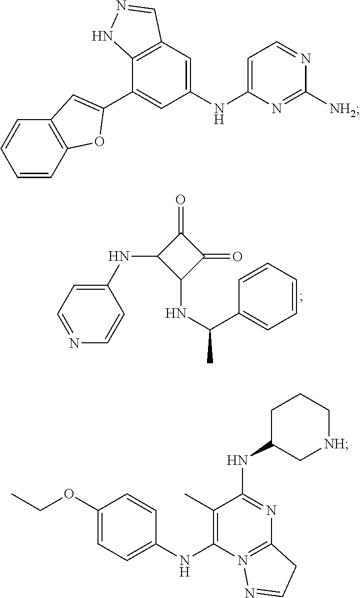

or a combination thereof.

According to another embodiment, the small molecule MK2 inhibitor competes with ATP for binding to MK2. According to some embodiments, the small molecule MK2 inhibitor is a pyrrolopyridine analogue or a multi-cyclic lactam analogue.

According to another embodiment, the small molecule MK2 inhibitor is a pyrrolopyridine analogue. Exemplary pyrrolopyridine analogues are described in Anderson, D. R. et al., “Pyrrolopyridine inhibitors of mitogen-activated protein kinase-activated protein kinase 2 (MK-2),” J. Med. Chem., 50: 2647-2654 (2007), the entire disclosure of which is incorporated herein by reference. According to another embodiment, the pyrrolopyridine analogue is a 2-aryl pyridine compound of formula I:

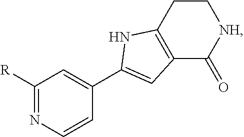

wherein R is H, Cl, phenyl, pyridine, pyrimidine, thienyl, naphthyl, benzothienyl, or quinoline. According to another embodiment, the pyrrolopyridine analogue is a 2-aryl pyridine compound of formula II:

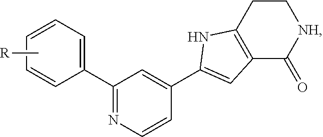

wherein R is OH, Cl, F, CF₃, CN, acetyl, methoxy, NH₂, CO₂H, CONH-cyclopropyl, CONH-cyclopentyl, CONH-cyclohexyl, CONHCH₂-phenyl, CONH(CH₂)₂-phenyl, or CON(methyl)CH₂-phenyl.

According to another embodiment, the small molecule MK2 inhibitor is a multi-cyclic lactam analogue. Exemplary multicyclic lactam analogues are described in Recesz, L. et al., “In vivo and in vitro SAR of tetracyclic MAPKAP-K2 (MK2) inhibitors: Part I,” Bioorg. Med. Chem. Lett., 20: 4715-4718 (2010); and Recesz, L. et al., “In vivo and in vitro SAR of tetracyclic MAPKAP-K2 (MK2) inhibitors: Part II,” Bioorg. Med. Chem. Lett., 20: 4719-4723 (2010), the entire disclosure of each of which is incorporated herein by reference.

Cutaneous Scar

According to one embodiment, the cutaneous scar can result from healing of a wound. According to another embodiment, the wound is characterized by aberrant activity of Mitogen-Activated Protein Kinase-Activated Protein Kinase 2 (MK2) in a tissue compared to the activity of Mitogen-Activated Protein Kinase-Activated Protein Kinase 2 (MK2) in the tissue of a normal control subject.

According to another embodiment, the therapeutic amount is effective to reduce incidence, severity, or both, of the cutaneous scar without impairing normal wound healing.

According to another embodiment, the pharmaceutical composition is capable of improving alignment of collagen fibers in the wound. According to another embodiment, the therapeutic amount is effective to reduce collagen whorl formation in the wound.

According to one embodiment, the therapeutic amount is effective to accelerate wound healing compared to a control. According to another embodiment, the therapeutic amount is effective to decrease wound size compared to a control. According to some such embodiments, the therapeutic amount is effective to decrease wound size compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 1 day of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 2 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 3 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 4 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 5 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 6 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 7 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 8 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 9 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 10 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 11 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 12 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 13 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 14 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 21 days of the administration.

According to another embodiment, the therapeutic amount is effective to decrease wound size compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 30 days of the administration.

According to another embodiment, the therapeutic amount is effective to reduce scarring compared to a control. According to another embodiment, the therapeutic amount is effective to reduce scarring compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration. According to another embodiment, the therapeutic amount is effective to reduce scarring compared to a control as measured by visual analog scale (VAS) score, color matching (CM), matte/shiny (M/S) assessment, contour (C) assessment, distortion (D) assessment, texture (T) assessment, or a combination thereof.

According to another embodiment, the therapeutic amount is effective to decrease scar area compared to a control. According to some such embodiments, the therapeutic amount is effective to decrease scar area compared to a control within at least 1 day, at least 2 days, at least 3 days, at least 4 days, at least 5 days, at least 7 days, at least 8 days, at least 9 days, at least 10 days, at least 11 days, at least 12 days, at least 13 days, at least 14 days, at least 21 days, or at least 30 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 1 day of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 2 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 3 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 4 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 5 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 6 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 7 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 8 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 9 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 10 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 11 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 12 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 13 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 14 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 21 days of the administration.

According to another embodiment, the therapeutic amount is effective to decrease scar area compared to a control by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% within at least 30 days of the administration.

According to some embodiments, the pharmaceutical composition is capable of modulating expression of a scar-related gene or production of a scar-related gene product. According to one embodiment, the therapeutic amount is effective to modulate the expression of a scar-related gene. According to another embodiment, the therapeutic amount is effective to modulate messenger RNA (mRNA) level expressed from a scar-related gene. According to another embodiment, the therapeutic amount is effective to modulate level of a scar-related gene product expressed from a scar-related gene.

According to some such embodiments, the scar-related gene encodes one or more of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD). According to one embodiment, the scar-related gene encodes Transforming Growth Factor-β1 (TGF-β1). According to another embodiment, the scar-related gene encodes Tumor Necrosis Factor-α (TNF-α). According to another embodiment, the scar-related gene encodes a collagen. According to another embodiment, the collagen is collagen type 1α2 (col 1α2) or collagen type 3α1 (col 3α1). According to another embodiment, the scar-related gene encodes Interleukin-6 (IL-6). According to another embodiment, the scar-related gene encodes chemokine (C-C motif) ligand 2 (CCL2) (or monocyte chemotactic protein-1 (MCP-1)). According to another embodiment, the scar-related gene encodes chemokine (C-C motif) receptor 2 (CCR2). According to another embodiment, the scar-related gene encodes EGF-like module-containing mucin-like hormone receptor-like 1 (EMR1). According to another embodiment, the scar-related gene encodes a sma/mad-related protein (SMAD).

According to some such embodiments, the scar-related gene product is selected from the group consisting of Transforming Growth Factor-β1 (TGF-β1), Tumor Necrosis Factor-α(TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD). According to another embodiment, the scar-related gene product is Tumor Necrosis Factor-α (TNF-α). According to another embodiment, the scar-related gene product is a collagen. According to another embodiment, the collagen is collagen type 1α2 (col 1α2) or collagen type 3α1 (col 3α1). According to another embodiment, the scar-related gene product is Interleukin-6 (IL-6). According to another embodiment, the scar-related gene product is chemokine (C-C motif) ligand 2 (CCL2) (or monocyte chemotactic protein-1 (MCP-1)). According to another embodiment, the scar-related gene product is chemokine (C-C motif) receptor 2 (CCR2). According to another embodiment, the scar-related gene product is EGF-like module-containing mucin-like hormone receptor-like 1 (EMR1). According to another embodiment, the scar-related gene product is a sma/mad-related protein (SMAD).

According to another embodiment, the pharmaceutical composition is capable of reducing infiltration of one or more types of inflammatory or stem cells, including, without limitation, monocytes, fibrocytes, macrophages, lymphocytes, and mast or dendritic cells, into the wound.

According to another embodiment, the therapeutic amount is effective to reduce infiltration of at least one immunomodulatory cell into the wound. According to some such embodiments, the immunomodulatory cell is selected from the group consisting of a monocyte, a mast cell, a dendritic cell, a macrophage, a T-lymphocyte, or a fibrocyte. According to one embodiment, the immunomodulatory cell is a mast cell. According to another embodiment, the mast cell is characterized by expression of cell surface marker(s) including without limitation CD45 and CD117. According to another embodiment, the immunomodulatory cell is a monocyte. According to another embodiment, the monocyte is characterized by expression of cell surface marker(s) including without limitation CD11b. According to another embodiment, the immunomodulatory cell is a macrophage. According to another embodiment, the macrophage is characterized by expression of cell surface marker(s) including without limitation F4/80. According to another embodiment, the immunomodulatory cell is a T-lymphoyte. According to another embodiment, the T-lymphocyte is a helper T-lymphocyte or a cytotoxic T-lymphocyte. According to another embodiment, the T-lymphocyte is characterized by expression of cell surface marker(s) including without limitation CD4, CD8, or a combination thereof.

According to another embodiment, the therapeutic amount is effective to reduce infiltration of at least one progenitor cell into the wound. According to some such embodiments, the progenitor cell is selected from the group consisting of a hematopoitic stem cell, a mesenchymal stem cell, or a combination thereof. According to one embodiment, the progenitor cell is a hematopoietic stem cell. According to another embodiment, the hematopoietic stem cell is characterized by expression of cell surface marker(s) including without limitation CD45 and Sca1. According to another embodiment, the progenitor cell is a mesenchymal stem cell. According to another embodiment, the mesenchymal stem cell is characterized by expression of cell surface marker(s) including without limitation Sca1 and not CD45.

According to another embodiment, the therapeutic amount is effective to reduce a level of transforming growth factor-β (TGF-β) expression in the wound. According to another embodiment, the therapeutic amount is effective to reduce messenger RNA (mRNA) level of transforming growth factor-β (TGF-β) in the wound. According to another embodiment, the therapeutic amount is effective to reduce protein level of transforming growth factor-β (TGF-β) in the wound.

According to another embodiment, the therapeutic amount is effective to modulate a level of an inflammatory mediator in the wound. According to some embodiments, the inflammatory mediator thus modulated can be without limitation interleukin-1 (IL-1), interleukin-4 (IL-4), interleukin-6 (IL-6), interleukin-8 (IL-8), tumor necrosis factor (TNF), interferon-gamma (IFN-γ), interleukin 12 (IL-12), or a combination thereof.

According to some embodiments, the wound is an abrasion, a laceration, a crush, a contusion, a puncture, an avulsion, a burn, an ulcer, or a combination thereof. According to one embodiment, the wound is an abrasion. According to another embodiment, the wound is a laceration. According to another embodiment, the wound is a crush. According to another embodiment, the wound is a contusion. According to another embodiment, the wound is a puncture. According to another embodiment, the wound is an avulsion. According to another embodiment, the wound is a burn. According to another embodiment, the wound is an ulcer.

According to another embodiment, the wound is an incisional wound.

According to another embodiment, the cutaneous scar is a pathological scar, meaning a scar arising as a result of a disease, disorder, condition, or injury.

According to another embodiment, the pathological scar is a hypertrophic scar.

According to another embodiment, the pathological scar is a keloid.

According to another embodiment, the pathological scar is an atrophic scar.

According to another embodiment, the pathological scar is a scar contracture.

According to another embodiment, the cutaneous scar is an incisional scar.

According to another embodiment, the hypertrophic scar results from a high-tension wound. According to another embodiment, the high-tension wound is located in close proximity to a joint. According to another embodiment, the joint is a knee, an elbow, a wrist, a shoulder, a hip, a spine, across a finger, or a combination thereof. The term “in close proximity” as used herein refers to a distance very near. According to one embodiment, the distance is from about 0.001 mm to about 15 cm. According to another embodiment, the distance is from about 0.001 mm to about 0.005 mm. According to another embodiment, the distance is from about 0.005 mm to about 0.01 mm. According to another embodiment, the distance is from about 0.01 mm to about 0.05 mm. According to another embodiment, the distance is from about 0.05 mm to about 0.1 mm. According to another embodiment, the distance is from about 0.1 mm to about 0.5 mm. According to another embodiment, the distance is from about 0.5 mm to about 1 mm. According to another embodiment, the distance is from about 1 mm to about 2 mm. According to another embodiment, the distance is from about 2 mm to about 3 mm. According to another embodiment, the distance is from about 3 mm to about 4 mm. According to another embodiment, the distance is from about 4 mm to about 5 mm. According to another embodiment, the distance is from about 5 mm to about 6 mm. According to another embodiment, the distance is from about 6 mm to about 7 mm. According to another embodiment, the distance is from about 7 mm to about 8 mm. According to another embodiment, the distance is from about 8 mm to about 9 mm. According to another embodiment, the distance is from about 9 mm to about 1 cm. According to another embodiment, the distance is from about 1 cm to about 2 cm. According to another embodiment, the distance is from about 2 cm to about 3 cm. According to another embodiment, the distance is from about 3 cm to about 4 cm. According to another embodiment, the distance is from about 4 cm to about 5 cm. According to another embodiment, the distance is from about 5 cm to about 6 cm. According to another embodiment, the distance is from about 6 cm to about 7 cm. According to another embodiment, the distance is from about 7 cm to about 8 cm. According to another embodiment, the distance is from about 8 cm to about 9 cm. According to another embodiment, the distance is from about 9 cm to about 10 cm. According to another embodiment, the distance is from about 10 cm to about 11 cm. According to another embodiment, the distance is from about 11 cm to about 12 cm. According to another embodiment, the distance is from about 12 cm to about 13 cm. According to another embodiment, the distance is from about 14 cm to about 15 cm.

According to some embodiments, the pathological scar results from an abrasion, a laceration, an incision, a crush, a contusion, a puncture, an avulsion, a burn, an ulcer, or a combination thereof. According to one embodiment, the pathological scar results from an abrasion. According to another embodiment, the pathological scar results from a laceration. According to another embodiment, the pathological scar results from an incision. According to another embodiment, the pathological scar results from a crush. According to another embodiment, the pathological scar results from a contusion. According to another embodiment, the pathological scar results from a puncture. According to another embodiment, the pathological scar results from an avulsion. According to another embodiment, the pathological scar results from a burn. According to another embodiment, the pathological scar results from an ulcer.

According to some other embodiments, the pharmaceutical composition is capable of treating a cutaneous scar associated with an autoimmune skin disorder. According to some such embodiments, the autoimmune skin disorder is selected from the group consisting of systemic lupus erythematosus (SLE), systemic sclerosis (scleroderma), pemphigus, vitiligo, dermatitis herpetiformis, psoriasis, or a combination thereof. According to one embodiment, the autoimmune skin disorder is systemic lupus erythematosus (SLE). According to another embodiment, the autoimmune skin disorder is systemic sclerosis (scleroderma). According to another embodiment, the autoimmune skin disorder is pemphigus. According to another embodiment, the autoimmune skin disorder is vitiligo. According to another embodiment, the autoimmune skin disorder is dermatitis herpetiformis. According to another embodiment, the autoimmune skin disorder is psoriasis.

Administering Step

According to one embodiment, the pharmaceutical composition is administered systemically. According to another embodiment, the pharmaceutical composition is administered orally. According to another embodiment, the pharmaceutical composition is administered intraperitoneally. According to another embodiment, the pharmaceutical composition is administered intramuscularly. According to another embodiment, the pharmaceutical composition is administered intravenously. According to another embodiment, the pharmaceutical composition is administered parenterally. According to another embodiment, the pharmaceutical composition is administered intraarterially.

According to another embodiment, the pharmaceutical composition is administered locally. According to another embodiment, the pharmaceutical composition is administered topically. According to another embodiment, the pharmaceutical composition is administered intradermally. According to another embodiment, the pharmaceutical composition is administered via intradermal injection. According to another embodiment, the pharmaceutical composition is administered subcutaneously. According to another embodiment, the pharmaceutical composition is administered transcutaneously.

According to another embodiment, the pharmaceutical composition is administered by means of an injection apparatus. According to some embodiments, the injection apparatus is selected from the group consisting of a needle, a cannula, a catheter, a suture, or a combination thereof. According to one embodiment, the injection apparatus is a needle. According to another embodiment, the injection apparatus is a cannula. According to another embodiment, the injection apparatus is a catheter. According to another embodiment, the injection apparatus is a suture.

According to another embodiment, the injection apparatus is soaked with the pharmaceutical composition prior to administration.

According to another embodiment, the pharmaceutical composition is administered at one time as a single dose.

According to another embodiment, the pharmaceutical composition is administered as a plurality of doses over a period of time.

According to some embodiments, the pharmaceutical composition is administered to a wound prior to wound closure. According to some other embodiments, the pharmaceutical composition is administered to a wound at the time of wound closure. According to some other embodiments, the pharmaceutical composition is administered to a wound after wound closure.

According to some embodiments, the pharmaceutical composition is administered multiple times per day. According to some other embodiments, the pharmaceutical composition is administered multiple times per day coincident with dressing changes.

According to another embodiment, the period of time is a day, a week, a month, a month, a year, or multiples thereof. According to another embodiment, the pharmaceutical composition is administered daily for a period of at least one week. According to another embodiment, the pharmaceutical composition is administered weekly for a period of at least one month. According to another embodiment, the pharmaceutical composition is administered monthly for a period of at least two months. According to another embodiment, the pharmaceutical composition is administered repeatedly over a period of at least one year. According to another embodiment, the pharmaceutical composition is administered at least once monthly. According to another embodiment, the pharmaceutical composition is administered at least once weekly. According to another embodiment, the pharmaceutical composition is administered at least once daily.

According to another embodiment, the pharmaceutical composition is administered to a wound site by means of a dressing comprising the pharmaceutical composition. According to another embodiment, at least one surface of the dressing is impregnated with the composition. Examples of dressings suitable for the purpose of the present invention, include, but are not limited to, a gauze dressing, a tulle dressing, an alginate dressing, a polyurethane dressing, a silicone foam dressing, and a collagen dressing, a synthetic polymer scaffold, or a combination thereof. According to another embodiment, other suitable dressings include occlusive dressings, including, but not limited to, film dressings, semi-permeable film dressings, hydrogel dressings, hydrocolloid dressings, or a combination thereof.

According to another embodiment, the pharmaceutical composition is embedded in a dermal substitute that provides a three-dimensional extracellular scaffold.

According to another embodiment, the dermal substitute is made of a natural biological material, including, but not limited to, human cadaver skin, porcine cadaver skin, and porcine small intestine submucosa. According to another embodiment, the natural biological material comprises a matrix. According to another embodiment, the natural biological material consists essentially of a matrix that is substantially devoid of cell remnants.

According to another embodiment, the dermal substitute is a constructive biological material. Examples of constructive biological materials suitable for the purpose of the present invention include, but are not limited to, collagen, glycosaminoglycan, fibronectin, hyaluonic acid, elastine, or a combination thereof. According to some such embodiments, the constructive biological material is a bilayer, non-cellularized dermal regeneration template. According to another embodiment, the constructive biological material is a single layer, cellularized dermal regeneration template.

According to another embodiment, the dermal substitute is a synthetic dermal substitute. According to another embodiment, the synthetic dermal substitute further comprises an RGD peptide of amino acid sequence Arginine-Glycine-Aspartate. According to another embodiment, the synthetic dermal substitute comprises a hydrogel.

Topical administration also may involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation. Patches suitable for use in the described invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated by reference in its entirety. These patches are well known in the art and generally available commercially.

According to another embodiment, the pharmaceutical composition is administered before, during, or after closing of the wound.

According to another embodiment, the closing of the wound is carried out by suturing, stapling, applying a surgical adhesive, or a combination thereof.

According to another embodiment, the surgical adhesive comprises an adhesive tape, octyl-2-cyanoacrylate or fibrin tissue adhesive.

According to another embodiment, the closing of the wound is carried out by subcutaneous sutures. Without being limited by theory, it is believed that the subcutaneous sutures are capable of taking tension off skin edges prior to applying the surgical adhesive.

Formulation

According to some embodiments, the carrier is a controlled release carrier. The term “controlled release” is intended to refer to any drug-containing formulation in which the manner and profile of drug release from the formulation are controlled. This includes immediate as well as non-immediate release formulations, with non-immediate release formulations including, but not limited to, sustained release and delayed release formulations.

Injectable depot forms may be made by forming microencapsulated matrices of a therapeutic agent/drug in biodegradable polymers such as, but not limited to, polyesters (polyglycolide, polylactic acid and combinations thereof), polyester polyethylene glycol copolymers, polyamino-derived biopolymers, polyanhydrides, polyorthoesters, polyphosphazenes, sucrose acetate isobutyrate (SAIB), photopolymerizable biopolymers, naturally-occurring biopolymers, protein polymers, collagen, and polysaccharides. Depending upon the ratio of drug to polymer and the nature of the particular polymer employed, the rate of drug release may be controlled. Such long acting formulations may be formulated with suitable polymeric or hydrophobic materials (for example, as an emulsion in acceptable oil) or ion exchange resins, or as sparingly soluble derivatives (for example, as a sparingly soluble salt). Depot injectable formulations also are prepared by entrapping the drug in liposomes or microemulsions which are compatible with body tissues.

According to some embodiments, the carrier is a delayed release carrier. According to another embodiment, the delayed release carrier comprises a biodegradable polymer. According to another embodiment, the biodegradable polymer is a synthetic polymer. According to another embodiment, the biodegradable polymer is a naturally occurring polymer.

According to some embodiments, the carrier is a sustained release carrier. According to another embodiment, the sustained-release carrier comprises a biodegradable polymer. According to another embodiment, the biodegradable polymer is a synthetic polymer. According to another embodiment, the biodegradable polymer is a naturally occurring polymer.

According to some embodiments, the pharmaceutical composition further comprises at least one additional therapeutic agent.

According to some such embodiments, the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

Combination Therapy

According to some other embodiments, the additional therapeutic agent is an anti-inflammatory agent.

According to some such embodiments, the anti-inflammatory agent is a steroidal anti-inflammatory agent. The term “steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflucortolone valerate, fluadrenolone, fluclorolone acetonide, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to some other embodiments, the anti-inflammatory agent is a nonsteroidal anti-inflammatory agent. The term “non-steroidal anti-inflammatory agent” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

According to another embodiment, the anti-inflammatory agent includes, without limitation, Transforming Growth Factor-beta3 (TGF-β3), an anti-Tumor Necrosis Factor-alpha (TNF-α) agent, or a combination thereof.

According to some embodiments, the additional agent is an analgesic agent. According to some embodiments, the analgesic agent relives pain by elevating the pain threshold without disturbing consciousness or altering other sensory modalities. According to some such embodiments, the analgesic agent is a non-opioid analgesic. “Non-opioid analgesics” are natural or synthetic substances that reduce pain but are not opioid analgesics. Examples of non-opioid analgesics include, but are not limited to, etodolac, indomethacin, sulindac, tolmetin, nabumetone, piroxicam, acetaminophen, fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone. According to some other embodiments, the analgesic is an opioid analgesic. “Opioid analgesics”, “opioid”, or “narcotic analgesics” are natural or synthetic substances that bind to opioid receptors in the central nervous system, producing an agonist action. Examples of opioid analgesics include, but are not limited to, codeine, fentanyl, hydromorphone, levorphanol, meperidine, methadone, morphine, oxycodone, oxymorphone, propoxyphene, buprenorphine, butorphanol, dezocine, nalbuphine, and pentazocine.

According to another embodiment, the additional agent is an anti-infective agent. According to another embodiment, the anti-infective agent is an antibiotic agent. The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Other examples of at least one additional therapeutic agent include, but are not limited to, rose hip oil, vitamin E, 5-fluorouracil, bleomycin, onion extract, pentoxifylline, prolyl-4-hydroxylase, verapamil, tacrolimus, tamoxifen, tretinoin, colchicine, a calcium antagonist, tranilst, zinc, an antibiotic, and a combination thereof.

Reducing Off-Target Affects

According to some embodiments, in order to enhance drug efficacy and to reduce accumulation of the polypeptide MMI-0100 of amino acid sequence YARAAARQARAKALARQLGVAA (SEQ ID NO: 1) or its functional equivalent in non-target tissues, the polypeptide of the present invention of amino acid sequence YARAAARQARAKALARQLGVAA (SEQ ID NO: 1) or its functional equivalent can be linked or associated with a targeting moiety, which directs the polypeptide to a specific cell type or tissue. Examples of the targeting moiety include, but are not limited to, (i) a ligand for a known or unknown receptor or (ii) a compound, a peptide, or a monoclonal antibody that binds to a specific molecular target, e.g., a peptide or carbohydrate, expressed on the surface of a specific cell type.

According to another embodiment, the functional equivalent of the polypeptide MMI-0100 of amino acid sequence YARAAARQARAKALARQLGVAA (SEQ ID NO: 1) is a fusion peptide comprising a first polypeptide operatively linked to a second polypeptide, wherein the first polypeptide is of amino acid sequence YARAAARQARA (SEQ ID NO: 11), and the second polypeptide comprises a therapeutic domain whose sequence has a substantial identity to amino acid sequence KALARQLGVAA (SEQ ID NO: 2).

According to another embodiment, the second polypeptide has at least 70 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2). According to another embodiment, the second polypeptide has at least 80 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2). According to another embodiment, the second polypeptide has at least 90 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2). According to another embodiment, the second polypeptide has at least 95 percent sequence identity to amino acid sequence KALARQLGVAA (SEQ ID NO: 2), and the pharmaceutical composition inhibits the kinase activity of Mitogen-Activated Protein Kinase-Activated Protein Kinase 2 (MK2).

According to another embodiment, the second polypeptide is a polypeptide of amino acid sequence KALARQLAVA (SEQ ID NO: 8).

According to another embodiment, the second polypeptide is a polypeptide of amino acid sequence KALARQLGVA (SEQ ID NO: 9).

According to another embodiment, the second polypeptide is a polypeptide of amino acid sequence KALARQLGVAA (SEQ ID NO: 10); see, e.g., U.S. Published Application No. 2009-0196927, U.S. Published Application No. 2009-0149389, and U.S. Published Application No2010-0158968, each of which is incorporated herein by reference in its entirety.

According to another embodiment, the functional equivalent of the polypeptide MMI-0100 of amino acid sequence YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11), and the second polypeptide is of amino acid sequence KALARQLGVAA (SEQ ID NO: 2).

According to another embodiment, the first polypeptide is a polypeptide of amino acid sequence WLRRIKAWLRRIKA (SEQ ID NO: 12).

According to another embodiment, first polypeptide is a polypeptide of amino acid sequence WLRRIKA (SEQ ID NO: 13).

According to another embodiment, the first polypeptide is a polypeptide of amino acid sequence YGRKKRRQRRR (SEQ ID NO: 14).

According to another embodiment, the first polypeptide is a polypeptide of amino acid sequence WLRRIKAWLRRI (SEQ ID NO: 15).

According to another embodiment, the first polypeptide is a polypeptide of amino acid sequence FAKLAARLYR (SEQ ID NO: 16).

According to another embodiment, the first polypeptide is a polypeptide of amino acid sequence KAFAKLAARLYR (SEQ ID NO: 17).

According to another embodiment, the first polypeptide is a polypeptide of amino acid sequence HRRIKAWLKKI (SEQ ID NO: 18).

Therapeutic Amount/Dose

The therapeutic agents in the compositions are delivered in therapeutically effective amounts. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. The effective amount for any particular application may vary depending on such factors as the disease or condition being treated, the particular therapeutic agent(s) being administered, the size of the subject, or the severity of the disease or condition. One of ordinary skill in the art may determine empirically the effective amount of a particular therapeutic agent(s) without necessitating undue experimentation. It generally is preferred that a maximum dose be used, that is, the highest safe dose according to some medical judgment. The terms “dose” and “dosage” are used interchangeably herein.

For any compound described herein the therapeutically effective amount may be initially determined from preliminary in vitro studies and/or animal models. A therapeutically effective dose also may be determined from human data for therapeutic agent(s), which have been tested in humans and for compounds which are known to exhibit similar pharmacological activities, such as other related active agents. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

According to some embodiments, the MK2 polypeptide inhibitor of the present invention is administered by dermal injection via an injection apparatus soaked with the pharmaceutical composition. According to some such embodiments, the injection apparatus is a suture. According to some embodiments, the MK2 polypeptide inhibitor of the present invention is administered by dermal injection via the soaked injection apparatus at a dose ranging from 50 ng/100 μl/linear centimeter of wound margin to 1000 ng/100 μl/linear centimeter of wound margin. According to some other embodiments, the therapeutic dose of the MK2 polypeptide inhibitor for dermal injection via the soaked injection apparatus is from 50 ng/100 μl/linear centimeter of wound margin to 100 ng/100 μl/linear centimeter of wound margin. According to some other embodiments, the therapeutic dose of the MK2 polypeptide inhibitor for dermal injection via the soaked injection apparatus is from 100 ng/100 μl/linear centimeter of wound margin to 150 ng/100 μl/linear centimeter of wound margin. According to some other embodiments, the therapeutic dose of the MK2 polypeptide inhibitor for dermal injection via the soaked injection apparatus is from 150 ng/100 μl/linear centimeter of wound margin to 200 ng/100 μl/linear centimeter of wound margin. According to some other embodiments, the therapeutic dose of the MK2 polypeptide inhibitor for dermal injection via the soaked injection apparatus is from 200 ng/100 μl/linear centimeter of wound margin to 250 ng/100 μl/linear centimeter of wound margin. According to some other embodiments, the therapeutic dose of the MK2 polypeptide inhibitor for dermal injection via the soaked injection apparatus is from 250 ng/100 μl/linear centimeter of wound margin to 300 ng/100 μl/linear centimeter of wound margin. According to some other embodiments, the therapeutic dose of the MK2 polypeptide inhibitor for dermal injection via the soaked injection apparatus is from 300 ng/100 μl/linear centimeter of wound margin to 350 ng/100 μl/linear centimeter of wound margin. According to some other embodiments, the therapeutic dose of the MK2 polypeptide inhibitor for dermal injection via the soaked injection apparatus is from 350 ng/100 μl/linear centimeter of wound margin to 400 ng/100 μl/linear centimeter of wound margin. According to some other embodiments, the therapeutic dose of the MK2 polypeptide inhibitor for dermal injection via the soaked injection apparatus is from 400 ng/100 μl/linear centimeter of wound margin to 450 ng/100 μl/linear centimeter of wound margin. According to some other embodiments, the therapeutic dose of the polypeptide inhibitor for dermal injection via the soaked injection apparatus is from 450 ng/100 μl/linear centimeter of wound margin to 500 ng/100 μl/linear centimeter of wound margin. According to some other embodiments, the therapeutic dose of the MK2 polypeptide inhibitor for dermal injection via the soaked injection apparatus is from 500 ng/100 μl/linear centimeter of wound margin to 550 ng/100 μl/linear centimeter of wound margin. According to some other embodiments, the therapeutic dose of the MK2 polypeptide inhibitor for dermal injection via the soaked injection apparatus is from 550 ng/100 μl/linear centimeter of wound margin to 600 ng/100 μl/linear centimeter of wound margin. According to some other embodiments, the therapeutic dose of the MK2 polypeptide inhibitor for dermal injection via the soaked injection apparatus is from 600 ng/100 μl/linear centimeter of wound margin to 650 ng/100 μl/linear centimeter of wound margin. According to some other embodiments, the therapeutic dose of the MK2 polypeptide inhibitor for dermal injection via the soaked injection apparatus is from 650 ng/100 μl/linear centimeter of wound margin to 700 ng/100 μl/linear centimeter of wound margin. According to some other embodiments, the therapeutic dose of the MK2 polypeptide inhibitor for dermal injection via the soaked injection apparatus is from 700 ng/100 μl/linear centimeter of wound margin to 750 ng/100 μl/linear centimeter of wound margin. According to some other embodiments, the therapeutic dose of the MK2 polypeptide inhibitor for dermal injection via the soaked injection apparatus is from 750 ng/100 μl/linear centimeter of wound margin to 800 ng/100 μl/linear centimeter of wound margin. According to some other embodiments, the therapeutic dose of the MK2 polypeptide inhibitor for dermal injection via the soaked injection apparatus is from 800 ng/100 μl/linear centimeter of wound margin to 850 ng/100 μl/linear centimeter of wound margin. According to some other embodiments, the therapeutic dose of the MK2 polypeptide inhibitor for dermal injection via the soaked injection apparatus is from 850 ng/100 μl/linear centimeter of wound margin to 900 ng/100 μl/linear centimeter of wound margin. According to some other embodiments, the therapeutic dose of the MK2 polypeptide inhibitor for dermal injection via the soaked injection apparatus is from 900 ng/100 μl/linear centimeter of wound margin to 950 ng/100 μl/linear centimeter of wound margin. According to some other embodiments, the therapeutic dose of the MK2 polypeptide inhibitor for dermal injection via the soaked injection apparatus is from 950 ng/100 μl/linear centimeter of wound margin to 1000 ng/100 μl/linear centimeter of wound margin.

According to some embodiments, the therapeutic dose of the MK2 polypeptide inhibitor is delivered once at the time of wound closure and then 24 hr after wound closure.

According to some embodiments, the therapeutic dose of the MK2 polypeptide inhibitor is delivered multiple times per day. According to some other embodiments, the therapeutic dose of the MK2 polypeptide inhibitor is delivered multiple times per day coincident with dressing changes. According to some other embodiments, the therapeutic dose of the MK2 polypeptide inhibitor is delivered daily. According to some other embodiments, the therapeutic dose of the MK2 polypeptide inhibitor is delivered daily coincident with dressing changes. According to some other embodiments, the therapeutic dose of the MK2 polypeptide inhibitor is delivered every other day. According to some other embodiments, the therapeutic dose of the MK2 polypeptide inhibitor is delivered every other day coincident with dressing changes.

According to some embodiments, the therapeutic dose of the MK2 polypeptide inhibitor is delivered by dermal injection via an injection apparatus soaked with the pharmaceutical composition once at the time of wound closure and then 24 hr after wound closure. According to some such embodiments, the injection apparatus is a suture.

According to some embodiments, the MK2 polypeptide inhibitor of the present invention is administered by dermal injection via an injection apparatus soaked with the pharmaceutical composition. According to some such embodiments, the injection apparatus is a suture. According to some other embodiments, the therapeutic dose of the MK2 polypeptide inhibitor is delivered by dermal injection via the soaked injection apparatus at multiple times per day. According to some other embodiments, the therapeutic dose of the MK2 polypeptide inhibitor is delivered by dermal injection via the soaked injection apparatus at multiple times per day coincident with dressing changes. According of some other embodiments, the therapeutic dose of the MK2 polypeptide inhibitor is delivered by dermal injection via the soaked injection apparatus daily. According to some other embodiments, the therapeutic dose of the MK2 polypeptide inhibitor is delivered by dermal injection via the soaked injection apparatus daily coincident with dressing changes. According to some other embodiments, the therapeutic dose of the MK2 polypeptide inhibitor is delivered by dermal injection via the soaked injection apparatus every other day. According to some other embodiments, the therapeutic dose of the MK2 polypeptide inhibitor is delivered by dermal injection via the soaked injection apparatus every other day coincident with dressing changes.

According to some embodiments, the MK2 polypeptide inhibitor is administered intraperitoneally at a dose ranging from 70 μg/kg to 80 μg/kg at one time daily, weekly, or on alternative days or weeks. According to some other embodiments, the MK2 polypeptide inhibitor is administered intraperitoneally at a dose of 75 μg/kg at one time daily, weekly, or on alternative days or weeks.

According to some embodiments, the therapeutic amount of the MK2 polypeptide inhibitor of the pharmaceutical composition is of an amount from about 0.000001 mg/kg body weight to about 100 mg/kg body weight. According to another embodiment, the therapeutic amount of the MK2 polypeptide inhibitor of the pharmaceutical composition is of an amount from about 0.00001 mg/kg body weight to about 100 mg/kg body weight. According to another embodiment, the therapeutic amount of the MK2 polypeptide inhibitor of the pharmaceutical composition is of an amount from about 0.0001 mg/kg body weight to about 100 mg/kg body weight. According to another embodiment, the therapeutic amount of the MK2 polypeptide inhibitor of the pharmaceutical composition is of an amount from about 0.001 mg/kg body weight to about 10 mg/kg body weight. According to another embodiment, the therapeutic amount of the MK2 polypeptide inhibitor of the pharmaceutical composition is of an amount from about 0.01 mg/kg body weight to about 10 mg/kg body weight. According to another embodiment, the therapeutic amount of the MK2 polypeptide inhibitor of the pharmaceutical composition is of an amount from about 0.1 mg/kg (or 100 μg/kg) body weight to about 10 mg/kg body weight. According to another embodiment, the therapeutic amount of the MK2 polypeptide inhibitor of the pharmaceutical composition is of an amount from about 1 mg/kg body weight to about 10 mg/kg body weight. According to another embodiment, the therapeutic amount of the MK2 polypeptide inhibitor of the pharmaceutical composition is of an amount from about 10 mg/kg body weight to about 100 mg/kg body weight. According to another embodiment, the therapeutic amount of the MK2 polypeptide inhibitor of the pharmaceutical composition is of an amount from about 2 mg/kg body weight to about 10 mg/kg body weight. According to another embodiment, the therapeutic amount of the MK2 polypeptide inhibitor of the pharmaceutical composition is of an amount from about 3 mg/kg body weight to about 10 mg/kg body weight. According to another embodiment, the therapeutic amount of the MK2 polypeptide inhibitor of the pharmaceutical composition is of an amount from about 4 mg/kg body weight to about 10 mg/kg body weight. According to another embodiment, the therapeutic amount of the MK2 polypeptide inhibitor of the pharmaceutical composition is of an amount from about 5 mg/kg body weight to about 10 mg/kg body weight. According to another embodiment, the therapeutic amount of the MK2 polypeptide inhibitor of the pharmaceutical composition is of an amount from about 60 mg/kg body weight to about 100 mg/kg body weight. According to another embodiment, the therapeutic amount of the MK2 polypeptide inhibitor of the pharmaceutical composition is of an amount from about 70 mg/kg body weight to about 100 mg/kg body weight. According to another embodiment, the therapeutic amount of the MK2 polypeptide inhibitor of the pharmaceutical composition is of an amount from about 80 mg/kg body weight to about 100 mg/kg body weight. According to another embodiment, the therapeutic amount of the MK2 polypeptide inhibitor of the pharmaceutical composition is of an amount from about 90 mg/kg body weight to about 100 mg/kg body weight. According to another embodiment, the therapeutic amount of the MK2 polypeptide inhibitor of the pharmaceutical composition is of an amount from about 0.000001 mg/kg body weight to about 90 mg/kg body weight. According to another embodiment, the therapeutic amount of the MK2 polypeptide inhibitor of the pharmaceutical composition is of an amount from about 0.000001 mg/kg body weight to about 80 mg/kg body weight. According to another embodiment, the therapeutic amount of the MK2 polypeptide inhibitor of the pharmaceutical composition is of an amount from about 0.000001 mg/kg body weight to about 70 mg/kg body weight. According to another embodiment, the therapeutic amount of the MK2 polypeptide inhibitor of the pharmaceutical composition is of an amount from about 0.000001 mg/kg body weight to about 60 mg/kg body weight. According to another embodiment, the therapeutic amount of the MK2 polypeptide inhibitor of the pharmaceutical composition is of an amount from about 0.000001 mg/kg body weight to about 50 mg/kg body weight. According to another embodiment, the therapeutic amount of the MK2 polypeptide inhibitor of the pharmaceutical composition is of an amount from about 0.000001 mg/kg body weight to about 40 mg/kg body weight. According to another embodiment, the therapeutic amount of the MK2 polypeptide inhibitor is of an amount from about 0.000001 mg/kg body weight to about 30 mg/kg body weight. According to another embodiment, the therapeutic amount of the MK2 polypeptide inhibitor of the pharmaceutical composition is of an amount from about 0.000001 mg/kg body weight to about 20 mg/kg body weight. According to another embodiment, the therapeutic amount of the MK2 polypeptide inhibitor of the pharmaceutical composition is of an amount from about 0.000001 mg/kg body weight to about 10 mg/kg body weight. According to another embodiment, the therapeutic amount of the MK2 polypeptide inhibitor of the pharmaceutical composition is of an amount from about 0.000001 mg/kg body weight to about 1 mg/kg body weight. According to another embodiment, the therapeutic amount of the MK2 polypeptide inhibitor of the pharmaceutical composition is of an amount from about 0.000001 mg/kg body weight to about 0.1 mg/kg body weight. According to another embodiment, the therapeutic amount of the MK2 polypeptide inhibitor of the pharmaceutical composition is of an amount from about 0.000001 mg/kg body weight to about 0.1 mg/kg body weight. According to another embodiment, the therapeutic amount of the MK2 polypeptide inhibitor of the pharmaceutical composition is of an amount from about 0.000001 mg/kg body weight to about 0.01 mg/kg body weight. According to another embodiment, the therapeutic amount of the MK2 polypeptide inhibitor of the pharmaceutical composition is of an amount from about 0.000001 mg/kg body weight to about 0.001 mg/kg body weight. According to another embodiment, the therapeutic amount of the MK2 polypeptide inhibitor of the pharmaceutical composition is of an amount from about 0.000001 mg/kg body weight to about 0.0001 mg/kg body weight. According to another embodiment, the therapeutic amount of the MK2 polypeptide inhibitor of the pharmaceutical composition is of an amount from about 0.000001 mg/kg body weight to about 0.00001 mg/kg body weight.

According to some other embodiments, the therapeutic dose of the MK2 polypeptide inhibitor of the pharmaceutical composition ranges from 1 μg/kg/day to 25 μg/kg/day. According to some other embodiments, the therapeutic dose of the MK2 polypeptide inhibitor of the pharmaceutical composition ranges from 1 μg/kg/day to 2 μg/kg/day. According to some other embodiments, the therapeutic dose of the MK2 polypeptide inhibitor of the pharmaceutical composition ranges from 2 μg/kg/day to 3 μg/kg/day. According to some other embodiments, the therapeutic dose of the MK2 polypeptide inhibitor of the pharmaceutical composition ranges from 3 μg/kg/day to 4 μg/kg/day. According to some other embodiments, the therapeutic dose of the MK2 polypeptide inhibitor of the pharmaceutical ranges from 4 μg/kg/day to 5 μg/kg/day. According to some other embodiments, the therapeutic dose of the MK2 polypeptide inhibitor of the pharmaceutical composition ranges from 5 μg/kg/day to 6 μg/kg/day. According to some other embodiments, the therapeutic dose of the MK2 polypeptide inhibitor of the pharmaceutical composition ranges from 6 μg/kg/day to 7 μg/kg/day. According to some other embodiments, the therapeutic dose of the MK2 polypeptide inhibitor of the pharmaceutical composition ranges from 7 μg/kg/day to 8 μg/kg/day. According to some other embodiments, the therapeutic dose of the MK2 polypeptide inhibitor of the pharmaceutical composition ranges from 8 μg/kg/day to 9 μg/kg/day. According to some other embodiments, the therapeutic dose of the MK2 polypeptide inhibitor of the pharmaceutical composition ranges from 9 μg/kg/day to 10 μg/kg/day. According to some other embodiments, the therapeutic dose of the MK2 polypeptide inhibitor of the pharmaceutical composition ranges from 1 μg/kg/day to 5 μg/kg/day. According to some other embodiments, the therapeutic dose of the MK2 polypeptide inhibitor of the pharmaceutical composition ranges from 5 μg/kg/day to 10 μg/kg/day. According to some other embodiments, the therapeutic dose of the MK2 polypeptide inhibitor of the pharmaceutical composition ranges from 10 μg/kg/day to 15 μg/kg/day. According to some other embodiments, the therapeutic dose of the MK2 polypeptide inhibitor of the pharmaceutical composition ranges from 15 μg/kg/day to 20 μg/kg/day. According to some other embodiments, the therapeutic dose of the MK2 polypeptide inhibitor of the pharmaceutical composition ranges from 25 μg/kg/day to 30 μg/kg/day. According to some other embodiments, the therapeutic dose of the MK2 polypeptide inhibitor of the pharmaceutical composition ranges from 30 μg/kg/day to 35 μg/kg/day. According to some other embodiments, the therapeutic dose of the MK2 polypeptide inhibitor of the pharmaceutical composition ranges from 35 μg/kg/day to 40 μg/kg/day. According to some other embodiments, the therapeutic dose of the MK2 polypeptide inhibitor of the pharmaceutical composition ranges from 40 μg/kg/day to 45 μg/kg/day. According to some other embodiments, the therapeutic dose of the MK2 polypeptide inhibitor of the pharmaceutical composition ranges from 45 μg/kg/day to 50 μg/kg/day. According to some other embodiments, the therapeutic dose of the MK2 polypeptide inhibitor of the pharmaceutical composition ranges from 50 μg/kg/day to 55 μg/kg/day. According to some other embodiments, the therapeutic dose of the MK2 polypeptide inhibitor of the pharmaceutical composition ranges from 55 μg/kg/day to 60 μg/kg/day. According to some other embodiments, the therapeutic dose of the MK2 polypeptide inhibitor of the pharmaceutical composition ranges from 60 μg/kg/day to 65 μg/kg/day. According to some other embodiments, the therapeutic dose of the MK2 polypeptide inhibitor of the pharmaceutical composition ranges from 65 μg/kg/day to 70 μg/kg/day. According to some other embodiments, the therapeutic dose of the MK2 polypeptide inhibitor of the pharmaceutical composition ranges from 70 μg/kg/day to 75 μg/kg/day. According to some other embodiments, the therapeutic dose of the MK2 polypeptide inhibitor of the pharmaceutical composition ranges from 80 μg/kg/day to 85 μg/kg/day. According to some other embodiments, the therapeutic dose of the MK2 polypeptide inhibitor of the pharmaceutical composition ranges from 85 μg/kg/day to 90 μg/kg/day. According to some other embodiments, the therapeutic dose of the MK2 polypeptide inhibitor of the pharmaceutical composition ranges from 90 μg/kg/day to 95 μg/kg/day. According to some other embodiments, the therapeutic dose of the MK2 polypeptide inhibitor of the pharmaceutical composition ranges from 95 μg/kg/day to 100 μg/kg/day.

According to another embodiment, the therapeutic dose of the MK2 polypeptide inhibitor of the pharmaceutical composition is 1 μg/kg/day. According to another embodiment, the therapeutic dose of the MK2 polypeptide inhibitor of the pharmaceutical composition is 2 μg/kg/day. According to another embodiment, the therapeutic dose of the MK2 polypeptide inhibitor of the pharmaceutical composition is 5 μg/kg/day. According to another embodiment, the therapeutic dose of the MK2 polypeptide inhibitor of the pharmaceutical composition is 10 μg/kg/day.

The formulations of therapeutic agent(s) may be administered in pharmaceutically acceptable solutions, which may routinely contain pharmaceutically acceptable concentrations of salt, buffering agents, preservatives, compatible carriers, adjuvants, and optionally other therapeutic ingredients.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein, the contents of which are incorporated herein by reference, are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### IC₅₀ and Specificity of MMI-0100 (YARAAARQARAKALARQLGVAA; SEQ ID NO: 1)

IC₅₀ (half maximal inhibitory concentrations) value for the MK2 polypeptide inhibitors of the described invention, MMI-0100 of amino acid sequence YARAAARQARAKALARQLGVAA (SEQ ID NO: 1), MMI-0200 of amino acid sequence YARAAARQARAKALNRQLGVA (SEQ ID NO: 19), MMI-0300 (FAKLAARLYRKALARQLGVAA; SEQ ID NO: 3); MMI-0400 (KAFAKLAARLYRKALARQLGVAA; SEQ ID NO: 4); and MMI-0500 (HRRIKAWLKKIKALARQLGVAA; SEQ ID NO: 7) was determined using Millipore's IC₅₀ Profiler Express service. This quantitative assay measures how much of an inhibitor is needed to inhibit 50% of a given biological process or component of a process (i.e., an enzyme, cell, or cell receptor) [IC₅₀]. Specifically, in these assays, a positively charged substrate is phosphorylated with a radiolabeled phosphate group from an ATP if the kinase is not inhibited by an inhibitor peptide. The positively charged substrate then is attracted to a negatively charged filter membrane, quantified with a scintillation counter, and compared to a 100% activity control.

ATP concentrations within 15 μM of the apparent Km for ATP were chosen since an ATP concentration near the Kₘ may allow for the kinases to have the same relative amount of phosphorylation activity.

In addition, the MK2 polypeptide inhibitors of the present invention may differentially inhibit a selective group of kinases that are involved in cutaneous wound healing or scarring in vivo.

Therefore, in order to identify potential intracellular kinases that are affected by the MK2 polypeptide inhibitors of the present invention, the specificity of MMI-0100 (YARAAARQARAKALARQLGVAA; SEQ ID NO: 1) and of its functional equivalents MMI-0200 of amino acid sequence YARAAARQARAKALNRQLGVA (SEQ ID NO: 19), MMI-0300 (FAKLAARLYRKALARQLGVAA; SEQ ID NO: 3); MMI-0400 (KAFAKLAARLYRKALARQLGVAA; SEQ ID NO: 4); and MMI-0500 (HRRIKAWLKKIKALARQLGVAA; SEQ ID NO: 7) were assessed by examining activities of all 266 human kinases available for testing in the Millipore kinase profiling service (See Table 5). For analysis, the kinases that were inhibited more than 65% by MMI-0100 (YARAAARQARAKALARQLGVAA; SEQ ID NO: 1); MMI-0200 (YARAAARQARAKALNRQLGVA; SEQ ID NO: 19); MMI-0300 (FAKLAARLYRKALARQLGVAA; SEQ ID NO: 3); MMI-0400 (KAFAKLAARLYRKALARQLGVAA; SEQ ID NO: 4); and MMI-0500 (HRRIKAWLKKIKALARQLGVAA; SEQ ID NO: 7) were determined.

As shown in Table 5, at 100 μM, MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) inhibited a specific group of kinases and showed very limited off-target kinase inhibition. More specifically, MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) inhibited in vitro more than 65% of the kinase activities of Mitogen-Activated Protein Kinase-Activated Protein Kinase 2 (MK2), Mitogen-Activated Protein Kinase-Activated Protein Kinase 3 (MK3), Calcium/Calmodulin-Dependent Protein Kinase I (CaMKI, serine/threonine-specific protein kinase), and BDNF/NT-3 growth factors receptor (TrkB, tyrosine kinase).

TABLE 5
Kinase Profiling Assay

| | MMI-0100 | MMI-0200 | MMI-0300 | MMI-0400 | MMI-0500 |
|---|---|---|---|---|---|
| | (SEQ ID NO: 1) | (SEQ ID NO: 19) | (SEQ ID NO: 3) | (SEQ ID NO: 4) | (SEQ ID NO: 7) |
| | (100 μM) | (100 μM) | (100 μM) | (100 μM) | (100 μM) |
| Abl(h) | 136 | 107 | 69 | 84 | 16 |
| Abl(H8396P)(h) | 130 | 121 | 101 | 105 | 51 |
| Abl(M351T)(h) | 128 | 119 | 90 | 121 | 61 |
| Abl(Q252H)(h) | 105 | 107 | 82 | 98 | 40 |
| Abl(T315I)(h) | 98 | 108 | 97 | 105 | 16 |
| Abl(Y253F)(h) | 104 | 102 | 86 | 78 | 29 |
| ACK1(h) | 106 | 97 | 104 | 95 | 64 |
| ALK(h) | 118 | 95 | 19 | 16 | 12 |
| ALK4(h) | 124 | 152 | 140 | 130 | 81 |
| Arg(h) | 89 | 82 | 72 | 84 | 22 |
| AMPKα1(h) | 107 | 108 | 71 | 87 | 35 |
| AMPKα2(h) | 121 | 88 | 54 | 58 | 9 |
| ARK5(h) | 108 | 93 | 78 | 69 | 20 |
| ASK1(h) | 100 | 101 | 80 | 69 | −4 |
| Aurora-A(h) | 120 | 107 | 92 | 119 | 110 |
| Aurora-B(h) | 94 | 166 | 128 | 150 | 5 |
| Axl(h) | 81 | 99 | 52 | 41 | 12 |
| Bmx(h) | 62 | 76 | N/D | 26 | 45 |
| BRK(h) | 70 | 127 | 35 | 18 | 41 |
| BrSK1(h) | 100 | 93 | 67 | 76 | 72 |
| BrSK2(h) | 129 | 102 | 83 | 86 | 84 |
| BTK(h) | 112 | 100 | 102 | 94 | 18 |
| BTK(R28H)(h) | 91 | 104 | 74 | 24 | 10 |
| CaMKI(h) | 13 | 21 | 1 | 0 | −1 |
| CaMKIIβ(h) | 58 | 53 | 2 | 11 | 3 |
| CaMKIIγ(h) | 106 | 94 | 5 | 3 | 3 |
| CaMKIδ(h) | 59 | 47 | 10 | 17 | 0 |
| CaMKIIδ(h) | 89 | 2 | 1 | 2 | 1 |
| CaMKIV(h) | 87 | 71 | 17 | 18 | -1 |
| CDK1/cyclinB(h) | 96 | 115 | 73 | 74 | 57 |
| CDK2/cyclinA(h) | 97 | 114 | 86 | 92 | 87 |
| CDK2/cyclinE(h) | 106 | 112 | 94 | 83 | 19 |
| CDK3/cyclinE(h) | 106 | 104 | 94 | 92 | 8 |
| CDK5/p25(h) | 114 | 97 | 89 | 192 | 66 |
| CDK5/p35(h) | 94 | 92 | 79 | 76 | 59 |
| CDK6/cyclinD3(h) | 103 | 100 | 86 | 85 | 23 |
| CDK7/cyclinH/ | 89 | 67 | 65 | 47 | 15 |
| MAT1(h) | | | | | |
| CDK9/cyclin T1(h) | 228 | 103 | 91 | 235 | 6 |
| CHK1(h) | 97 | 115 | 91 | 87 | 65 |
| CHK2(h) | 104 | 105 | 66 | 54 | 13 |
| CHK2(I157T)(h) | 97 | 85 | 43 | 41 | 3 |
| CHK2(R145W)(h) | 97 | 81 | 33 | 31 | 3 |
| CK1γ1(h) | 110 | 98 | 111 | 116 | 109 |
| CK1γ2(h) | 119 | 104 | 123 | 114 | 119 |
| CK1γ3(h) | 105 | 96 | 125 | 115 | 114 |
| CK1δ(h) | 115 | 92 | 92 | 93 | 78 |
| CK2(h) | 90 | 83 | 90 | 101 | 93 |
| CK2α2(h) | 104 | 88 | 105 | 96 | 103 |
| CLK2(h) | 88 | 97 | 103 | 116 | 116 |
| CLK3(h) | 108 | 76 | 61 | 84 | 76 |
| cKit(h) | 95 | 110 | 53 | 43 | 45 |
| cKit(D816V)(h) | 117 | 118 | 60 | 35 | 30 |
| cKit(D816H)(h) | 79 | 106 | 126 | 143 | 194 |
| cKit(V560G)(h) | 94 | 115 | 102 | 124 | 198 |
| cKit(V654A)(h) | 69 | 113 | 134 | 150 | 223 |
| CSK(h) | 70 | 33 | 49 | 16 | 2 |
| c-RAF(h) | 97 | 115 | 107 | 102 | 19 |
| cSRC(h) | 70 | 32 | 26 | 14 | 30 |
| DAPK1(h) | 97 | 113 | 46 | 36 | 0 |
| DAPK2(h) | 41 | 92 | 32 | 16 | 3 |
| DCAMKL2(h) | 146 | 131 | 81 | 70 | 56 |
| DDR2(h) | 105 | 104 | 94 | 95 | 79 |
| DMPK(h) | 60 | 66 | 59 | 54 | 12 |
| DRAK1(h) | 47 | 34 | 14 | 14 | 8 |
| DYRK2(h) | 99 | 142 | 155 | 195 | 127 |
| eEF-2K(h) | 113 | 136 | 91 | 43 | 43 |
| EGFR(h) | 95 | 83 | 21 | 16 | −1 |
| EGFR(L858R)(h) | 76 | 120 | N/D | 52 | 26 |
| EGFR(L861Q)(h) | 53 | 74 | 25 | 22 | 15 |
| EGFR(T790M)(h) | 106 | 113 | 100 | 106 | 70 |
| EGFR(T790M, | 93 | 108 | 85 | 78 | 53 |
| L858R)(h) | | | | | |
| EphA1(h) | 114 | 136 | 73 | 61 | 40 |
| EphA2(h) | 58 | 95 | 31 | 17 | N/D |
| EphA3(h) | 107 | 117 | 6 | 12 | 33 |
| EphA4(h) | 110 | 127 | 88 | 65 | 48 |
| EphA5(h) | 110 | 123 | 18 | 24 | 42 |
| EphA7(h) | 193 | 220 | 159 | 222 | 189 |
| EphA8(h) | 181 | 133 | 93 | 146 | 337 |
| EphB2(h) | 68 | 128 | 18 | 22 | 70 |
| EphB1(h) | 99 | 95 | 44 | 58 | 37 |
| EphB3(h) | 109 | 128 | 62 | 47 | 79 |
| EphB4(h) | 62 | 131 | 44 | 28 | 38 |
| ErbB4(h) | 73 | 82 | 40 | 0 | 2 |
| FAK(h) | 98 | 110 | 111 | 96 | 94 |
| Fer(h) | 117 | 101 | 130 | 108 | 196 |
| Few(h) | 44 | 74 | 20 | 16 | 23 |
| FGFR1(h) | 120 | 97 | 55 | 59 | 18 |
| FGFR1(V561M)(h) | 108 | 72 | 74 | 74 | 113 |
| FGFR2(h) | 49 | 73 | 14 | 18 | 12 |
| FGFR2(N549H)(h) | 95 | 104 | 116 | 112 | 105 |
| FGFR3(h) | 73 | 208 | 102 | 0 | 10 |
| FGFR4(h) | 67 | 75 | 28 | 19 | 3 |
| Fgr(h) | 54 | 71 | 60 | 47 | 109 |
| Flt1(h) | 109 | 96 | 69 | 48 | 27 |
| Flt3(D835Y)(h) | 120 | 115 | 80 | 71 | 65 |
| Flt3(h) | 104 | 99 | 84 | 18 | 17 |
| Flt4(h) | 135 | 105 | 83 | 89 | 73 |
| Fms(h) | 89 | 92 | 45 | 37 | 14 |
| Fms(Y969C)(h) | 126 | 88 | 72 | 91 | N/D |
| Fyn(h) | 71 | 75 | 74 | 54 | 83 |
| GCK(h) | 98 | 99 | 70 | 66 | 30 |
| GRK5(h) | 117 | 135 | 136 | 131 | 116 |
| GRK6(h) | 131 | 132 | 147 | 141 | 174 |
| GRK7(h) | 111 | 124 | 122 | 100 | 93 |
| GSK3α(h) | 183 | 119 | 157 | 164 | 175 |
| 6SK3β(h) | 113 | 132 | 205 | 202 | 238 |
| Haspin(h) | 127 | 71 | 48 | 36 | 25 |
| Hck(h) | 354 | 107 | 72 | 72 | 78 |
| Hck(h) activated | 58 | 100 | 82 | 81 | 67 |
| HIPK1(h) | 94 | 115 | 74 | 91 | 47 |
| HIPK2(h) | 98 | 102 | 73 | 90 | 38 |
| HIPK3(h) | 105 | 105 | 93 | 105 | 85 |
| IGF-1R(h) | 102 | 49 | 119 | 90 | 117 |
| IGF-1R(h), | 126 | 94 | 80 | 77 | 45 |
| activated | | | | | |
| IKKα(h) | 108 | 104 | 93 | 87 | 50 |
| IKKβ(h) | 105 | 109 | 84 | 84 | 71 |
| IR(h) | 112 | 90 | 96 | 85 | 95 |
| IR(h), activated | 127 | 105 | 79 | 59 | 90 |
| IRR(h) | 85 | 69 | 8 | 8 | 10 |
| IRAK1(h) | 97 | 101 | 95 | 93 | 5 |
| IRAK4(h) | 100 | 110 | 59 | 59 | 3 |
| Itk(h) | 99 | 98 | 77 | 63 | 7 |
| JAK2(h) | 89 | 131 | 133 | 119 | 49 |
| JAK3(h) | 150 | 117 | 121 | 122 | 95 |
| JNK1α1(h) | 91 | 106 | 97 | 98 | 109 |
| JNK2α2(h) | 114 | 109 | 98 | 96 | 81 |
| JNK3(h) | 104 | 90 | 89 | 70 | 171 |
| KDR(h) | 100 | 110 | 101 | 94 | 15 |
| Lck(h) | 346 | 113 | −2 | 228 | 359 |
| Lck(h) activated | 106 | 90 | 243 | 216 | 76 |
| LIMK1(h) | 103 | 109 | 88 | 92 | 87 |
| LKB1(h) | 111 | 99 | 101 | 89 | 51 |
| LOK(h) | 37 | 67 | 37 | 18 | 7 |
| Lyn(h) | 113 | 98 | 69 | 3 | 31 |
| MAPK1(h) | 108 | 97 | 107 | 100 | 102 |
| MAPK2(h) | 98 | 105 | 98 | 93 | 60 |
| MAPKAP-K2(h) | 19 | 35 | 5 | 5 | 9 |
| MAPKAP-K3(h) | 27 | 39 | 3 | 7 | 9 |
| MEK1(h) | 86 | 116 | 77 | 77 | 21 |
| MARK1(h) | 109 | 102 | 132 | 120 | 110 |
| MELK(h) | 74 | 59 | 16 | 17 | 0 |
| Mer(h) | 47 | 90 | 52 | 50 | 17 |
| Met(h) | 104 | 71 | 65 | 62 | 27 |
| Met(D1246H)(h) | 99 | 139 | 125 | 68 | 150 |
| Met(D1246N)(h) | 114 | 149 | 82 | 31 | 90 |
| Met(M1268T)(h) | 114 | 143 | 255 | 265 | 239 |
| Met(Y1248C)(h) | 77 | 141 | 84 | 36 | 73 |
| Met(Y1248D)(h) | 87 | 118 | 102 | 31 | 218 |
| Met(Y1248H)(h) | 88 | 153 | 117 | 63 | 126 |
| MINK(h) | 96 | 103 | 48 | 52 | 5 |
| MKK6(h) | 74 | 98 | 48 | 44 | 18 |
| MKK7β(h) | 137 | 117 | 100 | 94 | 102 |
| MLCK(h) | 85 | 103 | 2 | 1 | 0 |
| MLK1(h) | 77 | 84 | 40 | 33 | 43 |
| Mnk2(h) | 94 | 106 | 89 | 86 | 6 |
| MRCKα(h) | 98 | 103 | 104 | 97 | 5 |
| MRCKβ(h) | 103 | 102 | 83 | 71 | −10 |
| MSK1(h) | 52 | 50 | 32 | 28 | 8 |
| MSK2(h) | 105 | 88 | 56 | 52 | 14 |
| MSSK1(h) | 82 | 100 | 77 | 75 | 22 |
| MST1(h) | 85 | 72 | 14 | 6 | 3 |
| MST2(h) | 98 | 104 | 19 | 11 | 2 |
| MST3(h) | 104 | 95 | 45 | 36 | 4 |
| mTOR(h) | 102 | 110 | 91 | 93 | 135 |
| mTOR/FKBP12(h) | 117 | 118 | 145 | 125 | 140 |
| MuSK(h) | 85 | 106 | 93 | 93 | 27 |
| NEK2(h) | 102 | 97 | 78 | 61 | 0 |
| NEK3(h) | 100 | 100 | 92 | 85 | 20 |
| NEK6(h) | 109 | 98 | 82 | 85 | 49 |
| NEK7(h) | 97 | 96 | 84 | 87 | 89 |
| NEK11(h) | 102 | 95 | 53 | 33 | 2 |
| NLK(h) | 100 | 106 | 87 | 90 | 19 |
| p70S6K(h) | 89 | 84 | 35 | 33 | 3 |
| PAK2(h) | 71 | 69 | 65 | 59 | 44 |
| PAK4(h) | 92 | 98 | 94 | 89 | 86 |
| PAK3(h) | N/D | 50 | 140 | 121 | 102 |
| PAK5(h) | 97 | 100 | 110 | 117 | 125 |
| PAK6(h) | 121 | 105 | 104 | 100 | 107 |
| PAR-1Bα(h) | 62 | 110 | 113 | 109 | 97 |
| PASK(h) | 81 | 60 | 29 | 28 | 9 |
| PDGFRα(h) | 104 | 108 | 65 | 40 | 40 |
| PDGFRα(D842V)(h) | 103 | 107 | 114 | 118 | 170 |
| PDGFRα(V561D)(h) | 58 | 106 | 82 | 100 | 146 |
| PDGFRβ(h) | 116 | 137 | 81 | 53 | 40 |
| PDK1(h) | 144 | 143 | 135 | 159 | 178 |
| PhKγ2(h) | 62 | 86 | 46 | 38 | 16 |
| Pim-1(h) | 44 | 18 | 8 | 7 | 0 |
| Pim-2(h) | 117 | 74 | 76 | 92 | 46 |
| Pim-3(h) | 98 | 94 | 80 | 80 | 37 |
| PKA(h) | 138 | 110 | 119 | 119 | 118 |
| PKBα(h) | 140 | 110 | 57 | 67 | 30 |
| PKBβ(h) | 284 | 250 | 84 | 98 | 21 |
| PKBγ(h) | 105 | 103 | 20 | 41 | 20 |
| PKCα(h) | 94 | 100 | 89 | 86 | 3 |
| PKCβI(h) | 88 | 98 | 78 | 78 | 1 |
| PKCβII(h) | 102 | 100 | 82 | 75 | 3 |
| PKCγ(h) | 94 | 101 | 89 | 79 | 6 |
| PKGδ(h) | 100 | 101 | 101 | 90 | 61 |
| PKCε(h) | 102 | 98 | 79 | 59 | 23 |
| PKGη(h) | 105 | 101 | 103 | 98 | 45 |
| PKCι(h) | 110 | 97 | 68 | 46 | 7 |
| PKCμ(h) | 79 | 73 | 22 | 14 | 10 |
| PKCθ(h) | 102 | 101 | 88 | 76 | 62 |
| PKCζ(h) | 82 | 98 | 81 | 75 | 7 |
| PKD2(h) | 84 | 78 | 33 | 25 | 10 |
| PKG1α(h) | 82 | 70 | 64 | 58 | 25 |
| PKG1β(h) | 71 | 57 | 50 | 53 | 24 |
| Plk1(h) | 109 | 128 | 115 | 119 | 104 |
| Plk3(h) | 107 | 107 | 127 | 129 | 122 |
| PRAK(h) | 159 | 115 | 128 | 118 | 95 |
| PRK2(h) | 72 | 74 | 33 | 27 | 7 |
| PrKX(h) | 84 | 112 | 61 | 76 | 57 |
| PTK5(h) | 135 | 108 | 132 | 129 | 96 |
| Pyk2 (h) | 113 | 127 | 47 | 34 | 46 |
| Ret(h) | 108 | 96 | 140 | 145 | 174 |
| Ret(V804L)(h) | 113 | 100 | 79 | 73 | 20 |
| Ret(V804M)(h) | 92 | 105 | 95 | 87 | 36 |
| RIPK2(h) | 92 | 98 | 97 | 98 | 30 |
| ROCK-I(h) | 99 | 117 | 79 | 73 | 17 |
| ROCK-II(h) | 102 | 85 | 74 | 77 | 2 |
| Ron(h) | 117 | 120 | 93 | 79 | 46 |
| Ros(h) | 107 | 86 | 95 | 99 | 150 |
| Rse(h) | 109 | 88 | 88 | 89 | 63 |
| Rsk1(h) | 86 | 102 | 46 | 54 | 34 |
| Rsk2(h) | 65 | 101 | 51 | 38 | 14 |
| Rsk3(h) | 76 | 109 | 76 | 71 | 23 |
| Rsk4(h) | 99 | 125 | 90 | 91 | 29 |
| SAPK2a(h) | 110 | 107 | 90 | 85 | 52 |
| SAPK2a(T106M)(h) | 101 | 100 | 97 | 99 | 32 |
| SAPK2b(h) | 99 | 95 | 81 | 82 | 42 |
| SAPK3(h) | 106 | 97 | 84 | 79 | 24 |
| SAPK4(h) | 98 | 106 | 96 | 91 | 48 |
| SGK(h) | 128 | 115 | 48 | 54 | 2 |
| SGK2(h) | 103 | 119 | 56 | 98 | −1 |
| SGK3(h) | 95 | 58 | 10 | 8 | −3 |
| SIK(h) | 113 | 102 | 66 | 68 | 40 |
| Snk(h) | 94 | 109 | 114 | 131 | 122 |
| Src(1-530)(h) | 95 | 75 | 23 | 19 | 21 |
| Src(T341M)(h) | 98 | 56 | 70 | 76 | 59 |
| SRPK1(h) | 69 | 93 | 90 | 96 | 80 |
| SRPK2(h) | 92 | 100 | 106 | 97 | 80 |
| STK33(h) | 99 | 98 | 45 | 52 | 16 |
| Syk(h) | 45 | 36 | 24 | 9 | 5 |
| TAK1(h) | 116 | 124 | 122 | 177 | N/D |
| TAO1(h) | 99 | 105 | 82 | 73 | 24 |
| TAO2(h) | 95 | 93 | 70 | 74 | 15 |
| TAO3(h) | 45 | 102 | 77 | 67 | 12 |
| TBK1(h) | 106 | 98 | 37 | 39 | 16 |
| Tec(h)activated | 100 | 77 | 56 | 29 | 33 |
| Tie2(h) | 28 | 53 | 26 | 21 | 22 |
| Tie2(R849W)(h) | 102 | 89 | 117 | 108 | 106 |
| Tie2(Y897S)(h) | 99 | 85 | 83 | 87 | 80 |
| TLK2(h) | 113 | 129 | 114 | 151 | 133 |
| TrkA(h) | 74 | N/D | 25 | 17 | 24 |
| TrkB(h) | 4 | 7 | 5 | 8 | 12 |
| TSSK1(h) | 99 | 98 | 79 | 79 | 46 |
| TSSK2(h) | 107 | 91 | 98 | 94 | 92 |
| Txk(h) | 87 | 98 | 48 | 37 | 10 |
| ULK2(h) | 123 | 132 | 122 | 131 | 124 |
| ULK3(h) | 142 | 164 | 167 | 147 | 177 |
| WNK2(h) | 95 | 94 | 64 | 54 | 8 |
| WNK3(h) | 100 | 97 | 77 | 74 | 9 |
| VRK2(h) | 112 | 109 | 161 | 185 | 169 |
| Yes(h) | 49 | 93 | 67 | 14 | N/D |
| ZAP-70(h) | 79 | 58 | 75 | 33 | 1 |
| ZIPK(h) | 80 | 67 | 28 | 13 | 1 |

N/D: % activity could not be determined as the duplicates.
MMI-0100: YARAAARQARAKALARQLGVAA (SEQ ID NO: 1)
MMI-0200: YARAAARQARAKALNRQLGVA (SEQ ID NO: 19)
MMI-0300: FAKLAARLYRKALARQLGVAA (SEQ ID NO: 3)
MMI-0400: KAFAKLAARLYRKALARQLGVAA (SEQ ID NO: 4)
MMI-0500: HRRIKAWLKKIKALARQLGVAA (SEQ ID NO: 7)

The MK2 polypeptide inhibitor MMI-0100 (SEQ ID NO: 1) selectively inhibits kinases selected from the group Mitogen-Activated Protein Kinase-Activated Protein Kinase 2 (MK2), Mitogen-Activated Protein Kinase-Activated Protein Kinase 3 (MK3), Calcium/Calmodulin-Dependent Protein Kinase I (CaMKI, serine/threonine-specific protein kinase), and BDNF/NT-3 growth factors receptor (TrkB, tyrosine kinase). Table 6 lists IC50 values of MMI-100 (SEQ ID NO: 1) with selected kinases and off-target proteins (including off-target kinases and off-target receptors). As shown in Table 6, the IC₅₀ values for the MK2 polypeptide inhibitor MMI-0100 (SEQ ID NO: 1) with kinases selected from the group Mitogen-Activated Protein Kinase-Activated Protein Kinase 2 (MK2), Mitogen-Activated Protein Kinase-Activated Protein Kinase 3 (MK3), Calcium/Calmodulin-Dependent Protein Kinase I (CaMKI, serine/threonine-specific protein kinase), and BDNF/NT-3 growth factors receptor (TrkB, tyrosine kinase) range between 4.6 μM and 15.8 μM. In contrast, the IC₅₀ values for the MK2 polypeptide inhibitor MMI-0100 (SEQ ID NO: 1) with off-target proteins range between 34.1 μM and 180.6 μM.

Table 6. IC₅₀ Values of MMI-0100 (SEQ ID NO: 1) with Selected Kinases and Off-Target Proteins (Including Off-Target Kinases and Off-Target Receptors)

TABLE 6
IC₅₀ values of MMI-0100 (SEQ ID NO: 1) with Selected
Kinases and Off-target Proteins

| Selected Kinases and Off-target Proteins | IC₅₀ (μM) |
|---|---|
| TrkB (h) | 4.6 |
| CaMKI (h) | 11.7 |
| MAPKAP-K2 (h) | 12.1 |
| MAPKAP-K3 (h) | 15.8 |
| DMPK (h) | 34.1 |
| CaMK1δ (h) | 45.8 |
| LOK (h) | 47.6 |
| Pim-1 (h) | 71.7 |
| DRAK1 (h) | 78.3 |
| MSK1 (h) | 102.5 |
| Yes (h) | 113.7 |
| EGFR (L861Q) (h) | 127.8 |
| Fgr (h) | 140.9 |
| FGFR2 (h) | 172.0 |
| Fes(h) | 173.7 |
| EphA2 (h) | 180.6 |

### Example 2

### Evaluation of the Off-Target Effects of MK2 Polypeptide Inhibitors

Off-target effects of MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) were evaluated using Cerep binding assays, which operate according to the competition assay principle, with each assay utilizing a radiolabelled ligand and a source of receptor. Primary screening was performed at 1-10 μM in duplicate, followed by IC₅₀ determination when the test polypeptide inhibitor displayed more than 50% inhibition of control value. Each binding assay was performed in 6-control wells with or without vehicle plus an 8-point dose-response of the relevant reference compound. The MK2 polypeptide inhibitor MMI-0100 (SEQ ID NO: 1) showed less than 30% inhibition of off-target proteins Angiotensin 2, bombesin, melanocortin 4, neurokinin 2, neuropeptide Y, serotonin 2A, vasoactive intestinal peptide, and small conductance calcium-activated K+ channel.

Table 7 lists the % activity of off-target receptors at 100 μM of MK2 polypeptide inhibitor MMI-0100 (SEQ ID NO: 1).

TABLE 7
% Activity of Selected Off-target Receptors with 100 μM MMI-
0100 (SEQ ID NO: 1)
Table 7. % Activity of Selected Off-target Receptors with
100 μM MMI-0100 (SEQ ID NO: 1)

| OFF-TARGET RECEPTOR | IC₅₀ (μM) |
|---|---|
| Angiotensin 2 | 94% |
| Bombesin | 85% |
| Melanocortin 4 | 81% |
| Neurokinin 2 | 85% |
| Neuropeptide Y | 91% |
| Serotonin 2A | 86% |
| Vasoactive intestinal peptide | 82% |
| Small conductance calcium activated K+ | 73% |
| channel | |

### Example 3

### Evaluation of the Efficacy of MK2 Polypeptide Inhibitors Using a Mouse Model of Hypertrophic Scarring Induced by Mechanical Loading

Accumulating evidence has suggested (1) that mechanical stress applied to a healing wound is sufficient to produce hypertrophic scars in mice; and (2) that the murine model of hypertrophic scarring reproduces all the features of human hypertrophic scarring by augmenting the mechanical stresses on murine wounds to achieve levels normally experienced by human wounds (Arabi, S. et al., FASEB J. 21, 3250-3261 (2007), the entire content of which is incorporated by reference herein).

The murine model of hypertrophic scarring is useful to investigate the pathophysiology of human hypertrophic scarring. For example, like human hypertrophic scars, murine scars are raised and show epidermal thickening with adnexal structures and hair follicles absent in the dermis. In the murine model of hypertrophic scars, collagen is arranged in compact sheets parallel to the direction of applied mechanical load with fibroblasts aligning with the collagen fibers. Like human hypertrophic scars, mechanically induced scars also show a significant mast cell infiltrate; hypervascularity, a classic feature of hypertrophic scars, collagen whorls, which is often seen in mature human hypertrophic scars; and cellular hyperplasia.

The efficacy of MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) of the described invention in treating hypertrophic scarring was examined using this mouse model.

As a pilot study, the efficacy of MK2 polypeptide inhibitor MMI-0100 (SEQ ID NO: 1) was tested. Number of animals in each group was n=5 mice per control group (receiving phosphate buffered saline (PBS)) and in the experimental group (receiving MMI-0100 (YARAAARQARAKALARQLGVAA (SEQ ID NO: 1)). One of three doses (3 μM, 30 μM and 100 μM) of MMI-100 (from 57.75 m/kg to 75 μg/kg (about 2 μg per mouse) was administered to the experimental group mice by intraperitoneal injection daily from day 0 to day 14. 2 cm incisional wounds were made on the dorsal skin of mice at day 0. At day 4, sutures were removed, and a skin distraction device was placed on the back of mouse skin along the wound in each mouse. Because one mouse in the MMI-0100-treated group (MMI-0100-#5) did not heal the wound at day 4, the sutures of MMI-0100-#5 were kept in place for additional 4 days for that mouse.

Dorsal skins were distracted laterally along both sides of the wound edge by a distraction rate of 1 mm/day from day 4 to day 7, and then 2 mm/day from day 8 to day 14. For mouse MMI-0100-#5, sutures were removed at day 7, and skin distraction was loaded from day 8 to day 14. At day 14, skin wounds were photographed digitally, and scar areas were measured by Image J software. Tissue was harvested for RNA extraction, Florescence-Activated Cell Sorting (FACS) analysis, and histological examination.

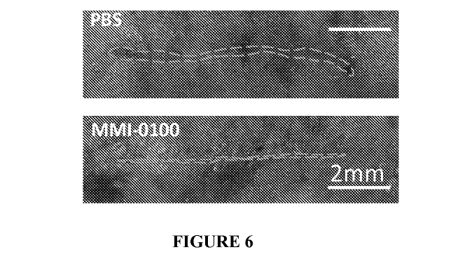

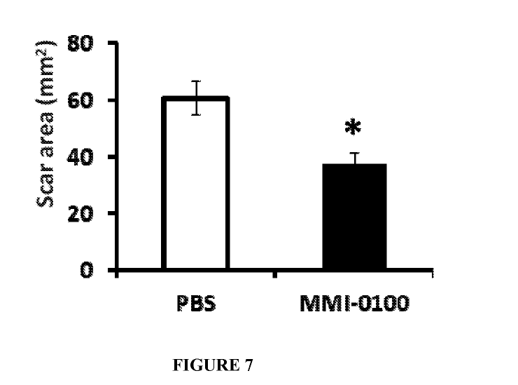

FIG. 6 shows a gross comparison of scar appearance in a PBS treated mouse with scar appearance in a MMI-0100-treated mouse. Scale bar=2 mm. FIG. 7 shows scar area comparison between control and MMI-100 (SEQ ID NO: 1)-treated mice. Scar edges were identified and scar areas were quantified using Image J software. Scar areas were compared using student's t-test; n=5; *, P=0.011. As shown in FIGS. 6 and 7, and Table 8, mice treated with MMI-0100 (YARAAARQARAKALARQLGVAA (SEQ ID NO: 1)) exhibited less inflammation and less scar formation, as compared with PBS-treated control mice.

TABLE 8
Measurements of Scar Areas in PBS-treated
and MMI-0100 (YARAAARQARAKALARQLGVAA
(SEQ ID NO: 1))-treated Groups

| | area (pixels) | area (mm²) | avg | Sem |
|---|---|---|---|---|
| PBS-1 | 858.703 | 50.29 | 60.75 | 5.830097 |
| PBS-2 | 899.292 | 52.67 | | |
| PBS-3 | 867.02 | 50.78 | | |
| PBS-4 | 1276.194 | 74.75 | | |
| PBS-5 | 1284.57 | 75.24 | | |
| MMI-0100 #1 | 710.227 | 41.60 | 37.53 | 3.914183 |
| MMI-0100 - #2 | 517.002 | 30.28 | | |
| MMI-0100 - #3 | 731.195 | 42.83 | | |
| MMI-0100 - #4 | 796.618 | 46.66 | | |
| MMI-0100 - #5 | 449.178 | 26.31 | | |

Wounds were harvested and subjected to FACS analysis in order to determine immune cell infiltration into the control and experimental hypertrophic scars. Tissue sections are digested with collagenase, sorted, and labeled with rat monoclonal antibodies against, e.g., macrophages (11b+/F4/80+), mast cells (CD117+), and T lymphocytes (CD4+/CD8+). According to other embodiments, other cell types may be assayed; and intercellular phosphoflow may be used in elucidating upstream and downstream kinase activity, in particular upstream p38 MAPK and MK2 activation/phosphorylation, as well as that of downstream substrates, such as HSP27/HSP25, TPP, amongst others.

Analysis of gene expression in skin samples was performed using qPCR with primers against chemokine (C-C motif) ligand 2 (ccl2), chemokine (C-C motif) receptor 2 (ccr2), collagen type 1α2 (col1α2), collagen type 3α1 (col 3α1), EGF-like module-containing mucin-like hormone receptor-like 1 (emr1), Interleukin-6 (IL-6), and Transforming Growth Factor-beta1 (TGF-β1).

Digital photographs were taken on the day of surgery and every two days thereafter. Gross scar appearance was assessed by tracing the wound margin and calculating the hypertrophic scar area.

### Histological Comparison of Scars

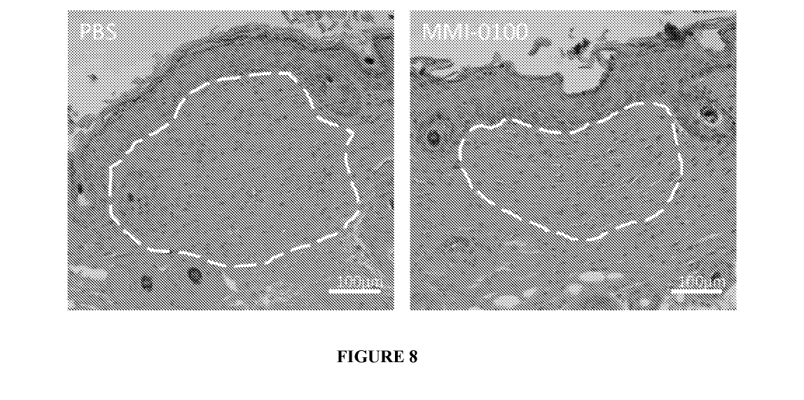

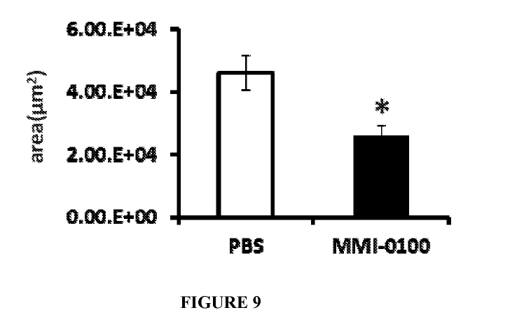

The scar areas of mice treated with MMI-0100 (YARAAARQARAKALARQLGVAA (SEQ ID NO: 1)) and PBS controls were analyzed histologically. Scar samples from each mouse were fixed in 10% formalin and embedded in paraffin. Sections were made and stained with hematoxylin and eosin (H&E) to assess scar tissue deposition. FIG. 8 shows a histological comparison of scars in MMI-0100 (SEQ ID NO: 1)-treated mice with PBS treated group. Scar areas were outlined, and areas were measured by using Image J software. Scale bar=100 μm. FIG. 9 shows a comparison of the cross sectional area of scars treated with a control (PBS) or MMI-0100 (SEQ ID NO: 1). n=5, *, P=0.015. As shown in FIG. 8, in contrast to PBS-treated mice, which exhibited whorls of collagen fibers in their scar areas, collagen fibers of MMI-0100 (YARAAARQARAKALARQLGVAA (SEQ ID NO: 1)-treated mice were aligned parallel to the skin surface in the scar areas. In some MMI-0100 (YARAAARQARAKALARQLGVAA (SEQ ID NO: 1))-treated mice, scar regions were difficult to locate microscopically. In addition, mice treated with MMI-0100 (YARAAARQARAKALARQLGVAA (SEQ ID NO: 1)) also exhibited significantly reduced scar areas histologically compared to control mice treated with PBS (FIG. 9; n=5, *, p=0.015).

### MMI-0100 (YARAAARQARAKALARQLGVAA (SEQ ID NO: 1)) Treatment Decreases Transcription of Scar-Related Genes

The effect of MMI-0100 (YARAAARQARAKALARQLGVAA (SEQ ID NO: 1)) treatment on the transcription of scar-related genes was examined. To this end, 2×2 mm² skin in scar areas were collected for RNA extraction. Because RNA yields were low in two skin samples from the PBS-treated group (PBS-1 and PBS-5) and in one skin sample from the MMI-0100 (YARAAARQARAKALARQLGVAA (SEQ ID NO: 1))-treated group (MMI-0100-#1), qRT-PCR tests were conducted using the RNAs extracted from three mice in the PBS-treated group and from four mice in the MMI-0100-treated group (Table 9).

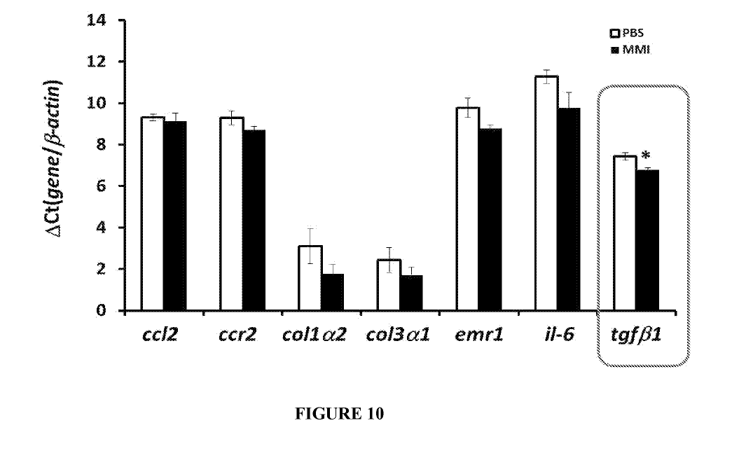

FIG. 10 shows quantitative reverse transcription polymerase chain reaction (qRT-PCR) comparison of scar related gene transcripts. PBS group, n=3; MMI group, n=4, *, P=0.016. As shown in FIG. 10, mice treated with MMI-0100 (YARAAARQARAKALARQLGVAA (SEQ ID NO: 1)) showed significant modulation in the expression of TGF-β1 in the scar area, compared to control mice (*, p=0.015), and consistent modulation of other scar-related genes (although not reaching significance p<0.05 in this small experiment). While the level of TGF-β1 itself was not measured, in other embodiments, protein levels could be measured. Without being limited by theory, this suggests that MMI-0100 (YARAAARQARAKALARQLGVAA (SEQ ID NO: 1)) can modulate TGF-β1 mRNA levels.

TABLE 9
RNA Quantifications for Skin Samples from
PBS and MMI-0100-Treated Groups

| | RNA | RNA vol | Ran | dNTPs | Water |
|---|---|---|---|---|---|
| samples | conc. (ng/ul) | (μl) | hxmrs (μl) | (μl) | (μl) |
| PBS-1 | 43 | 8 | 1 | 1 | 0 |
| PBS-2 | 168 | 6.0 | 1 | 1 | 2.0 |
| PBS-3 | 97 | 10.3 | 1 | 1 | 0 |
| PBS-4 | 122 | 8.2 | 1 | 1 | 0 |
| PBS-5 | 58 | 8 | 1 | 1 | 0 |
| MMI-0100 #1 | 31 | 8 | 1 | 1 | 0 |
| MMI-0100 - #2 | 114 | 8.8 | 1 | 1 | 0 |
| MMI-0100 - #3 | 156 | 6.4 | 1 | 1 | 1.6 |
| MMI-0100 - #4 | 258 | 3.9 | 1 | 1 | 4.1 |
| MMI-0100 - #5 | 144 | 6.9 | 1 | 1 | 1.1 |

### MMI-0100 (YARAAARQARAKALARQLGVAA (SEQ ID NO: 1)) Reduces Immune Cell Infiltration and Significantly Reduces Lymphocytes Infiltration

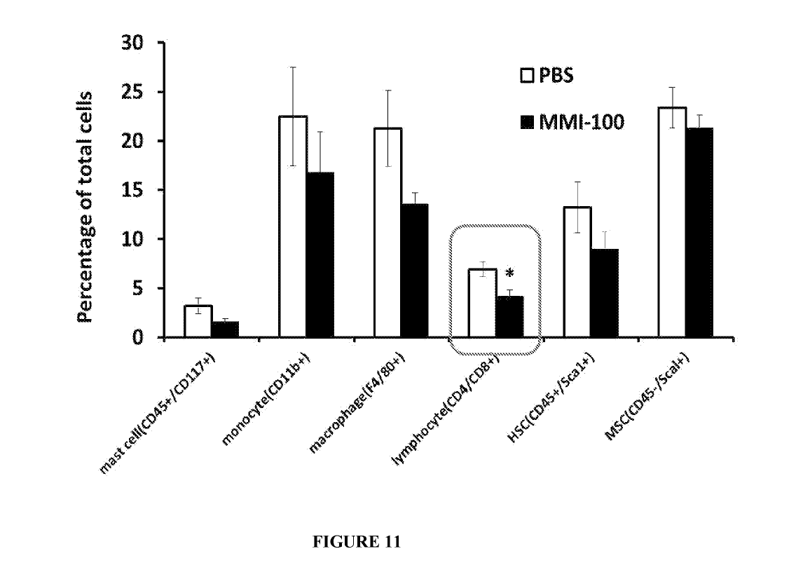

In order to further examine the effect MMI-0100 (YARAAARQARAKALARQLGVAA (SEQ ID NO: 1)) on infiltration of immunomodulatory and immune progenitor cells into wound tissues, skin samples (5×30 mm² rectangular) of scar area were collected from PBS-treated or MMI-0100 (YARAAARQARAKALARQLGVAA (SEQ ID NO: 1))-treated mice. Skin samples were digested with liberase, and the released cells were labeled with fluorescently tagged antibodies against CD45, scar-1, F4/80, CD11b, cKIT, and CD4/8. FIG. 11 shows a comparison of the cell populations in scar areas from MMI-0100 and PBS-treated mice. PBS group, n=5*, P=0.02. As shown in Table 10 and FIG. 11, MMI-0100 (YARAAARQARAKALARQLGVAA (SEQ ID NO: 1)) treatment significantly decreased infiltration of CD4+/CD8+ lymphocytes into the scar area (n=5, *, P=0.02), and consistently decreased mast/dendritic cells, macrophages, and other monocyte-related and progenitor stem cells, although it did not achieve statistical significance a p<0.05 in this small experiment.

TABLE 10
Population Analysis of Immunomodulatory
and Stem Cells in the Scar Region.

| | | | | MMI- | |
|---|---|---|---|---|---|
| | | MMI- | PBS | 0100 | |
| | PBS | 0100 | (sem) | (sem) | P value |
| mast cell | 3.196 | 1.5786 | 0.78 | 0.32 | 0.09 |
| (CD45+/CD117+) | | | | | |
| monocyte (CD11b+) | 22.48 | 16.744 | 5.03 | 4.20 | 0.41 |
| macrophage (F4/80+) | 21.28 | 13.592 | 3.86 | 1.10 | 0.09 |
| lymphocyte (CD4/CD8+) | 6.918 | 4.188 | 0.76 | 0.62 | 0.02 |
| Hematopoietic Stem Cell | 13.212 | 8.972 | 2.60 | 1.82 | 0.22 |
| (HSC) (CD45+/Sca1+) | | | | | |
| Mesenchymal Stem Cell | 23.36 | 21.32 | 2.09 | 1.32 | 0.43 |
| (MSC) (CD45−/Scal+) | | | | | |

Utilizing a set of the PBS control mice, one dose of MMI-0100 (50 μg/kg) is administered daily by intraperitoneal injection, beginning at day 42 (i.e., 6 weeks after control incision) and continuing through day 70. A parallel set of PBS control mice are injected control (PBS) for comparison. Dorsal skin is distracted laterally along both sides of the wound edge by the distraction rate of 1 mm/day from day 46 to day 49, and then 2 mm/day from day 50 to day 70. At day 70, skin wounds are photographed digitally, and scar areas are measured by Image J software. Tissues are harvested for RNA extraction, Florescence-Activated Cell Sorting (FACS) analysis, and histological examination, as described above.

### Example 3

### Evaluation of the Efficacy of Intraperitoneal Administration of MK2 Polypeptide Inhibitor MMI-0300 (SEQ ID NO: 3) Using Mouse Model of Hypertrophic Scarring Induced by Mechanical Loading

The efficacy of intraperitoneal administration of MK2 polypeptide inhibitor MMI-0300 (SEQ ID NO: 3) was tested, using n=4 mice per control group (receiving phosphate buffered saline (PBS)) and n=6 mice per experimental group (receiving MMI-0300 (FAKLAARLYRKALARQLGVAA (SEQ ID NO: 3)). The experimental group was administered 50 μg/kg MK2 polypeptide inhibitor MMI-0300 (SEQ ID NO: 3) daily by intraperitoneal injection daily beginning at day 0 through day 14. One mouse in the control group died.

Dorsal skin was distracted laterally along both sides of the wound edge by the distraction rate of 1 mm/day from day 4 to day 7, and then 2 mm/day from day 8 to day 14. At day 14, skin wounds were photographed digitally, and scar areas were measured by Image J software. Tissues were harvested for RNA extraction, Fluorescence-Activated Cell Sorting (FACS) analysis, and histological examination, as described above.

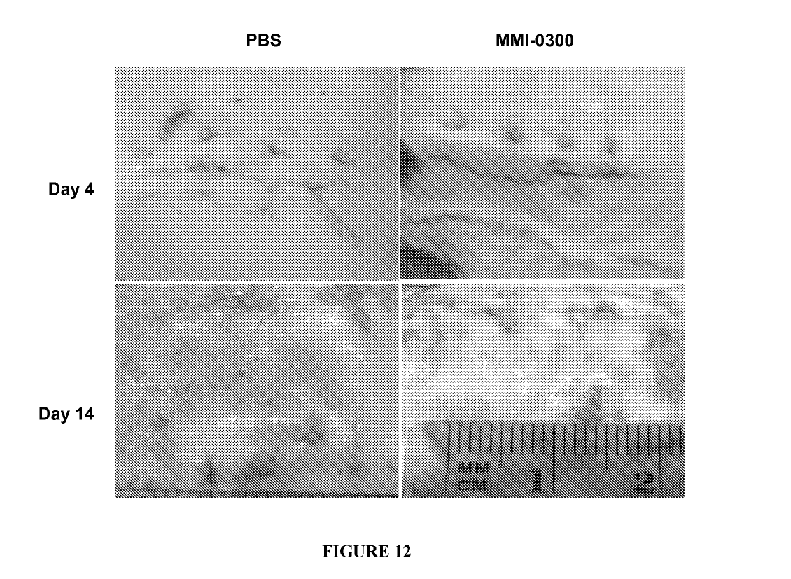

FIG. 12 shows a gross comparison of scar appearance in a PBS treated mouse and a MMI-0300 (SEQ ID NO: 3)-treated mouse on day 4 and day 14 (scale bar=2.2 cm). As shown in FIG. 12, a mouse treated with MMI-0300 (FAKLAARLYRKALARQLGVAA (SEQ ID NO: 3) exhibited less inflammation and less scar formation, as compared with a control mouse treated with PBS.

### Histological Comparison of Scars

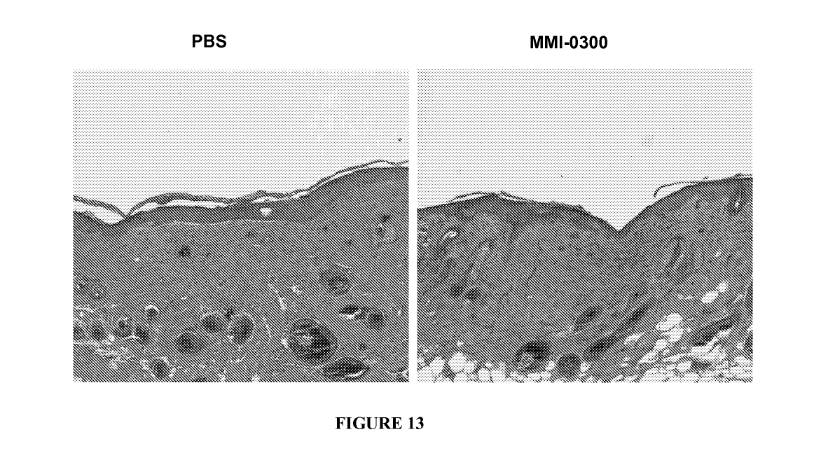

The scar areas of mice treated with MMI-0300 (FAKLAARLYRKALARQLGVAA (SEQ ID NO: 3)) and PBS were analyzed histologically. For histological analysis, scar samples from each mouse were fixed in 10% formalin and embedded with paraffin. Sections were made and stained with hematoxylin and eosin (H&E) to assess scar tissue deposition. FIG. 13 shows a histological comparison of scars in a MMI-0300 (SEQ ID NO: 3)-treated mouse with a mouse in the PBS treated group. Scar areas were outlined, and areas were measured by using Image J software.

### Transcription of Scar-Related Genes

The effect of MMI-0300 (FAKLAARLYRKALARQLGVAA (SEQ ID NO: 3)) treatment on the transcription of scar-related genes was examined. To this end, 2×2 mm² skin in scar areas were collected for RNA extraction. qRT-PCR tests were conducted using the RNAs extracted from the mice in the PBS-treated and the MMI-0300-treated groups.

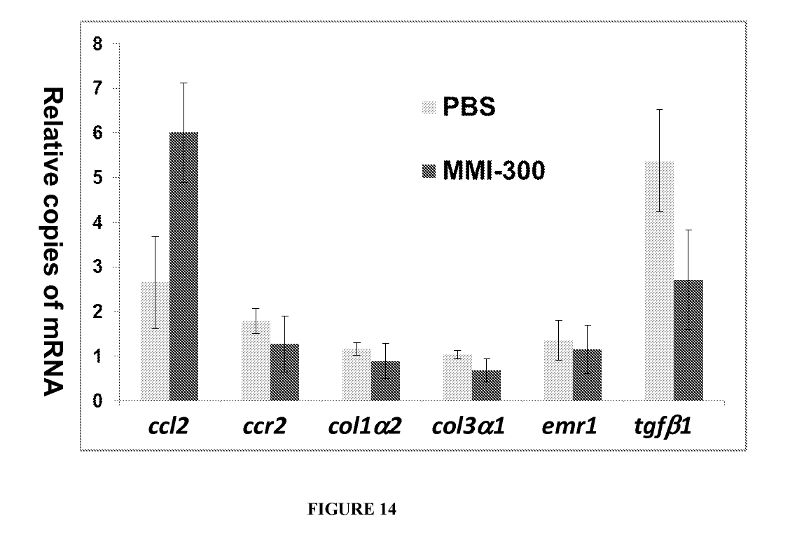

FIG. 14 shows a quantitative reverse transcription polymerase chain reaction (qRT-PCR) comparison of scar related gene transcripts. PBS group, n=3; MMI group, n=6. As shown in FIG. 14, mice treated with MMI-0300 (FAKLAARLYRKALARQLGVAA (SEQ ID NO: 3)) showed considerable modulation in the expression of ccl2 and TGF-β1 in the scar area, compared to control mice.

### FACS Analysis of Scar Area

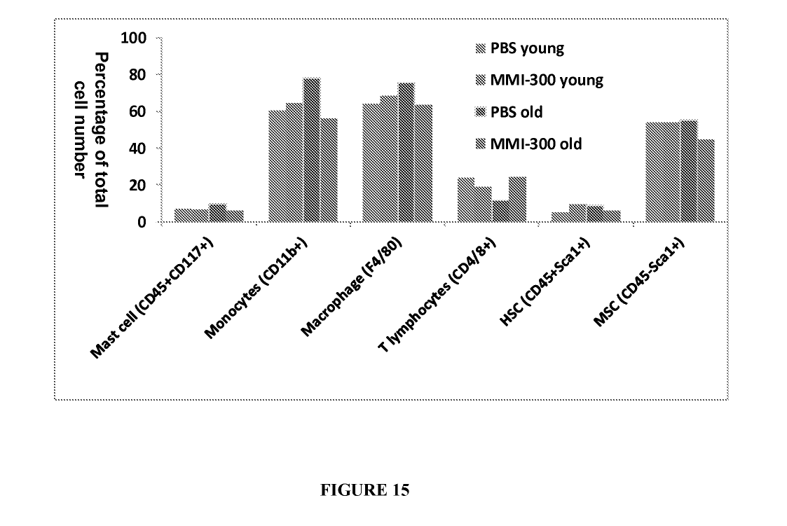

In order to further examine the effect of MMI-0300 (FAKLAARLYRKALARQLGVAA (SEQ ID NO: 3)) on infiltration of immunomodulatory and immune progenitor cells into wound tissues, skin samples (5×30 mm² rectangular) of scar area were collected from PBS-treated or MMI-0300 (FAKLAARLYRKALARQLGVAA (SEQ ID NO: 3))-treated young and old mice. Skin samples were digested with liberase, and the released cells were labeled with fluorescently tagged antibodies against CD45, scar-1, F4/80, CD11b, cKIT, and CD4/8. FIG. 15 shows comparison of cell population in scar areas with the young and old PBS-treated and the MMI-0100 (SEQ ID NO: 1)-treated groups. As shown in FIG. 15, MMI-0300 (FAKLAARLYRKALARQLGVAA (SEQ ID NO: 3)) treatment showed decreases in macrophages, and other monocyte-related and progenitor stem cells.

### Example 4

### Evaluation of the Efficacy of Topical Administration of MK2 Polypeptide Inhibitors Using Mouse Model of Hypertrophic Scarring Induced by Mechanical Loading

The efficacy of topical administration of MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) of the described invention are evaluated using the mouse model for hypertrophic scarring induced by mechanical loading described in Example 2. One of three doses (3 μM, 30 μM and 100 μM) for each MK2 polypeptide inhibitor is administered daily by topical administration beginning at day 0 through day 14 using a moist dressing soaked with the MK2 polypeptide inhibitor containing solution.

Dorsal skin is distracted laterally along both sides of the wound edge by the distraction rate of 1 mm/day from day 4 to day 7, and then 2 mm/day from day 8 to day 14. At day 14, skin wounds are photographed digitally, and scar areas are measured by Image J software. Tissue is harvested for RNA extraction, Fluorescence-Activated Cell Sorting (FACS) analysis, and histological examination, as described above.

### Example 5

### Evaluation of the Efficacy of Local Intradermal Injection of MK2 Polypeptide Inhibitors Using Mouse Model of Hypertrophic Scarring Induced by Mechanical Loading

The efficacy of local intradermal injection of MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) of the described invention is evaluated using the mouse model for hypertrophic scarring induced by mechanical loading described in Example 2. One of three doses (3 μM, 30 μM and 100 μM) of each MK2 polypeptide inhibitor is administered daily by local intradermal injection beginning at day 0 through day 14.

Dorsal skin is distracted laterally along both sides of the wound edge by the distraction rate of 1 mm/day from day 4 to day 7, and then 2 mm/day from day 8 to day 14. At day 14, skin wounds are photographed digitally, and scar areas are measured by Image J software. Tissues are harvested for RNA extraction, Florescence-Activated Cell Sorting (FACS) analysis, and histological examination, as described above.

### Example 6

### Evaluation of the Efficacy of Intraperitoneal of MK2 Polypeptide Inhibitors Using Mouse Model of Hypertrophic Scarring Induced by Mechanical Loading

The efficacy of intraperitoneal injection of MK2 polypeptide inhibitors MMI-0100 (SEQ ID NO: 1), MMI-0200 (SEQ ID NO: 19), MMI-0300 (SEQ ID NO: 3); MMI-0400 (SEQ ID NO: 4); and MMI-0500 (SEQ ID NO: 5) of the described invention is evaluated using the mouse model for hypertrphic scarring induced by mechanical loading described in Example 2. One of three doses (3 μM, 30 μM and 100 μM) of each MK2 polypeptide inhibitor is administered daily by intraperitoneal injection beginning at day 0 through day 14.

Dorsal skin is distracted laterally along both sides of the wound edge by the distraction rate of 1 mm/day from day 4 to day 7, and then 2 mm/day from day 8 to day 14. At day 14, skin wounds are photographed digitally, and scar areas are measured by Image J software. Tissues are harvested for RNA extraction, Florescence-Activated Cell Sorting (FACS) analysis, and histological examination, as described above.

### Example 7

### Fluorescence-Based Quantitative Scratch Wound Healing Assay

The effect of MMI-0100 (SEQ ID NO: 1) or its functional equivalents MMI-0200 (SEQ ID NO: 19), MMI-0300 (SEQ ID NO: 3); MMI-0400 (SEQ ID NO: 4); and MMI-0500 (SEQ ID NO: 5) on wound healing is analyzed by examining migratory phenotypes of human fibroblasts in fluorescence-based quantitative wound healing assay.

The scratch wound healing assay is an infrared fluorescence detection-based real-time assay for sensitive and accurate quantification of cell migration in vitro and utilizes a live cell staining lipophilic tracer, i.e., 1,1′-dioctadecyl-3,3,3′,3′-tetramethyl indotricarbocyanine iodide (DiR), for accurate imaging of wound closure (Menon, M. et al., Cell Motility and the Cytoskeleton 66: 1041-1047, 2009, incorporated herein by reference in its entirety).

In order to examine the effect of MMI-0100 (SEQ ID NO: 1) or its functional equivalents, an appropriate number of human fibroblasts are stained with DiR by incubating a fibroblast cell suspension with DiR at 37° C. for 15-20 minutes. DiR-stained cells are washed twice with PBS to remove any excess DiR and resuspended with complete culture medium. An appropriate number of DiR-stained cells are plated onto a 96-well plate. Once plated cells form a confluent monolayer (e.g., 12-24 hour after plating), scratches are made on prestained confluent cell monolayers using a pipette tip. Then, MMI-0100 (SEQ ID NO: 1) or its functional equivalents, or a control peptide, is applied to each well. The images of wounded cells are scanned at different time intervals using a fluorescent scanner. Images are analyzed using Image J software and the migration index is calculated.

### Example 8

### In Vivo Evaluation of Anti-Scarring Activity of MK2 Polypeptide Inhibitors in Red Duroc Pigs

The anti-scarring potential of the MK2 polypeptide inhibitors MMI-0100 (SEQ ID NO: 1) or its functional equivalents MMI-0200 (SEQ ID NO: 19), MMI-0300 (SEQ ID NO: 3); MMI-0400 (SEQ ID NO: 4); and MMI-0500 (SEQ ID NO: 5) compared to placebo was analyzed using full thickness wounds in Red Duroc pigs. Two doses (30 μM and 300 μM) of the MK2 polypeptide inhibitor MMI-0100 (SEQ ID NO: 1) were evaluated over 70-days, and wounds were assessed for both speed of healing and relative severity of scar formation.

Two female commercially raised Red Duroc pigs (40-50 kg) were fed antibiotic-free feed and given water ad libitum during a 10-day acclimation period, during which a comprehensive veterinary health inspection was performed to ensure health of the animals prior to commencement of the study. The day before surgery (Day 0, the animals were anesthetized with Telazol (Tiletamine/Zolazepam; 5 mg/kg, intramuscular); a small portion of the caudal dorsum was clipped with a #40 Oster clipper blade. A Fentanyl patch, Duragesic (50 ug/hr) was secured to the shaved skin for post-surgical pain management.

On Day 0, the pigs were premedicated by intramuscular injection of atropine (0.05 mg/kg) follow by Telazol (Tiletamine/Zolazepam; 4.4 mg/kg intramuscular) followed by intubation and inhalation of 2 to 5 percent Isoflurane mix with oxygen. The dorsal and lateral thorax of the pig was clipped with a #40 Oster clipper blade and washed with an antimicrobial-free soap. Full-thickness square excisions parallel to the spine (2 cm on each side, 4 columns of 5 excisions, at least 2 cm apart), were made on Day 0 by cutting down to the subcutaneous tissue using a #10 blade scalpel or using a square 2 cm trephine. Complete removal of fat domes is essential to proper scar tissue formation. Epinephrine solution (1:10,000 dilution) was applied on gauze sponges until hemostasis was complete (approximately 10 minutes).

Prior to treating the wounds, a 4″×4″ piece of polyskin was used to to cover the wounds. A tincture of benzoin was used to hold the polyskin and the test or control articles in place. Treatments were performed on days 0, 1 and 4. Pig #1 was treated with MMI-0100 (300 μM) on the left side and PBS on the right side. Pig #2 was treated with MMI-0100 (30 μM) on the left side and with PBS on the right side. The test material and the Control were injected into the wound space using a syringe with 26G needle. All wounds were covered with blue-absorbent pad as secondary dressing and wrapped with a layer of elastic bandage. To relieve post-surgical and biopsy wound pain, a Fentanyl patch was replaced every three days throughout the duration of the study. The dressings were changed on days 1, 4 and 7. On day 11, the dressings were removed.

Wound size measurements were performed using digital calipers on days 0, 1, 4, 7, 11, 13 and 28. For each wound, the calipers were used to measure the distance across the widest part of the wound as well as the narrowest part. Digital photographs were taken prior to and during surgery and prior to each day's measurements. (Photos not shown).

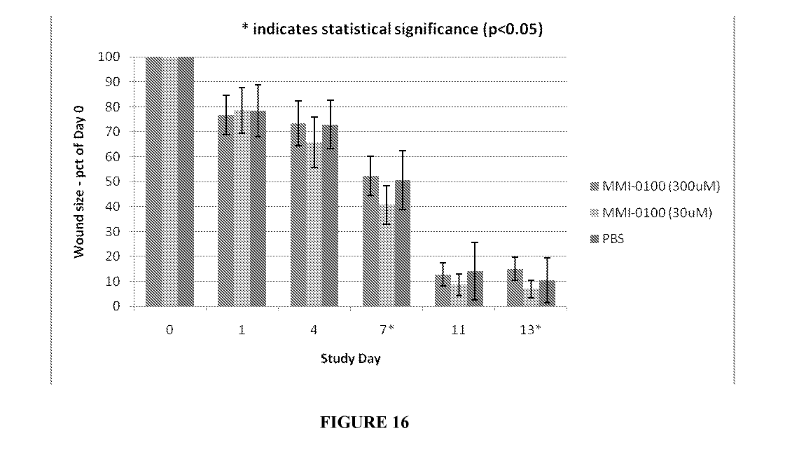

FIG. 16 shows wound size as a percentage (pct) of wound size in Red Duroc pigs at Day 0 at the wound sites treated with MMI-0100 (300 μM) (1^{st} bar), MMI-0100 (30 μM) (2^{nd} bar), and PBS control (3^{rd} bar). Asterisk indicates statistical significance (p<0.05). The clinical assessment of scarring indicated that 30 μM concentration of MMI-0100 performed at parity with the PBS control in terms of visual assessments of scar appearance. In contrast, the 300 μM dosage of MMI-0100 generated scars that were slightly worse in appearance than the other two test/control groups.

On days 56 and 70, the wounds were assessed clinically by visual scoring by a blinded veterinarian. As overall assessment was made using the visual analog scale (VAS) as a vertical mark on a 10-cm line with a score of zero (0) indicating an excellent scar and ten (10) indicating a poor scar. This score (cm) was added to the sum of individual parameter scores to give an overall score for each scar. Besides the VAS score, the scars were also scored by color matching to the surrounding skin (lighter to darker) (perfect match=1; slight mismatch=2; obvious mismatch=3; gross mismatch=4); as matte or shiny (matte=1; shiny=2); by contour (flush with surrounding skin=1; slightly proud/indented=2; hypertrophic=3; keloid=4); as distortion (none=1; miled=2; moderate=3; severe=4); and by texture (normal=1; just palpable=2; firm=3; hard=4). Table 11 shows VAS scores, and clinical measurements of color matching (CM), matte vs. shiny (M/S), contour (C), distortion (D), texture (T) on day 56 and day 70 for wound sites treated with MMI-0100 (at 300 μM), MMI-0100 (at 30 μM) and PBS.

TABLE 11
Clinical (visual) assessments of wound sites
Table 11. Clinical (visual) assessments of wound sites

| | DAY 56 | | | DAY 70 | | |
|---|---|---|---|---|---|---|
| | MMI-0100 | MMI-0100 | | MMI-0100 | MMI-0100 | |
| | (300 μM) | (30 μM) | PBS | (300 μM) | (30 μM) | PBS |
| Visual Analog | 2.6 | 0.9 | 0.8 | 2.0 | 1.8 | 1.5 |
| Scale (VAS) | | | | | | |
| Color Matching | 2 | 1 | 1 | 2 | 2 | 2 |
| (CM) | | | | | | |
| Matte/Shiny | 1 | 1 | 1 | 1 | 1 | 1 |
| (M/S) | | | | | | |
| Contour (C) | 2 | 1 | 1 | 2 | 2 | 1 |
| Distortion (D) | 2 | 1 | 1 | 1.5 | 1 | 2 |
| Texture (T) | 2 | 2 | 2 | 2 | 2 | 2 |

During the dressing change on days 13, 28, 41, 56 and 70, clinical evaluations of edema, granulation and erythema were performed. Edema was assessed according to a 5-point scale. (1=No edema (normal); 2=slight edema; 3=moderate edema; 4=severe edema; 5=very severe edema). Granulation was assessed according to a 4-point scale. (1=no apparent granulation tissue; 2=granulation tissue partially covering wound base; 3=granulation tissue completely covering wound base but not filling wound volume; 4=granulation tissue completely filling wound volume). Erythema was assessed according to a 5-point scale. (1=no erythema (normal); 2=slight erythema; 3=moderate erythema; 4=severe erythema; 5=very severe erythema). The animals were sacrificed on day 70 and biopsies were performed. Histopathology of scar formation was performed by numerical scoring. The pathologist assessed a number of parameters of the scars and general wound healing. Table 13 shows the pathology median scores of the test groups (MMI-0100 (300 μM); and MMI-0100 (30 μM)) and the control PBS group.

TABLE 13
Pathology median scores for the test groups (MMI-0100 (300 μM);
and MMI-0100 (30 μM)) and the control PBS group
Table 13. Pathology median scores

| | | | | Superficial | Deep |
|---|---|---|---|---|---|
| | Quality | | Epithelium | Granu- | Granu- |
| | of | Epithelium | non- | lation | lation |
| Groups | Scar | Coverage | uniformity | Tissue | Tissue |
| Control | 2 | 3 | 1 | 4 | 4 |
| MMI-0100 | 2 | 3 | 1 | 4 | 4 |
| (300 μM) | | | | | |
| MMI-0100 | 1 | 3 | 1 | 0 | 4 |
| (30 μM) | | | | | |

The results show that the 30 μM dosage of MMI-0100 accelerated wound healing on assessment days 7 and 13 compared to PBS control and the 300 μM dosage of the same material. The 30 μM dosage of MMI-0100 showed a reduction of wound size by 10% on day 7 and by 5% on day 13 compared to the PBS control. Scarring appeared to be slightly worse for the wounds treated with the 300 μM dosage on Day 56, while the 30 μM dosage was at parity with the PBS control. By Day 70, the differences between the treatment and controls were less pronounced but followed trends similar to those seen on Day 70. Pathology findings from the study did not reach statistical significance for any of the parameters measured, but trends were observed in the 30 μM dosage suggesting improvements in both total scar reduction and increased speed of tissue remodeling.

### EQUIVALENTS

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A pharmaceutical composition for use in treating a cutaneous scar in a subject in need thereof, comprising a therapeutic amount of a Mitogen-Activated Protein Kinase-Activated Protein Kinase 2 (MK2) inhibitor comprising an MK2 polypeptide inhibitor of amino acid sequence YARAAARQARAKALARQLGVAA (SEQ ID NO: 1) or a functional equivalent thereof, and a pharmaceutically acceptable carrier, wherein
the subject in need thereof has suffered a wound, and
the therapeutic amount is effective (a) to reduce incidence, severity, or both, of the cutaneous scar without impairing normal wound healing and (b) to treat the cutaneous scar in the subject, such that at least one of the wound size, scar area, and collagen whorl formation in the wound is reduced compared to the control.

2. The pharmaceutical composition according to claim 1, wherein the wound is an abrasion, a laceration, a crush, a contusion, a puncture, an avulsion, a burn, an ulcer, an incisional wound, a high-tension wound, or a combination thereof.

3. The pharmaceutical composition according to claim 1, wherein the cutaneous scar is a pathological scar, an incisional scar, or a combination thereof.

4. The pharmaceutical composition according to claim 3, wherein the pathological scar is selected from the group consisting of a hypertrophic scar, a keloid, an atrophic scar, a scar contracture, or a combination thereof.

5. The pharmaceutical composition according to claim 3, wherein the pathological scar results from a high-tension wound located in close proximity to a joint comprising a knee, an elbow, a wrist, a shoulder, a hip, a spine, or a combination thereof.

6. The pharmaceutical composition according to claim 3, wherein the pathological scar results from an abrasion, a laceration, an incision, a crush, a contusion, a puncture, an avulsion, a burn, an ulcer, an autoimmune skin disorder, or a combination thereof.

7. The pharmaceutical composition according to claim 6, wherein the autoimmune skin disorder is selected from the group consisting of systemic lupus erythematosus (SLE), systemic sclerosis (scleroderma), pemphigus, vitiligo, dermatitis herpetiformis, psoriasis, or a combination thereof.

8. The pharmaceutical composition according to claim 1, wherein the therapeutic amount is effective to inhibit at least 65% of a kinase activity of at least one kinase selected from the group consisting of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof without substantially inhibiting an off-target protein.

9. The pharmaceutical composition according to claim 1, wherein the therapeutic amount is effective to reduce either a level of transforming growth factor-β (TGF-β) expression in the wound; or number of at least one immunomodulatory cell or a progenitor cell infiltrating into the wound, or both.

10. The pharmaceutical composition according to claim 9, wherein the immunomodulatory cell is selected from the group consisting of a monocyte, a mast cell, a dendritic cell, a macrophage, a T-lymphocyte, a fibrocyte, or a combination thereof.

11. The pharmaceutical composition according to claim 9, wherein the progenitor cell is selected from the group consisting of a hematopoitic stem cell, a mesenchymal stem cell, or a combination thereof.

12. The pharmaceutical composition according to claim 1, wherein the pharmaceutical composition further comprises at least one additional therapeutic agent selected from the group consisting of an anti-inflammatory agent, an analgesic agent, an anti-infective agent, or a combination thereof.

13. The pharmaceutical composition according to claim 12, wherein the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

14. The pharmaceutical composition according to claim 12, wherein the additional therapeutic agent is selected from the group consisting of rose hip oil, vitamin E, 5-fluorouracil, bleomycin, onion extract, pentoxifylline, prolyl-4-hydroxylase, verapamil, tacrolimus, tamoxifen, tretinoin, colchicine, tranilst, zinc, an antibiotic, and a combination thereof.

15. The pharmaceutical composition according to claim 1, wherein the functional equivalent of the MK2 polypeptide inhibitor of amino acid sequence YARAAARQARAKALARQLGVAA (SEQ ID NO: 1) has at least 80 percent sequence identity to amino acid sequence YARAAARQARAKALARQLGVAA (SEQ ID NO: 1); and is a polypeptide of amino acid sequence selected from the group consisting of YARAAARQARAKALNRQLGVA (SEQ ID NO: 19), FAKLAARLYRKALARQLGVAA (SEQ ID NO: 3), KAFAKLAARLYRKALARQLGVAA (SEQ ID NO: 4), YARAAARQARAKALARQLAVA (SEQ ID NO: 5), YARAAARQARAKALARQLGVA (SEQ ID NO: 6), or HRRIKAWLKKIKALARQLGVAA (SEQ ID NO: 7).

16. The pharmaceutical composition according to claim 1, wherein
the functional equivalent of the polypeptide YARAAARQARAKALARQLGVAA (SEQ ID NO: 1) is a fusion peptide comprising a first polypeptide operatively linked to a second polypeptide,
the first polypeptide is of amino acid sequence YARAAARQARA (SEQ ID NO: 11), and
the second polypeptide comprises a therapeutic domain whose sequence has at least 70 percent sequence identity to amino acid sequence KALARQLGVAA (SEQ ID NO: 2) and is selected from the group consisting of a polypeptide of amino acid sequence KALARQLAVA (SEQ ID NO: 8), a polypeptide of amino acid sequence KALARQLGVA (SEQ ID NO: 9), a polypeptide of amino acid sequence KALARQLGVAA (SEQ ID NO: 10).

17. The pharmaceutical composition according to claim 1, wherein the functional equivalent of the polypeptide YARAAARQARAKALARQLGVAA (SEQ ID NO: 1) is a fusion peptide comprising a first polypeptide operatively linked to a second polypeptide, wherein
the first polypeptide comprises a protein transduction domain functionally equivalent to YARAAARQARA (SEQ ID NO: 11) and is a polypeptide of amino acid sequence selected from the group consisting of WLRRIKAWLRRIKA (SEQ ID NO: 12), WLRRIKA (SEQ ID NO: 13), YGRKKRRQRRR (SEQ ID NO: 14), WLRRIKAWLRRI (SEQ ID NO: 15), FAKLAARLYR (SEQ ID NO: 16), KAFAKLAARLYR (SEQ ID NO: 17), and HRRIKAWLKKI (SEQ ID NO: 18); and
the second polypeptide is of amino acid sequence KALARQLGVAA (SEQ ID NO: 2).

18. The pharmaceutical composition according to claim 1, wherein the pharmaceutically acceptable carrier is a controlled release carrier.

19. The pharmaceutical composition according to claim 1, wherein the pharmaceutically acceptable carrier comprises particles.

20. The pharmaceutical composition according to claim 1, wherein the therapeutic amount is effective to modulate an expression level of at least one scar-related gene or scar-related protein in a wound selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD).

21. The pharmaceutical composition according to claim 1, wherein the pharmaceutical composition further comprises a small molecule MK2 inhibitor, wherein the small molecule MK2 inhibitor is a pyrrolopyridone analogue or a multicyclic lactam analogue.

22. The pharmaceutical composition according to claim 1, wherein the therapeutic amount of the MK2 polypeptide inhibitor of the pharmaceutical composition is of an amount from about 0.000001 mg/kg body weight to about 100 mg/kg body weight.

23. A method for treating a cutaneous scar in a subject in need thereof, wherein the subject in need thereof has suffered a wound, comprising
administering to the subject a pharmaceutical composition comprising a therapeutic amount of a Mitogen-Activated Protein Kinase-Activated Protein Kinase 2 (MK2) inhibitor comprising an MK2 polypeptide inhibitor of amino acid sequence YARAAARQARAKALARQLGVAA (SEQ ID NO: 1) or a functional equivalent thereof, and a pharmaceutically acceptable carrier, wherein
the therapeutic amount is effective (a) to reduce incidence, severity, or both, of the cutaneous scar without impairing normal wound healing and (b) to treat the cutaneous scar in the subject, such that at least one of the wound size, scar area, and collagen whorl formation in the wound is reduced compared to the control.

24. The method according to claim 23, wherein the wound is an abrasion, a laceration, a crush, a contusion, a puncture, an avulsion, a burn, an ulcer, an incisional wound, a high-tension wound, or a combination thereof.

25. The method according to claim 23, wherein the cutaneous scar is a pathological scar, an incisional scar, or a combination thereof.

26. The method according to claim 25, wherein the pathological scar is selected from the group consisting of a hypertrophic scar, a keloid, an atrophic scar, a scar contracture, or a combination thereof.

27. The method according to claim 25, wherein the pathological scar results from a high-tension wound located in close proximity to a joint comprising a knee, an elbow, a wrist, a shoulder, a hip, a spine, or a combination thereof.

28. The method according to claim 25, wherein the pathological scar results from an abrasion, a laceration, an incision, a crush, a contusion, a puncture, an avulsion, a burn, an ulcer, an autoimmune skin disorder, or a combination thereof.

29. The method according to claim 28, wherein the autoimmune skin disorder is selected from the group consisting of systemic lupus erythematosus (SLE), systemic sclerosis (scleroderma), pemphigus, vitiligo, dermatitis herpetiformis, psoriasis, or a combination thereof.

30. The method according to claim 23, wherein the administering is topically.

31. The method according to claim 30, wherein the administering is by means of a dressing comprising the pharmaceutical composition.

32. The method according to claim 31, wherein at least one surface of the dressing is impregnated with the pharmaceutical composition.

33. The method according to claim 31, wherein the dressing is selected from the group consisting of a gauze dressing, a tulle dressing, an alginate dressing, a polyurethane dressing, a silicone foam dressing, a synthetic polymer scaffold dressing, or a combination thereof.

34. The method according to claim 31, wherein the dressing is an occlusive dressing selected from the group consisting of a film dressing, a semi-permeable film dressing, a hydrogel dressing, a hydrocolloid dressing, and a combination thereof.

35. The method according to claim 30, wherein the administering is by means of a dermal substitute, wherein the pharmaceutical composition is embedded in a dermal substitute that provides a three dimensional scaffold.

36. The method according to claim 35, wherein the dermal substitute is made of a natural biological material, a constructive biological material, or a synthetic material.

37. The method according to claim 36, wherein the natural biological material comprises human cadaver skin, porcine cadaver skin, or porcine small intestine submucosa.

38. The method according to claim 36, wherein the natural biological material comprises a matrix.

39. The method according to claim 36, wherein the natural biological material consists essentially of a matrix that is sufficiently devoid of cell remnants.

40. The method according to claim 36, wherein the constructive biological material comprises collagen, glycosaminoglycan, fibronectin, hyaluonic acid, elastine, or a combination thereof.

41. The method according to claim 36, wherein the constructive biological material is a bilayer, non-cellularized dermal regeneration template or a single layer, cellularized dermal regeneration template.

42. The method according to claim 36, wherein the synthetic dermal substitute comprises a hydrogel.

43. The method according to claim 36, wherein the synthetic dermal substitute further comprises an RGD peptide with amino acid sequence Arginine-Glycine-Aspartate.

44. The method according to claim 23, wherein the administering is intraperitoneally, intravenously, intradermally, intramuscularly, or a combination thereof.

45. The method according to claim 44, wherein the administering is via an injection device, wherein the injection device is soaked with the pharmaceutical composition prior to administration.

46. The method according to claim 45, wherein the injection device is selected from the group consisting of a needle, a cannula, a catheter, a suture, or a combination thereof.

47. The method according to claim 44, wherein the therapeutic amount of the MK2 polypeptide inhibitor of the amino acid sequence YARAAARQARAKALARQLGVAA (SEQ ID NO: 1) or a functional equivalent thereof for intradermal injection ranges from 50 ng/100 μl/linear centimeter of wound margin to 500 ng/100 μl/linear centimeter of wound margin.

48. The method according to claim 44, wherein the therapeutic amount of the MK2 polypeptide inhibitor of the amino acid sequence YARAAARQARAKALARQLGVAA (SEQ ID NO: 1) or a functional equivalent thereof for intraperitoneal administration ranges from 70 μg/kg to 80 μg/kg.

49. The method according to claim 23, wherein the administering is in a single dose at one time.

50. The method according to claim 23, wherein the administering is in a plurality of doses for a period of at least one day, at least one week, at least one month, at least one year, or a combination thereof.

51. The method according to claim 50, wherein the administering is at least once daily, at least once weekly, or at least once monthly.

52. The method according to claim 23, wherein the pharmaceutical composition further comprises at least one additional therapeutic agent selected from the group consisting of an anti-inflammatory agent, an analgesic agent, an anti-infective agent, or a combination thereof.

53. The method according to claim 52, wherein the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

54. The method according to claim 52, wherein the additional therapeutic agent is selected from the group consisting of rose hip oil, vitamin E, 5-fluorouracil, bleomycin, onion extract, pentoxifylline, prolyl-4-hydroxylase, verapamil, tacrolimus, tamoxifen, tretinoin, colchicine, tranilst, zinc, an antibiotic, and a combination thereof.

55. The method according to claim 23, wherein the administering is before, during, or after closing of the wound.

56. The method according to claim 55, wherein the closing of the wound is by means of at least one subcutaneous suture, at least one staple, at least one adhesive tape, a surgical adhesive, or a combination thereof.

36. The method according to claim 56, wherein the surgical adhesive comprises octyl-2-cyanoacrylate or fibrin tissue adhesive.

37. The method according to claim 23, wherein the functional equivalent of the MK2 polypeptide inhibitor of amino acid sequence YARAAARQARAKALARQLGVAA (SEQ ID NO: 1) has at least 80 percent sequence identity to amino acid sequence YARAAARQARAKALARQLGVAA (SEQ ID NO: 1); and is a polypeptide of amino acid sequence selected from the group consisting of YARAAARQARAKALNRQLGVA (SEQ ID NO: 19), FAKLAARLYRKALARQLGVAA (SEQ ID NO: 3), KAFAKLAARLYRKALARQLGVAA (SEQ ID NO: 4), YARAAARQARAKALARQLAVA (SEQ ID NO: 5), YARAAARQARAKALARQLGVA (SEQ ID NO: 6), or HRRIKAWLKKIKALARQLGVAA (SEQ ID NO: 7).

38. The method according to claim 23, wherein
the functional equivalent of the polypeptide YARAAARQARAKALARQLGVAA (SEQ ID NO: 1) is a fusion peptide comprising a first polypeptide operatively linked to a second polypeptide,
the first polypeptide is of amino acid sequence YARAAARQARA (SEQ ID NO: 11), and
the second polypeptide comprises a therapeutic domain whose sequence has at least 70 percent sequence identity to amino acid sequence KALARQLGVAA (SEQ ID NO: 2) and is selected from the group consisting of a polypeptide of amino acid sequence KALARQLAVA (SEQ ID NO: 8), a polypeptide of amino acid sequence KALARQLGVA (SEQ ID NO: 9), a polypeptide of amino acid sequence KALARQLGVAA (SEQ ID NO: 10).

39. The method according to claim 23, wherein
the functional equivalent of the polypeptide YARAAARQARAKALARQLGVAA (SEQ ID NO: 1) is a fusion peptide comprising a first polypeptide operatively linked to a second polypeptide, wherein the first polypeptide comprises a protein transduction domain functionally equivalent to YARAAARQARA (SEQ ID NO: 11) and is a polypeptide of amino acid sequence selected from the group consisting of WLRRIKAWLRRIKA (SEQ ID NO: 12), WLRRIKA (SEQ ID NO: 13), YGRKKRRQRRR (SEQ ID NO: 14), WLRRIKAWLRRI (SEQ ID NO: 15), FAKLAARLYR (SEQ ID NO: 16), KAFAKLAARLYR (SEQ ID NO: 17), and HRRIKAWLKKI (SEQ ID NO: 18); and
the second polypeptide is of amino acid sequence KALARQLGVAA (SEQ ID NO: 2).

40. The method according to claim 23, wherein the therapeutic amount is effective to inhibit at least 65% of a kinase activity of at least one kinase selected from the group consisting of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof without substantially inhibiting an off-target protein.

41. The method according to claim 23, wherein the therapeutic amount is effective to modulate an expression level of at least one scar-related gene or scar-related protein in a wound selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD).

42. The method according to claim 23, wherein the therapeutic amount is effective to reduce either a level of transforming growth factor-β (TGF-β) expression in the wound; or number of at least one immunomodulatory cell or a progenitor cell infiltrating into the wound, or both.

43. The method according to claim 42, wherein the immunomodulatory cell is selected from the group consisting of a monocyte, a mast cell, a dendritic cell, a macrophage, a T-lymphocyte, a fibrocyte, or a combination thereof.

44. The method according to claim 23, wherein the progenitor cell is selected from the group consisting of a hematopoitic stem cell, a mesenchymal stem cell, or a combination thereof.

45. The method according to claim 23, wherein the pharmaceutical composition further comprises a small molecule MK2 inhibitor, wherein the small molecule MK2 inhibitor is a pyrrolopyridone analogue or a multicyclic lactam analogue.

46. A dressing for use in treating a cutaneous scar in a subject in need thereof, wherein
the subject in need thereof has suffered a wound,
the dressing comprises a pharmaceutical composition comprising a therapeutic amount of a Mitogen-Activated Protein Kinase-Activated Protein Kinase 2 (MK2) inhibitor comprising an MK2 polypeptide inhibitor of the amino acid sequence YARAAARQARAKALARQLGVAA (SEQ ID NO: 1) or a functional equivalent thereof, and a pharmaceutically acceptable carrier,
and the therapeutic amount is effective (a) to reduce incidence, severity, or both, of the cutaneous scar without impairing normal wound healing and (b) to treat the cutaneous scar in the subject, such that at least one of the wound size, scar area, and collagen whorl formation in the wound is reduced compared to the control.

47. The dressing according to claim 46, wherein the dressing is selected from the group consisting of a gauze dressing, a tulle dressing, an alginate dressing, a polyurethane dressing, a silicone foam dressing, a collagen dressing, a synthetic polymer scaffold, peptide-soaked sutures or a combination thereof.

48. The dressing according to claim 46, wherein the dressing is an occlusive dressing selected from the group consisting of a film dressing, a semi-permeable film dressing, a hydrogel dressing, a hydrocolloid dressing, and a combination thereof.

49. The dressing according to claim 46, wherein the dressing further comprises a dermal substitute embedded in or on a surface of the dressing with the pharmaceutical composition, and wherein the dermal substitute provides a three-dimensional extracellular scaffold.

50. The dressing according to claim 49, wherein the dermal substitute is made of a natural biological material, a constructive biological material, or a synthetic material.

51. The dressing according to claim 50, wherein the natural biological material comprises human cadaver skin, porcine cadaver skin, or porcine small intestine submucosa.

52. The dressing according to claim 50, wherein the natural biological material comprises a matrix.

53. The dressing according to claim 50, wherein the natural biological material consists essentially of a matrix that is sufficiently devoid of cell remnants.

54. The dressing according to claim 50, wherein the constructive biological material comprises collagen, glycosaminoglycan, fibronectin, hyaluonic acid, elastine, or a combination thereof.

55. The dressing according to claim 50, wherein the constructive biological material is a bilayer, non-cellularized dermal regeneration template or a single layer, cellularized dermal regeneration template.

56. The dressing according to claim 50, wherein the synthetic dermal substitute comprises a hydro gel.

57. The dressing according to claim 50, wherein the synthetic dermal substitute further comprises an RGD peptide with amino acid sequence Arginine-Glycine-Aspartate.

58. The dressing according to claim 46, wherein the wound is an abrasion, a laceration, a crush, a contusion, a puncture, an avulsion, a burn, an ulcer, an incisional wound, a high-tension wound, or a combination thereof.

59. The dressing according to claim 46, wherein the cutaneous scar is a pathological scar, an incisional scar, or a combination thereof.

60. The dressing according to claim 59, wherein the pathological scar is selected from the group consisting of a hypertrophic scar, a keloid, an atrophic scar, a scar contracture, or a combination thereof.

61. The dressing according to claim 59, wherein the pathological scar results from a high-tension wound located in close proximity to a joint comprising a knee, an elbow, a wrist, a shoulder, a hip, a spine, or a combination thereof.

62. The dressing according to claim 59, wherein the pathological scar results from an abrasion, a laceration, an incision, a crush, a contusion, a puncture, an avulsion, a burn, an ulcer, an autoimmune skin disorder, or a combination thereof.

63. The dressing according to claim 62, wherein the autoimmune skin disorder is selected from the group consisting of systemic lupus erythematosus (SLE), systemic sclerosis (scleroderma), pemphigus, vitiligo, dermatitis herpetiformis, psoriasis, or a combination thereof.

64. The dressing according to claim 46, wherein the dressing is a mechano-active dressing further comprising an anti-infective agent, a growth factor, a vitamin, a clotting agent, or a combination thereof.

65. The dressing according to claim 46, wherein the pharmaceutical composition further comprises at least one additional therapeutic agent selected from the group consisting of an anti-inflammatory agent, an analgesic agent, an anti-infective agent, or a combination thereof.

66. The dressing according to claim 65, wherein the additional therapeutic agent comprises EXC001 (an anti-sense RNA against connective tissue growth factor (CTGF)), AZX100 (a phosphopeptide analog of Heat Shock Protein 20 (HSP20)), PRM-151 (recombinant human serum amyloid P/Pentaxin 2), PXL01 (a synthetic peptide derived from human lactoferrin), DSC127 (an angiotensin analog), RXI-109 (a self-delivering RNAi compound that targets connective tissue growth factor (CTGF)), TCA (trichloroacetic acid), Botulium toxin type A, or a combination thereof.

67. The dressing according to claim 65, wherein the additional therapeutic agent is selected from the group consisting of rose hip oil, vitamin E, 5-fluorouracil, bleomycin, onion extract, pentoxifylline, prolyl-4-hydroxylase, verapamil, tacrolimus, tamoxifen, tretinoin, colchicine, tranilst, zinc, an antibiotic, and a combination thereof.

68. The dressing according to claim 46, wherein the functional equivalent of the MK2 polypeptide inhibitor of amino acid sequence YARAAARQARAKALARQLGVAA (SEQ ID NO: 1) has at least 80 percent sequence identity to amino acid sequence YARAAARQARAKALARQLGVAA (SEQ ID NO: 1); and is a polypeptide of amino acid sequence selected from the group consisting of YARAAARQARAKALNRQLGVA (SEQ ID NO: 19), FAKLAARLYRKALARQLGVAA (SEQ ID NO: 3), KAFAKLAARLYRKALARQLGVAA (SEQ ID NO: 4), YARAAARQARAKALARQLAVA (SEQ ID NO: 5), YARAAARQARAKALARQLGVA (SEQ ID NO: 6), or HRRIKAWLKKIKALARQLGVAA (SEQ ID NO: 7).

69. The dressing according to claim 46, wherein
the functional equivalent of the polypeptide YARAAARQARAKALARQLGVAA (SEQ ID NO: 1) is a fusion peptide comprising a first polypeptide operatively linked to a second polypeptide,
the first polypeptide is of amino acid sequence YARAAARQARA (SEQ ID NO: 11), and
the second polypeptide comprises a therapeutic domain whose sequence has at least 70 percent sequence identity to amino acid sequence KALARQLGVAA (SEQ ID NO: 2) and is selected from the group consisting of a polypeptide of amino acid sequence KALARQLAVA (SEQ ID NO: 8), a polypeptide of amino acid sequence KALARQLGVA (SEQ ID NO: 9), a polypeptide of amino acid sequence KALARQLGVAA (SEQ ID NO: 10).

70. The dressing according to claim 46, wherein the functional equivalent of the polypeptide YARAAARQARAKALARQLGVAA (SEQ ID NO: 1) is a fusion peptide comprising a first polypeptide operatively linked to a second polypeptide, wherein
the first polypeptide comprises a protein transduction domain functionally equivalent to YARAAARQARA (SEQ ID NO: 11) and is a polypeptide of amino acid sequence selected from the group consisting of WLRRIKAWLRRIKA (SEQ ID NO: 12), WLRRIKA (SEQ ID NO: 13), YGRKKRRQRRR (SEQ ID NO: 14), WLRRIKAWLRRI (SEQ ID NO: 15), FAKLAARLYR (SEQ ID NO: 16), KAFAKLAARLYR (SEQ ID NO: 17), and HRRIKAWLKKI (SEQ ID NO: 18); and
the second polypeptide is of amino acid sequence KALARQLGVAA (SEQ ID NO: 2).

71. The dressing according to claim 46, wherein the pharmaceutically acceptable carrier is a controlled release carrier.

72. The dressing according to claim 46, wherein the pharmaceutically acceptable carrier comprises particles.

73. The dressing according to claim 46, wherein the therapeutic amount is effective to inhibit at least 65% of a kinase activity of at least one kinase selected from the group consisting of Mitogen-Activated Protein Kinase-Activated Protein Kinase 2 (MK2), Mitogen-Activated Protein Kinase-Activated Protein Kinase 3 (MK3), calcium/calmodulin-dependent protein kinase I (CaMKI), BDNF/NT-3 growth factors receptor (TrkB), or a combination thereof without substantially inhibiting an off-target protein.

74. The dressing according to claim 46, wherein the therapeutic amount is effective to modulate an expression level of at least one scar-related gene or scar-related protein in a wound selected from the group consisting of Transforming Growth Factor-β1 (TGF-β1), Tumor Necrosis Factor-α (TNF-α), a collagen, Interleukin-6 (IL-6), chemokine (C-C motif) ligand 2 (CCL2) (or monocyte chemotactic protein-1 (MCP-1)), chemokine (C-C motif) receptor 2 (CCR2), EGF-like module-containing mucin-like hormone receptor-like 1 (EMR1), or a sma/mad-related protein (SMAD).

75. The dressing according to claim 46, wherein the therapeutic amount is effective to reduce either a level of transforming growth factor-β (TGF-β) expression in the wound; or number of at least one immunomodulatory cell or a progenitor cell infiltrating into the wound, or both.

76. The dressing according to claim 75, wherein the immunomodulatory cell is selected from the group consisting of a monocyte, a mast cell, a dendritic cell, a macrophage, a T-lymphocyte, a fibrocyte, or a combination thereof.

77. The dressing according to claim 75, wherein the progenitor cell is selected from the group consisting of a hematopoitic stem cell, a mesenchymal stem cell, or a combination thereof.

78. The dressing according to claim 46, wherein the pharmaceutical composition further comprises a small molecule MK2 inhibitor, wherein the small molecule MK2 inhibitor is a pyrrolopyridone analogue or a multicyclic lactam analogue.


SEQUENCE LISTING (22 sequences)

SEQ ID NO: 1 - protein, 22 aa, Unknown
  mammal
YARAAARQARAKALARQLGVAA

SEQ ID NO: 2 - protein, 11 aa, Unknown
  mammal
KALARQLGVAA

SEQ ID NO: 3 - protein, 21 aa, Unknown
  mammal
FAKLAARLYRKALARQLGVAA

SEQ ID NO: 4 - protein, 23 aa, Unknown
  mammal
KAFAKLAARLYRKALARQLGVAA

SEQ ID NO: 5 - protein, 21 aa, Unknown
  mammal
YARAAARQARAKALARQLAVA

SEQ ID NO: 6 - protein, 21 aa, Unknown
  mammal
YARAAARQARAKALARQLGVA

SEQ ID NO: 7 - protein, 22 aa, Unknown
  mammal
HRRIKAWLKKIKALARQLGVAA

SEQ ID NO: 8 - protein, 10 aa, Unknown
  mammal
KALARQLAVA

SEQ ID NO: 9 - protein, 10 aa, Artificial
  mammal
KALARQLGVA

SEQ ID NO: 10 - protein, 11 aa, Unknown
  mammal
KALARQLGVAA

SEQ ID NO: 11 - protein, 11 aa, Unknown
  mammal
YARAAARQARA

SEQ ID NO: 12 - protein, 14 aa, Unknown
  mammal
WLRRIKAWLRRIKA

SEQ ID NO: 13 - protein, 7 aa, Unknown
  mammal
WLRRIKA

SEQ ID NO: 14 - protein, 11 aa, Unknown
  mammal
YGRKKRRQRRR

SEQ ID NO: 15 - protein, 12 aa, Unknown
  mammal
WLRRIKAWLRRI

SEQ ID NO: 16 - protein, 10 aa, Unknown
  mammal
FAKLAARLYR

SEQ ID NO: 17 - protein, 12 aa, Unknown
  mammal
KAFAKLAARLYR

SEQ ID NO: 18 - protein, 11 aa, Unknown
  mammal
HRRIKAWLKKI

SEQ ID NO: 19 - protein, 21 aa, Unknown
  mammal
YARAAARQARAKALNRQLGVA

SEQ ID NO: 20 - protein, 9 aa, Unknown
  mammal
RKKRRQRRR

SEQ ID NO: 21 - protein, 16 aa, Unknown
  mammal
KKXXXXXXXXXXKRRK

SEQ ID NO: 22 - protein, 7 aa, Unknown
  mammal
LLKRRKK